SEQUENCE LISTING (200 sequences)

SEQ ID NO: 1 - DNA, 838 bp, Zea mays
  misc_feature (158)..(158): n is a, c, g, or t
gaatgtggcaaactacatgggtcaaatggccatggctatgggaaaacttggaacgcttgagaatttccttcgtcaggtattaaccctcttttccaaatgcatctttgtgacatagataaagggttctgtgaatgattgtactccctcccaaaaagaangctcatcttgcatttcagggatagcttttttaagtttggccagacagaaaaaagtactaannnnnnnntatttatttatgaatgcgtagtaagtatcgttaagttaatcatgaaatatatattgataataaactcatttgtaccttcaattgttggtactattctttataaacccaatagcacttaaaaagatttgattatttaatatgagggcattagacttgaatatatatagcgatatacatgatagagaccgtgtatagaacaataactttatatatgagatgaaataaaacaccgacatatgcagagacatctagcaatgacacttatgtatgaaactgaactgaatacgacaaggacaacctgcggtgcctaaatcctgctacttctctttgttgtatgtatcaaggctgacaacctgcggctgcagactctacaacagatgcaaaggatcctgaccactagacagtcagctcgggcgctacttgtgataagtgattactcttcacgtctccgtgccctaagctctctctggcttgctcggcccaaggaatagcaagaacatgctatttgactgcaatacttttcacaatttggttttcaatggtcaggagatttgacaaggctgcgacagacttgattcggacatatctgcacatactatgacccaaatgaag

SEQ ID NO: 2 - DNA, 545 bp, Zea mays
  misc_feature (511)..(514): n is a, c, g, or t
agaagatcaacggggagatgttaagagacggaaaccacacgctaaccgcaatgtggatgggggacacacggcgaaagctttgcaaacctgtcaggcgcttcctctttgttgataagtgaaatgttttttgaggagagaagttgaaagaacttggcagcatcaccttccttcaatctctcgtaaatcttcgttaggaactggttgaattcctgtggttcctgatcacggatggcgatgcccagtgtaaacagagaggcaatagctaatttgctacgacaactgaccagttttatggagaccgagcaagcatgctaatgaactaatagttagcggcccctctaaccaatagttggcaggtctattagcaggcatgttttgatccatacctattaatttaagctgctagctattttagcactactcatgtaactatcatgagcttttcgagaaaaaaaatgtaactatcatgagtgcttacctgttcaataatacaggccttccgcagagaagnnnnacannnnnnnnnnnnnnnnnnnnnnnnncca

SEQ ID NO: 3 - DNA, 549 bp, Zea mays
  misc_feature (2)..(21): n is a, c, g, or t
gnnnnnnnnnnnnnnnnnnnncaccaccgcgcgctctgacgccagcttcgccacgcggtccattgccagcttttccggtggctatatattactgtntgtccgaaactagctagcactcacagcctctttgggtgtgtgaggtacgtagatgacttggaacttggaaggtgaatgatgtgtgtggttgctgctcgaaatgttgagcggcttcttgacttttataggctggaaaagactcgcacgtactaaaacgtaaacttagtgtggattattgagatttccttggcttattcacttcttgcacagttgcacggtgcctacttgcgctgtggaataacattctttaatttagactttttctctttgtaggtatgatgattccgggacttgctaagcacatgttaaaaagcacccctccccctcccttctcctttgcaagacgcacacgtatattatattacgaattaaacttcaaattaaaatctttgactttacctnataatttnnnnnattnnnnnnnnnnnnnnnnnnnnnnnnnnnnntnngtna

SEQ ID NO: 4 - DNA, 679 bp, Zea mays
  misc_feature (47)..(47): n is a, c, g, or t
ggaacacgaacaaacacatatatgaaaaggtgagatgtcgcatcagngttgttccttgtcttgctatgctgcacatgagcacattggcgcttccaatatcttgaatgacagggcattctcggatcatgcagttgcatctattctagtgctgttcttctgtcttttcccagttatattctagtntggacaattgctgaaagttcccagntgctgcttaagcttaatctaatgaattcttgtttttatcaaagtttggagtttagatagtgtagcatttattttcatactgatggtgtattgtccttatcatttcttttnaatcttcagccaaacctaangcaacaagaaatctgatattattaagaataagcagcctctatcctgaaagagngggggnaaaaggaagggtgacantatcatctgtgatcgtgtttgttgcaaatcttcttcatatgcacctttataatctaaatatactgcgctcctagcgactttgttcattgcaaggctttgtgtgaaatcattgctccaatcatgaatctaggaaatatactttaagctctaaaataagtgcatttatagaatgttttggagacatcaatgctactatanaattacatatatacactcctanngtnnnnnnntntnntaanngcactnnnnnnnnnnannnnnncaa

SEQ ID NO: 5 - DNA, 593 bp, Zea mays
  misc_feature (100)..(100): n is a, c, g, or t
gtaacagaagtcactgagctggaagaatcgcctgggctctcagcctcagccttggatgaggaacccaacgcttgtgttcaagcagtgcttctcagggagncaaacncactaggacacaatcaaagaactgtagttcctagatcacagcatgcatctaatgtactggctggaaatgagttggtgattgttggacctcgatatgctggttctgtgccacccattgcaacaagaggtctggaaaacagcaaggagagcggtggaaggggcttcaaacccaaaccttgtaacaacattttccaaaatggctcttcaaagcctgaaagggaaccgggcaattcgtcaaacaaaagaacagctggtaaaacggnnnaggaccttggtcacaaggatagttcanctgaagtgtcctacgagtactgtgtaagggtggtcaggtggctggagtgcgagggctacatcgagacaaacttcanngtgaagtttctcacatgnnnnngcctgcgtgccaccccgnnnnngagaaagatagtcggtgtctatgtggatacccttattgaggaccctgtnnnncttnnnnnnnnnnnnnnngacag

SEQ ID NO: 6 - DNA, 593 bp, Zea mays; identical to SEQ ID NO: 5
  misc_feature (100)..(100): n is a, c, g, or t

SEQ ID NO: 7 - DNA, 658 bp, Zea mays
  misc_feature (90)..(90): n is a, c, g, or t
atcactacgcaaccagaagcaggatgcgctgtgcacgaactacacttcatggaccaatattacttacaaagttacaagtcgatgaaaagnaccacctgggagaaaagaggttatagcttttgaatcacatcannttgtcntcaaagaaatccgataaaaaaaacatggtcnccataacngtaantggtcaatagttgcaactaactcatgtttgaagcaactgacctagcctttctactcctaaaaacatatatctagcatagaattttctcaacgacagcctgtagactgcctagaacaaagagcatcataagaaaacatgaagcttcaggcatagcaaatacttatcatacaataatcagcagagattaatgcaacatacacattcccgaagctgttcttcgcatatgaaccaccatggtgaccaacatacatgaagagggaaccagaatgcttgagtctaacatgagggaagtgatcatcataatcactgattacctcacacaaataaacacccattttttattttaaagaaaaaccaatatgtgatacataccccaaagtttgcaagcacatgggaatgggacaccatcnnnatcattctgntnnnnnnntgtcatcnnnnnnntnnnnnnnnnnnnnnnntnnnncntttttt

SEQ ID NO: 8 - DNA, 460 bp, Zea mays
  misc_feature (40)..(42): n is a, c, g, or t
gcagtgtcctcgggtacccacttaccttgcgcatcctggnnnattctgcttctgttcgaactctgcaatctgtctaacgtgtggcctctctgtttgatccgtttgcagagtgattccgagagcactcatgccgcttgaggtgtgtattaaaattattaactgtccttttcttctgttctcttctttgtgtgtaccattggtaattctactagttattactctgctctagcggctaactggttcacatttcacaagagagaattataatatctagctaactgcgaaacaactctcagctaaaaactagaatctggctcgcctttggttttgcacctgcttacagctgttccatttgaaatctatccatgaattctgccctggatgttggtttcacatgatcttgctctagtatgtagcagctcacgtagtcctatgtcagtagtgctctccaggtcaccat

SEQ ID NO: 9 - DNA, 423 bp, Zea mays
  misc_feature (25)..(25): n is a, c, g, or t
ttcttcctatatatcagcaggtatntatttgtgaattttcatttataatggtgtacgcatatttaaatttcgagtgtaaggaagtgcagcacaaggttaattttcaggcatgacgacatctgacacatttctattgagctccagggtttaattagtgggtcggccgagacaaaagaacaggcagctgaaggtctaggagagttaattgacgtaacaagtgagaagactcttaaggaagttgtagtgccaattacagggtatgtgttttatccttttgaaattgcttcagttttgtgattgcccttttgaatggttcacatatgaggatttgtgctgtttgaggaaagcccaacatcattgcaaccatgaggcatcccaccacatgtccagctgacagccgaacagctccttaagatctcaacc

SEQ ID NO: 10 - DNA, 707 bp, Zea mays
cgacctgcaggtgtgactacctgcaaccatttgacatcagaagtaggaaatacatggcaaataaaaatgaatatgactcaaccagcatgttattaccttaacatctgaaaattctatatggtttgcttcactgacataatttccaggagaactgtgatccagtgatctatcacgaaccccagacagctcacgtgaaacatccaataactcacgaatccggtctgcatagccactgtgaaattacattgaaagtccatacgcatcacaatcatgacggtgcaaagtttaaaactcaaaagcagaagagcataaaatgcatctggaacacaaaaggagcacaggactccaccaaagtattccaaagaagttacaatgttttttgtatgtaagtcaacattctccagctgtgctatgactaaagtatggttcctaccaaaacgatcatagatcaagtattgttcctaccaacgcaatcataaatcaagtatggttcctgccaaaacagtcataaatctgtggcggacatcagtgcaaaactttaatgacactgaaattctcgtatactggaatgtagaaaagccacctacacagcatgcaacaggtcaaattatataaaaaataatgtcataattgggcagaatttgaaaactaaaagggcatattgggagaggatgttaatgatttccagtctccgtcaaagcactaaa

SEQ ID NO: 11 - DNA, 666 bp, Zea mays
  misc_feature (165)..(165): n is a, c, g, or t
caaagtgaactttacttatcaaagtagcggttcaaattgtgccactgaggatccttgcaacaatttccaaagcgaacaacttctgtagaaaacactttcaactcttccctgttggaccaaacaaaaacttcagtaaccaaaaaaggaagacccagcgttggaggnttccacatgagccnggtntgggaaaggaataaatcgaggcaagccttcccccacaaatgtggagaggctgcttcgaacccgtaacctgatgacttagtgagacagctctcaccattgcatcaggtctgcccttctatnaaaaacttcagtaactacaagcatataaaaacaagttggagtacanttttttctatataaccagnttgaaacgacttaaanaaaataaataaatacatagagcatcagagattatttgcgaaccttttgtcagcagcaacaatcttgagtaattcatccatatctttggatataagattctgaacaccttcagagtgcagaaccgtttcttttaagtactttatgctatcttttgannnnncatgcattagnnngcaacctttgactangntattggcnacctcaaatgctnnaatatatatnntnnnnnnnnnnnnnnnnnnnnntgannnnnnnctacttatannnnnnnctgtcatgcta

SEQ ID NO: 12 - DNA, 691 bp, Zea mays
  misc_feature (106)..(106): n is a, c, g, or t
aggagagaccggccatggcgaaaagacggagcagaatgctgaggtgacaactgatgtgacgaccttttgtgcaatggggaccgacagactcgtcgtcgtcgtcgtngctgcctcgccgtctctgcttctctctcctctgtctaggccccacgaggctgatatctgcgccgcatgcgggacccccgagccttatcagtgtctccataccccatgttgcactgcaccttgtatcacctgtcacctctctccctctccctcctttcttctcagcttgctggccatgagaccgctctcctcaacccaccatatttatctgcctcctctcgccacatgctctcctcctctgccggctcagtcgtgggacgagtnccgatcccgacccctagcttccgctcccgatccgtcgtctcatcctctcctacnnnctggtacgctctctcgccatgcgntgcgcctccactgnccaaatccgtcgaccgtctttgtgnncttgcaatcttgcatgcaagtttctaaaagctttcngtgatctcgctcttcttgcttcctcctcttgcatatcgtacactgggtctgcatgcatnnntgtcaccaccatattctatgcccagcnnacnncnnncactgtntncnnntctactcctgcgcatnnnnnntacnnnnncttcnntcgntngatcgatcnngcntt

SEQ ID NO: 13 - DNA, 755 bp, Zea mays
  misc_feature (1)..(5): n is a, c, g, or t
nnnnnttggtgcaatgccttttggttagtggtaacaaagcctcctatcatggcntatagtacttcatgttctcttgtaaagcttcttcatggacctatactctaatgaaccaacttagctaatagtttgttttggcaggcacacctcaagatctcaattatgccaatgttacttcgattagaactactggattttctggtgaccctcttccaacagaccagaaacaacctgcctggaagccctacctgtacaaagggaggcaaaggattctgttttctagcttggagactataaatgctgcactgtatgaccgtgatgatgttccagatttcctttctgtttcaggacaggtgacctgtagggctgaactagaaggactacctgatatttctttgccattgactggtttaaaaactgctcatattgaggtttcatcattccatcactgtgttcaagcttcagagcccactgctaataagcaaaccctnntttttcagccaccattaggaaattttgtattgatgcattatcaagcaccatgcaacattgctcctcctgttaaanngttctatcagttgtccatggtttctgaaaatnnnnnnncttttctanntaagctgacattgatnnanngnnacaagtnnnnnnncattatggacttnnnnatgnnnnnnntnnnannnnnnnnnnnnnnnnnngcatcatatgacnnnnnnnnnncaattngnnnnnnnncnntnnnnnnnnnnnnnnnt

SEQ ID NO: 14 - DNA, 582 bp, Zea mays
  misc_feature (1)..(5): n is a, c, g, or t
nnnnncccaagccaaccatccaacaagcagtagataaacaagaagcagaaacagatgcgtctcctgcaccttctaaaggagagctttcaaggcggctgagtgtgaaggacaggataaacatgtttgagagccagaaaaaggagcagactccaagttctggtaacagcaactctgccggtactggtagagtggttccagggaaaggtgagcaccgcagggttccttctggcgcctccatggagaagttagttagaaggtggagcagtgttagcgacatgagtattgacctcagcaacaatgagagtagtagcttaaatgacaaaaaggataatggaactcctgccgggactccaacatctactgatttggaggccgactctaaagtaagagctaatgaggactctaatggacagaaggattcaatcacgtcacactcttggccttgtcaaaaggataatgtaccaatggatctggccaccacagatttgtgcccaccctcaattttaagtaatacacttgctcctcataaagagagtatatatggtgatggtgcagnaaacgacannnnnnnnnnnnntnnna

SEQ ID NO: 15 - DNA, 1005 bp, Zea mays
  misc_feature (398)..(398): n is a, c, g, or t
aggagactcttcattttcctcctcttctatagcacctacagcagttggttcagccttcttttttttctttgggaaagggtcaaaattcttgtgccggagctccaaaaatagatctgcccttttcctgttgttaactataatttcattatcaacaacacagcggataaacctaactttgttttcaagcttctttaagtccagcttgatattcttcaacaatgcttcctgcacaaataaattggttaatatggctttcagttaatattctaaaaatgacaaagttcatcacatgtacctttctcttacaatagaattcaagccttaattgaaagaattcctcaagtactgcaaaaatatcacactcagttattaaataacaagaaaacaggggtctgtancaacncntaatacgatacatnacttactttgctcnggagtgtcatanttccgaattttaccatctgaatcaaacaagtgcatntttgntgtcccaattgttgttgtcagcttaaacttcttcactagtccttcttccatagctanattcatatttntctcacttaatntgaactgaatgtaaacatcttcattatcaccttgacatgtgacatcctgtaaaatagttgcaattgaggaatagcatctatgacggtggaaaatttatgaacacaattgtaagcgtttttgttttcaacgacctctatgaatggtactttgtccttgttcttagtatctggagccaaagtctcaagataatctttgtaatcctgagtccaacggcggattggcaattcagtgatcctcagtgtagtgttgtcaagaacctcgatgattccagtgacagtatatgtggaaccagctacctttgtattgactgtcttctctatagagccctgtaaatttgggcanannnnnnntncnntnnnanancnnnnnntttatatagtactcnnnnnnnncntttttatntnnnnnnnnnnnnnnnnnnnnnnnnnnnnnnnncag

SEQ ID NO: 16 - DNA, 1437 bp, Zea mays
  misc_feature (303)..(303): n is a, c, g, or t
gttagcagcccaaaagcaactgccagcttctcactatgttcagccagtgcacgttccttagattgctcatcaatgtcttgcaacactaaacctgtcattggcttatacccagcttgccttaagcgatccatcatctctcccaacattttgtagatctccttggaccttggatgcgatctatcaccagaaataaactcgtgaattgtattctgccattcaatcgagcttcttccaggaattctctggattctctccctcctaagcgtcctcctcatctctgcaacaccttcccaggaattcgcntgtgcatatatgtttgacagtaagacatagtgcccatcggcatgaggatccaatactcgcagtttcgccatagcttcctctgcaacttctacattcttgtagatgcgacaagcccctagaagtgccctccagatgactgcatctggctcgaatggcatgtccctgataagctgctttgcctcctcaatatgaccngatcgccccaaaagatcaaccatgcnnnnatagtgctgannctnnnngnttacaccatgcactacactcattgagctnanntatttcnnnnnnncncncnnnnnnnnnnnntgagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntnnnnnnnnnnnnnnntgatcatcgagctccaagtcagcacannnnnnannnncatagagnnnnnnnntnnnactgaattctcaatgtnnnnncacttggcatacatgtcgatcagagcggtcccaagcttgacatccaattcaaccccattgctctccanaaactgatgcacttcagcaccaacagcaagcgcccccatatcactgcacgccgagagcacgctcaccatggtggtgcngtccgggttgacacaggcngcgcgcatctcccgccacagctccagcgcatccttggacctccgccccttggtgtacgccgacatcatggagctccacgagaaggcatccctgtcgtccatgccgttgaacacctctctcgccaggtcgacctccccattcaccgcatacccatggatcatcgtgttccaagagaccatgtccctctcgcgcattccatcgaacactctcctcgcctccgcaacctcgccacgcgtaacgtatgccgcgagcatgacgttgcagaggaacacgtccctgcgcggcgcctcgtcgaaagcggcgcgcgcgaggtccgcgcgccccgccttggcgtaagcctcgacgagcgcggtgcgcacgaagaggtcggctgcggcgaagccggacttgagcgcgcgcgcgtggagggacgcagcgccgaggccgaggtcacgcatagccggaacggaggccgagagcgcggcggat

SEQ ID NO: 17 - DNA, 645 bp, Zea mays
  misc_feature (247)..(248): n is a, c, g, or t
tatgccgttctctttcttctgctatacattcttgcggtaagttaaatggttttaaaacgagaaaattcaagttgcatagcaaataaatcatttctcttatgcagttgtattttgcaggacaccactttcatattttattacctaaagttatggagtgcttatctgcaaatttcttgctatttcaaaggcacgactgcttcttgaggacaggcaaggagctatacttttatgcacaacgatttttttnncttccaaaattcaaacttaaaagattcttctttgcacctcgtcataacaagggccaatagttgatcataatgtaagtttggctattgttgatctcttaaacttgcaaatatgaatgagcttatgttgtattcaactcaatccatatctttaagttaaaagccttagggaacacttacatgactttgttatggttggtgtcacctactaacgatgccaacacgcacgtggctaggcaatgggcccaaattatcctttgattgntttannnnnanttaatttccattaaataggagntcaaatcnnnnnnnnnnnnntnnnnnnnnnnnncacatannnntnnnnnnnnnnngagaaaatcannnnnnnnnnnnnnnnnnnnnnnnnnnnnnnnnnntg

SEQ ID NO: 18 - DNA, 751 bp, Zea mays
  misc_feature (124)..(124): n is a, c, g, or t
catgcctgcagctcagctagcctcactgtcgccctcgtcagtcttatatattttatgtgctctcttgttcccttgtgtgatgtctcctcttcctctccctatccttgctttctgtgcctctcancaaanggcagngttccttgctatgtcatctagctagatagcacagagcaagctgcctgggcaggcagctagaaaattaacaacaaaaaaaaanggtcaggagcagtgggatttggtagcttgctgttgcttttggcttcgtctccgggcacatctcactgctgaaacctcatggggagggctccgtgctgcgacaagaacaatgtgaagaaagggccatggtctcctgaggaggacgccaagctcaaggagttcatcgagaagcanggcaccggtgggaattggattgctctcccncaaaaagcaggtagtatatatatgtgcatgatttatcaacctacatatacatagagtcatagatgtgatgtggcttttcatcagctggtttggggatttcatgatgttaacagttgagtttgagtccaaacagtctaggttcttggtgctgatgtctttctttttttttctttctaatttnnntncccccttatttatgttttttctgcnnngatctnnttgagatcgatgacctcaaatnnnnattgtncngannnnncnnnnnnnnnnnnaaannnnnnnnnnnnnnanatannnnnnnnnnntnnnnnnnnnnnnnnnnnnnnnnnca

SEQ ID NO: 19 - DNA, 615 bp, Zea mays
  misc_feature (86)..(86): n is a, c, g, or t
aaaaatattgtgcattgcgtgaagtggaatcagaatgggaactgggtactgactgcctcgaaggatcagattattaaggtctgttncttgtncttttgtactttngtctgtttttcttgtcttcaaagaactgatggcagtgtacacgctataacnaaaattctagtgctgtctatggagatgggccaatccatccttgtgagcaaatgatggcagtaaattattaacttttggtacgtttgctaatgctatcactaattcgttttgcagctatatgatattagatccatgaaagagctgcaatccttccgtgggcacaccaaggacgtcactggttagatagctgttccaatatcaagatttttataatttatataccccttcacatattttaagcttcaaaaattgacacaagttttggatgtactnnttcatttgttcattgtaaccatacatnnnatgattgttagaacagtggcnnnaaaatttctagaannntatgntncccacannnntnnnnnnnnnnngtactnnntatgtattnnnnnnnnnnntttttannnnntnnnnnnnncacccannnnannnnnnnnnnttnnnnnnnnnnnnnnnnna

SEQ ID NO: 20 - DNA, 452 bp, Zea mays
  misc_feature (16)..(18): n is a, c, g, or t
tgtaagtcatacatannnaannnngcggactaaaaatgctggtgttactccctccatcccggaatgatatattcatgtaaaagtcaaactttacgaattttggccnncnattattcaaattatataaaattttaaggcataaaaaaggctttgatggattcatatttgaattgcctctgatctgatatcggaactctttagcaaacgaaaacatacagtaagaggaattaatggtcaaaatctaatttgtttgacttttcctgatatgattaattgattatcattccaagacagagggagaatattactccctcngnnncaaattaaaattcgttnnnnntatttannnnnnncatannnngantaatnnntatgtttggtntatatgtntagatncatcatctatttgaatatanncataannnnnnngtgctaaannnnnnactaatatg

SEQ ID NO: 21 - DNA, 506 bp, Zea mays
  misc_feature (201)..(201): n is a, c, g, or t
acatccacggcagtgtcaaaggttcttgtgaacaacaattcgaagggctcctcaggatacagctctttccaaactttttctgactcaagtgtcgactttgtttgagtggacgactcaacgttgttattgttcagtattctgccatacactttcttgcagtcccttatgtactgaacctgaaagatatgtgctcgaataatnaggtccctcggttctctgaatagcatcatcaacaagggtacaggcatacaagatgcttatagatcatttccaaatanagtcatcatatntatcagtaaaaaccatgcagttggcngtctgattcancgttnagntcattcntaacacaccnaagggtacgtgaattacagatgatcatggtgagtagggtgtgtaccgggttaaggcgatggcagtgccatatccactcacaatcaagnggaacaaccnntggaccanctacannnnnacccttagtgtnctnnnnnnnnnnnnnnngnnnnnnt

SEQ ID NO: 22 - DNA, 636 bp, Zea mays
  misc_feature (43)..(268): n is a, c, g, or t
gcatgaatgatggaacggacttgttgacacacttttagggcgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttgtnttncctgtttgcaagngtaatggcaccggattcacaagtttgcgctgcatttctctgcgttcggtacaatggtacatgtgtacaatgaacagaaaaatagtattagtcagacttcctcctgcatttacaccggttctaaactagagccagttttcttccatttcctttgaattatccattttccaccagccacaccttttatatagatgctgtctcgaccatatgaacannnttcttgctagataggagagtnccatacaaacnnnnnnnnnnnnnnngcnnnatcnnnnntgatgatnnnnnattaccnncnnnnnngnnnnnnnnnnnnnnnnnnnnnnnnnnnnnnnnnnnnnnnnttng

SEQ ID NO: 23 - DNA, 629 bp, Zea mays
  misc_feature (17)..(21): n is a, c, g, or t
taatcctgattctcaannnnngcaaactcaagaaacaccgggtgctgttgcttctggtccaagctcaacttcagctgttgcaaaccgtgtgacttctgtggctgagggcaaacaagaggcaacggactgctccttgcaggtggctccatccaagccaaatgatccatctcccgctgatctggngaaganactgtcaggttcgatagagcagattgaaaagatggtgaggcgccatatgaaggaaactcatccaaaggctgctcagccttccaaggtagttgtccaaaggcgcatactgatgtccatgcgatgagctcagcaacgatttctcttgtctgctgcgttcttggccgctagtcaaggtatgtctagggatggggattcctggttctctgcttgaggacactgtggtaggagtaaaagagggatttctagcttctctgcttgagagcatggctatctgttggtacaacttgccatggtatgcttactgcatacgacttcagttggcttcaccagaaacgaaccactnnnnntcacgtctttctnnntntgnngtatangacntcactgcgtagtagnnnnnnntactnnntatttgnnnngtttnnnnnncnnnnnannnngag

SEQ ID NO: 24 - DNA, 625 bp, Zea mays
  misc_feature (388)..(389): n is a, c, g, or t
ggccttcttttttggctttaatctctcacaaggcaacttttttgacgaatgtgtaggacacagcataacaaagttctcctggagcaaatcacatttgttcagtgatatggtcaacaaatactttaccacaaaagggcacgcactttttgtttaatacacaatggacagtgcagatttaaggaacaatacttacttgatcccatttgcagcctttgatgttgtaggcacatgggacatgaaaacttttgcggcagctcttcacaaggcagccaagtgctgcgccttttaagccacaaacactgcatttgatcttagaagcacgggccaactcaggccccaagttgtttgcggtgtcaccagtaaagaatgcttgaggagccctgtattnntattataacttagtgcagcaagttttattttggaaatagtacactaaatgatcactacacaggtacagaatgcaaaaaaaatcaaactttgcacctaaaaagaaccaganaacacaaactnnntgtctcagaatatggagtatgtgtnnnnatcnnnnnnangangtatgnnnnnnnnnnnnttatcatgannnnnnnnnnnnnnnnannnnnnnntnnnnnnnnnnnnnnnnngc

SEQ ID NO: 25 - DNA, 475 bp, Zea mays
  misc_feature (16)..(17): n is a, c, g, or t
catacacttctcaganntaaatgatataattaataacacgactgtncgnnnnattacctgtcggaagaangnnnnnggcnaaagcactgctcnnntctannnnnnacacagtaggggnnnnntcacctaaccnntatgagaaaantatggcatttagnntataataaatataaaaannnnnntnncactcactttatgaccatttttatacttacnagtnnatcaccagtatctgcaatctcaggcttgggaggcaaagctatttgggaagaaggaactggagcaatttcttcaacaacttgcagtttctccgctacaattggaacggtatcacgaacttcttcttctgnnnnnnntgtcanaagaagccgctcaggaagctcctgntgaaataagnnnnatagtnngtatttaagnatnnattccacaataagtaaatcggcnnntataaacttgacttgacgntnntcattta

SEQ ID NO: 26 - DNA, 638 bp, Zea mays
  misc_feature (47)..(51): n is a, c, g, or t
atcttcacatatctctaaggcagtgtgtgtttctgccttgctttagnnnnnatgcaatttatcttgttcaaatttcttatcatctgccttctatatatctctttgcatggatattcaatcatttctttcttttgaaaacagtgggcctcctggagatctctttgtttgcctcgatatagaggagccatcagatatcaagagggatggtataaacttgtattcaactgtgtcaataagctatcttgaagctattttgggcaccattaagaaggtaagatatttcattatcagttgtgatctttaattagtttcagtcggcgatcactatcttgttcagtaaacagctagcttttcctcatacagcagaaaaatatctggtactnatccacttcacttgataactggagaacagaaacatgcaaaacttggtgtccttataagaaaaatagcctnnntactttattccantnnngatgcantcagcaaccttcaatttgatgtgtatgnnnagcccttttcgctnnnctttaacacaatatttgttgannnnnnntnnnnnnggnnnnnnnnnnnnnngattgctctttcnnnnannnaaagtnnnnnnnnnnnnnntttcnnnnnnnnnnnnnnnnnna

SEQ ID NO: 27 - DNA, 603 bp, Zea mays
  misc_feature (131)..(131): n is a, c, g, or t
cctaaggtgacaggctgctactctagattcttcatgccttttgtttaagctcccttgtgcctcacaagtggacacgaaggcaacgcaactgtgacatgcatgttagtattacaggcagctagctacagtcnttttcccttagtatataattaattaaactatgcagattgcgccgcgtcagcttggttaggtcaggtttcgccaggcctctctacccataccactgctctgaattatttgaccgacggccggnnnnnnnnntggtcctgggcccaaagaccagcttgtttgcctcgtactacaaagcctttccgacgtcaggactaaggaggactcaggaagagaagggcaggcagacagatctaatctaagcgtccatctttcgttcgatacgtacgtgctgctgctgctgcggggaatctcgcccctgccctgtctgcaacctnnntgtaaccacgaccgtcagctnnnncagccacagccantatccttatttactatagattagggcaaaactgagcccacttggacactgnnacttnnnnnnnnnnnnnnnnnnnnnnnnnnnnnnnnnnnnnnnnnnnnnnnnnnnnnnnnntcatg

SEQ ID NO: 28 - DNA, 655 bp, Zea mays
  misc_feature (66)..(66): n is a, c, g, or t
atatctgttattacttaatatcttagttaccatattttttttgaggggaatagttaccatatctgncngctatgacacagtctgcggtaaaagcatatgtgttcactgtagattttgggagggaaattggcttacctgtactctcatacattgttgccttgctggcaacatggttaaccaatgacataacatgcaaactgtatctttttactatttaccttaaccatgccagctttttatttggttctaggcatctttttattcttccttgctgtaatctactggattgtagcatgaacaccttcatgngttttagcttatgtttnnnntnnncaaccagtggtggcctgtgatatgtctactgtcaaatctgggacatactatgttgcaggcttctctagtccgtatttggctccaatctgtactagaacatatactcatttatctggtctactggtttaccaaatcatattacagaatagttgtgtgtattttgtgttccagcactttgttcactgaaatttgaaatcccagtttcttactcacattaattannnnnnnttnnnnncnnnnnnnanngggcggaaanngtnnnnnnnnnnnnnnnntnnannnnnnnnnnnnnnnnnnnnnnnnnnnnnnnnnnnnnnnnnat

SEQ ID NO: 29 - DNA, 476 bp, Zea mays
  misc_feature (1)..(2): n is a, c, g, or t
nncgccgagatgtgtgttactgctcacacgcttcgcggcacgcacgtcctgtctgaacgtgagcggcagggcccggggtggcctgctgctggtggtcacagattcagcgtatatatgcactgaatgtaacgacgtccagtgacgttagtgcccttcatgaacgaacatacccgacctcccaccagagatctttttngagacgtgagctgtggtcgcattaaaggctgttcttgtacagcactagtacaaagcttcataaccgttttctgggggggtctggattaaacaatctccgtaacagatgccctcaatttgtccaataacttagacaagcaatcccaataaaacagtggcgtgcgtgcgtgcgtgtcctttaatccaggccttgctaagcttaaccgggacaagcgaatccagggcaggtgctatgctacctagctagctaggcgacgcccatctcggttcttggaaaccag

SEQ ID NO: 30 - DNA, 667 bp, Zea mays
  misc_feature (5)..(6): n is a, c, g, or t
tcacnnganccangtaactaattgatcagcgctatatataatggctacttataagtttgaaatgtgtttctatcattacanttttctcgtgttggggtgggatagggggagcatgataatttggggaaaatgtatgttccaaagaggataagcatacctatgttcaatattggacctncggtctgataattccttatccttatgatgaggtttatctgttccaaagaagacaatagctttctaatcaagatccatgagtacaaaaggattgtatctcattttctaatttcaggacaaccatgattttatagaaacttgaataaattatgctacctgctttagaggtttcacgagggcaaacntcaccaagctctctacgttttttaaggatttgtgcgcgcatcctgttatcaaaatcctcttcattctcgtcttcaggaaaatcatcagataatggagcattctcttgctcccttgattcaactttctttgagatcaaagcatccctgacagtttgaacagtaacctttttcatctcctcttgctccttggactgcaaaatgtgagtaagcacaatgctattcaaatttggcagagcataacaagttacagcanntaacactataattgaaaaaactgacnnnnnnnaangatncnnatcgcaccc

SEQ ID NO: 31 - DNA, 684 bp, Zea mays
  misc_feature (172)..(172): n is a, c, g, or t
agcgaaagagcctaggaagacggataatgtcctgcgatcatgcgtcatgggacatataactagagggaaaagacttgcattaggatgtactcaaatacagtagtaatatgcgtccactctgtcttagcatctatatttctatctcaaaattaataaaaagatgttagaaagncaagctcaatgttgactaataaatacaaataagcatggcatttgcagaaaaatagaattatatagatggactctttgcacatcagcaatatactgaacttggtgcacattccagtgaagatttaagtgactggncattagttatttgggggaattgtatggttgcccatactcangaatgtagcttcgtttttaccnntgttttttaaagtttgcacgtatgtccctccttttttgaaacaaacagacaacncttancggtgttaactcagcccataaaaagacgttcatacctttgtactatatnccctcttcttttatnnattttctgcaaatttgtcctaagttttatatgaaagtttaggatanaattgtgacataaatctgaccnnnnnnnnnnnnnnnngnacaccattttnnnnnnggattcctatnnnnccaaaggaaaatgaactaatttcccttgaaaaannnnnannnnnnnnnnnnnnnnnnnnnnnncnnnnnngnnnt

SEQ ID NO: 32 - DNA, 707 bp, Zea mays
  misc_feature (133)..(133): n is a, c, g, or t
gccaggccgcttgtcccgccgggttttgcaaatgcattcgctgacaagaagcttcagtcacagtcgtctaacatcacacatgagccgaaggtgtgtatctagatgccgtttcaatagataatagaatgacagnaattacacacaaaatctcctaccttncaatccttgataccttttcagaaattgttgtgttcattgaagatagaaccttcagttcttcnnnnnnnntctccaatagaatcatttccttacgaaaagagggataaccccaccaatgctgtagtgatacttaatttgtttctactttgtagtgtcataatgctactactgaagctaacatgttgactattgcatggcttgggggtcagctagaggatgaccagtcagcaacagggttcacatctgaaagtaaagagaagggagtttctggtaacgatgctactatgggtccaaagcacacgcntccacctggtagtgttacctcttcagctgaattggcttctagcgttctgaaagggagcgaggattgggaagctgatgtaatggataagtattctattggaaaagnnnncaaatctaaaaatattgatccagttannaaggatgattcagtagcaatcntnnnncnnnncntnnncannnnnttannnnaaagnnnnngnnnnnnacnnnnnnnnnnnnnnnnnnnnnnnnnnnnnnnnnnnnnt

SEQ ID NO: 33 - DNA, 645 bp, Zea mays
  misc_feature (303)..(303): n is a, c, g, or t
cagctcctcaacgccgatttcggcggccgcaagatcatccgccgcgccgaaatcgaggcacgcgagctccagcgcatccgcgaggtcgagcgccagctcctcctccaaaagcagcacaggcattcccgcccgctgtcctccccctccagttcctcctcaagctcctcgcccacctccgcagccgctgacgccgacgccgacgcgtcgcgggcagagagcggctccaaggagtcgctcccgagggaggaggtcatccggcgcctgcgcgtgctgcggcagccggccacgctcttcggcgaggangacatcgcgcgcctccgccgcctccaggtcctgatcgaggacnccggcgcgctcgccgacatcgacgcggcggagatcggggagggccagaccaacgacttcctccgcganatccaggcgatgcgtgctaaagctgcgactgtcacgaagcccaaggcggccggcgcggagnccgagcgnggggagggcgacagcgtatcgatagatgtgccgttcgaggagctctgngacgaggacaagatcacngcgttcttcnnnagnnnnntgnacgagtggacccnnnnnnnngacnntatgcctgnnnnngaacnnnnnnnnnnannnnnnnnnnnnnnnnnnnnc

SEQ ID NO: 34 - DNA, 629 bp, Zea mays
  misc_feature (95)..(95): n is a, c, g, or t
ggtgtttactgtgaccctgatccctcaaaacaagttgatcataccatcactgatccaaatgttgtattcttctggggtctcatctacagaaggantattcaaatatgtacaaattctctcaaagaagatttcatgaaaagtgtctgcaataacatctatgggaaggaaaaatgtgcccattcagacatcacaatggccgttgttgggcctccccagctgccaaaaaagtgtctgtgatctctcagtttactcagaaaagctgccacaacatatgatggggaccaactaatgaagcagaactacaatgcagtcagttaggctcagctgtcatattgcgtgcaagcttcgattgtgaatattncaacagtaagaagccgccgcttcgattgtgaatatgacaacagtaagaagccgccgttgaagtcattttcaacatcagatcaggattttttgctgccctttttaaggggcttgttcatgacatggaatgggagattttttttttgtgaagtgnnnnnnaaannnnggaggctcnnnnnnnnnnngnnnnnnnnnnaatagggccannnnngnnnnntattcctgnnatgnnttntnnncnnnnnnnngcannnnnntgnananancnt

SEQ ID NO: 35 - DNA, 662 bp, Zea mays
  misc_feature (88)..(91): n is a, c, g, or t
taaaataaaatgtgaatgttgctcctaactgctgaacttggtaaataaagggagacagcatgctatgctacatgttgtcaatgttacnnnnttctaaaaaaatatttagtgctgaaagaaaggtttgtatatattgactgatctaactaggcagaagatggtgtgtatgtgccactttcagcaaattagttccagtatctccatgttatattgttagaaacagaagttngacatgaatccctaaactacttcagaaacctgagttgggaaaggaccaagatacgggtgtatgatttcaactatttaantgctgcttagctttcatcagaaaacttggatggaactggtcaacaacaattgtgcctgtatgccgtgtatggcatgaattacctggtacggttgaattgtggaagctcatcagattttactgatcagatagttttgcatcagtttgaantggggtgtagagaaagaaggtagagttgcagcaagtatgtttttatgtttctgcaatgacaaatggaagnacaagtaacaaaaggacangaaaatacatgctgagcccagtnnacacagncnagcannttattaatttatcatatgacatgnnnnnnnncctgacaccnncnaatngnnnnntgnngannnnntatctatggatc

SEQ ID NO: 36 - DNA, 729 bp, Zea mays
  misc_feature (27)..(27): n is a, c, g, or t
ggcgagcagaatataagacgggcagtncctcttgctcttggtatactctgcatatctaatcccaaggtaactggataaatttatcttgtacactaataaatctctgatacgtgagatctactggatacatttttnatgagtgtagtttaagaacttcaggtttcaagcaaaactttatgccccacatggnaattttctgtcctngttgataaattgctcaaattatgttttnatgnttaagaaatgtctctaatgagaccatcctaatggaattactgtgtgatagtgctaactatttgatgcatttgcatcagcattcaaacacaaatgtgtgcttgcattctagacagctgtttttcccaggaaaaaaatgtgaccatgaaacaaatctaccgaaagtcacntctatataagttaactttgttttattgtgcctgtaatataagtcttttcctaatactcttggacccagaacccgtcaagttttatccagtgccatattaaaaatatatcatatcatagcaagccagatttacnnnngtgcctttgtacaaatagattctatcacacgtannnttttttgcatggcgctgnnnttaatggccaaatgaccanntcnncactgcaagctgtactnnatcnannnnnatcacatgtnnnnnnnnnnttttttgncannactctacnnntttnnnnngtnnnnnnnannnnnnnnnnnnnnnnnnnnna

SEQ ID NO: 37 - DNA, 803 bp, Zea mays
gagctcccaagtcgtgcagctgtatgtaaacctagctagctagcaacaagcgaagctagctgctgatgcagttgctgctgtctctcggagaggcactactactggagggggggtgatcgagatgagagggagcccatgagtggtcacataagtgagggctttataggggaatggggaaggagctatagcagcagcacactagaagcaataagggatgagtgggagcgagaggggatggatgaggaggaggtggaagaagaagaaggagaaggaggctgcttgacgagatgataaatgcataatgtcatgtgcaagaaagaggtgaggtgttgtatactagcttattagtagtccggcctccttcctactagtactagatatgcatggtagctagtgcagcaggtgtcatgtgtgtgtgaggctacaagaacaaaggttaagggcacaggcaaagggctggagagagagagagaacttgcaagcttgtcatggtgctgctctgctcagatcaggggccatatgcattgcatgcttagaaggtggggcaaagtttgccttaacatctagatctaggtaccacagcgcctatacatgatcctctctctctctctctctctctatgcatgccttgacggttactagctagctaattgacaatccctatatacaatatagagtagctagctcttgtgttgtcccctttctatctctgcatatagcttgtgcttgtccctggaaaaaaaactctctacatatcggatgtaaattcattctatggcatacccctacattttcgacctacg

SEQ ID NO: 38 - DNA, 727 bp, Zea mays
  misc_feature (5)..(9): n is a, c, g, or t
tgacnnnnnatcatgtggcctgtttacccttttgggaccttattatatgatatttggcttgaagcaaaatatggcacaccaaaccttagaaaaattaacattgccaaaagtttgttaccaataaacccatgggctgtccaaacattagcaagaaaatagactatggtttcacttggcaagggaaccttgtcgaatttctgtgggaaaacagatgcttgccaaatctggttaacacagccagattctgctaatgaaacattggctcagaatcttgtagaccctgatgtcatggccccatggtactgtggtctagtgcttgtatctttttagttggtacaactcatatccgtttgtttctaagatatttttaagattctactatgatttctttagcctcaaaccaccatgggtcctcttctgagctcatacaccatttttccaactaacatgacaccaagcaaacacttgggcatatacattctgctgcatggctgcatcatcatatttccacaatattcttagatccccaaatatatttcttggagctnnntttnccttcatttatgactcttcacagtattaccaatataaagnttaaaatncacatgcttnnnngctgnntttgcaatttacatatnnnnnnnnnnncttttannnnnnnnttaannnnnnnnnnnnnnnnntnnnnntgttnnacatgctnnnnntgnnnnganannnnnnntna

SEQ ID NO: 39 - DNA, 735 bp, Zea mays
  misc_feature (2)..(9): n is a, c, g, or t
cnnnnnnnntggacagacattcggtctgcaccaanaattttgtgtgctataatgctagcacatagatcacattcaaaattgtagtggtgataagagaattgaagtaactaacagaaaaaatnttataacatgctagtctccttttcaatggcttgctcaaaccattggcatactcccaacagagagatactgtaagaagtaaatcctaataaattggacagatattgtgaattagggagcacattagtaagatcatatatgctattggacttgcagagatatgttatatcattccgctatatgcataataataaacttgaaatgcatactgatacaaagtaaggaccaaaatacatcatataacattaactaatgtcacaagtttacatctactgtcaacttcttgtgttcttcaagcatgcgacacatattgacatatgtacttgtcagaatagatgtgcagcaattcaaagcatgaaaacacgactaaattgaagatcatgaacactctatgctggttctgataatttgtttcaacatgaaaaaaatcatgtcaaattgaaaaggagnataacaagctataagcacaaagtgatgaaattaactcacatttcacgcaatgtattgattgtacccaatgtcnnnnnnnccgatnnnnntagctgatttncntgcnnnngnctacnnnnnnnacnntnnnnnangtaatgtacannnnncnnncacacanacnnc

SEQ ID NO: 40 - DNA, 667 bp, Zea mays
  misc_feature (125)..(125): n is a, c, g, or t
tctactggtgttcaattggtttctaacatatgaaaagatgtgccagccaaaattctgttttgatctgttagatgatgtatacatgcccatgtgatattggagttacatatggctaatataaaaangacacctgaaccttaggcaagatgaaaggcagctttcttcttgtttccattnattgttcctgttttgactcatttatgctgaaattgctgatgctatttacttcttttctgaccataaggatgggggttgcaacttctttgagtggtgcgatgctccatctcccgcccctgccaatgcacgaaataacatggttgtacactcagagacatcagcaacagatatgctttgcccatgcagtgctggaacttgcttaattctcaccacaaagacagggaaaaatgttgggaggcaattcttttgctgcccattaaatcaggtaaactcaggattcaccaatgttacctgaattcatgacattttgccgtaccanatagcggcctatactggtttgtttttctggaagaacttggcccctttttatctgacacaacttgtttaattataacattgaacatctgcaaccctactcntnnntgaatttgagtantcaacatttgtggnattttgaacnngnntnattataacattgaacatcngcnnc

SEQ ID NO: 41 - DNA, 756 bp, Zea mays
  misc_feature (66)..(66): n is a, c, g, or t
agtcatacaagtggaactatcaatgagccacgtgcaaagaggaagaaactgaaatgagccatacgntttgcctccggactcaaggtaccaatagctcttantttttctagattgattgaatatacttgacacattttgctttcacaataagagtaattgtgcctgaaagtgacttgtatttttacnttttttttgtatgtatgcttgcctacatatttagttgattatttacatcaccacattgtcagaatatatatattcccctactccgtccctttaaaattgtaagtcgttttagctttttaggtacataacttctgttgtacacctagaatcctagatattattatgggcatgtttggttttgacccctgacaaaaggtgcctagcccaggcagctcccaggccgtcgatttccagcacctgccctggcnnnnnnggccggatctgcctggtcgagtgccctaggccagtgtcccaactagacatatgccctatgtctataactgattatctacgccacaaaatattgccctcattgtctatagtatatgctgatgtggactccctccattcctattgcaagttattttgggtttttcaggtatagcttttgctatgcataatgcatcaatatatatgccaacacagcaacacctgctttgcaccaagcgctnntacannngtctnncnnnnnggacngcgcngnnnnnnnncnnnatncttgntcnnnnnnnnnatgnnnnnnnannnnnt

SEQ ID NO: 42 - DNA, 610 bp, Zea mays
  misc_feature (39)..(39): n is a, c, g, or t
ggctcctgacatgaaggtgaagtcgtcttccgctgtttntgtacgtgcactcgactaattcggccgattcagggttgcatcgctcacgcatggactaaaacgccagggaatgaatctaatattgtaagaacgatgggattgaatagcgcacgnctcgcacacatgtcagtcgttcatgtgacagagcttgcttgtacacntccattgacgtgcaagttattatcgtcatgaagaggcgctggtcccatgttaagttccggcacgtgatccgcgcacggccagccctgctgcacctgacctggcctgttgagtaattaagtcatcatcgctcacgtagagtgctcaagtaaaatactcctccctctgcccttcgnagccacaaattctaagcgacgcgaatcgcagccacacaccaccagcagcaccaagagccagatcagatcagcctgatgaactcactgccaccactcaaggcgcgcgggccgtaccaatgagcatcgtctgcccgagtagagtatacaagtattataagccggcgagagcataacgtcgcgtataaattaatattcatcgtacgatgccttgcgcgacnnnnnntgctacccacgcg

SEQ ID NO: 43 - DNA, 677 bp, Zea mays
  misc_feature (213)..(213): n is a, c, g, or t
tgcctgcagtaactagtgccatgacccatgcattaacctatgcaggaaaaggttgtccttttacttcatggatttcttggtacaagtgaagactgggttcccacgatgaaagctcttgcccccagtgcacgggtaattgcaattgatcttcctggtcatggcgagtcaaagatactgcaacgtcacaaaaattcagaacaacctgctgtaacngttcaactagttgcagatttgttgttgaagttgatataccacataactnatggtgaggtggtggtggttggctattcaatgggtgcaaggattgcactccacatggcactaaatcaagatcacaaggtaactgtgtttttacattttgtgtaaactttcttttctgaatgttctataaagcttgttgtgccaaacagatncgtggagctgttacaatatcaggaagtcctggactgagagatgaagcaagcagaagacgccgtattgctattgataaatcaagagcccagttcctgatgtgttgtggacttgaatgttttcttcagacatggtactctggaaaactgtggaccnngtaattatttgatgnnnnnactagcttgtttgattattgcctgtacaaaatggtaaatttgtatgcancnnnagcactcatttnnnnnnnnnnnnnanttctctnnnng

SEQ ID NO: 44 - DNA, 693 bp, Zea mays
  misc_feature (104)..(104): n is a, c, g, or t
cctgcagaccatatatgtttatggcatgccaatatctagtaggaacttagtaatagaaacgaataaattgccttcttagttttaagaataaaagttggtgcatnacatgcagccgctggaacaaagttttggctcaaagtcatatggtattgtgaaagcatcactaatatctagggaaatacttcttgaagaagtgaagaagatcagtaatgctgttggtagcactcttgaggatttggatcgcactgacttaacccttggtaaatatgagacagttcaaccatcaaagtcagcttcgcccagttacagttatgggcaaggtacgcccacaaagtgtagtccccagatgactggcatcttacgtgattttcttgaggtatacttcactttttttnctgaaaaacgttttctatctttttgaatatctgtattggttgatgcatctcaaacttgtttcagagttctggggttgtggttggaagcactgatgatatcttgctgtatactctatctgaggaagaattgtttgaactatttcaaattgtcagcagccaactctcatttatatggaatgagttcttgaaattccataggttagttatcttnncatgcatctcnnnttagnnnatnnntgtannnnnngantnnnnnnnnnnnattcttannnnnnnntannnataaacatatnntgac

SEQ ID NO: 45 - DNA, 674 bp, Zea mays
  misc_feature (62)..(62): n is a, c, g, or t
tggaccattttgtttctttgatttagatactgaagcattactgaatatattcataagacaangccatctgaaaacataacanggcctttgtttaaattttgattttgaccatttcatatggaatattggtttgatcacaaatacatgatcttcacataactcctctaagaatgtgatcttcangtggaaactgctatcgccatgacntggcagagattgtcgatcttccttcagccatgcttggtgntnnnnagnnnnaagcgacaaacagaccaagttgttcatccgatgtcgaagccagaggagtttcaggaaaatcggcttatgttnatgttttattgttatgtcattggacgagtgtgtatcaggataaatntagnaaaaaagaantttttnncttaaaatttggtcatgtactcaattatttctttgccgcaggaatatcatggagcatatgtgctcttcctggttgatggactggttctacctttactgcctctgcctgagcccccacctcatgcctggtccatctccaggagttcactgtccaccacggcatctgtgcacatggaaacgaccacctcgtgaccgatgtcttacttttacgatcaacactgnntccnnnnnnnnnnnctgatnaccnttnnnncnnnnnnnnnnnnnnnnnnnnctca

SEQ ID NO: 46 - DNA, 652 bp, Zea mays
  misc_feature (103)..(103): n is a, c, g, or t
tcggctgatgcagaaacaaataattaggttacatatgcatgactacaaatatactactcatcaaaatgataacatgaacagataccaaacttattgtacgcanaaanaaaaaaaatagttgaactttgcttacctggatacaaggtattccaatttctttgaaacaaaagaagctgcttcttcaggcgtaccaataaaatctaaatcagaaacttcaacggctaatgctagtgccgctgctatggctattgctggagcaccacggacaaccatgtctcttattgcattcctgggtaatatttaggggtcagtataaaacaagcattatccagtggtatttataactagttacatttcaatggttgcagtaggcctatcaccatgggagcatataacacaattgctggaaattatgtgattgttttttcttctctagtgtgatcaaggtcaatccagaataccgaaatttctactgatcacagattcaatatgaatatatgatagccttgtacacctctattgatgaaattaccnaaaaaaaantgccactgttttacttgaacacaaanntnnncnncnnnnntaaccannnagcagcnaaanctnnnttagannnnagnnacacgnngnaaattaacagttnnnncaagnc

SEQ ID NO: 47 - DNA, 531 bp, Zea mays
  misc_feature (169)..(170): n is a, c, g, or t
tcgtagtataggagcaccagatccatgatcgaatcaccccaaccaccagagccaacttttagctgtaaaaccctgagacagcgcaggtgtcactgggaaaggcagcgcgtaaaaaactacaagtactcagataaacaagcgtattagtacaccttctggcgagaggggnnaggatcggagtggactagatcatgagcttcgagtatgctttaggtgcacacacacaaagaagnnggaatcgaaatgggnaccnnnngtggtagccttggccatanaaaactggcggcatgataaccgtcctagaatctgagcacacacttttccggtgccggattttatattgnngtcgccnaaaaaaaaannncgctnatgcaaagangagatgcttagcggttcgacgcaatgatctaataagtgtactttattnnnaatcaaaccgagtaaaaaggcgaggagaacatgannngttgggnnnnnnntnnnnnaanggaaaaaaaaagannnnnaaaagaatgctnnnnannntttaca

SEQ ID NO: 48 - DNA, 667 bp, Zea mays
  misc_feature (151)..(151): n is a, c, g, or t
cttgcgcccagatttgtaagcatcaacgattcttcgatgcaactaatgcccgttcttcttcactctgaacttttgatccccttttgaagctgaatgagaagctgtcagttaagaagccttctggtcaaattaagttgaaagttctaaaggntatatcaaaagagcatcaaattgtttgggataccactaagagtgagcaggagcttttgaaagttgacctcctgaagagttgattatatgcncngctgaacctctattgtgctgagatcaaagcttgtcaattctagtattaaagattagcgtcgctggttttacattaaaattagaaatatatactcaatatttacattttcatcttattataaaattagtggaagcactggtagagtagatttttggaagcanggttagtattacatattcccttgaaattattagtctatacttgatttgagattttaagtgaactttagaagtttataatggtacacaggcttcgattccaggctggctccaccactggaaatggcggcactggtgccggagtgggctaccaannagccctgnnnnatggggatcgatgannctnnncnnnnncctgtnnntnnnnnncgcnnnnnnnnnnnnnnnnnnnnnnnnannnnnnnnnnnnnnnncnnttttnnnt

SEQ ID NO: 49 - DNA, 558 bp, Zea mays
gaacgtctggagagggtactccgcttggttctgtgtgtcatctgggtgaatcaacctcatgtcccttatcacgatcgtgtgcttgcagttcccctgaaaacgtgcagcagggtatcagaaaacatggttgaatgatctgataactctgttttgctgttggcacggtaaggctgcgccatgaataatgcagtgcagctgaagctagtaatgagcaaacatcaataatcatccgcattgcagttcagataatgaatgttagattccatcaaaagcatagcataagaaagcaacgtatgctcccattttacacctttttatctttttattgaaggaaagaaccaagcctgtctggttagcacaatgtgacataaagcaaccctgcccaatgtttttgtatctcatattacgaccgacaaacattcaacttcaacataagatatgacacccaaatgaattctaatataaaagatatgaaaaaacgaaggaaatcaaatccacgcacaaacttttcaccaaggaaaatggaactatctgaaacagtaaggcgaaagagaagac

SEQ ID NO: 50 - DNA, 715 bp, Zea mays
  misc_feature (97)..(97): n is a, c, g, or t
ttcttgagagccatcctcctgcgtctggatcctcacacaaaatgcacctgcacaacaagagacgccaccggttgtaactaccaacaagaaaggctcntcttctcttttgttgacgagcccacaaaggtccagatcgcaggcgcaaacaagagatgcacaaacaaatagaagcacctgataatctaatttcctggataaggnnnnnnnnaagaacnggtgatcaagaaccgaaccaagaaattaaaaataaaaacgaaaagaaaagaaaaaggattantgggatgaaatcgttcgtcttggttggagttggagnnnnnnaatcccaacgcggcaagtatctccctttnctttcaagnaagtagcgtgctgttttccaagaacgggtggcggtgaggcccccnagttggcagcggccgatcttggagcccaatcagtgcggtgcgtgcgtgacgccaggtcggtcggtctctctctatatanatntcggnccagtgctccagtgaccagtggaggcgagacaccgcagggccaagtttttcaattcaagatgagacaaaaagaaagaaaaagaagtannnataatnnagagagcgagaccccnnnnnncgganngataagctcgagccgtcagcgtcgagcagcangagcannagcangagcactgnagcagcagcgcatcnctatnnnnnncccncnnnnannnnccannnngnaa

SEQ ID NO: 51 - DNA, 715 bp, Zea mays
  misc_feature (28)..(28): n is a, c, g, or t
taagataataataattttaattggcacnaanacattgtataagcaatatattctctggcatctcaactaacaattctgaacatgaaaacttaacaaacagaatgaatcaatcaccatctgcgctgaattatgcaaaatgtatttcttttacccaacatattgatattgaaataaacnaagaagcnttgcacaagtaggggaaatttaaacaagcatacacactggcaatttttttggcactggtacatatgccataaactgatagaaatgtctattctacagaaggccacatccaatcatctcttttcagttttcacatagaatcatccatataaaacatatgtttgacaagattaaaattatttatgtcaatttntcgactcccaaaatgatgtcacatagaagctggagaatgcaaaattgatgnttcacgaatatctcacacctttgtaaaatattcaggattgtctgatatgaatgatttgtcactgccatcaaaaggcaacataagaatacactgaaatctataaagaacaaaagtttaagtaaaccaagatcattgtttccaagcaagacatcctcacatcatacaggatcacttttacacagagtaaccttaaaatggctagnctctgnncngtgtgtacttgtgtacgattgaaaaacttgaatannnngnnnnnnnncagtctcantgcannncgnnnnaacannc

SEQ ID NO: 52 - DNA, 630 bp, Zea mays
  misc_feature (6)..(6): n is a, c, g, or t
gttggngctgagaacaacctactgctgctgcactcaaccacggccntattagaaancttgggaccagtaagataatcaatcgacaagctatatatgctggnagccaagaagacaagtattagtagtagnnncagtagangaccatgcttgtgaaaccactactgctgctgcactcaacattagatcttctgaataggtaggatccttcagggtttcattctcaaaaatctcaatagctaaacangtctgtgctgactagtacataaatataagaaaatatgctggatatttttcngttctcatcagttatgctggttgnaaaattttcctcgcacaactggaagatanttctacaccaacttcatggactcattctcattatatctnnnaattcaaaagctttcgngggnggaactcccctcaaancgtaaagnngtaagggntngtttgatgannagggnattcaagtgaattgaatagcaagagatttncttggtttnannagcaagaggattcctctcntatggatcccttcaatncncngntcaccaaatcagnnnnnnannnnnnnnnnnnnnnnnnnnnnnnnnnnnnnnnnnnnnnnnnnnnnnnnnnnnnnnnnnnnnnnnt

SEQ ID NO: 53 - DNA, 553 bp, Zea mays
  misc_feature (457)..(457): n is a, c, g, or t
cctgggggagtgatgaacggaggatagcacaggagcagaggagagggagggatagaaaagccgggaggggagccctatcctccgatcgtgtttggttggaccttggcagcaggggaaaagacgatcggggccgaaagcgagttgctgcaccgggatctttttctacaagcatcatatatatacgagcattcctctgctcgtcagatatttattttcgtcctctttcaacactcgcccacacattcagattcctcctcgtctctctccgcagaaaagaaaagtgcaaagggaatcggagaagagagagaacaaattaaaagagatgaatttacttgtgattaaccaggcgcactgacggggagttccatcatcattccgagcatcattagagcacaggacagaataatcacccccaaggcgataacagcaacatctgcaagcaagcatcacacccccnttccgcggttgttgctgttgttactgtcaactcccctnnnnnnctttccacaagagctgctggatnnaccacnnncactgactannntnagcacct

SEQ ID NO: 54 - DNA, 653 bp, Zea mays
  misc_feature (146)..(146): n is a, c, g, or t
ccatgtcgtcgtcgtcgtccacggcagcgaacatctccgagcgtccgatctctccggacaccacccgcgtggcctgggtggggacgggcgtcatgggccagtcaatggccggccacctcctcgccgccggctacgctctcaccgtnttcaaccggacggcatccaagacccagggcctngtctcccgcggcgccagcctcgcggacannnnnnnnnnnnnnnnnnnnnntgccgatgtcatcttcctcatggtcggcttcccctccgacgtccgctccaccgccctcgatccatccacaggcgccctctcnggcctcaccccaggcggtatcctngtcganatgannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntcttcaagctcatgggaaacgcgctgtacatnnnnnnnnnnnnnnnnnnnnnnnnnnnnaagctgggcaacnnnancnnnatcgcannnnccatggtagggctcgtngnnnncannnnntacgcnnannnnnnnnngcnnnna

SEQ ID NO: 55 - DNA, 695 bp, Zea mays
  misc_feature (566)..(567): n is a, c, g, or t
gtaaaactttggttcgtgagtacagtattcaaaactcaaaaaaaaatactacggtttaattttgaggatactgtggtgttaaaaactgtagtttacaaaatccaaacagacacctcatagcttacaatagctcgattttctccaacaccgtcgtatcagagttatttaaagacacctcatagcttacaataatagctcggttttctccaacaccgtcgtatcagagttattaaaagacgaccccttttcaaacgctctccgcactccacgcgatctcggcctcagctgttgttgacgcgtagatgcagacacgtgttttcattcactccatcggaatccaaatgtgagttcaatgccccggacccaaaaactccatccaatcgagctctgagcgcacagcgcagtccgaggttcccatcactcccacactgtccatccctcttctcctccaccaccgccgcctcgcctcaccccctcccacgcctgcagcccttcctgacgacgggggaggcagggcccgacgccgctgccagctcccgcagcttcgccacttcgcttttctccgnntccacagatcgccgacgccctcttggccctctccaccgcgtagcaatggnnnnnntggatgcaccaccgagcgagnnnnntacgcccgtgctgaagcggagtnnntnnnnnntnnnnnacgcgcgcgc

SEQ ID NO: 56 - DNA, 626 bp, Zea mays
  misc_feature (6)..(11): n is a, c, g, or t
ccgccnnnnnncgtggtggagggcctcgtcccggggacncagagctaccgcngcgtggcgcagtaccnnnaggccgtccnnntgccgggctnnntcgtcgtcggcgtcgagtncgnnntctacttcgccaactccatgtacctggtggagcgggtcatgcgctacctccgcgncgaggaggagcgcgcgctcaagtccaaccacccctccatccgatgcgtcgtcctcgacatgggcggtgagctgcctgcctgcttcttgcgccatgggcnatgccatcgatcaagtagaaaccttttagctgatcgatcttgttggtttctctcccgcagccgtcgcggcgatcgacacgagcggtctagacgcgctgtccgagctcaagaaagtcctggacaaaagaaacatcgaggtacagctgccgccgctggccnnngtgtngctgcaactcctgttcattcgtaacatgcatgcatgagctttggtgaattttannnnncagctggtgcttgccancccggtggggtcggtggcggagaggatgttcannnnnnnnnnnnnngagagcnnnnngnnnngcnnnnnnnnnnnnnnnnnnnnnnnnnnnnnnnnnnnnnnnnnnnnnaaag

SEQ ID NO: 57 - DNA, 723 bp, Zea mays
  misc_feature (651)..(652): n is a, c, g, or t
tcagaggcatcatcagcatatacaggagctgactttttaggcaagaactcttcatcaggatagatacttgcaaataactgtaaatgaaaataagaaaacaaatttatcaggatagatctaatggagtaaacgaagcattattatccattctaaggctgtcacaacagagcatactcttgatttatattacgaagaaaattgttataaccatttgatactgacagaagtacagatcaaccagagttgtgtacctaatttctcatgcaggtacatgtctgcatcatacaagaaacagaaaaggtgtcacagtatgtaccttttttgttgatcccagtacgtttctgtagtccaggtattcatcaggcatggggcatgacaataaagaaccagatgttccctgatagtgtaagacaggacatcaaatgctttaaatataaaaacaaacacacatatatataacctgaaaggccaatgtgtttctagtattgaaaccaacatgaatacagtccatattgatatttgataacaaatagaaatgcacctgtgtgatctcctgaacatacaaacagctccagattagtgttggcttcacatcctcagtgctttcattggtggtttcaaaatggccttctgaaccatctgaagcctgannactgaaaagagtgtctanngcnttttttatgnannnnnntccggctgnnncatcnntacanggagtagaca

SEQ ID NO: 58 - DNA, 536 bp, Zea mays
  misc_feature (264)..(264): n is a, c, g, or t
gaggaatcattaactggaggatcatttgatgaaggaaatatggtcaagaacattgaagcctgtgactggttaaccagtgctataaagaacctagaagcgtccaacatagacccaatctatgtaaagttgcgtgctgtaggttccctctcttcgtacaaatagcatttgttttcgtattgaaggtttacatacctgagatttttgttaatattacaaatcagtggaccttaacttagatacacagttagatgcatagtttttcgngatgcatagtttttactatgcacttagatatacactatatctagatacacaacaaaagcaatgtatatagaaaaccaaaatgtcttataatttgaaatggcatgagtatntattagtatgctgctctggtagaacttgaaggatttttttatgacatggaaaggtngatatattgagtagnnnnnnnnnnnnnnnnnnnnnnnnnnnnnnnnnnnnnnnnnnnnnnnnnnnnnnnnnnnnnnnnnnnnnnnnnnnnnnnnnnntaaacgaca

SEQ ID NO: 59 - DNA, 694 bp, Zea mays
  misc_feature (530)..(532): n is a, c, g, or t
acacctcggccagctccagcttctcggcagtcaggctggtggagtccatggtcttgctgaggaccttaagcgcgagggcgacggcctcctcgcgggtcaagccgtccttgtagtcctgcttgagcatggactgcgcggcctggctgttggccccaacagcggcggccttccacccgccgtagttcccggatgggtcgctcatgtagagctggaagccgtggtgcttgtcccacccggcgaataggaaggagaccccgaaggggcggaggcctccgaactgggtgtacccctgcttggtgtcgcagagggactggacgagctgctcgacggggatgggttcctggtaggagagcgcgtagcgttgggcgtggaggcgggcggtgttgatgaggatgttggcgtcggacatgatcccggccacggcgcacgccaggtgggagtcgatcttgtacatcttctcggcggagcgcgaggtctggaggagcttggaggtcaccttcttctcgccgacgaggaccacgccgtcggcnnntaggatcccgagggctgacccggcgttgccgatcgcctnnatcgcgtactccacctggtagagncgccnntccngcgagannntnnnnnnncgncngncntagcnncnnnncannnnnnnnnnnnnnnnnnnnnnnnnnnnnnnnngnttgngnnnnctnna

SEQ ID NO: 60 - DNA, 686 bp, Zea mays
  misc_feature (69)..(69): n is a, c, g, or t
gcagctgaaaaaattgacagagcatcatcatctgaggtcagcaaccctgatactagtacatcagaaacnggttctccattctatcagcttagaacagatgctacaaaacttgttgcacaaacatttcaaagagggcgaagaaatctttggcagctggcaacaagtcgcttatctgttntattatctagttcggctgtttgttcaactagcacataccaattnctgaagaattatgaagatcttgccatnttcattttggctggcgaagcattttgtggatttgaagctagtgagttccgccagaagttgaagactgtctgtttgaactacatggtgtcctttcaccggcaaaatgtatatgtatgtcaatanctctgactcggttcttaatgatnagtgctcaaagttcaaacatgtgaattttgaactgctatcatctttttaactgcttaaaatgtgtatgcagttcatgacgtgtttgnagtctttacgaacattcttcntatttcttttcttttcttcattggtagtgctggtttcacagtagcccatttttgtaactgattttgatataagctaccagtgcttttatgtttctacaaaaggagtttgcagctgatatgnncattagtcagtgcnnnnnnnnnannnnnnnnngcattgatatgcaccatannnnnnnnngt

SEQ ID NO: 61 - DNA, 680 bp, Zea mays
  misc_feature (11)..(11): n is a, c, g, or t
ataaaacaganattggtttcattttcagaggaactgganaatgttggcacaaagaaaccagtcngttatnnnncagtaaaangagcatcaactgaacttatananttcgaagtcatgaaccaatctatactccaactagagcttaacangctaccgcaaaattagcnnccccgggttcaccacttactcgcaaccgcannnnnnnnatatctgagcaacaaaaccaaaatcacncnnacacccagcttgcaattgtaaaatcccaatctgcattgctgtcttgttcctcattagtntccaacatatcactgtccaactgagccaccaatcaattggatggtgatgtaacccatctcgcctaccgctggaaccgtgggacatcagacatgagtcaaatttagtaactagcggtcaattactcggtttagggattagataggaaacaaccaaacagagaggaagaggaacgactantatcccataaactnnnnnnnnnnnnnnnnnnnnnnnnnnnntgaantgaggttgctcagactccaatttaggggtggacgtgtaaccaactgtgtataccttgacgatgacctnnnnctcgtacnnnnnngcnncgccccagatgatgagncngnngncnnnnannngnnnnncnnnnnnnnnnnngcngngnnngatctnnnnnc

SEQ ID NO: 62 - DNA, 710 bp, Zea mays
  misc_feature (171)..(171): n is a, c, g, or t
cacatcaacccggcggtgaccttcgggctgttcctggcgaggaagttgtccctcaccagggcggtgttttacatcatcatgcagtgcctgggcgccatctgcggcgcgggcgtcgtcaaggggttccagcaggggctgtacatgggcaacggcggcggcgccaacgtcgtngcgcccggctacaccaagggcgacggcctnggcgccgagatcgtcggcaccttcatcctcgtctacaccgtcttctccgccaccgacgccaagaggaacgccagggactcccatgtgccggtgagtacagtatcagcctnnnntgccttcctgcttattcgnctcgtccgtccgtgcaagctcgacttaccgatggtttctttgtggacgaatgaacagatcctcgcccctcttccaatcgggtttgccgtgttcctcgtccacctggccaccatccctatcaccggcaccggcatcaaccccgcgcggagccttggcgccgccgtaatttacaaccagcaccatgcntgggctgaccacgtgagtggaaaacaactttcttctaccttcntgttgctaactagtttcactgttnnaagtagtatgangagtctnaannnanntagtnaantnaagnntnaannnnnnanataaaacangaaannnnttnnnnnntatnaannnnnntnnnnnnnacnnnnnnnnnnnnngcnnannca

SEQ ID NO: 63 - DNA, 640 bp, Zea mays
  misc_feature (15)..(15): n is a, c, g, or t
ctcggacggaggaanctccagtcgacagcaangactactggggcgcgcactcacttccacggccactgggtctcggactcaccgtgaagccacngcaatctgatggacatggactaggttaacatctcctnagcctgaaacgtgacagctgacatgggtnagagtgaaaanaaaattagtttaggcattaggggacacgacgcagaacggggtggctcgaggccaagcgggcggcggacccgagcggataaggacgagctcggncctcaactgtcgctgnnntggcgcatgacacaaacacaaagcatcgtttcctgttggtaatttaccaccactagcgctagcctacnactggtatgttttttgtttttgtttttttttgcattggatcgtagaattataaactttataatatgccataaaagctgaaggttgcagtattagtttaaattattctatttgttaatttaaaacgttgtcgcaggctcgcagctgctgtnnnnntctgctttnncctcccccggtctacaaagaccncnnnnnnnnnntcnnnnatgctcttcttcctnnnnnngcnnnnnnannnnnnnnnnnnnnnnnnnnnnnnnnnnnnnnnnnnnnnnnccntnnnnnctacata

SEQ ID NO: 64 - DNA, 628 bp, Zea mays
  misc_feature (489)..(490): n is a, c, g, or t
attccggcgacggcgtgggcgcccttcttagggtactgcccgcccaacttggagtccatgatgcggtacagacggcccttgtcgctcaagtagggtctcgcccactccaccaggttctgctcggttaatggtttcgacttgtctagcgcccgccgtcccgtcagcagctccagtagcaccacgccgaagctgtagacatctgccttcactgagagccgacctggagcagtttttaacaagacgatgcatgaggaaaaacaatcttgccaaaatgaatagtgtggccgagcagattatcaatatactggctttatgcccaagcttaagatttgatctggtacatgtggagatctctctactctctaatgcgtactcctacgtgcctgcacgaattagagggggtattccctgctgtgttccacttccttcaaaaggattgaataaaaggtatctgagctccccacggagatcacgaatggaatataattnntgaatctacccttttgaactgttcaattgcatgaaaatttagaccctatttagcacaacttattttcagcttcttcgcagatttaaacagangnnnntaccaaacagntatcccttgcagtagnnnannnnnnnagct

SEQ ID NO: 65 - DNA, 766 bp, Zea mays
cttgcatgcctgcagtggttatgaaattccacaggccaatttattttaattaatttctgcccagcaaacttgagaacataatctggaaatggtaattgaccacatggcatttgttcgtaaccacataaaaacgtaatgcaagcacctgatctgaggaatcacatatgcaataaagcaaagcaaaaacacaactgacctgaggaaccacatatgcaacaagaaacaaagggatagtcaggacattgaggtatattctatagtactgcaaacaagaaacaagtattttcagaaaaaagacagatttttacataaaagtaatgaaaatagaacacatacaaagtaaacagtttttttaacacgcaggagaactgcgtatcatttcattaagaaaggagaaagtacacaaggaaaggaatctgtatacaccaagacaaactaaaaatgttctaacgcaccgaccagccaccctgagaacaggaggtgacaacgcaacaacactagcctctgttcgagacccttacaaccagctgctggggccccttggccccagccgaacaccagagcactccatcgctagcaatgtcctgtaaagccaccaccaaatctggcatcatcccatcgaagacacatctgttatggtgtttccaaagttcccaagccaccagaatggtgagagagttcatccccttcttcaactcctttggaaccaacttttcaccaaatattttcatccgcatacagagaatcccgtgttgaagaacccttg

SEQ ID NO: 66 - DNA, 698 bp, Zea mays
  misc_feature (250)..(250): n is a, c, g, or t
taagatagaaagcaggagtcaattctgaataacactaggagctaagtctgacaatgtatatattttgaaggtaaaagatttattaaatatgagtagcttcgtagctgggagagtgatcattctcataatgaaacatggaactagtattgtatcatgataagcaataagattagaataattgaagaaagatcatacaaatgctccaggagcccattctcgatatgtgataccttccgcgctacattggaanataagatacaaagttatttgctagtatataggataaaagataaacacttcatgttaaagaatacctgcgattaaatccaaacttctcataactacgggagaaggcttccaagcctccttcatgttcatcaatgtctgaacggattcttctatagaggctgtacctgttcttttattcaaggaagttgaaagcacagtgagatggatagtggtgttcgcatgggttatatataggaatccttataagaagaatacagaagggatggaaatgaaggttgccaaagaattagaacccgctgttgcctctatattgtctgnctaacatttctgtaaatatcctctgaangagnacatatcttgtncgttcaanngtaatgcnnnntgaaaccannnnnnntacttnnnncatcaaagcagtatgnaagatttcnngtgaaaacgnnnctgnc

SEQ ID NO: 67 - DNA, 741 bp, Zea mays
  misc_feature (166)..(166): n is a, c, g, or t
atgacagtgtgcttaatctttagtgttaggccgtgcgcttatatgtactgtagagtctactgtttatagagtgaattttgaaatggggagtgggataggaaggctgctggaaacagccttaatgcatataagaatacattttggattagatgtttttgctgtggcnaagatttgtttgttctatcaattttgcatttgttttgcagctcttgttatcctatctgtctagcctctactctattgtctctactgagcattatatcatgatattaatgcataactttatacaatcattgaattttgtatatattcgtgtattatactacacatcttgtgagtttataacctcatccagtctattgtttgttatcatcattattgtttgtcatcagcattgttgtttgctatcagcattatatctcacctagtgtgtcagtgctcttggtattacgtctagatagtgtttgcttgggcacatgtngtcttgaactctgctgatgtaattttgttcctacagttgcctttttgccatcagcagtgattattntcaattgtgcatctcaactatagtctgcaatgaattttctnntacttgtnaagttgtcatataatgtgtcatggcctactatcatttagacatgtacacacattttatttactcatgaatncagntantnnnnntttattttcnnncnnnnngtctttcntncttatatttannnatgnnannnnnnnnnnnct

SEQ ID NO: 68 - DNA, 1157 bp, Zea mays
  misc_feature (91)..(91): n is a, c, g, or t
aacagcaaagtaacagaaatcaggaacataccattacaaacaccatcacatgtctcctgcatttcgtcataggttcctcggtgcgattcancttcganaaaccaacgtagatccgtccggtccgatgccactgcacaattcaggtttaagccttgagaagtgaggacagtttacccgtgcgaaggcgtgcgattcaaggccgctggtgaggaggtcagcgacgtccttcttgaggaacttgaccatcgcttgcttcttccgccgcagcagatccagccgggtccgggtgcacttgatcgcgtgcttgctacaccaaacagcaactaaatcagcggttggggtagtagaaatcaaacaaatacaggtgacaggtcaaatcgagaatgcaggaatctgganaaacagcgggaaagcngaacacagatcaagaacaggagcaggaacgctcgcaagtccggggaacagttccttggattcgcagnnnnagatgagaagtggagctaaannattcggatcgagggacgagcggaatgcgnnnnnnnnnnnattcgcccnnnnntnnnnnnnnnnaaacagaaaagctgaacagatcnnnnncgaaggaagctcaaaacaatcgatttcgagctnnggtntncaganannnnnnnnnnaaaaannnannnnnnnnnnnnngannnnnnnnnnnnnnnnnnannnnnnnnnnnnnnnnnnnnnnnnnnnnnnnnnnnnnnnnnntaccatttgttgtagannnnnnngttgagcagactgtcnannnnnntgcnnngnnnnnnnggtgnnnnnnnntccccnnnnnnncgcttctctctgctcccttctcttcggctcttctcctacagctccgccctcggcttttagccagtggnagaggaggtggtgggaggcacgcagagcggcagaggcagaggcggggcggccaaagctgctgcgagctgtggcgtcaaagcatcnnnnnnnnnnnnnnnnnnnnnnnnnnnnnnnnnnnnnnnngtggtggactggtggtggtggtggcctggtggagtgggggaagatggctgggtggaggtagtggagaaggaaggaaaatatgagctgttgatgaggagaagccaggtggccatgaccagcaggtatcattctgctgcatgcac

SEQ ID NO: 69 - DNA, 1064 bp, Zea mays
  misc_feature (167)..(167): n is a, c, g, or t
ggctgcgagatgcataaacccgccggatgaaggatggagtgctttcacgttattgccgccggcagctgcgagcaagagtttccctgggtcagcgagggtcacggcagaggaagccgaaacggatccaaaggacatggaaggggttgcggacgaacctgacatggcgntctgctggtgcgatgcctgctgctgctgctgctgctgcgtcttgtcgcctggtcgcttctggtagaggcctagggcagcagcacctccggctccnggagccggaggagcgtangtgttggatatgaagaaatggccttgcgggaanggctgctgtttcatcgatgctggctgcgtatgtgagtgctgcttgctcccaggattggaaccaggcgatgcattaggctgctggccaaggatggatggcatggccatactgaaatgattcgtgggagtagtcctggagccagacgtcgacgcggagttcttggctgactgctgctgtggtgatggcagttggacaggctgctgaacaccctttgcatttcctgtggcgggtgggctgccaccggtacttttggacaccgaattggatggtgaaccagaagcaacaggggcntttgaagatcgagatagatcccctcctccggtagaaaggttngttagagaaggtcctacctttgatgaacttaaggaagtctgtgattggtgaccaggagtgtagagagcctgcatagggacttttgcttgttgctggtggagactactgttcttcatcataatcaaattagggggtgtggcggcaggggtagtcgtagcagtagatgaaggtgtccctctccctgaggatgccttcaactggggggattgaactctcccttgggcagcacttgatgggtacatcatattctgaaaattagccatgtttatacggtcttggtacccacctgcactgttctgtgtacttggtgcagcagatctgggccgattcagatggtgatgctgaattannnnnnnnnnnnnnnnnnnnnnnnnnnnnnnnnnnnnnnnnnnnnnnnnnnnnnnnnnnnnagaaatgc

SEQ ID NO: 70 - DNA, 1233 bp, Zea mays
  misc_feature (1)..(16): n is a, c, g, or t
nnnnnnnnnnnnnnnnagtctatgataaagcttgtgcaaaccttgttagtgttaagaaaaaggttaaacttttttcctgaatatttttgtatctccaatgattttctgatgattgccaaatggctattgtattcaaatagtgtacatctttatgttgtggataatctccactgaatagctaaaactttatactctaaactttggcatccatgctatcatcatatcacacagattcaggtactttctatcgagtgttctgaagaggctaggaaggtggagcacgcgctagagtgggaggaagctttgaagcagacggtgtctgatgagaaggcgaaacagctcgaggtcatcagtgaagtcgagcaagcaggaaaatcattcacccgagaggcttattcaaggtacaagacagaaatggctgccagcatgatctgtcaggataaggtgcagattgttgatgcgattttgacaaagagcagaagttgcaggcggtattcgaagagagatatagagcttgccactgacaacttctctgaagaaaggaagatcggtgagggtggttatggcaatgtgtacaggtgcaccctnnnncacactgnnntagctgtgaaggtcatncannaaaactccattgacaaaactgatgagttcctgaannngntaactgatngttcctnnanncgnngacaccattcaatattcacactacattcaatattcacactactggagtcttttgatacgaagtcaaagttgtctgaaccaaaaannaaatttcncgcaggttgagattcttagccagcttcgccatcccaacctggttttgttgcttggtttctgtccngaaattggctgcctggtgtacgaatacctgaagaacgggagcctagaagaccagctcttcaacagcgaagggtgccaaccactgcactggttcctccggttccaggtcgtcttcgaggtgtcctgcggactcgcattcctgcacgcgagaagcccagagcctgtcgtncaccgcgacctgaaaccggccaacatcttgctggacaggaactacgtgggcaaggtcggcgacgtcggtttcgcgaagntcgtctctgatctcgtgcccgactggcagaccgagtacaaggacacgancgtcgccggcacgctgtactacacggaccccgagtaccagcagaccggcaccgtgcgtcccaagtccgacgtgttcgcgctgggagtcgtcat

SEQ ID NO: 71 - DNA, 1152 bp, Zea mays
aacagatccgtttcgccgatgtggggtgcggatttggcggtttgcttgtagggctctcgcctctcttcccagatacgctcatgattggcatggagctcagggacaaggtaatcctattattttgccttttatttctcccttgatttatttggacgccttgtagactgttccaagcttaaagttactggtagtattatgaagataaacaatgcgtttgacatgcttttgaatataaaaatgttaaaatcttagaaataaatcaaatttgaccacatgcttatcatgtaacattacgagtttatatgccaatcatctgcaacatgttccagtttggtttgtgatacgaatttagtattctgtgacaccaactttctttcttacataattattatttgcttcagctttgcagtaaatgtgtggttaaaatttagtctgctgtagatttctacttagaaaacgatgtggttttagaaagttgttttgttcagaaatttcaaatagcctagtttaggatccactgggaaatatctgttactcattttgcttgtcatttgtgatggtacaggtaacggagtatgtgaaggagaggattttagctctaagagcggcaaacccaggacagtatgataacatatccgttgtccgcacgaactcgatgaaatacattccaaattacttcagaaaggctcagctcaccaagatgttcttcctgttccccgatccccacttcaaggagaagaaccacaggaggagggttatcagcatgcagctgttagacgaatatgcttacgtgatggaagtgggaggaatcatatataccatcactgatgtcgaggagctcggagagtggatgcggtcgtgtctagagaaacacccgttattcgaaaccgttcctgaggaagagattaaggctgaccctgttttcaaattactgtctactgccactgaagaaagccagaaggtggcaagaaatggaggacagactttctatgccatcttcaggcggatctccttacaagaagaataagtcgcttagagattataagctagatcgtgttacgtgtgtttgtcagttttatacattttttttttaaaaaaatttagcagtaggtgtacttggtactaaatattgcaatgttgctttgcaaggtt

SEQ ID NO: 72 - DNA, 1108 bp, Zea mays
  misc_feature (261)..(261): n is a, c, g, or t
ccattttttgaagagaaacatatctcaattttccgggtttgtttggacagacaatcaagtaagtctttccatgacagtactgttagactattaattaggctgatcatgcctgccatatgtgccttggtgtttatggtagttgatcatagattaggcaagaatctctattgattgggtgctttttataaaccttagacgatccttgcctatattatacaccccttgtacctaggtagctcatattctatcaagtaccatacntgtgtaaaagaaaattcttccccatgtcatgtgcttagctctatcattagtggatcacctcttttaagattcatggttgttgnaatactatactagcctaatggctataatgatatgacatagaatgctctaagaagtgatgagttttctttattatttcagtcgtggttatctcatgtatattctgtattaatccaatcatgacactgttgttagctttgttatactttttaataagatgaccttttttttgcaactatgcttggattatttaggctttgtgtgttgttggtcatgtttgcgctacctgtatgtgatgttcatcaggaaaagcacaggaccaaaataaaggaaaagcttgagaaatttaacaaggagaagttgctagatttttgtgaaattctggacgttattgtaaaagtaactacaaagaaggttagttggctgagctttgtttatgcatgagcagtgagatctttagtaatggtcggtgatttgctgaaattcaggaagaagtttctgccaagctcttggaatttttagagtctccttgtgttaccagggatgttattctcactgataaaaaggtataacttttggaaatgctatgcatgaaatactctggtgatacacctatgattttttttggcctggggcctcctaaagtaatgtgattggctggttatgtccttaagggcagcactatatgcttagcagtactcaacttactagctctgagcttggcactgtgaactgaagtgtctgtcctgttctctgtcattgtagaaggggaagaaacgcggaaggaaatctaaagtaagtcgtgaggtaacttctgaaggtgctt

SEQ ID NO: 73 - DNA, 1102 bp, Zea mays
ggaataattaaatgctttgtcaataaagttttagatagttacttggtgaagatatatatatatatatatacacacacacaataaatgtgtaaaaaccgagatttaaatcaaaatgttgcctttttcagcaatagttttctgagatatatagtcaaaattccatgtggacaacaataagaatttgctatggaatgctgaaagtgaactttcctgcttcatatattagcttatttgtggtagaaaggtcacctatttcattgcaaatacttactcgtatccaatggcatatttgatgagagtcatgatcctgttcctaacagagtcatatgcacctaagaatccactgtgaacctaaaacatggtgtgaaaacatgtcaggtagcagaaatatgagaacagacacataaatagaaggaagagtgtagacgactaacttgaacttcttgtttgaagtcaccacctagcctctcagggtttagtctgcaatcaggttcaaaacatgttgtcattgttgtgtcaaaggaacacaaaataacaagaaaaaatactactaccatagctacaatttcttccttaaatcttacccagcaggtacaagcattaagtcggttcgcaaatccttccaccttgactaggtagacatgcagaaatcaatgcaaagcaaagtgaataaagcttgaatatatgccaaattataaagtgaatttgccattaatagaacacaaggctttcatgttaatgcatacttgttcggttccacgaaaggcaacaaccaaccttcttcgagaagaatcgcaccatatggcaacctgaacatgttaatataagctaccaaaacaaaggagactggaaactcattgataccgcaacaaaactcacttgtgtgtctgttgaaatattgtcaagaaaacatatcttttcaaagtccgacttgatgaaactgttacgtcccagtgaagtggcaagcatagcccatgcctccatggcagtctctgcacttgcaaacaagcgccgcatatcctctgcttcctgtgcatcaatggacagtaacgcagatgaaacagtatcagccttttgaacagaatctgcaggcatgcaagct

SEQ ID NO: 74 - DNA, 1201 bp, Zea mays
  misc_feature (757)..(757): n is a, c, g, or t
tgcatgcctgcagctagccaatgccttcgtatagaagcaaaaaatcacagaaaagtaagggccatgctgcccgctacttgttactatgattgctacctactactgtacatgttactatagcccactgctgtcatattgactaggaggaaggaagtagtcactgttcctatgagttatgacaaaagagagatgtgagagtagcagattattttacaatgacctaccacaaagatgcacgagtactccagtagtatagattctaatatagtgtatccacagttcaatgctgaaacaaagcttcacaaggttataaattcatcggcagaaacctcacttattacacaacaatataatcaggtagataagttgtactagtctgaagttaggccatggaatatttaaaaacgcttgccagcacaacattgccttgatttacataaatgtttaaagtatatacctgcatcaaaccccgctgataccagaattatatcaggatcaaaaactttagtgacaggaagcagtacatggtcccatgcagcaatataatcagcatcaccacacttgccatgctcccagggaacattgatgttataccctttaccagcttcttccccgatgaaacaatgagaagcatccccttcagcaggatagaagcttccataatcaaatctacaaaacaaacattagaacaataaacatttattatatctttagaacaatagatattttattatagcttcattaaggtgactacaantgatagagataaacagaggttttgtggcagattttctattccattaagccacaccacaaaccatggaaggattgggtacataaagaagaagcaaacttggaaactaagaaagcttcagcgtatgctatagtagatgctaatctaggattctaggtgctaagaaagataagaagataangtgctgacatatgcaccaactaatcctaagataagcacaaggcttagtctatcaacaaacacacactttctttttttnctcgaacacgcaggagagttgtgtatcattatatttaagagnaaaacaaaagtttaggccaaaagatggaccaaatacaacacctccttatggaggccagaagcaggcatacagaaaaagatccatcaagaaaaacagttaggcgacctctctcctaaacnnngaactatggccataatattctaaga

SEQ ID NO: 75 - DNA, 1049 bp, Zea mays
  misc_feature (46)..(47): n is a, c, g, or t
ttgcagctgtgctttagctaaagcgaacgccgtgtatgtttttttnntctggtcaactaaacattgttgtgtatacatacataaacagcaatcgaagcagacaataggcaacaaaataacgaattaatgctgttatcaaatgagccagcaacggataaacttgagatgctgccctagtagccgttctcaaactgaagccctcaaggcttcaaacccatcaattgcttgcaactccagccacaatcatactaataattacacttgagcgcagccaaaatagcaataaaaaagttgagcaaagagtgcacctgctttgcttaattgctctgcacgctcacgacgtccaaccgacgctacagcctacagcggcgtgaaaaaagaagagctgttttttaactccacacacggaacaggaattaccaaccgaccgacccagtgtacccgattcggaccagatcttcgtggaattcaccagatctatctagagaagaaacaaggaaacaggaaaccagtaccttcccgccggtcgatgagctgatacgtagcgcgctcaggcgnnnnnnccatggggcggctggcctcaggcggggcaagctaaaaagagccctcccgcccgccgcggttcgggctctccttcctgaacaaggtcgagntcggctccggatccggctgggccacggggaagtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacgggtgaggccnnnnnnnnnnnnnnnnnnnnnnnnnnnnnnnnnnnnnnnnnnngtggagtttgacccgannnnnnnnnnnnnnnnnnnnnnnnnnnnnnnnnnnnagtgtagggcgcggtggcgtcgaggggaagggtgcggcgcggtgggtggcggccagcctcgggatccgggtccagcacgggcgcacgcgcacctggcgatcggatgggcgtnnnnnnatacgaatatgcacnnnnacctggcgnnc

SEQ ID NO: 76 - DNA, 1078 bp, Zea mays
  misc_feature (371)..(372): n is a, c, g, or t
caggttgtactggccagctggtgcaatgtaacttattttccccatagaacccggtggaagagcaacatggtgctgcatcaatgtgttctcaaaaacagtctgcaacacacacccagtccgcaatcattaggatgtaaacacgggttaacagcatgcatttacatatcacagacagctagtcttcagaatgaagccatagatggaagtcaatgtcataagaacttacagcgtatagatctccacctgtgatgacatctccaacacctggaaatatagcacaagaaaacctatttagatagcatcaaagttcaaatacaacttagagataacaaaaacagcatgcaaaaccatttcaacaatggaggtactannttaagtcactacctagcttngttggctgaaattcccacaatacatctttgtcaagggcaggaactgaaacaccacgaggaatatacacatctcctgacttaatagcgatggtttttagaggtcgctgcaaacataatagagggcgagtgaggataatagacaaaaaataatgtaaatggccgcaaaagtaagccctgtcgtgttcagcacacctggataccgtcaaaaatgtttccaagaattccaggtcccaattcaactgaaagaggctgcatgggaatagccattacatgttaaatgatagcaaaagaatgatattaaaagatatagtaagatctgaagacaactaaaacaaacctttcttgttctcaatacagggtcattaaccatcagtccagctgtttcctcataaactgcaacacgcaaatgacggcactcgcgaggttagtaccgtgtattcaactggaaaggagcttttcatgcccatgggtgaataatttattatagatgatctgccatgttgcataccttggattgtagctgaatcgccctcaagacggataatttccccaatgaggttatcatgtccgacacgaacaagttcatacatggcagcaccacccattccatcagccacaaccacaggtccagagacctgcaataacccagcatcaaggctaatcaaagtggggcttgttatatacat

SEQ ID NO: 77 - DNA, 1183 bp, Zea mays
  misc_feature (102)..(102): n is a, c, g, or t
aaaggagtgacgggcacgtcggcgtggcaggtggcgcggcgccattgggcggggccgccgatcgacgacctgcgtgcgcgcgcggcgggagaggctgttacnaaaacagagaagctcacatcggccaagtggtttcgccatctccgccgcccctggatcactcggtgctctcctctcctactctcccattcagattctntaanaaaaaanngtctagatctagcgagatcgtgntgtagtannnntngnnttgcttggcgcatcgttgtttgttaacattacatgcttactgttgcccgtgtgcgtgtatcaccactgtatccatccatccatgtttgtttctgcatgaaacggtgcgtgcgcgcgtgttgtcccgggtgtagaaggctgccgcgcgctctggacgaagcaggctcatctcgtgctccagtgcggcagtgcaatgcaaaaactgcaaggccaacctgaagccttgaaaccatggggccggcaaggcggcaaggcatcggcacgnccgaaagttgatggatcatgcgtctcgcgtctcatgtgtattattcgcatggcgaaatcgccncnnnngtgnntatatataattatttatttacgcactgnnnnncgaccgannnnnnnnncgtgcgtgtngncnggtagggggggtggcaaacgctttggcgcgtggtccggtgctcggttgcttaccagctagagnnnnaaaggtgagacgctgttgtttttagatgcatgccttttgcgtgcagnggagagcgnnnnaaggatagctagnnaganggccnctccattgcctgtagtgcagggtgttcatggctgctggtnnnnntggtcatacagtatgatcgacgacggcgagacacattcatttggtagaacatacgccncccgccccgataagaaaaaaaaaaancgcnnnnntaggttgccgagaatgcacgccttcaggttgtactccctccgtttctttttatttgtcactggatagtgtaattttgcactatccagcgacaaataaaaagaaacggagggagtataaattattaatgctatataggacatggagttcagagttttttatttcaaagaaaaaatcagagagataaagagagagagcgctgctgatctgtatatagaaagtagctaggtcgnnncttcctannncctacta

SEQ ID NO: 78 - DNA, 411 bp, Zea mays
  misc_feature (266)..(267): n is a, c, g, or t
atccaatacataccactgtctagcacatcagaatacaagccagcggccaaccaatatttcatgaagcccttcttgacccgtttcataagctcaattatgtaatcacctttactattgtacttggtacagttatcccatatgaactgtacatccttatatacatcctctgaattcatatacttgtcaccacgttccaagttttgacatattgtgccaaaatccataggagtctcaataacatcaaaataatcctacaaagttcgatnngtgttatgagttgggaaacaggtgacntgaaagaataagcaaaagatatatattcatcaacatatacttatatagctaggtacttgggttaagacaaaggctaggagacatagatagaaaaatccaagaaacaaaaggaaaata

SEQ ID NO: 79 - DNA, 823 bp, Zea mays
  misc_feature (583)..(591): n is a, c, g, or t
gtttaattatattaccatgttcgcgtgtatcgtaagattaatttgggctttgattctagttttgctccacatatgcttcttgcttctttcttctctcggcagtttcattctctttgtagcttgtttttggatctttttttctttccgatttattagataggtcagtatatatatggctagaatttgtagtaggctaaggatcggaggtggaatgggctgatttaattaatcagtgcaactatgtgttcatgatctgatacgtatacgtatatatcctaataagcaaaacaagggcgatttgaggccatggtgatggtttcctagcttgctacaataaggctacccgcagtcacggacttcagaggtcactctatccgtttacagtccacgatcgagtgtgtaatcagctgcagcggctgatggcaggacagattcagacgcgcagagaatacgtacaagtcgccgaagtcggtatccattccaaccaggttctccaccaccagattcagaaaccgcctggcctgttcatcctctccaaaacaaactaaaatcgagaaacgcgatcgtaaggtagcggcggcgnnnnnnnnntggcgacagcaggagtatgattggcccagttcaatcgcgcgggcgcggccaatctggtaggcggtcatcatcagaatccagaatccacataccggcccatttacagcaaggaaccgcgaagcccaagctcgtcttctttcttggaagaggggccgcttcactcgactgaaagcgcaatcaactctagtctctagccacaactcgaagtacgagtanctccggagtccggatg

SEQ ID NO: 80 - DNA, 387 bp, Zea mays
  misc_feature (1)..(19): n is a, c, g, or t
nnnnnnnnnnnnnnnnnnnttgcccccgcaatgtgtacatcctgaccagcagaagtgatccactacagnnnnnaagaacaaagctagagtgagaagaacanggaggaactaagctaactcccggcctaattatgggatgggttggacctgctcttgggcgtcgtcttcaagctctcgaacaacaccgtcgccggtgccttgaaggcttggctgaacccgcccacaggcgtcgagcaggagtcacggcattgcgtcgacagaagccttctgctcctcactctcacaacagacggttgcagctgcaacggctggnncganggcgatgaagaagaannngaagaagggccacagagcaggacaaagaagagaaggatcagcagagctgag

SEQ ID NO: 81 - DNA, 438 bp, Zea mays
  misc_feature (21)..(21): n is a, c, g, or t
gagctcagcctcagccgccgngacggggacagggaaagatggccgacggcagtgcctccgcctcgccgccttccgtttctccgcaccagtaccaccgcgacgccatcaagtcctcaggtgcaccctcgcgcttctctcctgtctcctcccgcnggcgtcgcttcgtcatcgcgtcgaacactaagcgcgcgcggccgtggcagaagccctaacctgtgtactccctcctgagctggcgtggcggttctgttgctcccggaaccangagcggtgagcgcgctaagtgctctacngcctactgctatagccttatttgggaataacttcgcctacaccggggttcgataactgtttgctcgagcagtcgagctgattagaatcgggactcatcttattagcaggnttaggtggtaagggacttggtttgaggggnttatt

SEQ ID NO: 82 - DNA, 420 bp, Zea mays
  misc_feature (327)..(332): n is a, c, g, or t
cctgcccacgcagaagatcggcctcgtccagccgctggaggagctgctcatccactcgttcccgctgccctccttcctcatctggccgggctactacgtgctctaccgcttcatcgagaagcacggcgccgaggccgtggcctacgccgaggcgcagcacggcatcggcaagaaggacgccatcaacaacatgctgttcgtgctcggcttcaacgccttcggcggcttctccgtgttcctgcccttcctcgtggccaaggtcggcggcgccccggcgctgcgcgagcggctgcgggacgaggtgcggcgcgccatggtgggcaaagnnnnnnacggcgagttcgggttcgccaccgtccgcgaggncatgccgctggtgcggtcgacggtgtacgagatgctgcggatgcagccgcccgt

SEQ ID NO: 83 - DNA, 377 bp, Zea mays
  misc_feature (30)..(30): n is a, c, g, or t
cataatcggctgcatgtcgacatgcgacgncccactgaagaacggctcatgctccggcaccgctggctgctgccaggcggagctcccgccgggcgtccagttttaccaaggcttcttcaactcgctgcacaacaccaccaagatatggaagcaaacaccctgcaactacatcaccgtgatggagagcgcggccttcagcttcagctccacctacctcaactcgacggtgttgtacgactccgacgacgggaggacgccggtcgtgatggagtggggaatcacacggcagangtgcgaagaagccaaagccaacaagactgcgtacgcgtgtgtcagcnaccatagcgactgcgtctacagcgatgccgccggctacc

SEQ ID NO: 84 - DNA, 392 bp, Zea mays
gctgagaaaaagcgtaaagaagaggaggaagctgtagcaagggcatcccaagaagcagctgagaaggaagctgctcttgcaaggaggaggcaagagaaggccatggctcttggagctgaacctgagaaaggaccaggtgttactcgggtatgtgatgtctaaccctctatgcttgtgtctttaaactacctttttccaattcttactgcaagatagcatgcttcatgttgtgccgatttttaaggatttgtctatttgagtgcgtcaaatatcactgttatgcctgcaaatatctgtttcatgtacgatatatttacagtgattgcaatgaatccttcgttactatctgagttaattgttagttttggctacgtagtgcctgaatttgatat

SEQ ID NO: 85 - DNA, 535 bp, Zea mays
  misc_feature (11)..(19): n is a, c, g, or t
atggacaacannnnnnnnnaaaccctgcnnnnnnnnngtgagtagcnnnnnnnctttagaaatacacattngacatattcctccactacgcatgtttacatatcctactgcaaattctctctctctctctcnngatgagcaggggctggcgtttcttcatgattaaactctggaaccacagccttcttgacgcccgcgccatgaatgcttgcaacacaatccttcaaggctaccaagacggaagtgcggaccccaagtaaatctacaatccagagaaatcgttggccagcgtgtgtaagatctgccggttccggcggttatttctgaaccccaaaacagctatgtgaagaactggaatggggccatagcggcatttgttttaaggtttagggttacnccatttttccaagggaggaaactaatttnccttggaaaaatgaaaatccattgggaaaaatgagtttnnnnnnnntagccattaaaagggtagcttcctcccacctagggggaaaaaaactaggagggaaaagntgga

SEQ ID NO: 86 - DNA, 665 bp, Zea mays
  misc_feature (55)..(55): n is a, c, g, or t
caaatgtgacactatttccttccaaacacaaaatggaaacgtaaaggctcaactncttcacgtcttcttctggtctctggccttgcattagtagtagtgtgntactacaataatgaaacaaacttggtccttcctccagntccagtcaaaaactgtgttctcaactttgaanttttaaagnggcatggcatggcactggcctgacagacggcggaatggacatgtnttctcttncgtccccgaagcgccaagaagattcaaaaccggcatgcaaatcgtcagaaacttgcagcaacgctttattctttcctgctgtatgatttggttcatacacttntacagacttcattcagagcagcactgcaaaaatggccatnagccnatgaccaannnnnnnnggaaggggcaaggggngnagcaaaaggatgttccccnccaccacgnaaaaaaannnnngaggagttcctaaattaanctaatcgcgtccgtgtancttttatccacagcttaattcatgacctaatctacgcacaatgttcatgannnnnnngaagggtctcttgagccccttgaaatctaccgtctcaatgcactcaacgccgtctctcgacccattcaggagcctgagcagctcgctggccctctccttggtcacgcccatgcag

SEQ ID NO: 87 - DNA, 660 bp, Zea mays
  misc_feature (439)..(439): n is a, c, g, or t
cggaacagctcccttctaagcagacagcacaagggtcgatacaaacaggtgactggtttagatacctccatccacactccctagagaacgaaatgacacaattcattagatgttttctagacaagcaatgcaaaatggaccactatgataggctgacaagtgttggaactcatactacatggtcaggaaaacaatttaagggaaagcgttcataattaggcaactatagggagtatacagatgaacaagatcacatggcaacctataagaaacaatgagcagtaaccagtaaattactatttaagccatgcaggcgacaagaccagggaactaatggcacatagctgcataactaaattagagacatggtcacctggcaatgttcatgatactcatgagtggaagacagagataaggtgaaggaagagtgtgtatccgnccctctggttctctacttaagatttcttcaactgaatttctttgccaggaacgagccaccattaaaggggtccatctgcataacagaaangaacatgtacttaganaaatcatagtgctaacaacaatagcaaaggctatttgccatgtggcagangttgggaatcgcacccgctagcattttgcacagcaatgcaagcncctcgagcaattaggagctgc

SEQ ID NO: 88 - DNA, 611 bp, Zea mays
  misc_feature (24)..(24): n is a, c, g, or t
aaactgaaacgaagccgtgttgcnagtagtaggatggaacagtgcctcccatttggccagaatgccagacgctaaactggtggagatttgtcanattcattcacaggacaggacaaaactatcaacaggctcactgatgacagancaagagaaaacaaaggaacgacacttgcacaggtcagttccaccccaaagtaactcctcgctcctatatatgtatnnnnacacctagttatgtgggagcttcatttcagacgcaccagtgncnnncttcccttcccttgagtgctgtgttcgcacacagattttcaccacctcctcctcttcctcctcttgtcctccgacttgtcagccgcgttcttgagctgcatggcacaccagcagttcagtcggtcgatcagacaccatacaccaattnnnncatgtcaccnaaagaaatgctcagtagtagatagctctcaccatgatgacaaggatgcggacaaggacagccacgaagtcggtgaagagggtgagggcgtgcttgatgtagtccatgtcgccgtggtgcgccctctcgatgacctcctgcgtgtcgtacaccatgtagcccaggaagatgagcagcccaa

SEQ ID NO: 89 - DNA, 714 bp, Zea mays
cctaacgagtcgacctgcaggccgacgcccgtgagcatgccgccccacatcccctgcaccccgccgcgcagcgggaaggcgatgatgtacccgacggggatgccaacgaggtagtagcagccgaggttgatgtacgccaccagccactgccagccggccccgacggcaacgcccgacagcaccggctgcacgctgttgaggagcagcgagaaggcgaagacgacgccgaggctggccacggcgcgcaccacctcggggctttccgtgaagggcgcgccgtacacgtcgcggaaggccaggacgaggacgaagaaggccaacccgatggccaccgaagacatgagcaccaccaggatcgcgaacttggccgcgcggggccgcccggcgcccagctcgttcgacacccgcacgctgcatcagattttgtagaatataatagtcatgagaacgcgaggcgtagcgtacgttcctgcttgctgcacgtacgtcgatggcattggcaatatgatcattattacctgatggccgcgttgaatccgaagaacaccatgatctgccagccgaacaggttcgtgctgtgattcgtatacagtggagaagcaaacggcgcggcgaccgagcaggcgttagaacttgctggaaccgcagcagagcaaccattccatctgaaaacacgtacgcgacacacgggagagcactactcaccaaatggag

SEQ ID NO: 90 - DNA, 1185 bp, Zea mays
  misc_feature (545)..(545): n is a, c, g, or t
ttatggatgggtgagctcatctataatataaacgtgaactacatattcactgcaaatagaataaaagttatagaactcacctatttaaaagtttcctcataatatttatcttttgtgaatgtgaactactgaacttgggaatcaaataaaaaagcaacattcctttatatgtattcctttgaatttctgattgaatgacattcctataatcttttttctacgtttacatattactggcagtcacctctatgagttagaacaacaccttgaccgcttcctgaagtctgcatcgatggccaaaataccattacctttcaatcgctcaacaattagaagcatacttattcagactgtgagtgcatcaaattgcacccaaggatcactcagatactggttgtctgctggacctggagacttccagttatcttcatctggctgtacaaacccagccctctatgctgttgttattgaaagcccatccttacaagtaccgtcctgctgcagaatcgccatatgcccaactagatcagatagtgggtggccangcggggtatgctgatacaatcctaagctacaacctaactgcaacaaactgcttcagcaatgccaggagtccaggactgaacagcactgaancagtcaatctggatcgaattgaacaagtgctggacagtcagtttccacatgaatcagcaccgaaacatccggatgagcaaaaggagaagaaggcttgtgtggtcgtgcggaggtaaagctttgacctttgtgcggcttcggcctggttctggtagaacttggcgaagcggccgcggttggctggcttgggtttagagggctttttcgggcgcaggccggcgatctcctgcaccagcggcacgtgcaccagcaccgtcctccacgccacgtaccagcctggggcgacgaagcggatcagggaagggaacccacgcaactanncaactgaggngataaggggaggggagtcatactgaggaggatgagggtgaagacgaaggcgatggcaaggacagggcggatgaggaaaacgacggcgacttccgacgcagccgccaggccgccgctnnccatagcgccggcggcagctcagttccggcctcgcaaggangggatccgctggcgaggttattgagcctatggcatgcgaggcccgcggaagcaagcaaa

SEQ ID NO: 91 - DNA, 637 bp, Zea mays
  misc_feature (1)..(2): n is a, c, g, or t
nnagtaatatgatggtgcaatgnagaaacaaggcatgaacaacagaagaatttaaaacaagcaagtcaagaaccaactcatttcttcagncacataaaccttagcaacttcacaaatcagtataagctcagatcctgaccaccaatgtaccagcggaaacaaaggtcaaacacagaatctaaaatacacctgctagtgtcagcagtacttaatgatcgtgctcaagtactcaacaagcaactatctacaaggaaatgaccaaattataattttccatcgttcagtcacagttaacaagcaccagacactttgtaccctcaaataccaacacgcagaactacagatacaactaccacataaatcgtagacaaattggtagggcagattcaaaattagcaacgtaacaaacacctaacgagacacacttagcgaccagcctgcgnaccgcacggcgacatcccgacgctgaacacagccacaactaccagatcaggcgtcacgagcacggacaagngaacaggaaccaggcagcatggaaatggaatcacacgagcgcggtcggatcttaccagtccagaggcgctggcggtggagatggtcagcttctggtcgtaagtgtagtccttactgagtagat

SEQ ID NO: 92 - DNA, 640 bp, Zea mays
  misc_feature (2)..(4): n is a, c, g, or t
annntactaatatatcagaaaaatatttggtgtaatggcattatgatttcttggccatatgggaaatgagaaatgacttgcaagttttgatgtttacacacaaatttgggcaggaacgcatagttttattgtaaaagtgttaaacaagatgatttggtattcacataagctttactcctgaaatgtcatcacaggactaaaacagcatctactttgcaagaccttcccctgttcgaagtgccatgttcagattcgctagtgaaagaaaaaaccctgacgacctaattttagccccaaacaaagaatcgaaaggagatcacagttacctggccggtccaagaagcagaacacggggcggtcgaatccggggcactcacgccgaccaatcccgtcaggaagctccccgtcggcggcaagcagagcatggaagtcaactacggctgacagatcagcgccacctccctctccgctagacggcctgcatcctgacctggacctggacctggnnnnncccttgacgttgagtgtcttgtaaagcttgtactgtttctccgcacgccggaaagccgtgcgctccgcagctgtggggtcggaagtgccgtacatcgccggcagcgcccgagtgagttctcgccgccgt

SEQ ID NO: 93 - DNA, 642 bp, Zea mays
gtcatctgcctcggctcgccgccgcacatgatgggctggttgaagccgccgttgaacgccacgccgaacacgtcgttcggggccatcttgctcgccgccgggttgtagaacagggtcagcccctcgccctgccaataacaactccatcccatcccattcgcgcatcagattcagtgttgacacacaacacaagcaaggcccgaaccccgagtcccatcccatccaaaacaacaactcgagtccacaacagtactcaccggggaggacggcaggccgttgctggtcttccagtacaccggcgcccgcccgagctcgaactccgcccacgtcggcagcgacaccgacaccctgcttggagtttggaggtcgatcagaagccgtgcgttcacgtccagcagcagactcgtcgtcagccaacaaagctacaagagcagcgagcgatggcatgcagcagacagcttgtttttgttacgccacttaccccgtgttctgctgctctacggcggccggcgccgcagtggcagcggcggcggccgtgcgaatgctcgcggaggagctccgccggctggcgacgaggactcctgcacgggtcggcttcggctgccggcggatagccgcggcgccggtgccgctactcaggcg

SEQ ID NO: 94 - DNA, 947 bp, Zea mays
  misc_feature (40)..(40): n is a, c, g, or t
agcaacacaatgtgccccctatgcatccaccgggagcacntgccctagatggcgatatcaaaacaagtataatgacaacaatacactagacttcttgttgatgtctaaggtcgctgtatacaaaggttgcaanctgatgtacaaaggnatgagctaacatgtgggcaccatgagaaaatctgaaacaatncgntantnttgacagtgaaagaacatgtaaccagatannnngtaatgaatcaacaccaattatatttcacaatttgtgactaaagaaaaaaanantnctagcattaattattctcatcttccaaaacataactctggacttatcactagcantggccattgtgcacttatcattagncaaaaaaaaaagtgcatgcacaannnganaaaaaaaacttcnnatacaacaaagaatttagccattatcagattcaccttancatcatagcatagtagtttcaaaatggcaaatatgtagtactactgctccacacccgaattcantagctgactacatataccaaagctgaaatacttcaaanatagcaaacaagatcagaagacatggcattcatgtacttacggatgcaagaagtgctcaaacagccatgagcgaagaacagacactgcacgctcaggaagccctctttgaggcctccaaatgttgtgaggctggctgaacgcgttcacgctgtttcccctcaggaggcctgctccaccacccatgagcgcgaagttcgccatatcgtccttggcaataatnccatggttggcgatgaccttgctcgtgttccgtagctggctcatcaccatccccttcagacacttgaagtgcttcgacatcgtccgaagagccatgaaagcgaagggggccgcgttgctcagccctgcaacagtctcgaacgaggagatcacggattgcagctgctggtagtact

SEQ ID NO: 95 - DNA, 933 bp, Zea mays
  misc_feature (122)..(122): n is a, c, g, or t
cctgcagcttcacaatagcctgctcaatttcattcactatttgatcaacaacttccagagctttagcagaagataatgaacgtttctcagcaactcgatcaaatccttctctgaggctgtcnatctccattagcagctacctggatatgaccaaaaacatcatgaaccaattatttcatcgagaaaatcactatcaagaacattagaaagatgaatcacatgaaatcaaactgctaatagatcactatacttttgaaataaaaaatcacagcataggttcaacctttaagaattacaaatgatgcttgaaaaggatcaaaatgaacatggaagctagcccaatgcatgaccgacctcgtcagcagataggatagcaacttatatggtctacaatttaaacagcagataagtttgcaaatcacattaacgcagnaactaatatgttttcatgaagaatatttaagagctactagaaaggtaacaaaccaatgaagtaccaatgttcttaaggttagcaaggcaagaaatggtaacaaatgcctacgtgacaccatacacatcataagaaacagcaaagcagaaacagnnnnnnnnnnnnnnnnnnnnnnnnnnnnnnnnnnnnnnnnnnnnnnnnnnnngtcaaatggcttgtttaggactgaatagctttgttgntgtagtagtaaaaagagaagggtgaaggagaagagatggggctggcccagacaatactgttagcaataatcttgttcatgcatggacgcgtccaggttcaccaagtgtctaggttttttgctatcagtgctacctagaattcagcttatacataataacggacagatcataaagacattgaatgcattaattaggagattattatataccatacttgaatgtaacattcagctctgtataaatagaggaaacagaaaa

SEQ ID NO: 96 - DNA, 997 bp, Zea mays
  misc_feature (85)..(85): n is a, c, g, or t
tgctcgagcccctccagtgtgtggtgcccgcctctgctccaggcctccaccggagtccaccctccagcgcacggccgtcctccantgatccactccgcctcctccattaaacttcttcagagtgaattagtttagngtctgttctgaacttcagaaatcagaaattcagagttcagacttcggcgtccagagaacttcagagntcagacttcttctgagttcacagtttagactttcagagtctgttgttctgctacagctataatatattgttgtcctgctacaactatagtgtactgttgtattgcagtactgctacagctatatatattgatatatatttatacatatgatcttgatataggtggacgactannnnnnnnnnnggagtcgactaggctcgantnatcgagcaaatcgatgactaatcgcgattagtcnccttattggtgcttaggtgactagggtcgactcgccgactttaaaaccttggtctccataccatatcccacatgtatccattatgggatacaggngcgggtgacctgatgtatccatgcagctcatcataccatatccaagaaaaaccaatatgcactgatatcctgatagaaaatatcaaanccaccatagtgcccacttaatctggatttgccacaacaatcctgcatccataggtattctaatcatttactaattgctatatttcatagcaggaaaaccacaagctgaagtgtttctcagtgctctactgatgaatcattaaaagtaactttgagaaagtagcataattgtaagatttaaatcaaatgaaactacttcatgtttgctattgcatacttggaaaaaagatataaacccttgattatgcccccacccaactatccacagtattataaattaccaacacggtcacttggaagtaaaagaaacaaaacaagacaacacactatctaatcaagcatagtaccccagtgaacaacaagactgcaggcat

SEQ ID NO: 97 - DNA, 890 bp, Zea mays
  misc_feature (136)..(136): n is a, c, g, or t
aggaatgccatgacacgcaactggtgacagcaaccgatggtggatggatggacatgccgtcgacctttccgctaattaccaacctgtcctctaagcaactggtgacagcgagcaatccagaacggatagagtatancacttcacttgctttgttttagcacgtagtagtatttttttcccctatatctcgttgtgtatgtatgatgatgacgagtggatctttagctggtgtctggctggctggttgcggcacagcgaatggatgtcgtgcaggatcggagccaccggacccctttgtttatcgttgctgctatgctcattgctccgcattgtttagtacgcgtcggcacgaagcttgccgcggcgcgacacgcgcaaaaggtaaattaatctcgcggcgaggcaaaatcgctttgcccggagcccccccnnngacgggcgacggcgccgggagggaggacggacgtgaactgctnnncctcccggtggctggctggcnnnnnnnnngcgacgccattacttggtcttgtacacatcatgtccgtagtcgagctgtccatcttacttgcaataccatgctgggcctacggtgcggtgtggtgcagctaagcttaggggatcgttagtggattctcgagtgctgcgtatcacggcgaacaacatgtgatgcgatcagaccagagtttcgtacgtcgtgtggtgtcagtacatggaggatggaatcacggaaatggctgtactatatatcctcagaatctgcgttttttgttacaccattgtcttatgtatacagcggccgggcctacggggttcactttgcgctctgttnnaaaattctgcctcgaccacaactccagatgcagactggccattagcgtgcgtgctggatt

SEQ ID NO: 98 - DNA, 840 bp, Zea mays
  misc_feature (31)..(31): n is a, c, g, or t
ggcgtaaggtaccaatgctcgctccgctgcnacgtttccgtntgtccttccttgggataccctgcgtctcagtgttgctctggtgttggtgccgtagctattcggtggggaagtcatcggcgctgccgttcaggaagctctcgtcatcagacttcgacccgcgggagaaggtntggacgcggttcccgccagaggggtccaagctcacgccgccgcatcactccgtggatttccggtggaaggactactgccccgcagtattcaggtgccanactattgctctgctgatggntctggattttggtcatttggagaagaacggcaatgttttatgcgcatggatacattgcctgactgctgtgtgatgccaggcacctgaggaagctgttcggggtggatcctgcggactacatgctcgccatctgcgggagcgacacgcttcgcgagctggcgtcaccaggcaagagtgggagctgcttcttcgttacacaggacgatagattcatgattaaaaccgtgaagaaggcagaaatgaaggtcattcgtagtgacaatttgattttttctagactaaatatcttagtttatgagctagtcacatgcatgttttgagttttcttcgaccactttgatcatctttaattctgtgcctgaacgatttctgcaaaatgaaatctgtcgatacaaaatagcttttgtgatacccatttttagccgacacctgctccttttttttttctgtctctagaagttagattcgttgtccatctttattatcccaaattcagcttgtttgtaggtaacagtattcattgttattagttggcgacataaacatna

SEQ ID NO: 99 - DNA, 532 bp, Zea mays
  misc_feature (3)..(9): n is a, c, g, or t
ccnnnnnnntccagcctccgccgccgcctacccaagggcgcttcgaaatcgttatcaacaacgacaacatccgaacgctcgacctatcgccagtcgaagcagcactcggcgacttgagctccttgacaccaggttcacagccagcatacgcccatgccattagctttaacttgcattcaccgaccttattacagnnaaatttatgtgcattgttcagctgcttcaagaaccctgctggaccagaccgtcgggttcaccatcagctacgagagggaggacgagtacgacacgcgggagctgtcggagttccccgacatacggctgtggttcgtgaggctcgacgccgcgtacccttggttcccggtcgtgctcgactggagagccggcgagcttgccagatacgccgcgatgctcgtccctcaccaggtgantatttctcagaaaagcttanatnnngcacatacaagtcttgtgcngttgtgcgccctttgaaaactatacatgcggtgtacgaaacggtttttgtggatgg

SEQ ID NO: 100 - DNA, 507 bp, Zea mays
  misc_feature (308)..(308): n is a, c, g, or t
gcgcgttctccatctgctcgcggtggtacggctgcccgcactgcggcaccgcgcatctccactcctggccctggagtgccgagtcgcggcagagatccaggtcccggcaatcgttgcaatagctgccagtgtagttgtttcataaggtttgctaacatcaagattcaagagaaaaaataatgaaaatagctcttgaatctgtccgttatggttctaagattggaacaatttccagctaaagcttacatttacaaattaagcagtaaggtgagaactggcaagaggagcaagaaacactatttggttcngattctctgattaacattntcctgtttgttgntttttntnnnnnnnnnnnnnnnnnnnnnnnnnnnnnnnnnnnnnnnnnnnntccagctcatgtgctttgatgcaagcacaaagataaatagctaaaatactatcacaaacgatcatgcaaggatgcaacaaactttccacaaacaaatctggttcatagataaaata

SEQ ID NO: 101 - DNA, 669 bp, Zea mays
  misc_feature (41)..(41): n is a, c, g, or t
acaaatcactcgctactcttgcctacaccctcacaatcatntacgaatactagtgactgctttcctctcctcagcatttttggcaagtgttgtgctggcgtgccgtgtgtggagaggaacgctatataaagcaacgtctaaaaaagaaaaaaaatactatatattagcatactagtatataaatataagagtaactccaatagttttctaaaagactctctaaattaataatttaagtaactaaactaaaagctcctctccaacggttctctaaatgaacttcataaatttagctactnctcatctaaccttattttctctctacatttagnaacnatttaccaactncntaaacaaaaaaaaattgacngtaatttttgtatttcgctgcctttttcactttatagtaacgatatattaacatagcccatgcgtcgaacaacgacagtcagctagagattaaataattgccaatacaatagccgcacgtncacntgtcggaaataaataaataaacaattgcaacngtaaatnaaaagatcaacacaactcaccaagttgaatatgccatcgatnatggtcccactcagatgagtgacatgttaaattttaacatatttagaaagtaatatatatataactnntnnannagatgcgttttttnnntat

SEQ ID NO: 102 - DNA, 707 bp, Zea mays
  misc_feature (86)..(86): n is a, c, g, or t
tatcagcaccaacatcggtcttactgggtggctgccagcctcaatagtgaatctctcgacgaccatgcaaggcctcatgttggctnacacagggatcgtaggaagcatccccttagaaattggcaatcttttcaacctccagttcttcagcgtgtcaaanacttccgtgtctggtgcggtaccngatagcattggcaagctggtaaacttggttgaattanncctgtacaataccaatctntcagggcttataccttcatctattggaaatctttcagagttagctgcgcttgntgcattcaatagcaatctggagggaccaattcccaaaagcatagggaggctgaagaacctctatgcccttgatatntcaagtaaccgcctaaacggttcaattcccntcgagattttccagctaccactcctttctagatacttaggcttatnacataattcattatcaggtaccctacccgctgaggttggtagcttganaaaccttaacatcctnncgcnnnctngaaaccaactgtctggtgagatacctggtagcattggggactgcaccgtgttgcnnnaacttgggctggatgacaacttattngnnnnngccatacctcnntctctgagcaatatanannncctnnctggannnnnnnnnnnnatgnnnnnnnnntctnncgtcatnnctgnnnnnnnnnnnagca

SEQ ID NO: 103 - DNA, 777 bp, Zea mays
  misc_feature (543)..(544): n is a, c, g, or t
ggagcaccctgccgcacaccgcgcaggacgcgaactcgccctccgcccggaggcggcggacagcgccgcagtggccgcaccagcacgtcgccgacggtgccatcgacacaacgccggaggaacgcgtctggccaactcaagatggatttatgtgccggcggctgagttttgcctcgggttcagatgtgccaggactacattatatttggatggaaaggttgttgggcgcttggctcggtttcagttgggctattgcatcgcgttggtttcccgtggcagaattcggaatgtgccgccgcgtcgaattcggtgttccttatatgtagtgcacggacgagtcagcatcgaggcacttccaagaaccgagtggaggaaacttgacgtcatagatgaagatttcctcttttttttgtttaagaaaatagctgaaggtttcttggcagccaaggcccaagtttcctgtatttacaactcaattgaagattctgtttggttcatcaccgtaaaataatgcagaaagatccggtaacccaagtttcctannaccaggcgtaagatgcaagctagtactgtatcatccaatgcgtcccacgaagtnnngctggaaaccagtcgctcgtttcaaaaatatcattctaaaaaaaataaaaataagttgattttannngactttcgacnnnnnctcnnnnnaaagtaagatctcatacnnaanngnnnntnnnnnnnnngatntatcaatnntnnnnnnngcnnnnnnatatgcatttgactcntgna

SEQ ID NO: 104 - DNA, 537 bp, Zea mays
  misc_feature (465)..(465): n is a, c, g, or t
acaggacaacggaggtcggaggccccttccaagcaagcaatgccaagaaccaacaagaacacggcaagaactagcatcactacagttgcaacaaatactaaatcgccatcgatctcgacggcaaattatccgctaatcatcttccacctccagctaagttctctgagaaccatcattgcaaaatgtgctgccacaagcatgcaaacattactcccgccataagcatgcaaacaaggacatcttttcttatctctcttgccccccatctgtcacggcaatgggcaaaatagttcgttctcctgctcaccgacgcgagccaatgatgctccttgatctccattctccaccgccaccgaacggcggtctcgccataggcaccgtttccggcacggtgcgtcacgcggcgcaggtggacggccaggcgtcgcggctctcctcgtcggggggtagctgatgcgggacctngaccggtggcgcgaccccggctgacgccgacgccgacgccgacgccgnngctgcgcgcttgggggcccgacc

SEQ ID NO: 105 - DNA, 707 bp, Zea mays
  misc_feature (563)..(564): n is a, c, g, or t
atcgccaagtacaggcccaccatgcctgttctttctgttgtcattcctcgtctgaagacaaaccaactgagatggagtttcactggtgcttttgaggtacgtgaaccccaatattctttgtgatctttcctgttagatgttttctttttatgcaacactgacattatctattattagcatgttgatcatttcttttgtgaatggtaacattggtctttgaagtttctcttgtagatattgaacatgtgtcatgcaatgcttaagagagattactttatttgagaagaattaagaaccttgattgccttctcttgtactgatacttggcaccatgcaactcatttgttgtttctttttcttgcaggcaagacagtcgctgatagttagaggcctctttccgatgcttgcagatcctcggcatccagtaagttaactgaagtctttcagttcactactttgtataagcaacacatactggctgcactgctcacttgtacacatgagcaactcatttctagcatcgttggggatggaaagattttgcaaacacatccccattgccnntgtattcttggacaagcattttttttattcatctattttggtnngatatnnnntgaaaacttgannnnnnttgnnnnnnnnnatgnnnctnnnnnnnnnnnnnnnnnnnnnnnnnnnnnnnnnnnnnnnnnnnnnnnnnntca

SEQ ID NO: 106 - DNA, 784 bp, Zea mays
  misc_feature (68)..(69): n is a, c, g, or t
ggagctccatgtcttgccgagatcatccgtaagatgggtagcgggctcctgtgtgcgtcgtcagttannggatgggaagattgtaagaagtctgacgtagtaatttagtttgattgttggagaagggattaaatataatcatgcnnnannnatgtgagcgaacacttctgtannncctgatgatcacggcaatgctggagccaggaagggaggctggacgacgannngacgaacggcgtccgaggcagcgccgccttcgtcgtntcgatcctcgcggcggagcagtcgccgccgcagcatctggagatcagcttgctgccgnccaaaagcggccagcaagacgctggtccgttcccatccgtgtcagatgagcccgctttcatggactgattgaccgtgcctagannnnnngcactgcggcagaaagaacaaacgggacggaataagattccagtgtcattncacgactccacatatccatagaatcagctagctgatatgctagctttgccggtgtacgtccagtaccacacgcattnnntttggagttttgtactttttggctatagtaataaaccagtttcttctttttttttggtatcggtaaaaccatagagctgacaatatagtaagcaaagtgaaggcaatatatgcctnncnngtatacaattttgaaccnnacnnnaggggagtgcnntnnnnnnnnnnatgtgnnnnnnnnnnnngannnnnnnnnnnnnnncnnnnnnnnnannnnnnnnnnnnnnnnnnnnntnnnnnnnna

SEQ ID NO: 107 - DNA, 726 bp, Zea mays
  misc_feature (501)..(502): n is a, c, g, or t
aagagccggattgccaggtgcttgtttcgtttcgggctgtgtaggtgctcggatgctgatttttttcttctttcttttgtgatgaattgtactgggccgatgaaaccgtatcggattgggttctctgacgaggaaatacataactaataaatacacatacactgagctgaggttgaccttgcacattcctgttgatagagtttatattgctgtcagcaggttcagattagtttgtgttgtgctagtaactaatgcagagcacagcgagtagcaattaaaactgtgagttgcagaggagagtgggtgtattggttgctcaatcaatctatgttggagccagtaaacattcttctctccctcctgcactggctctagatttgttccttccatctctttcttaactcttgcctgacatgattttacttcattcacgagttgaaactttaatcagtataaggcagaagccaaaatgatcatgctagctaaggaacaactgctctnnatgaaattactgtttacatggcattagcatcagtatgttcaatccaatatgattgttgctttctggaaattcngctatgatatacatcactccgattgtctttatagnnnnnttnnncnnnntctgttnnannnnntngaagcanttagttattttggaagtctanttgatgnatggatnnnnatctttgacatattnnnnntcnnnnnataattttgagtctc

SEQ ID NO: 108 - DNA, 696 bp, Zea mays
  misc_feature (79)..(79): n is a, c, g, or t
aagagcagagtctcgttcaaaggctaaggtcccccaagacgaagaagagagcggtgatgacgatgaggatgaggaggcngacgagcacaacaataccttgtgtggaacttgcggaactaacgacagcaaggaccagttctggatctgttgtgataactgcgagaagtggtaccatgggaagtgtgtcaagatcacgccagctcgagctgagcatatcaagcagtacaagtgcccagactgcaccaacaagcgggccagggcatgagcggcaancatcggcatcgggcgacctttgataggaaggaagaaacnacctcctggcttcaagatccctagatatggcgtaggtccctagtttacgangtctgcattttcatgtgttcattaatatatctgctgattagctgttcgacctagcagtgtctaaaacgtgccgtgtgtcttctgtgctgagtgttgacatggggcctcctccctccagcctgtaaacgctgtcactgaaggtacctctcaactgaccatncttcattgttggtgtcgtgcagtgagnagtggccgtactcntnnnnnncagttcctagcatttgaattgctcaaatgcgtaccttgctgnnnanggccnnnnnaaaatgannnnnnncnnnnnnnnnnnnggtcnnnntnnnnncnnnannnnancaccactgnnccantttt

SEQ ID NO: 109 - DNA, 755 bp, Zea mays
  misc_feature (1)..(1): n is a, c, g, or t
ngcctgcagntgcttaaactcaccgaaacaaatatatcggtcccagctggtatttcatatccctcttttgctccattgcacccgcctgggtaaatagacgatcacggcggcagagttagaaggcatgtaccagcgaaggcaacgcaccctgcttgtcactaaactaattcattgaaaaatagttgtgtgtgctgatcgaaggatacaatttgcnaacatccatccacaagtatagaaaggctgtcatnnnnncaccaaggtctcgataaacatattaccagtaacaagaacagcgacttcaaccaaactagtctaaatgnnnnnnnnnnnnaccactacacaccntttgtattgctcaggaaagaacttacgacattacagagaagannntggtacctggcaacttgtctggccggagagaacgcctgattaacaatggtggctgaggatacaagcgaagagcttcaagaataatcagttttatgtacctagaggncaacacaaaggtttcatgagagatgcaaattcatttcaaatgtatagatatagctagtaatttcagagaagtcggataccatggatatgctactgttaataatctgataatattttttatgaaacaataatccttattgataatatttttatgaaacaataatccttannnnnnngcaccatacacctatccctcaataaatggaaaatannnganncaattctagatgnntnaaaaatnatgnnnnnnnngacatact

SEQ ID NO: 110 - DNA, 740 bp, Zea mays
  misc_feature (10)..(10): n is a, c, g, or t
accacaatgntagggcatcatggcaccgacaggaattggaacaggtacaaacccatatgagggatggctcatactgtcctttctatgggaagaannnctacttgcaccagcatcttccctagcactactggagcaaatgatgtcagcaccctccatgctactgtccagagtaattcgagcttgccttgtactctcacctgtattatggtcattgggaggcagcaagaccccattgggttggaaaactgggtccttgccatgaacatgctgctcttgcccaacaatgcaacgagaaggaagaaactgtatctcacctgatgattctatccttctattgccatatctgtgtcatgttgaagccatggacaagttaaataaagctagtcagagttatgcaatacatttgagcagttactaacatcgacttatcgccttatcggtctatagaagtactaaacatgtaacctcataaagaaacaaatgggcatggacagaaagccatgcatttcagataatttccagccacnttcaagaactagatanccaatcaaaattttagactaaagttnaaaaaatccatcagcacaaagtacctcgannaggcagaagaattcgagtgattaaagatatctttttccctgaattcttgattcttgtnancattacgatnctnnngntttnncnngntggtctcgnnnnnnngnnannggtnngaancnnnatgacntannacagcactc

SEQ ID NO: 111 - DNA, 785 bp, Zea mays
  misc_feature (22)..(22): n is a, c, g, or t
ttgaacgttccccagcgccttntcgccgtcgtcgatgagtagcttctttcggagggccagcagctcctccccggcaccgcagatgtaggccgacgttcgcctgccggagagagttgctccttccatgctgtcaatgtagtcctgcatgtacagcacaaataagaccaatgtaccatgtcctaccgatgtttgcatgttgcaagataggaggaaaaaaaacattccaaggtgttcagacgccatgaaattgaaatgaaattgaaatggttgtttctgatggttttctgagccattggttaaaacaaactcaaggcattcagacattagttttctgggcctgttgatggctgtagctaggtcgatgtttttnatgtacaacaatacctgtttcgtgtaagatccagagaggaagaagtttttaacgggcgtcttctgatcaggcctgaatgggtcgtttccaggagcctcacggtacagcgattgtccgatctttaccacactggaccatgtaacttccaagccccgggaagatgggaacagttctacaacctgtgggaaaattacatgtgtttattactgangtgagctttggaagaatacaaaataacataagcagaagaaatctctctnttttnaagtgcattgaaatggggtcttgctcagntatgtgtaaagggaacagcagaaatttgancnaaacagaagctatttacaaacctacctgcttttgaaccttactaatgatctcctcgtttggcaatggcatgtatggatnnnnaggagtc

SEQ ID NO: 112 - DNA, 845 bp, Zea mays
  misc_feature (650)..(650): n is a, c, g, or t
acaaaaccgcacccttttaatttgttgctaggccaggacacacacgaagcttcttacaagagaacaaaatagtgacacgaacaatatgcttattagctaattgtcttcgttgcataaactaatgctctatcagatacgtgcttccccaaatgaaagtggaatacgtactattctgtcaacaaaacacggtgcaagtggtgagatatgcagtgtggaacgaaggaaaataatcagaccggtgaactgatgagagcacgcgtctggaaacaggaattcggcgaacacaccactgtagtttgatcattagctagataacccgtttcttatattccacaattatgaccttttgtatcgactgagctataccaagtaaaaaacaaaacctgttcatttgagcacgttgtggaattagttaactaaaccgcatggctgaatattcagataggccaatttcttattccataattctgacccaattcgtacttatatcatatgaactaccaaccaaacaaacagcaagcgtcaatgcaggacttcattaacaagctagtgggaggatcagctcctcctcgattggctgacttttcgagcaaactaagtcatgcttcttgcatatgatttccatctcaaaaaaagcccatgcgtctacnaaaccattaatttggcatccagctcaacaacaaaagtcgcaacgctgaactcgaattagtcttttagctagcttggaaccttcagaaagagtgcgcatgcatgaagttaaactatccccagcggaattaagcacgtttcagaatacaaatgccctcgaccaacgcacaagactagcgtgcaccttgcccatcagt

SEQ ID NO: 113 - DNA, 770 bp, Zea mays
  misc_feature (3)..(3): n is a, c, g, or t
gcngataagtttcaggaataaaggggtattatgcttctagaataaatttaagangatatagctgaagagatgctccaaacatcactaatgtatttttaatataggatatcctncntcgactatatatcaatatcatagcaagtccaataaaaaggttgtagtagtcacaaatatatcatatttgtttttacagatttccatgttaagtttaatatcaaattcatatccaacatactacaagtgctctctattctacagtaaatgatagtaatcagatacagaggntctgttaccaattaataaagctataacatttggattagtaatcatatggaatgtaagcaacggatggctgaatataacatgaactcatataatacttggccaatgggggaagaccgcccccacagtctttactccgagaaacaatgtaagtcaaaccaacccataaaacttctganccccgcttcaccctatggagctgcattgtaacctggctctgnctcagacccaactcgatgggctgccaaccactgcaactacccaaaaaccaaccacaggtgccacaatcccaaatcctcctactgtttttaaggttgccatcgaaggaattgttttttggtgccatatactagaagaattcaagtacaataacaacaaagccttttacggggtgtttggcatggctccgctttaacccagagcaactctgctctaaaactccaggatggagcanntctgaagtttttttggatttcggagcagcanat

SEQ ID NO: 114 - DNA, 883 bp, Zea mays
  misc_feature (747)..(754): n is a, c, g, or t
taactagcaaagctaaataactttgtccatgttgtcttagtattgttctctgtgtaattttatattctgaaagtaaggaacatgtgcataagtactctcaatttagattgtagaatcatttatgcctttttcttgagaactacatccattgcacaatcgataatgttcctctcccgttgaaatattggaccagagaattatggtctcaagaaatgctgttgttggtgcttcttacggccttctctaccctaaataggtgaatggtgtttggcaaacgaagcagccgaatatgatggagctgtgcactgaagcgagaaagctaaaagagaacttccattcctttgagatcatccatgttcgacgggtatgcattgcatttatagttttacataagcttgcagactctttggttttgcatcatagacattattggtcaaaacagatattctttgcaccacaatcgatgttatttgcaagtgctggccacctgtatatgctttagttaagcaagctaccacagcaatggttgctttcatcagattgaaagaaaataataaaaaaaagaagcattagtttctattgttcttctaagtgctctgctgattttacccctttcctgcttctgaatgaaggagtggaatgctgaggcggatcgtcaggcgaacatcggtatcacgcttgcaagtactacacaacatccatcatccctcaattccagttttcttttcttattttcataaaaatcannnnnnnnctgactacgctacattgcaggtggcgccgtgtttgaggagcgtggtgacatctgatacatagctagcaacgtatgtagttaactaactagcttgacctttgacttgcaccttngtcgagcnnctatgct

SEQ ID NO: 115 - DNA, 891 bp, Zea mays
  misc_feature (139)..(139): n is a, c, g, or t
ttctcttctagtaatgaacagtcagtgatgagagagagagagagagacagggggcacaccgggtcttgttggcgtcttgctggttccgggttgggacagcttcgccttcgatcgatcatgccatcctaattgactcctnattcggcgtctttcacttttattttttttcnngtaatgttgtacgaaagttcaaatgaaagggaggctatcattaggcagcgggcaccaacagaccggcagcgctagcagacagacacggacagagaactcgattccacctttcctttccatggggcaaaaataaaaaaaactgatcactcaacagctttatttgttgcacaaattgtgtaaacaatagtattataaatttataatgtgtagaaacaagacgccagccgtcccatgcaacgcaaaacggcagcgcagtgcgtacgcgacacacacaggcccgtctcaaaaccggccggggtcactgtgtggtggtggtggnnngcgggcgttcggtccggcgtcaacctcagagccctctctttaaatttngcctcnccccngtcgcgttggatccatcatccgttcccgatcgtccgcgctgacgtcgacgcgcgccacgattaatcaagctgacagacgtactgaagctgcgctggctgctctctgtttggaggatcatcatcatcaaacgaatgcatgatgaaggccggaggaggctgagcttatattagcgagtagttgacccgagcccgctgctgcactgcccacgcgcgcacgggtgcaggaagagcggaggacacatacggtttgctcaaggagagccgcctttgactcccatcccatggctgatcatatcgtcgacaataatgtgctcgcgaatgcgatagtgattctgtacct

SEQ ID NO: 116 - DNA, 866 bp, Zea mays
  misc_feature (17)..(17): n is a, c, g, or t
ataagcgcccagttgangccctcaaagaaggggtgctgctttatctccgcggcgcccctcttcacgcccagccggctctggggctccttggccagcaggcctctgatcaggtccctgctcgcgttgctcgtgccggggcagtccgggaacctgagctgctggccgacgacgttgaacagcgtggcgcggttcgtctgccccttgaacggcgtcttgccgtacatgagctcgtgcaggaagatgccgaacgtccaccagtccaccgcgctgccgtggccctcgcccttgatgatctccggngccaggtactcgtgcgtccccacgaacgacatggaccgcgcccccgtcggctccacgacgagctccggcaggcccgtgggagcctgctgctgcctgggctcgccgcccttggatttggatttcttggcgcngctgctcttcttgctcctctggccgaagagcttgggcatgaagcatgtgggctggatgcangcagcctgcgcgttcctggagtcggagttcagggaagatctcaccagcgtcggcgacaccgcgcaacgaagggagaggtcgaagtcggagatcatgatgtggccatcttctctcacgagcacgttctccggcttcagatctctgtagaccactccaagcatatgcaggtactccagcgcaaggagcacttctgcggcgtaaaacctggcagaaaagaanaaaaaaaaagagagagatcatacaccncagaattggcgaccattctgtttattcgaagtttcagactccggcaaagaatnnnnnnnagttcaagaagcaatgatgaaaatgcgcnaacgatataggagtaataaacaatgtgtctatcctaatct

SEQ ID NO: 117 - DNA, 823 bp, Zea mays
catcatttgagaaccagctccggccaattgatagatatgcaatgcgctttatggaactctgggatccagtaattgacaaagctgctttaaatcatcaagtaaatgttgaggaggaagaatgggagcttgatcgtattgaaaaattcaaagaggatttagaagcagaaattgatgaggaccaagaaccactttcttatgaatgtaagtactttgtgtctgctataatttctttcttggcactttggagcagtctgttcattatcactttggcattctgcattttagctggagctttgtgttcacctcaatttaggtatccttcctttttatgctttagtaatattgagttattgacatcatctttatttcttgcagcatgggatgttgattttgctacgacagcctatcggcaacatgttgaggctttaactcaaaagcaggtcgttattgacttgctaacttgtattcttacgtgtttcacttgaaacacagtgcctttcttctctcttctgacttgttataatacgtgcagttgttagaagaacaggaaaggcaagctcaggaagcaacaaaagagttggaggagaagaatgataatatgaggtactaattggaaaattattttcctgaaacacgaacaatggttgcaaattttgttttgtgattgtctcagttgctctttcttatatgtcaattggcaatattagcatgttatgtgcccacattatttctaatggatgtttttctgtctgttgatttttttttgcttagtctatagcttccctttgatatactattgagtattgactgtacattttggcat

SEQ ID NO: 118 - DNA, 628 bp, Zea mays
  misc_feature (467)..(467): n is a, c, g, or t
caagaacctttccaagaccttgaaggacgtcgtctggaaatccgacgatcttcaaacgcaacatgcttcttagcagaacctcagagaggaggaatggcaaactattttgtgcaccatcctttcaagcaaactattctgcatgtaacggtgggtgcccaggaaattgcgctggtgctccaccatcgttgtatatgctgcgtcttgagttgcttgctgtagctgaagggaagttcattcagtaggttttgagtatgagttttgtatatccgtatatgcctatgactatgagccgtgctggaacactatgatcagaaactgcgaaatggagcccagaatgttcttgccatcttgctccagttctcagcaccgggcttaacttgccttgcattttttgaagaagaaaaatggactactgaattgggaactcgtattagtactcggccatcatttcagtagtcttttttttngntaccatgtacaccgcgaatcatgcaggttcttnctttgcttgctgttagttacattnggttctcctgacagctagaaactaanntggcagnntgagtagnnnnnnnnnnnannnnannnnnnnnnnnnnnnnnnnnnnnnnnnnntgncncannnnnat

SEQ ID NO: 119 - DNA, 1082 bp, Zea mays
  misc_feature (11)..(20): n is a, c, g, or t
tgcctgcagtnnnnnnnnnntctggtcagtgcttaaaaatggaatagtatatatatacatatatgcgcaaaaatatacctcctaagagacacgagataatcctccttggatatcatctgcatctcttcaaggtctcttgcatagtcagatacctaaacaagcaaacatataatgaacataagtctgaaaaatataacacagctctaactgactactgtgataaagcatggaacagagatagcataatactctacattggaactcacgtacagttatcacttcaagcaattctatatgttgatacatttgttttgtcacactaaacagaagatattgttgtttctattgtacttatcagtccaccgacttgaatcttccaaaaaaaagagaaaacattgtactcaagtacaactgtgcaattatgatatgcagacatccatttccaaacatcactagaaaaaacaatacaatttatgaatagaagtacatgttgggaagtgtaacttcttgcccatcatttcataatgtgaatacaaaggagtacagcaggtacttgtgcaagcatgatatatcagcaccactgtcaaattcaaaataaaggacaaaaagaaggcaacaaccatgttgtagacacatcttaaatctggttacagtctcaaaacctccaatttggaaaagaaaaccatgttgctcaatagcttttgtcaagatttctttgcatataagaccaatgggtcaatgttagaaacaaagagaagaattccttttaccaaaaatatgatacccacaagatgcttccttaattaaacaatttctattagcggaaagctgatcttaaaatcttattgatcaatgttagaaataaataagggtcgttttacaaaaaaaaaagtttccacaagatgcttcctgaatgaaacaatttctattaacagaaggctgatcttcacgtcaagtgcgagaacttttacagcaatttaaaacacgagtnnnaagcatnaccacacaagtaaaaatgactcactgggaaatttatttgtgttccagctccccannnttttaatgcagcaaggtcataggcc

SEQ ID NO: 120 - DNA, 671 bp, Zea mays
  misc_feature (1)..(3): n is a, c, g, or t
nnnacttcggttggactgcctgtactgccgtagcgggctagagcagggagagacaggcagcgacatgttcatccgaacattggatgtgttcctggacgaaagagagatcggttgagacacagttcacaatatgtaaattgatataaatctgacagaaggagaacatacaatgctgcgatcctgcgacttgatgtcgaaggaataggggatgttggtcctggcaagtttcttccaaggatatcaggatctcttaaaggagataaacttctgtatgacgaagtttcaattttcacctgcaagagttgtggtcagcattctgttcgttgccctggtcacttcggtcatattgagttggcaaaaccattgttcaatcctttgctgtttatgagtctgaaaaaccttctccatgtcacgtgtttccattgtcacaagttccgcntgaacaaggaacaggtgagttcattttcatcttgctacttaggttttcattttgggctaagtttttgcatgccactgcacaatcttattgtgatttcactttcttaggttgacagatatgtgaatgaacttgaacttttagtaaagggtgatattgtctgtgctaaaaatttggaagactcagtagaagaagcatatctttccaaggaagatgaaaacatgaacaagaccac

SEQ ID NO: 121 - DNA, 514 bp, Zea mays
  misc_feature (49)..(49): n is a, c, g, or t
cctttctaatgtcaaagttctgctccttgcgctctccctaaacaagcangaagtatcaagcacaaaagtctatatctagaacaangcatatgatttaaaaccatcagatgccagccagatataagttacctggacatagatatcgaaaaccctcctccctcagcttctccaagactgcatgtctggaaagtcaacctctgcaaattgaagtgtgaccgtgtaatttccattctcaagtccaatgccatagtatctcagngatgatggngacatccttgctgtttggaacagtgcagagtctagggtgttctcgaactggcgtgaactgtagatgatgtaacttgcattgggtgcatctgcgtccaagaataacccaacgctgctaacggcccatgtaggtgcacccgcaacatagtangatgcagcactaaggttggcattatcagcttgatagacngaattatctgaacctgatattgctctactaccaccacagtccaccgcaaaggacgca

SEQ ID NO: 122 - DNA, 485 bp, Zea mays
  misc_feature (279)..(279): n is a, c, g, or t
catgggtacaaaaaggctcccatttcccagtagggtgtttgatgttgttcattgtgcacgatgtagggtaccgtggcatattgaaggtactttctatcacgagaatcctgctctagcacaatacagtccctttgtgttagctgcttctgtgacactatgcctgttagttatttggcaggcggtaaactcttgcttgaactggacagactgttacgtcctggtggttactttgtgtggtctgctacacccgtataccagaagctgcctgaagatgttganatatggcaaggtattccatgctttaatttcttctctacagttattacagtgaattacgaatttcatgccccaagagttatctttgttctgccttgctgtagccatgtctgctctaaccagttcaatgtgctggaaaatggtcaacaaagtaaaggatagggtgaatagagtgggtatagcaatttacagaaagccaacagacaaca

SEQ ID NO: 123 - DNA, 605 bp, Zea mays
  misc_feature (6)..(6): n is a, c, g, or t
cggcgnggtctcgctaaccctagacaccagcgcagacccgccgctcggcgcctgccggttcatcgacgacgacgcgctcgancggggactcgccgccgtcgccgctagcttccccaacctcagccgcctctcngccaccgccgcctctgagtcnggcgggctcatggccatcgctgtcggatgcccgacgcttcaggagctcgagctccaccgctgcaccgacctcgccctccgcccggtttccgccttcgcgcacctccagatcctccgcattgtcgccgcgtcccccgcgctctacggcaccgcngagggcggcggcgtcactgacatcggcctcaccatcctggcccatggatgcaagcggctggtcaagctggagcttcagggttgcgaggggagctacgacggcatcgcggctgtggggcgctgctgtgcgatgcttgaggagctcaccatcgctgaccacaggatggacggtgggtggcttgccgcgctagccttctgtgggaacctcaagaccctgcggctgcagagttgcagtaggatcgacgacgatcctggcccagcggaacaccttggtgcttgtctcacgctcgagagcctgc

SEQ ID NO: 124 - DNA, 434 bp, Zea mays
  misc_feature (297)..(297): n is a, c, g, or t
aaagttgttttctttgttcgatgaactaaataatgagtaaatttcagtgaactatgtgactggtcctaaagataattttagcttaacccatatccaaaaccacacgtgtgtccatattaccacgaactacgctttgtaactagaataacaacattgatttggatgtggactgacatactgggtccattgtaaaacagttgcagctcacagactaacagtgtcatatactcatatatgtctaggcccaatatgtcattgacatttgaactaacagtgttagctaacacgttcattttntctatcatatgaaatgaacaaatattatgtagcattgagcctttgagaaatttgttcatatttttttgataatctagactatatatatatcgtttagttattaccagtaattactgaagatcgttcagtaattacca

SEQ ID NO: 125 - DNA, 328 bp, Zea mays
  misc_feature (26)..(26): n is a, c, g, or t
acaagtcatttcaacttttcgaatanaatttctcgtaagatggattttgttttctcgatttgtaagatggcttgaaaggacgtggattccttcgtagttggacacaacgtggactcctttctgttggatatgaatgttaatccatcagcaggtctnnnnagtgctaatcctaatctccttgnaattacaattaatattccntttgtatatagnatgagtttacagancggtgtgcaangtgttttccatctaagaagnnncagggagtctcagtccaagaaagagaagaatnacacggtactggtaatgcacttattagcacctgana

SEQ ID NO: 126 - DNA, 517 bp, Zea mays
  misc_feature (3)..(3): n is a, c, g, or t
tanttttatattccttatgtactgttttcaaggatattgcctcatctttcaatttatttcatagaccccattttttttnnacagntttgctattatttgcgacagttttngttacctaagttatggtctcctgagatctcaagtttcnaacaacggttaaatttgccaatttggtatcctgttaacagatctttcntggggggcatgtcttttttgttttttcttgttcttatgctggctgacattaccaatttgctatcctttaaagccattactatctggggcattctggntctttgtgtttagtgcantgattctggtgaaaatgtccttaactgagttctgntgattattagacttcccctggttcatggtgtctcacaggtatctctgatgtcggagttgccaaattgggagatggactgncatctctacagtcccttgatgtttctcgctgcataaagctgagtgacaaaggtttgaaggcggttgcactagggtgcaaaaaattgagtca

SEQ ID NO: 127 - DNA, 466 bp, Zea mays
tgacgcgctcattatatatccccattgttggattatgtacatgatggatgtcaataatatcatagtgggctacactattttacttgtggataaaacgttagcttctgccagctccgtaagcaagtgtggcaagcgtttgtgcgcctagtctttcattcctaataaccaccctgcttctgctcctgcccatagctattctctctcttgcttgcttgctgtgttgcctttgcctgccttggcctgagactgtaagacttcctgctgtaaggaaccaagcatcagcaagccagtcagtttccatgccctcccttttcgttcttagccatcctgatcccatgctaatatgcaatgcaacaaacagggaggacactacattttgtgccacaagctgctcattgttgtttttatttcttttttgtaaagatcgacactacatacacctcttgtacaacaacattctcagtca

SEQ ID NO: 128 - DNA, 466 bp, Zea mays
  misc_feature (34)..(34): n is a, c, g, or t
taacttgtattttggcagtacagcctgattcatntcaagctccatacttacttcataataacgacgaaaatatgaagcgtcccccaaaacccctggatttgtgaaattcaccattgaaaagaattcttccaaatcattctgcagcaccagaaaacattgagtacagcaaattccaagtcaattgttctaggataagcgtgcaattctattttgtggtaggtaggtggagggagaccaaaatgtacctgcattggggtaccggacaaaagaatacgccgtgtacatggaagggcagccaaagcctacacattatgcattttaacgtttcagcagaacaaagccagagagggaccncaagtgtagatgaatagtgtgtgtaaatgacatcagtaaactgacggaccttatttgtcagtgtctgatcatttttcagcctgtgngcctcatcacatatgagaaggtcaca

SEQ ID NO: 129 - DNA, 479 bp, Zea mays
  misc_feature (21)..(21): n is a, c, g, or t
tcaccgaggtcctgccatggnggatatgtcgatgctgctgatctttggaataactcattcagtttcacttcctctctttgctgctatcttggcagaaggagccaatctcaagcttataagcttgtcactcttgatgaatcaagtcagatttnatttgcaaggtcttttgctaactcttnctaggtccagttgaacccattcaagtcaattttcttgcagttctgtggcccatcagtggtatcattgatgttcagatccagttttgctgccttccgcgtcatactaaaacttcctctctcacgcctgtctagttggcctggaggcgtcatgcttagcttgtcaagaaacaaatcctctcttcctctttctttctcttgtatatcaaaggtcatgcctcctatctcctttcctctgtgagcggaaagactgattgacaggatatcttgatctttctgtgagccgtcacaggcagttaagca

SEQ ID NO: 130 - DNA, 519 bp, Zea mays
caagtgaacgtcgtgcccgacttactggattgaagcaggcagaggacataaagaagttagagatgtcagcaacgccgaccacaacagtgtgtatttcatcggtagagcaacagggagctgcttctttaagtgcgaagattaccaatgcttctgtttctgaaggacagaagaatcctggaaattatatgccttctgccatttcaattcccgtggggagccatgttctgggcctgggcgcaacaagtattgaagaaacaactgccactatgataactcaggctcctgcagtttcaaaatcagaacgaagaaaacttccaggaggcagtcaacaaggtattatttattctactcagatcatgatcaattttcttggtccagatgagaagagtggcataatctttgctattattttccatggtaacagtaacagtggctgacatgacttgtacatgttattgttttccctttttcgtaggtattcagtttgagagttcagcatcaaaaacaaagatggtatca

SEQ ID NO: 131 - DNA, 433 bp, Zea mays
ctaaccagacttggttttctgaggacctgatccttaactctatctttgaactgggagtcccgatcatgggtgtaagaatgacgccatcaacttttcttttggtcatttgttttagggtaagtatccgtggtacacttggtataatactggctgtcattgtcacaacttgttctctccttgcactacgcaccaacttatcctgctttttccctacttgacatgtattggtttgtcattcagttgatcaaaccgagcctgattgggattgcattctccggtgatacttacatgcgttcatgtccccatcacagccatctgtacatagttttatggtctctgtcacccatcttcagccaagacgagagaggtaggggggaagtttcttgccctccgcgttgatcaagcaaagagtagaaggaaacgctctttgcag

SEQ ID NO: 132 - DNA, 511 bp, Zea mays
  misc_feature (12)..(12): n is a, c, g, or t
gtttccttatcngaaaatctccantcntctgccttgctgctcgttcctgcagctcagacgtcgcctccnttcnatcccttnnnncncntctctctcngtctcctcagctcactttgccggcagccaggtttgttagccgagcccctccaattggtgtgctcgacgtttcacacngtttcctagccgtccganacacaggcgtccagctcttcaccattccatcgcttagctcctttcctcttnctcggacattgcattttcctagcccctctgcgccttgggtgtctcggcgctgccatcatcaccctgntctacatcttgttgcccagcnatggcgtctctattttcttagttgttccatgccggngtccgtgcttgcntttgccacagccggcgtcatcaacttcctggtgcccgaccctgctgtccaagctctacagatgtggggagacttgcttggtgcttgcttcccggccaccatgggnatctcgccgtcganntatagcntgca

SEQ ID NO: 133 - DNA, 543 bp, Zea mays
  misc_feature (2)..(2): n is a, c, g, or t
tnattttttctgtaggtgggtgcaatgctgtacttgctcattgctatggtaccactggaagcaaccatggcaatggggcggggaatacaagaggaaatcaatcaataaatgtcaagatcaaggggtggctttgactataaccccncttttggtaaatgtctggtggtagtttgcttcgtacttgtataaactgtatctgtatgtgtgcgtcgcaatttgtaaacaaacaggacgagtataagtgttgcccagacatgttagaccanacaaactgaaggctctgaagttgaacataattttgtttcggtgncatcatgctgctctgctcacacagtgggcaaggacagcattggcgggaagccgaagtagggccagcacnnnnnnnnntgccggcgcaccgcgcccggcagatcttctggagctggtcatngataccgttntttnnnncctcgctgacnncaatgangaggcatccgttggctgtgcctnggaggcagagagnccggtggtctgacagcccnggcaccgggatcgtcttctcca

SEQ ID NO: 134 - DNA, 506 bp, Zea mays
acggtgacctcagagccatcgtcgacgacgccctcacccaccacgacgagctgttccagctcaaggccatggcggccaaatccgacgtgttccacctcatcactggggtgtggacgacccccgccgagcgctgcttcctctggatgggtggattcaggccctccgatctgctcaaggtatggattcgtttcagtactctgaaatagaacatcagatatcaaaaaaaatgtgtgcatgcacaaggtaggctgacactgactggagcagggaactcaggcggtgttactttttcattactaagatcacaagaactgttccaatgtaaatcagatttccccttagtacaagaagaatttgcagttccaataaagtatacgaaattccattctgttgcagacactgctgccccagctagatccactgacagagcagcaagtggtcggcatatgcagccttcagcagtcatcacagcaggcagaggaagctctctcgcagggcttggagca

SEQ ID NO: 135 - DNA, 459 bp, Zea mays
tttctccttcaattctcttcgtgggaaaatcgttaacgtaagttgtaactttttttttggcacttctcgctattgtttcgacatctttatttcttaagaagtcctgaacctgaattgtaatgcgattttcttgcacctgtctacccgtcatgcagcgggattttgccagcaatcaacttgataggcgacagcgtcattgttggggtgagtgagtgagtctaccgctattattcagtctgaatccagtaaatcagaattccacaggataatagtacctatatttcctgcaaagacaaatttttacatacatcaaaagcatctcgcatggcatgttttatgtggacacgacaataactccgttcttggatgttaaaactattttgacaatctccagatcttctacttcattggctttgcattgttctgcctggaggctttgctaagcgtgtgggtcattca

SEQ ID NO: 136 - DNA, 462 bp, Zea mays
ctgaaaaaaactgttgcaagtcaattgctgtgagctgcttaaaaaactgatcgaaggcacttgcctttaaggcctatcaaattgcatgtcatgtatatgttctttaagtgagactgaaccaggagcatcatggtattttttgggtgtagaaagtaatttaacatcatgtttatctaatgcatgcaattttggaagctatatagctttagttgctcagctttcacattttcaacataactatggtgacatagtcctatattcttgtttaataagaatgtatttgccttgttttttgggtgttatctatggtaacacttcactgatcactggcaggaatcattggtgaaatcttccatagatactcatagtccaggaagtcctcgaagcgatgaacagttggcaggggcaagccagggatggttaaactggctttctcttggaatgcttggtgttggtggaaca

SEQ ID NO: 137 - DNA, 520 bp, Zea mays
  misc_feature (489)..(490): n is a, c, g, or t
cagagtaataaaacacagtgaagaaaaaggagtaactgttacgcaactcgtcaggaaaaaaatgccaataaactacaatcgaaatgttggtgagaactgagaaaatatgacgcggccaaacgaattattgcatacatgcagaaacgctagtgttggatagttccagaaggaattaattatacatgttattgtgctccgagatcaaaataatgtaatgattctctatgcgtcaaaccttataacatccaaattccaccaatggtaattaagctgaagccgtgctacatgttcagcatgagcgtaaactaagctcagattactagtcttaagataatctaaggacaacgacgatcatcccaaacaacttaagtaacagttatacagcgacaacaccagtacagggggcgcctcaaggaccaaaaccgttctcttctctacagtagtgacgtaacactggacggatcaagagacaatggccaaaggtagtannacnnnnnnnnnnntnnaaacctacnnnnna

SEQ ID NO: 138 - DNA, 429 bp, Zea mays
  misc_feature (31)..(31): n is a, c, g, or t
catgtgcatcacaagttgatgcgaagaacantattaaangggcaatccaccgcatgattggttggcaagttgtcattnggtaacacaaaatgnactttggcgacctgtcgtcgtcttttgccccgagagattgtgacacgaaaaatcaagttgcgngtaccaaagtttatttgtggttctctggagctgcttctctcttcnaaaggactagtagataataagtctgtggctgtgccttnnntntgntcgtggccagtaatggtctctgagtgctctattgctagagacatanacggtgctttaggttcttcgcacgtggagtancgtgcacagatnagatcatctcangatggattgtactcntgttgtcgtgggattatttgtagcttatgttgctttgtcccatgtnaagcggtactgagttttaca

SEQ ID NO: 139 - DNA, 434 bp, Zea mays
  misc_feature (99)..(99): n is a, c, g, or t
ccaagataactgaagattctacatttgataatgatgtgttttctttccaaccccacttaggttctgaacaaacagggttctctactgcagaaaaggtgntttccttgattgttagtatctacctgtattttncctcctttttgcagtgcagngttataatactatttggcttccntctattgccaacctcactacaaaatttcctctttttgtttttttggtacaaagcacnttttaattgattcatgtgccagaaaaatgttttttatctacaggactatggtgcctatgagaaaaagcagtccttgtcgaatattcatcagcaggaatccagtctccagtcaagctttaccgcagtcaaggataacactagtgcaacaattgttaaagcgaagccatcttctagttccatgttcagtgatagtcactattca

SEQ ID NO: 140 - DNA, 553 bp, Zea mays
ctggcgtcatgtctcccgtgcgcacacgctgacatttcctgcatgggcacccgaggccgagtggcgagtgtactcgaaccatgtgttgtgtcgaagtcgaaccaaatgcaagccgtggtgtacataacatgcttcaccgtgaacagatcgctcaagcaaaactgaggaatctcaggcccaacaaaagatgggagagactcggtctttaactctgtagggtatggtcaaattgctcctttcgtaaaccaaccacagcagtagacaggcttcggtatcaggagtcccagagtgagggtgcagtaggcttttcatacagatcagaggagtcgagagtgagggcctggtggaagccacctggcccacatgacatcatcaggtcatcacaagaaaagtccttcgcgttttttttttcctttcagctacatatatatgattaaacctgaccacaggaaatgtgccggttcaggtcaggcaccttatcgtgtcgtctttagtttcagttgttgggcatggagggttcatttcacctcagaaacaaatgcctcaaacacca

SEQ ID NO: 141 - DNA, 511 bp, Zea mays
  misc_feature (19)..(19): n is a, c, g, or t
aattagagatgaccacagntcgacaactatacctgcctgcnttcttagcacagtacattgatctcctttaccgtgtttgaaaaattaggacctgttctctagnttatanagttngatttgtctttctatgcatactaggaaaatcttgtgccagttgatcctgcattatataagcggtatttgctaatctggacccatttatgatgccttgcagcgtgagctaacgaagaagcaaatagagaaattgaaaaaccaaaaggagacacttgagtccctgtgtcgatcactacaggcagaaaggaaacaaggccgctccgccagtattccagacgccccttctagccaagaagacatgccagcgacaagtcaagaatcttagcatatgtgcttgtcaatataccgttggtgtccgttacagcttgccgggacttctgaagcctgagaaatgaatatgttactgatatcttgttttgcttgagtttgcgggagttctggtcccatttaacacata

SEQ ID NO: 142 - DNA, 621 bp, Zea mays
  misc_feature (192)..(192): n is a, c, g, or t
attgaggaattttcagcagatgacatttgtctgggatctcattttactgaaacaccttcgaaatctgcagcacaaaatggaaaactgcaccacaaatctatggaggtgatcggtcttgttcgtgatagataaggaacaacgaaacacctgtgaaattttatcagtttgttcttatattactatgatggcacntctgcaggttattccattcggatttgtttttgaagatgatactctccttgaagcatctgacagctnagtagaacctcacttgcgacatctgccatgtaacagcgttcttgatgttgaccggcttcttaattcggtatctacttatctcctggttcttctacattgttcaaaatgctctggaaaaangagcatttttaatagaagttaataaattctaagannnnttatantttatgtataggttttggaaacatctcagcatgttggaaggatgtcagtttcaacagaccaggatttgcctttcaaggaggtagccaaccaatgtgaagcacttctgattgggaagcagcaaaagctatctntctgcatgagtgttcgtgaaaaaaaggttagagatcgtgagcagcttgagctgtccacacaggggga

SEQ ID NO: 143 - DNA, 614 bp, Zea mays
  misc_feature (325)..(325): n is a, c, g, or t
atcaaacctctgaaaagtctagacacgccaagaaatatcttcccttgctccacttcgtcctcctgcagtccatcgccatcccaggttacagcaaattcaaattcatccaggctggaagaagaacacggcacaccaactcgacacctgcaatgcatcatcgatgcgtgaagggaacgacagcagagtcatccgaagacggctgcagcgcgctgatcctgaccacgtagctcttctgcctcgacgcgacgctgctgcggcccgagccggagctcgggcgcgtgtcatccgaggagccgtcaccgccgaagaggctggaggagctggngtnccnnnnntcctcctcctcctncccgtagnnnnnngcgctgccgatgtgccccccgcacttncggcagagcaggcgcgaccgcctcctgaagaggccccaggagcgggcggagcggaagtagggcgtgcaggtgagctcgtgggccagcgtgaaccggctctcgtcgaccgcgacgaaggcgacgacgcccttcctgatctgcttgcggtacttgggcccnatgccctccgtgtcncgcgccgaggagctcaggttnagcgcgtacccgcagtacccgcaggggnat

SEQ ID NO: 144 - DNA, 596 bp, Zea mays
  misc_feature (6)..(6): n is a, c, g, or t
cctggntttcgtgctagcgtgactatatcgttcggtggcgtggatgatagagcatcccactgcaggcagcctaatataggatagataattttgctgaccagcataggnnnnnnnnnnnnnnnnnnnnnnngaatttggagtagtatagatncggatagccttataacagtatcactgtagcaacctacctcagaacaaaacttttttttttgcctttcctggcctacgtttctctctcccccctcttttgtgaaaggtacctgtgcttgtcttgactcttgtgtttgtatgnttgtggccttgattgatccgctagaggacctccatggctctatgacaattgtcatgttcatgttctgcacgaaccattccttctgaaaatccccatgctcatccaatgtccatgtcctggcaacaatttcaatctggaccttccagtgtgtcagtaactcccattccattcctccacnttatgggtttgatttnttggactgntcggatgaagggtnaaaaaaanngcttttccgtgttatcaaatggtaaatagtaatgtacctggtggctggtgctgagctcgatcaagagtttggtaggca

SEQ ID NO: 145 - DNA, 640 bp, Zea mays
  misc_feature (418)..(418): n is a, c, g, or t
ggcgcatcatcttccagtcagctctgcacgtgccacactgccactgccaaatctccgtgatggtggtgggctgcatgtttgcgcaaactttctgccgaggccgaataccaacgagtcgagtagacgaaggaatcaccggcgagttcgatcggccccaatgcaaggttctctgtgtgtgcgtgggtgtgtggacggtggtggagaggtagatggcgaggcggaggagatggagatggggagatctatttatacatgatgatgacgcgtgcggaggctgcgatcaccagtcggtcgccaaacgagagcgtttcccacagcgtcgtttccaagcgacccttggcacctgttcagttgcttccttcagtcggaaggtccctctgcttccgacgtgaatcgcacgcagggtttagaggttcantctcngtacgcagcctaatctctcacaataaggagaagacgccaatttgatctctctcatnncnnntataccgtttgacaaacatggtaatgctcttgccacacccgtgacatatctttgcctttatcttttatgttcggtcggttacgagattcntcgctactcacattcacacgactgactacgactacacgagaggcggttggccttgatcagacagac

SEQ ID NO: 146 - DNA, 626 bp, Zea mays
  misc_feature (80)..(153): n is a, c, g, or t
tgcctccgcccaagcagcaattcaaacaggagtgcgtctaaatatttctttaaatatttcttcacagcaagttagtaccnnnnnnnnnnnnnnnnnnnnnnnnnnnnnnnnnnnnnnnnnnnnnnnnnnnnnnnnnnnnnnnnnnnnnnnnnngcgtaaaatttagatcctattaccatccctacgtgcgacagcggacagcacagacggtgccacagcgcagactgagaaaaatgatcaaatttaggccaacaaaacaattattggctttgaccccgacgaaatatttttgagtacaagcatgattctgttacagttcactgttctgccgtctgtatgagtgtatgactgtcatgctaatacggaaagcctagacaattcttagctgccgcttgcacttacagactccgcagactctgaaagcagagggcgataattagaatccctgtttttacgtgagccagtcagtagattagtgagattggctcccgccgactccagattcccagaagttctgatagatccattgcgctgctcgacctgctggagtatgttaacggcgctaggctgatacaggttcttcaagtacctgaaagccaccaccggtcccattcctactgcaaccg

SEQ ID NO: 147 - DNA, 668 bp, Zea mays
  misc_feature (659)..(666): n is a, c, g, or t
gcaggcacaactggaacaatgaaagaagaaaccataaatagctgatgaagtcaaatgcaatatcacaaaacaaaagcagcaaagaaaacaattaaaggagtccaagtttatggtgcataaaggctcaaaatcgttttgtcagaacaagcagaaacaactctaacctactgcaaaatgcacccccacgctcagagttctcatgccaatgtggttttgcatatgaaaacaaacacaactacggtcaatcctgcaattgctgtatggcaattaaaaaagcttccatcaacataaagacatcaaatgtgtgagtacacacactctatccagctttaacccagcttaacacctcgggaatagtacaataataatctagtaacacctacatacacaatatctaagatatcaatgaatgcataacaaaatggacagactacataagacattgtaaatccatacagaatagcaataccaaatcgtagaacaaggaaataaacaatcatgatgtttattgtacagtgacaactctggccacatcacttacaaggacgccatttgcagaaaagattaaaaaataatcagtgtacagggtagcagaaacaccacacatgaacagcaatcagataaaaagtcgtagcaaataggagacagcaaaatgcagnnnnnnnngg

SEQ ID NO: 148 - DNA, 726 bp, Zea mays
  misc_feature (39)..(39): n is a, c, g, or t
accggcccgtggtttatattgcctctctggctcgcgtantnnccggtacacccattttctcctgcaaagattctttataaccattttgacggtggtgagcccgaagccagcgaaaaccatcaatcccattcctctcctccgctagtagccgccaaatcgacgagaaggagccgaggccaggcctccaccaagtatcagattccacgccctacgaatcatcgcaatggtgagtattcgcccgcggcggccccgtcgagctatgtgtggcttactgctcgcatagcctcttccgccgtgggcttggattgacggcccggttgttngctgaggtgaggtcctgctgtgtgtgtgttttgtgcagacgaagaancagcggagggaggcgggggcgatgcgacggcgagaccgacaccaccatcaccggcggcagccgcgggacgagcgctgcgtttcctgcaccacgtttaacatcctggcgccgatctacaagcgcatggacaacgaggtgggttggatcttgctctgctttcccttttgacggggaatctatttagttagttttggtttggtagaaaattctggctcgtagtatattgacggtcggtgtttggcattgggagcagaattgcagggagagccagtacagggcctactggttcagccgcaacgagaagattattgaccgccttctcgctgatcactcctccatcatctgnnntcaggggt

SEQ ID NO: 149 - DNA, 754 bp, Zea mays
tgcatgcagttgcagccgtgtttgccgtttagcaacttcaactcgtcgtcgtacaactacggacagacgtgtacgcgcttgttgcgttcgtccacgacaagggcatgaagggcgcgctcgacgatttgaaagtgatgctcacgaggaacgagcccatcaccggcctcgccaaggtggtggcggtcctcgtgatcttcgcgctcggcgtcgtcgccggcctgtgggtgtctgccggggtccgccggtcccagcaggaaagcgtcgacccgaggagcaccgggttccatggcggcggcggcggcggcatctgctgccggcccgagcccaaccccgacttcgaagagttcgtggccccgacgcgcctcatgcacgacatgacggacagggagctgttctggcgcgccacgctggtgccggcggccgccgccaggtacccgttcgagcgcgtgcccaaggtggccttcatgttcctggccggccgcggcgtcctgccgctggcgccgctgtgggagcgcttcttccgaggggctgggcacgaggagcgcttctccgtctacgtccacgcgccgcccggggtggcgatcaacgtgtccgaggactcgccgttctacggcaggcagatccccagccaggttagtgattgtcgtctcgtcgcacatatgctgcatgcatgatgagtactacttgctctgtcaacgtgtccgaggtcgactctcgcttcgtctgatctgatcaggggtaccgagctcgaata

SEQ ID NO: 150 - DNA, 725 bp, Zea mays
  misc_feature (285)..(285): n is a, c, g, or t
cacttgttgtaaaccgacaccattaattcaagtattactcgaagtaaactttgcttgataaaagaaggaacgattgcttgataaaagaaggaacagacaggggtaggatacctaaacatctcctgatgttggagcttgctttgataggctgttaaagttcaacggcgttgtgcttggtttaaggtctttggtgccagtcgcagtggtgtttgatgtcaagcatgccaggctgccagttcgtcacaatatgaacagaacagtggtgctttccattttactgtttgnagatttaggttaaatctggtagcacatgtttgcagaataaatccgatgtagtttccttcagcctnttcagatacgagnngagatgacatatacgtccggttccaatttgcgcttgacgtgcgaggagctttgaagccaaaatagtttcctaacntgttgaaacaaccgctgtaagagtcaactccgaccaatgatttnaagtcgcctggtcgaaccctgagactgaccaggtgacgaccaaggtgattagtcgaccatttaggattttaggattagagcagtagcagccattagttcggcactagcaccgacagaggtaagccatagacggtgtaagccatagagacagacaaaatcacataacagaactacagagaagggggaaccttgagatgatgagctcaactcgtcactagtgttgtagacgctcgtgatataga

SEQ ID NO: 151 - DNA, 758 bp, Zea mays
  misc_feature (7)..(7): n is a, c, g, or t
ctggctnccacggnccgccagctcggaccacgccctcgcggggccccgcgtggcagccgctgcacggtggccgccgtacctcgcgccgtcgcgctcgcaggggcgggannnnnnngcggtgtttcgattcggtggcgttctcgtaataaggtacgnnaaacggagggctttctaggcgtcgggntagatgctgcagnntgcnnngnngtgcagnggctccaaaaaggacgagaccatggttgctggctcgctcgctcgctcgaggctcgagtgcgtttctaacgctgcccacaccacaccncacaccnnnnnagcctagtcgagaagaaagaaacctccggtctctgggtcaggttctcgccgagcacgcgatttcacccgtcctttcggttcaggtaacctcctcccgtgtcttggtctggattctgattcccacctgctgctgttattggcttctgttcccccgagtttgtttgatctgataggttgcngtgtgctcgctgcgaccggtttgcgatctctgaccatgctttgctttcgttttcttttcnttnnnnctcgtcagatcttgtttcggattcgacaggcgagggtgggagttatagaggagagccggacatttcggcgcggtatgtctcttggttctctcagtttttatctggtgtttcttgtttccgcgattacgtgggctgcgctagtcaagaacttctgtaggaatcttggatggatcggtttgtggcctgcaggtncnnanntgg

SEQ ID NO: 152 - DNA, 736 bp, Zea mays
  misc_feature (378)..(384): n is a, c, g, or t
atcagccactggtataataaatgtttctggtgcctttttttcttttaattaatgtacatagtagataactgaagcactaatcttaattgtgtggcttgcattgcaggctgaacacgcacgcggtgatcgagccgttcgtaatcgcgacaaaccggcagctcagcgtggtgcatcccgtgcacaagctgctgagcccgcactaccgtgacacgctgaacatcaacgccctggcacgccagacactcatcaacgccggcggcgtcttcgagcgcaccgtgttccctgcaaagtacgcgctggggatgtcggcagacgtgtacaagacctggaatttcaacgagcaggctctcccagcagatctcgtcaagaggtacgtannnnnnncatacatagatcgactacacgtactgaggtgcctatagaaaactgttcggttcttgacgtggttnngtngtntgcgtgcgttcagaggtgtggctgtgccggaccagtcaagcccatatggtgtccgactgctgatcaaagactacccctatgccgttgacgggctcgtcatctggtgggcgatcgancggtgggtcaaggagtacctggacatctactaccctaacgacggcgagctccagcgtgacgtggagctgcaggcgtggtggaaggaggtgcgtgagnnnnngcacggcgacctcaaggaccgagactggtggcccaggatggacaccgtccagcnnnngtaccg

SEQ ID NO: 153 - DNA, 448 bp, Zea mays
  misc_feature (316)..(316): n is a, c, g, or t
agtacaaccctgacggcgccatctggggcaacaagatcgcgtggggccacgccgtgtcccgagacctgatccactggcgccacctcccgctggccatggtgcccgaccagtggtacgacaccaacggcgtgtggacggggtccgccaccacgctccccgacggccgcctcgccatgctctacacgggctccaccaacgcctccgtccaggtgcagtgcctggccgtgcccgccgacgacgccgacccgctgctcaccaactggaccaagtacgagggcaacccggtgctgtacccgcccccgggcatcgggcccanggacttccgcgaccccaccacggcctggatcgacccctcggacggcgcatggcgcgtcgtcatcggctccaaggacgacgacggccacgcgggcatcgccgtcgtctaccgcaccacggacctggtgcactt

SEQ ID NO: 154 - DNA, 273 bp, Zea mays
  misc_feature (85)..(85): n is a, c, g, or t
agctcctgctcacttgataatgatgcttttgtagcttcgcatcaacctcgagctggtgattagattcttcttgattttggaaggncattatatagcncacagcagcaacgctgtaggacgaaattgtacttgatctatttgaagagcaactatatgcgtgaattcagcaatgatacttggaatgttcaatcatctgttggctacatgaacatgctgcagtttattagtcttgcacncacttgtcacaacagcagaagaaatagcggtaatact

SEQ ID NO: 155 - DNA, 469 bp, Zea mays
  misc_feature (80)..(80): n is a, c, g, or t
cctactccccgcccccgtcgccttccgccgccgtcttttgcgctaacgcttcgcctttcgatcccctttctgacttcttntagcatcatcccaagtcctcgcccggactccgcagattctctggttgctacgagttcttcgcctggnttttgggcaaggtgagctgctcctttctccattcctctggaaatgttccgtttgcttccccttttccttctcgttctggccttctgattgctgacggctcaccttttcgcaggccaaaatttctgatttttttccaccgccgcaaacccctttcctgtgccataccgccattccattcctaagctgggcgtgggcgggtgaagaaaggaagcgaggccggctgtttcgctgaaaccccgccgcctttttttccttttcttttctggacctgattgaagaaccgtagcagattaggggagggaggagcggcggcggccggtcg

SEQ ID NO: 156 - DNA, 842 bp, Zea mays
  misc_feature (14)..(14): n is a, c, g, or t
tgttgccgagacantggtcttgattggaacacctagtttgctgcacaaatatattacattacanaatgaggcaaatatatattacattacataacaaggcaaataatatcgagtaaagcagaatagcagatatatcatctttaagctacctaaacagagcagcaaacactgtatttacagtattgagacttgataggtccagctccagttcctgactatctgcttcaatggcctggacagtaacaaaaacattagtcacacgaaaaaaaacagtcagctcaaatgctcaaccatgccnccaatcacctcaaaccctgatgcatcatactggcgcctcttgtctggatctgacaggatattataggagaatgtggcctcttgaaacttctctgaggcaacagggtcgtctgaattcttatctggatggtacctgcatgacaaattttactagatatataaatactcagacagctgcatngtcaccatcaatttatcagtaaacaagatagctaaacagtctaaactaccaataacntacacataaggcaggacagagcttcttttttttctttgcaagacagagccttgcttgttgttccnatttataatgcacgattcagaagctanataatactggtctttacgtggttccaaaaggagaggaatttattcaagataataaaacaatatacagctacgcatactcggaacaaaataagcacaaaatgcgaactaatctacaaaaggaatttgaacacagncattccctctgaggaaaataagagcacgcgaagtgcccaaatccaagaccattgcagacaatacttacttgagtgccatgc

SEQ ID NO: 157 - DNA, 828 bp, Zea mays
  misc_feature (101)..(101): n is a, c, g, or t
cctcccatttgaagattcgaacctgatgtcgctctacaagaaggtttgacagtctctaccatgacaagagcttaagaagcgcttggttttccttctttttntnntccttaatggttgcttgacacttggtctgtttttttcatgcagatcttcaaagcggatttcagttgcccgtcctggttctccacaagcgcaaagaagctcatcaagaagatacttgancctaatgctaacaatgtgagttctgctgttcgtacataaccatcatttttaatccattctgttcagcanctctgtaggtatactaactgtgtgccatcgttttgtggcctgtgcagagaataactattgcagagctcattaacaacgagtggttcaagaaggggtatcagcctcccagatttgaaacagcagatgttaatctggatgacgtgaactccatcttcgatgaatccggggtaagcttttngcaccatacagttcatatttntgtattatctcacanctcattgnatgggcaagttnnnnnnaaaaaaanntngctttcaatgcaggagccagcacagcttgttgttgagaggagagaagaaagaccatcagtgatgaacgctttcgagttaatctctacgtctcagggcctcaatctcgggacgctcttcgagaagcaaacggtatgttcatgaccttaannnnnccgatgnccctgaacttcggatgataaagaaatgaaatccactagactgcaaccaaccacgatancctttgcctaactgaatgccctgcaatgcctctgtgttactagggttctgttaagcgagannacaaggt

SEQ ID NO: 158 - DNA, 564 bp, Zea mays
  misc_feature (144)..(144): n is a, c, g, or t
aatggcgaaaagagcacagacgaatcctgatatcgcaatgcagtaagggagtttcagaactccatcatcactgaacagtccatacgtggcctgcaagtggaaataacagtatatgatattatatggcaaagagagagagagagngnaatgaaaataaatggtatgagtcgtcgtcatacggtacggtacccctccctaccttgagagcttgtccagctaagatgataaagccagtgttgatcatgaaaaggttaatgtactgcagagcccatgtaagcccgtaaattttcggtcctgaagtaaaatgaaatcaaataaaaagaagaagaagaannnactagtgagactattctatataatataggtagggagtgcaagtgcagagcttattactgactgaccatatatgtgtccagcaaggtctctgtatctgatatggcgtttgccaccgacttcatgaagccgtgcaagaagagcattagcgtacatggatatggcggcagctaggaggaggccgcatgtgccgccgatccagcctagagggaccatgattgatccagagta

SEQ ID NO: 159 - DNA, 367 bp, Zea mays
  misc_feature (62)..(62): n is a, c, g, or t
ttcggtctgtatggctcgatccccttatgattgggcatggcggccgggcgtcgtctggcttnttctggctcaacgcagcggagatgcagattgacgtgcttgcaccaggttgggatggggtcactgaccatganaacgggcggatcgacacactgtggatggctgaggctggtgtcatcgatgcattcttctttgttggttctgagcctaaggatgtgatcaagcagtacataagtgtcacaggcacaccttcaatgccacaacagtttgctactgcgtatcaccaatgccggtggaactaccgggatgaggcagatgttgacngtgtagatgctggttttgatgagcatgatattccatatgatgt

SEQ ID NO: 160 - DNA, 847 bp, Zea mays
agagcaacatttgagataaatgcatatttcatctgtggaggagaacaagtcaagtacacaaaatcttgctcaggtaacaattgtgctcagtaagacatataccttgcacttgtccaattgcgtcggaggcaagcatcggggtgcagtgaagaaaggtgtttttggatcgtaaaaatcaaacttggaaggctgaagagagaaacatattaggtaaatttgtgtttaaattctagccaacaactattaaatgataagctaattgaagaaaacaggatggaactaagatggtctaacacattctatgcattgaattccaggtaatatatatgcagtacagaatacatgacagagtacctgctcagtgagggccaagtttgcatcaaagaatgatttgattgttccaacatcctcccaatagcccgtaaaaatgcatgcctaacacaatacgtaaaatgtattgttagtatgtatgatgcatggaagcgaaaatacataaatcgtgcattggtaaataaaaactagtggaaactggaaacaaaaacaactttttgacatgaagaatttacagtttacagaacacatactccagacagatcagacttacctgcacactatgatctagtacagctcttgggaggatttcagatccaaagtcatgtaattgagtatattttgacctaagaattgacactgttagaaggtaaactaggagttggatagaaataagtcacaggaaagtgattacttgagaaggtctaaaagtgcatctttcttgaagacataaatgcccattgatgcaaggtatggatatttctgtgcatcatctatagcatagctcaagaagttg

SEQ ID NO: 161 - DNA, 1256 bp, Zea mays
  misc_feature (14)..(17): n is a, c, g, or t
atctggtgatgtgnnnntctctctcnnnagtggnnnnnngaaattgcaaaagagannnnnnatatattttttacagaccgcgaaggtagaagaaacacagaatgcctttcctgggggattgacaaagaacgagtccttcgtgggagaactggcattgaggtattgggtcacacttggtaaattttctcctctcttcatcttttgacaagaaataggagcaccattctccttatattactttggttagatcttagattgtcattttcccatctttctgcactgtggtacctacaatttaatttagaagatagcaaaaccattttccttgtattgaaacacttaactttagtgtaccttctacctagctttgtttgaatgccatactataaatgaagaaaaaaactagtacaccgagccttaacaagatcatctgcaggtctgttttgatttcatgaggagtttccatatggaattcagaaacttatctgaagagggccttgtttcttctatcgaaattggattgggtgcttcaggggagctaagatatccttcatgtccagaaacaatgggctggaaatatcctggtattggtgagtttcaggtatttactannngtacttaaataccaagttgaaatttgcaaggggtttatttgggagtaatctttggaatccacctaaactcttacgatctggacataaacaagctctgtttgcattcagtatctgtgtcataataattcttaaataaacatggtttttggtgcatcaacatgtcaagcatgggctatttaagctgctgtcctttttacaagggactagttcttgcatttggataaagcgacatgcctcacaanagatctnnnnnantaaaacaagtaataactaataaaactacatttaaaataggttatnnnanacagcaacagatttgatcgctatacatnnccatgtctctgtctgtctgattctgtctgntgtgtgacntggcttactactcgaattcaaagactgcatatttggtgtgtgatagtgtaatgcatanggtatacattggcttcatttcctttgttgatttccatcgttgncagtgttatgacagnnncntgcaaaagaanntacggcaatcagcattgtcccggggtcatttgttttgggcacgngggnnnnnnnacnnnnnnnnantnnnttcaagaccacannnannnnttttttnnnnnnnaggcgnnnnnnnnannnnnnacggacgttttttcc

SEQ ID NO: 162 - DNA, 1302 bp, Zea mays
  misc_feature (3)..(9): n is a, c, g, or t
ggnnnnnnnaaatnnnnntcctaaatggactgggttactgcctaaatctggaaagatggttagtgcttgaattctttatannnnnnntanggncttacannnttnnannnanactttttattcnnngtnatggtnttnacnancatnagaancgtgtaaccatgaaatattattcttggtgctctaggtaattaatacagagtgggggagcttcaaatccaacaaacttcctctttcagaatatgacaaagccatggactttgaaagtttgaaccctggagagcaggtattgttgctctggcggttgactttnccatttcaggtgactgcatgaatatatgtggataacttangngtggcttctgacagatatacganaaaatgatttctggtatgtatctcggagagattgttcgaagaattttactgaagttagcacatgaagcttctctatttggggatgttgttccacctaagctggagctgccatttatattgaggtatgctttcttgtcctatggacatccagctgttcaagcttgtttgctacattgttggtatggaaaagttgtttatgtctctttaataggctaagttagatgtcacatcagtaagtaatccaaagaaggcgacatgatacaatattttttttggtcaactctgtttatttcaattggttgcaataaacatggtctctgatatgctgcaattttacttttgaataactatcttgatggcatgagaaaatgtgtgcctagaaacagcttgcttcagggagctttatattagattagatttcagggctaataaagtatttacctggagctaaaacaaacggtcaccttgtaactctcgttagtctattaacaggtacatgtattgggtttgaggcatgttgatgcttaacatctttgtgtgatgcttaacattttctttggcaccagctctttctgtgccctttttatgcttattagtaagttgaaacctatgtatcaattagtacatgttcgatgaatacattcgttgtggtatcacaggacgccagatatgtcagccatgcatcatgactcctcacatgacctcaaaactcttggagctaaactgaaggacatagtcggggtacggcttgcctgtgccaaattggcttgttgttcataannngtcagtcagtgtnctctcggtcccttacggcatatacatttgttctcatgttcaggtcgcggacacttccnnnaagtaaggtacatcactcgtcacatctnnnnncttgtcgcagagnnnncagcacnnnnnnncgccgca

SEQ ID NO: 163 - DNA, 484 bp, Zea mays
  misc_feature (33)..(33): n is a, c, g, or t
ctcctgttgacttgcagcggagagaccaggagncatggtgatccctccaccagcaagggcagctagagtcacccgtttcctcaagccctacctattgaggatgcatttctcaaacaagtatgtatctgctcaggtcgtccacaccccaacagcaactgttgcatgttctgcaagctcgcaggagaagctgttgagaccaaacatggagtcgacccgngacgtcgcagcagctgcnaagattggaaagttgctcggcgagcgcctgctcctcaagggaatacctgcagtgtccatccacatgaagagagaacaaaagtaccatgggaaagtgaaggccgttatagattcagtcagagaagctggagtcaanctgttgtgattgcgatgttgaaaacattggacttagcatttgtgcaatggctgagctgtaatntttaagtgtttatcaatgcaaaatggctggacgttgctgttctgaaattct

SEQ ID NO: 164 - DNA, 602 bp, Zea mays
  misc_feature (229)..(229): n is a, c, g, or t
gtggttcctgtttgggatgtggttagccatgtggccttttttgtttgagaagattaacaagaccaggtttgttttctctggtgaaagtgtgccagcaaaagagcgtgttctgttatttgctaaccacaggaccgaggttgactggatgtacttatgggattttgcattgaggaaaggccgcttgcagtgcatcaagtatatccttaagaaaagtctgatgaagttaccngttttcaactgggcatttcacattatngagtttattccggtagagagaaagtgggagattgatgaagcaataatccgaagcaggctttctgaatttaagaacccaaaggatcccctttggctggcagttttcccagaaggcactgattatacgtaagatctctttccantctttncttcccacatgcttgcctanatcactgacatgatttggntttgcgctccaggcatntggaanaataatttgaatcattcacctncaatanttttnctttttggcttctatgcttcaactgtnnttttatctatctgtattctaactagtcctaatgttaatatctcagtgagaagaagtgtatcaaaagtcaagaata

SEQ ID NO: 165 - DNA, 1076 bp, Zea mays
  misc_feature (6)..(10): n is a, c, g, or t
tccgannnnnaaatgcaagnnnnnngtataaggcagtattctttacatctatggttctttctgattatccactaaattacatttcttctaaagtgattaaatacttagctgttataaacatgcattcaggtgtaccatccaaatattgatttggaaggcaatgtctgtctgaacattctgcgtgaagattggaagcctgttctcaacatcaacaccgttatttatggcctgaatcttctttttacggtatttcagctatcttgtttccccaattgttgaaataatacatttagaaattgggaagatgaaagaatagtgtttgcatttttgcatgcattgttgtcatccgattacttcatttcatggactatgctgttcgagagttgctgtttaaagagtaaccactattgtgctcgcaaggatgttacctccaaaatagacataccaatctgagcaagacctaataggttctgttactngtaaaactgaatttcatccttaaaagattctcatgaaagatgagttgaagggtgcttctaactatgttaatgaatatttttgctcttaggtcttgattggatagcgtagtnnnnnnnanncatgaaattggtagnntnnnntttnnnnacncnnnnnnntgcaaaaccatggtttnnnaaaaaaatgtctagagtggattttttttttgaaatccaaataggctacagatttagctatactatggttttcaaaactgcaaagtttgttgcatcccatgttttattatcattgaccagtctactgttttgtccacgtgtataagcaaaactacttggaacttgcattcgtgacagtttaaattgtaagtttggttctgaaccttgttttcgaggacagcatatttaatactatggtttggttggaaaagcatctaaanaaaatnaaccttatctaatgtttatccttctttgtgaattatcgaatgtttacatacattgttacctgcagcaaccaaacgacgaggatcctttgaaccacgaagctgcagctgtcctccgtggcaacccaannnngtttgaggcaannnnnnaagagcc

SEQ ID NO: 166 - DNA, 678 bp, Zea mays
  misc_feature (39)..(39): n is a, c, g, or t
cccgattcggtagttggcttggggatttcactgcaagtntngggattttctgtaggnaagttgatcgagaagtcttggtagacatggagcagatccagtactccgagaagtactncgacgacacctatgaatacaggtagatgcgaaaccctcgccttcacgatcttcacccttccacatcttttccgtcatngntggttggcaaaaccctatcgggatcccctttatccgcgcaggcacgtcgtcctcccgcccgaggtcgccaagctcctccccaggaacatccttctctccgaggtaaaacgcctgcagtttcacccgcattcctatagattgcgttctcgtcctgttgctttggttgtctnatgcctggcctttgtgggggatccccccnccccnnntctcctgtgcagaagcagtggcggttggtgggtgtgcagcagagccgcgggtgggtgcactacgcgatccaccggccggagccgcacatcatgctgttccgccgcccgatcaactaccagcagcagcaggaggaagcggctgccgcgcatgtgctgcccaagtgaagcctctgctggcgaccaggaatgttaaaaacccctagccctcttttcatctctaaaaggggcgtcgctgttgtgttgattaatccctctggctgttggttggaaactnggg

SEQ ID NO: 167 - DNA, 1218 bp, Zea mays
  misc_feature (2)..(11): n is a, c, g, or t
tnnnnnnnnnngacggcgttgagaccgcggaacttgatcgcggcgatgtcgtaggcctccgccgcctcctcctcggtgcctacgtacgagggaatgtgcacgacgattagcttcacagcacagggtgcatgattagggttgtcgttccccaatgcannnnnnnncgnancnnnnnnnnnngnnnnatattgcatgcatgcagcaggccgggtgcacactgtgccattcaaagcactagggttttctgctcgacccctttgaccgcatgccccgattttctcagacaactttgagacaaaaacttggaaaggtacatttgggacatagttaatggtgtagtcactattttatgctcccatagggcaacatatagaatatctaatcttcagttctacgagacgaagatttcaagaaatttgactaaacaagtacggcggggcgagatgacttagatcttggggacatannntataacactatgagtagtcattaactaacctaaggatcnccattaaccgagcaattagtttttatgagttgcattaatcactaatcaatgggtgctagcttcactgagttctcaaaacaaacaaacaagaaaagaagncagctagctagcntactgaatgtgcccnngnnnngatncttgtttcctgcaactctccctatnnttgcttgcnatctcccatgctgatgatgtctgatcgnnnnnnnncattgatgtcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttnnnnnnnnnnnnnnnnnnnnnnnnnnnnnnnnnnnnnnnnnnnnnnnnnnnnnnnnnnnnnnnnnnttnnnngnccnnngannanntnnactgcnnnnnnngnntgtataagatcatnnnacngttagtcaaatcatccatggangtnnnncaatgagaaggntacaancatcaaaataatacnnnnttaggtatgcaatgtactcctgtnnagtcatatgtttcatttcttctagctctttttcgtagttgcttatctgcaacaatgaaattttggcnaaaaaantaaattcaagaacaaagattaatatatatagtgtttaaaaaacaagtcaaaatgcttctgttataagcacccaaagaaacaccatatagaactttgcgtac

SEQ ID NO: 168 - DNA, 464 bp, Zea mays
ttgcacctgtttgatccagcagatacagctacagaaacaatcgatgaggatataactgctgagacattcagtgtcgagaaactggcaaagttaaaaacaatgtctgaaaaggttgtcatgatgcgtagccatgagtcttctgaaaaggatgaacgagcatttgagatcaatccaaatatgactgatgacagtggaactgtgattagaagggcttctgattctatccgcattgatcctgggctaaatgaagctgcatacctatcctgggtcaagaagttcaaagaggcttccatttctagtgaagatgccacagttggatttggaaggcaaagagcagcgcctgaagaaaagctcctgaaacatgatgttaacaagcaaaaaatagaagaaaagagattggccaaatgggaaagtttgggttaccagacactagcagttaaggaacctgatatcacagcaagcca

SEQ ID NO: 169 - DNA, 374 bp, Zea mays
  misc_feature (135)..(135): n is a, c, g, or t
ctaggagtttttgattggttcattcatcttaaatttagtttggccgctcaaggattatggtttcgtcaagtagttgcctgaatggaacaacaaaacatgcacactactcagtgttccatataaagagagagaganaaaannntaacaagcagcgtcctgcgagccagtgttgcgattccagttcatgcaattggcatccgtaaggggaatgcaagcgcaagacactagagtccagggatgctggtgtaggtagagagggggaacaaggaatttacatacggcatagagatgagagctctctcgtaccattttgtaccttcaattcgcaatgaactttaggcaaggattgccttgggcaattgaacatttgtcgg

SEQ ID NO: 170 - DNA, 1273 bp, Zea mays
  misc_feature (157)..(159): n is a, c, g, or t
cagagcgttctaatttgcttcttcatatagtaagcactagcaaacttgtccatcaaatagtaactatacagaccagcattacacaccttcacaactatttctgctccgtcaaatgcaagagtagcaagcgacagcacctgatcgacatgatccatgnnnactccagagtaccagttannnaaaaaacgtccatagtagctgtcatagtcgcctccatcacaaaaaaagccagtttcatgtggtcttgaattataatagccagcgttatcnnnnnnnngtgccnnnaacaaatgaccccgngnnnnngctgnnngnnntnnnnncttttgcatgnncnngtcataacantnnnnnnnnnnnnnatnnnnnnnnnnaatgnnnnnnnnnnnnnnnnnatgnnnnnnnctatcnnnnnnnnnnnnnnnagnnnnnnnnnnngnctagtaagccangtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnannnnnnnnnnnnnnnnnnnnnnnnnnnngacagcagnnnnnnnnnnnnntgcttgacatgttgatgcancaaannncatgtttnnnnnagannnattatgacacagatactgaatgcaaacagagcttgtttatgtccagatcgtaagagtttaggtggattccaaagattactcccaaanaaancccttgcaaatttcaacttggtatttaagtactagtannnaatacctgaaactcaccaataccaggatatttccagcccattgtttctggacatgaaggatatcttagctcccctgaagcacccaatccaatttcgatagaagaaacaaggccctcttcagataagtttctgaattccatatggaaactcctcatgaaatcaaaacagacctgcagatgatcttgttaagcctcggtgtactagtttttttcttcatttatagtatggcattcaaacaaagctaggtagaaggtacactaaagttaagtgtttcaatacaaggaaaatggttttgctatcttctaaattaaattgtaggtaccacagtgcagaaagatgggaaaatgacaatctaagatctaaccaaagtaatataaggagaatggtgctcctatttcttgtcaaaagatgaagagaggagaaaatttaccaagtgtgacccaatacctcaatgccagttctccc

SEQ ID NO: 171 - DNA, 362 bp, Zea mays
  misc_feature (46)..(46): n is a, c, g, or t
ttgacttgaccggacagtgctgttcggtggctcggccgcgatgccngactccgacaacgagtccggcgggccgagcaacgcggagttctcgtcgccgcgggagcaggaccggttcctgccgatcgcgaacgtgagccggatcatgaagaaggcgctcccggccaacgccaagatctccaaggacgccaaggagacggtgcaggagtgcgtgtccgagttcatctccttcatcaccggcgaggcctccgacaagtgccagcgcgagaagcgcaagaccatcaacggcgacgacctnctctgggccatgaccacgctcggcttcgaggactacgtcgagccgctcaagctctacctncacaagt

SEQ ID NO: 172 - DNA, 1236 bp, Zea mays
  misc_feature (21)..(21): n is a, c, g, or t
ggccacaaaataaaccctatntaacatattattaaatttgaatanttnttgcaaaaanttctcatcttttgtggggtccttcgagttatattttttgattgccaataagcctcactgttngttctcttattatgcnnnncccatggtagtgattgtggcatagtgaatgtgaacatnncnactaatggtgctgaaatnnnnnnnncnnnngnnnnnnnnnnnnnnncnnnnnnnnnnnnnnnnnnnnnnnnnnnnnnnnnnnngnnnnnnnngnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngagtnnnnnatgannanttagttagannnnnnnnnnnnnnncannnnnnnnnagnnnnnnnnnnnnnnnnnnnntaggnnnnnggagacccannnnnnnnnnannnnttggnnnnnnnnctccagnntttttantgcacactttgcaactgcaagtctgcaacacatctttactggatattctttttagtatgtactacctccgttttcnaatatttatcgttcgctagttcatttttaaactaaaatgcgacaaatagaaaagaacggaagnnntagtaaaatancgaatatacatnagaattacatgcnttacaagtaccctttgttctgaacttctgattgtcccactcatctgtgggttggtatttgtaacgattcgttactgcaaaggttgcaagttttctgtcgnaaggacaattttcagttcgantttttttactaacacccattcnacacatttgcagtacaatcaactatgggagcgagctacctctagcccagggaatcaattttggttaatcctgcagaaatgttgcgcgtggatctcagtgttttctgcagacgtggatgccgtanggaaagaactgatgcaaccgcatttgagatctcagcagacttgagcagcatttattggcgtcagttgtgaaacttaaactgcaggagcatcttgtacataaataaaataagatgtttgttgctcgtgggccgtggcacnttttggtaccggtgtccagtctactaataaaatctaatgcaaaacggtcatannnnnnnnntcatgcagt

SEQ ID NO: 173 - DNA, 715 bp, Zea mays
  misc_feature (1)..(7): n is a, c, g, or t
nnnnnnncatgatgggcggtgtttcagctgctgtttcnaagactgctgctgctcccatcgagcgtgtgaagctgcttattcagaaccaagatgagatgattaagtctggtaggctatcagagccgtacaagggtattgttgactgcttcaaacgtaccattaaggatganggtttctcttccttgtggaggggtaacactgctaatgttattcgttacttccctactcaggtagcccacaccttccatcatattttttactcngtaacatctgtaaatatgttgaaacaccatgatctttcttacctaagtggttgaatgttcctggtggctttgcaaggtatacaagttaccgactacactttaacttgtacattattaatttgtcttcaaatctttgtcccaggctttgaactttgcatttaaggactacttcaagaggttgttnaacttcaagaaggatagggatggntattggaagtggtttgctggcaacctggcctctggtggtgctgctggtgcttcctctttgttttttgtgtactccntggactacgcgagaacaaggttggcnaatgangcgaaggctgccaagggaggaggtgaaaggcagttcaatgggcttgtngatgtctaccgcaagacactcaagtctgatggtattgctgggctttaccgtggatttaacatctcctgtgttggaatcattgtttatc

SEQ ID NO: 174 - DNA, 562 bp, Zea mays
  misc_feature (1)..(7): n is a, c, g, or t
nnnnnnnagctcgtanggcaggagttccaaaacgggcacgcaaagagaagcaactcagtacttgtagtccttgacgtggaggctctcggtgtcggcggcagcatcacccttgtaggtgccgagggtagcctcagagttggccttgcacctggcgaggaaggcagctctagccttctccaagttctccaccttgccagcccaggccttgagggtgctcgcctggagggcacggccgaaggagaaagacagggaccacggcttcttggtgctgagcttgttcatggcattgaggttgcgggtggcctcctcctcgctctgtccaccagagaggaagacaacagcaggcacagcagcagggacggtcctctggagggtacggacggtgtactcagcaatcacctcaggggtcaccttcttggagtcggagcctggagtcaccatgttgggcttcaggagggtaccctccaggaggacatggtgctcgttgagcgccttgtagcaggcagcaaggacggtctcagtgacgtaagcgcagcgatcaatgtcatgagggccancaaca

SEQ ID NO: 175 - DNA, 1312 bp, Zea mays
  misc_feature (2)..(6): n is a, c, g, or t
gnnnnntgtgactannnnnngactacggccgcctcgtcgtcatcgtcgatgtggtcgaccagaacagggtatgtactgagctctctcataaatgcgtgtcgttctgttcgctgttgtgatttcgatctagagctttagaggaatgcttttgatatagtgtttggttccggagtatggtgggacggctcctaatttgttgttgtttggattggttccatgtagtacagagtggttccaaaaaaggaaaattatgctcaaatgtcgaaccatcccgctctctcggacggagccgctccctggaatgtagcctttctatcccaattggctccggaaccaancactantgttagttggttggtatccgctgctcttcgtgagaattatgaaaacgttatatgcctgtatttggcatcacctgatgcaatttatttgaattggcaaagtagcgtggcgttgtccgcatctagcgtttattaatacntactcttaagatcaatggatgactannagtgccctagcatttggcagtgccaggagnatnngggttgcttgccaaacttgttcccttaatgaaccaatgactaattctcttgtttagnttccttttaaggcacaangaatgttcgatatttaacaatatatatttgtggagcctgtgcctcagtaattgatttggttaggcattcaagcactcatggccttgatgcattctatggaccaggcacttgtggacgcccctgatatggtcaggtgccagattaacttcaaacggctctcacttacngacatcaagattgacatcaaacgngtncccaagaagtcaaccctgatcaaggctatggaggaagctggtaatgatgancctcatntagtggacntcttcagcaangttgtgctttattcatctatattctagatgaagataatagtgacaattaattgatgcnttgttattcttttttagatgtgaagacnaagtggganaacagctcatggggcaagaagctgattgtccagaagaggagggctgcnctcagtgactttgacaggttcaaggtcatgctggcaaggatcaaggtcaggaacctccctgtcanatgtctagttcctgttggcactgagtatattgnttgctcacacntttttcctttatgtaaacagaggggtggtgctatcaggcaagagctcnccaagctgaagaaggcgtccacngcttaagtcccttttggtggatgncatgttagtttttggtttcatgttnnatcagcantttgttttgagtgttgtcaaagccagaannagtannnnnnnc

SEQ ID NO: 176 - DNA, 600 bp, Zea mays
  misc_feature (69)..(75): n is a, c, g, or t
aagggccaaaaccgtgacagggagaaggtaggtggtatttcgtcaagaccaggatcctgtataccctcnnnnnnnctttttcttaagatgaaaacctgctcgccctttttttatatttctttcttttgcgaggttatatcatcttattttctcgctttttttgctagcttttcctagcaaaccaggaaaagttccatgctggtgcngataagcaatactggaaggcgatatctgaacttatcccacatgagattgctaacatcgagaagagaggggcaaggaaagacaaggagaagaagcctgggatcgtggtgatncagggaccgaagcctgggaagccaacggacatggcacggatgcggcagatattgctgaagctgaagcacactcccccaccacacatgaagccacctccgccacctgccgcagcnaccggaaaggatggagcaccagctgccgccgggaaggatggagctaagccagcaaccgttgccaacggcagtgtccctgagatggaaaaggctgctgnggaggcancagtgccagcggcagcaccaccagcagcaactgagccgatcgcagctgcttaatctnnncatc

SEQ ID NO: 177 - DNA, 26 bp, Artificial Sequence
  Assay Sequence
tcctaccaaaacgatcatagatcaag

SEQ ID NO: 178 - DNA, 15 bp, Artificial Sequence
  Assay Sequence
ctaccaacgcaatca

SEQ ID NO: 179 - DNA, 14 bp, Artificial Sequence
  Assay Sequence
accaaagcaatcat

SEQ ID NO: 180 - DNA, 23 bp, Artificial Sequence
  Assay Sequence
gactgttttggcaggaaccatac

SEQ ID NO: 181 - DNA, 19 bp, Artificial Sequence
  Assay Sequence
cggagctctgtttgttgcg

SEQ ID NO: 182 - DNA, 14 bp, Artificial Sequence
  Assay Sequence
tttgctcggcatgc

SEQ ID NO: 183 - DNA, 14 bp, Artificial Sequence
  Assay Sequence
tttgcacggcatgc

SEQ ID NO: 184 - DNA, 19 bp, Artificial Sequence
  Assay Sequence
gcgctatgtggcgtcagaa

SEQ ID NO: 185 - DNA, 23 bp, Artificial Sequence
  Assay Sequence
aggagctatagcagcagcacact

SEQ ID NO: 186 - DNA, 17 bp, Artificial Sequence
  Assay Sequence
actcatcccttactgct

SEQ ID NO: 187 - DNA, 16 bp, Artificial Sequence
  Assay Sequence
ctcatcccttattgct

SEQ ID NO: 188 - DNA, 20 bp, Artificial Sequence
  Assay Sequence
ttccacctcctcctcatcca

SEQ ID NO: 189 - DNA, 24 bp, Artificial Sequence
  Assay Sequence
aaagcaaagcaaaaacacaactga

SEQ ID NO: 190 - DNA, 15 bp, Artificial Sequence
  Assay Sequence
aggaaccacatatgc

SEQ ID NO: 191 - DNA, 15 bp, Artificial Sequence
  Assay Sequence
aggaaccacatgtgc

SEQ ID NO: 192 - DNA, 25 bp, Artificial Sequence
  Assay Sequence
gtcctgactatccctttgtttcttg

SEQ ID NO: 193 - DNA, 25 bp, Artificial Sequence
  Assay Sequence
ttgccttttatttctcccttgattt

SEQ ID NO: 194 - DNA, 15 bp, Artificial Sequence
  Assay Sequence
acgccttgtagctta

SEQ ID NO: 195 - DNA, 16 bp, Artificial Sequence
  Assay Sequence
ccttgtagactgttcc

SEQ ID NO: 196 - DNA, 29 bp, Artificial Sequence
  Assay Sequence
acgcattgtttatcttcataatactacca

SEQ ID NO: 197 - DNA, 24 bp, Artificial Sequence
  Assay Sequence
cagggtttagtctgcaatcaggtt

SEQ ID NO: 198 - DNA, 15 bp, Artificial Sequence
  Assay Sequence
tgttgtgtcaaagga

SEQ ID NO: 199 - DNA, 16 bp, Artificial Sequence
  Assay Sequence
catgttgtcattgttg

SEQ ID NO: 200 - DNA, 34 bp, Artificial Sequence
  Assay Sequence
ctatggtagtagtattttttcttgttattttgtg

(19) United States
(12) Patent Application Publication
(10) Pub. No.: US 2009/0031438 A1
(43) Pub. Date: Jan. 29, 2009

(54) Methods & Compositions for Selection of Loci for Trait Performance & Expression

(75) Inventors: WAYNE KENNARD, Ankeny, IA (US); ARNOLD ROSIELLE, St. Louis, MO (US); DAVID BUTRUILLE, Des Moines, IA (US); SAM EATHINGTON, Ames, IA (US); KEVIN COOK, Ankeny, IA (US); TREVOR HOHLS, Wildwood, MO (US)

Correspondence Address: MONSANTO COMPANY, 800 N. LINDBERGH BLVD., ATTENTION: GAIL P. WUELLNER, IP PARALEGAL, (E2NA), ST. LOUIS, MO, 63167, US

(73) Assignee: MONSANTO TECHNOLOGY, LLC, ST. LOUIS, MO (US)

(21) Appl. No.: 12/144,278

(22) Filed: Jun. 23, 2008

Related U.S. Application Data
(60) Provisional application No. 60/945,760, filed on Jun. 22, 2007.

(51) Int. Cl. A01H 1/02; C12Q 1/68; A01H 5/00; A01H 5/10; C07H 21/04

(52) U.S. Cl. 800/267; 800/298; 435/6; 536/24.3

(57) ABSTRACT

The present invention provides novel methods and compositions for the identification and selection of loci modulating transgene performance and expression in plant breeding. In addition, methods are provided for screening germplasm entries for the performance and expression of at least one transgene.

### CROSS-REFERENCE TO RELATED APPLICATIONS

This application claims priority from U.S. Provisional Application Ser. No. 60/945,760 (filed Jun. 22, 2007), the entire text of which is incorporated herein by reference.

### INCORPORATION OF SEQUENCE LISTING

A sequence listing containing the file named “54008seq.txt” which is 3110 bytes (measured in MS-Windows®) and created on Sep. 17, 2007, comprises 200 nucleotide sequences, and is herein incorporated by reference in its entirety.

### FIELD OF INVENTION

This invention is in the field of plant breeding. In particular, this invention provides methods and compositions for selecting preferred combinations of one or more transgenic traits and one or more germplasm entries. Methods are provided for identification of transgene modulating loci for use in marker-assisted breeding activities. Methods are also provided for evaluation of germplasm entries for trait performance.

### BACKGROUND OF INVENTION

The heritable differences in genomes that contribute to the range of phenotypes observed for any of a number of traits form the basis for decisions in plant and animal breeding. Typically, any one phenotype will be modulated by multiple genetic factors and differences in these genetic factors between individuals can be associated to a phenotypic outcome. In the instance where the phenotype is the product of a transgene, it is expected that genetic factors in the organism's genome may contribute to the phenotype of the transgene. A goal of transgenic plant breeding is to meet a product concept, or efficacy, for a transgene or a stack of transgenes while preserving at least baseline equivalency of the transgenic plant with respect to the non-transgenic version.

Transgene efficacy may be impacted by constitutive genes in the genetic background of the host plant. Allelic variants of constitutive genes, including copy number variants and deletions, may modulate expression of the transgene or enhance the performance of the product concept of the transgene. Thus, a need exists for methods and compositions for identifying and selecting loci modulating transgene performance and expression in plant breeding. Further, methods for screening germplasm entries to determine the performance and expression of transgenes or to determine genetic background are lacking.

### SUMMARY

The present invention provides methods and compositions for identifying and selecting loci modulating transgene performance and expression in plant breeding. The identification of genes or QTL that affect the performance of a targeted trait or modulate the expression of a transgene provides the basis for management of these effects through marker-assisted selection strategies. Most traits of agronomic importance are controlled by many genes. Traits such as yield, moisture, drought tolerance, seed composition, and protein and starch quality are quantitatively inherited by multiple genetic loci. Superior alleles at multiple loci can be selected and genetic backgrounds improved for all quantitative traits, including those traits that have been improved through transgenic modification.

When identifying transgene modulating loci, markers can be used to directly or indirectly select for beneficial alleles of modulating genes and/or quantitative trait loci (QTL) to enhance trait performance and expression. Methods for identifying transgene modulating loci include, but are not limited to, genetic linkage mapping of controlled crosses and association studies of unrelated lines in which all loci are in linkage equilibrium except those very tightly linked to the trait of interest. The same markers used to identify transgene modulating loci conditioning improved performance or expression can also be used to select individuals that contain a maximum frequency of desired alleles at the identified loci. In addition, the markers can be used to introgress one or more transgene modulating loci into at least one genetic background without the transgene modulating loci, i.e., into an elite germplasm entry with preferred agronomic traits. Also, the markers may comprise phenotypic traits that are correlated with at least one transgene modulating locus, wherein plants can be screened on the basis of at least one phenotypic or genetic characteristic.

The present invention further provides methods for rapidly screening multiple germplasm entries to determine whether genetic background effects impact transgene performance. In the case of genetic background effects, methods are provided for identifying preferred combinations of at least one genotype and at least one transgene. The present invention enables the rapid screening of germplasm in breeding schemes involving the crossing of inbred lines with a tester that has at least one transgene in order to identify preferred inbred lines for the at least one transgene.

The present invention includes a method for breeding of a crop plant, such as maize (Zea mays), soybean (Glycine max), cotton (Gossypium hirsutum), peanut (Arachis hypogaea), barley (Hordeum vulgare); oats (Avena sativa); orchard grass (Dactylis glomerata); rice (Oryza sativa, including indica and japonica varieties); sorghum (Sorghum bicolor); sugar cane (Saccharum sp); tall fescue (Festuca arundinacea); turfgrass species (e.g. species: Agrostis stolonifera, Poa pratensis, Stenotaphrum secundatum); wheat (Triticum aestivum), and alfalfa (Medicago sativa), members of the genus Brassica, broccoli, cabbage, carrot, cauliflower, Chinese cabbage, cucumber, dry bean, eggplant, fennel, garden beans, gourd, leek, lettuce, melon, okra, onion, pea, pepper, pumpkin, radish, spinach, squash, sweet corn, tomato, watermelon, ornamental plants, and other fruit, vegetable, tuber, and root crops, with transgenes comprising at least one phenotype of interest, further defined as conferring a preferred property selected from the group consisting of herbicide tolerance, disease resistance, insect or pest resistance, altered fatty acid, protein or carbohydrate metabolism, increased grain yield, increased oil, enhanced nutritional content, increased growth rates, enhanced stress tolerance, preferred maturity, enhanced organoleptic properties, altered morphological characteristics, sterility, other agronomic traits, traits for industrial uses, or traits for improved consumer appeal.

In other embodiments, the present invention includes methods and compositions for identifying preferred genotype and transgene combinations and methods for breeding transgenic plants. Specifically, the present invention provides methods for identifying transgene modulating loci for use in marker-assisted breeding, marker-assisted introgression, and pre-selection. The present invention also provides methods for evaluating transgenic trait combining ability for measuring transgene performance in multiple crossing schemes.

In one embodiment, the present invention provides a method for identifying an association of a plant genotype with a performance of one or more transgenic traits. The method comprises screening a plurality of transgenic germplasm entries displaying a heritable variation for at least one transgenic trait wherein the heritable variation is linked to at least one genotype; and associating at least one genotype from the transgenic germplasm entries to at least one transgenic trait.

In another embodiment, the present invention provides a method for identifying and breeding a plant germplasm entry with a genotype that modulates a performance of a transgenic trait. The method comprises crossing at least two germplasm entries with a test germplasm entry comprising at least one transgenic trait; and measuring a modulated performance of at least one transgenic trait in a progeny of the cross.

In another embodiment, the present invention provides business methods that enable greater value capture for commercial breeding entities. Instead of licensing only transgenes, the entity licenses packages of at least one transgene with at least one genotype, wherein the genotype may comprise a kit for detection of at least one transgene modulating locus, germplasm recommendations for deployment of at least one transgene, and/or germplasm sources for conversions to introgress at least one transgene modulating locus.

Further areas of applicability will become apparent from the description provided herein. It should be understood that the description and specific examples are intended for purposes of illustration only and are not intended to limit the scope of the present disclosure.

### DETAILED DESCRIPTION

The definitions and methods provided define the present invention and guide those of ordinary skill in the art in the practice of the present invention. Unless otherwise noted, terms are to be understood according to conventional usage by those of ordinary skill in the relevant art. Definitions of common terms in molecular biology may also be found in Alberts et al., Molecular Biology of The Cell, 5^{th} Edition, Garland Science Publishing, Inc.: New York, 2007; Rieger et al., Glossary of Genetics: Classical and Molecular, 5th edition, Springer-Verlag: New York, 1991; King et al, A Dictionary of Genetics, 6th ed, Oxford University Press: New York, 2002; and Lewin, Genes IX, Oxford University Press: New York, 2007. The nomenclature for DNA bases as set forth at 37 CFR § 1.822 is used.

An “allele” refers to an alternative sequence at a particular locus; the length of an allele can be as small as 1 nucleotide base, but is typically larger. Allelic sequence can be denoted as nucleic acid sequence or as amino acid sequence that is encoded by the nucleic acid sequence.

A “locus” is a position on a genomic sequence that is usually found by a point of reference; e.g., a short DNA sequence that is a gene, or part of a gene or intergenic region. A locus may refer to a nucleotide position at a reference point on a chromosome, such as a position from the end of the chromosome. The ordered list of loci known for a particular genome is called a genetic map. A variant of the DNA sequence at a given locus is called an allele and variation at a locus, i.e., two or more alleles, constitutes a polymorphism. The polymorphic sites of any nucleic acid sequence can be determined by comparing the nucleic acid sequences at one or more loci in two or more germplasm entries.

As used herein, “polymorphism” means the presence of one or more variations of a nucleic acid sequence at one or more loci in a population of one or more individuals. The variation may comprise but is not limited to one or more base changes, the insertion of one or more nucleotides or the deletion of one or more nucleotides. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found, or may exist at low frequency within a population, the former having greater utility in general plant breeding and the latter may be associated with rare but important phenotypic variation. Useful polymorphisms may include single nucleotide polymorphisms (SNPs), insertions or deletions in DNA sequence (Indels), simple sequence repeats of DNA sequence (SSRs) a restriction fragment length polymorphism, and a tag SNP. A genetic marker, a gene, a DNA-derived sequence, a haplotype, a RNA-derived sequence, a promoter, a 5′ untranslated region of a gene, a 3′ untranslated region of a gene, microRNA, siRNA, a QTL, a satellite marker, a transgene, mRNA, ds mRNA, a transcriptional profile, and a methylation pattern may comprise polymorphisms. In addition, the presence, absence, or variation in copy number of the preceding may comprise a polymorphism.

As used herein, the term “single nucleotide polymorphism,” also referred to by the abbreviation “SNP,” means a polymorphism at a single site wherein said polymorphism constitutes a single base pair change, an insertion of one or more base pairs, or a deletion of one or more base pairs.

As used herein, “marker” means a detectable characteristic that can be used to discriminate between organisms. Examples of such characteristics may include genetic markers, protein composition, protein levels, oil composition, oil levels, carbohydrate composition, carbohydrate levels, fatty acid composition, fatty acid levels, amino acid composition, amino acid levels, biopolymers, pharmaceuticals, starch composition, starch levels, fermentable starch, fermentation yield, fermentation efficiency, energy yield, secondary compounds, metabolites, morphological characteristics, and agronomic characteristics. As used herein, “genetic marker” means polymorphic nucleic acid sequence or nucleic acid feature.

As used herein, “marker assay” means a method for detecting a polymorphism at a particular locus using a particular method, e.g. measurement of at least one phenotype (such as seed color, flower color, or other visually detectable trait), restriction fragment length polymorphism (RFLP), single base extension, electrophoresis, sequence alignment, allelic specific oligonucleotide hybridization (ASO), random amplified polymorphic DNA (RAPD), microarray-based technology.

As used herein, the term “haplotype” means a chromosomal region within a haplotype window defined by at least one polymorphic genetic marker. The unique genetic marker fingerprint combinations in each haplotype window define individual haplotypes for that window. Further, changes in a haplotype, brought about by recombination for example, may result in the modification of a haplotype so that it comprises only a portion of the original (parental) haplotype operably linked to the trait, for example, via physical linkage to a gene, QTL, or transgene. Any such change in a haplotype would be included in our definition of what constitutes a haplotype so long as the functional integrity of that genomic region is unchanged or improved.

As used herein, the term “haplotype window” means a chromosomal region that is established by statistical analyses known to those of skill in the art and is in linkage disequilibrium. Thus, identity by state between two inbred individuals (or two gametes) at one or more loci located within this region is taken as evidence of identity-by-descent of the entire region. Each haplotype window includes at least one polymorphic genetic marker. Haplotype windows can be mapped along each chromosome in the genome. Haplotype windows are not fixed per se and, given the ever-increasing density of genetic markers, this invention anticipates the number and size of haplotype windows to evolve, with the number of windows increasing and their respective sizes decreasing, thus resulting in an ever-increasing degree confidence in ascertaining identity by descent based on the identity by state at the genetic marker loci.

As used herein, “transgene modulating locus” means a locus that affects the performance or expression of one or more transgenes. One or more transgene modulating loci may affect the performance or expression of a transgene. One or more transgene modulating loci may affect the performance or expression of a stack of two or more transgenes.

As used herein, “haplotype effect estimate” means a predicted effect estimate for a haplotype reflecting association with one or more phenotypic traits, wherein the associations can be made de novo or by leveraging historical haplotype-trait association data.

As used herein, “genotype” means the genetic component of the phenotype and it can be indirectly characterized using markers or directly characterized by nucleic acid sequencing. Suitable markers include a phenotypic character, a metabolic profile, a genetic marker, or some other type of marker. A genotype may constitute an allele for at least one genetic marker locus or a haplotype for at least one haplotype window. In some embodiments, a genotype may represent a single locus and in others it may represent a genome-wide set of loci. In another embodiment, the genotype can reflect the sequence of a portion of a chromosome, an entire chromosome, a portion of the genome, and the entire genome.

As used herein, “phenotype” means the detectable characteristics of a cell or organism which can be influenced by gene expression.

As used herein, “linkage” refers to relative frequency at which types of gametes are produced in a cross. For example, if locus A has genes “A” or “a” and locus B has genes “B” or “b” and a cross between parent I with AABB and parent B with aabb will produce four possible gametes where the genes are segregated into AB, Ab, aB and ab. The null expectation is that there will be independent equal segregation into each of the four possible genotypes, i.e. with no linkage ¼ of the gametes will of each genotype. Segregation of gametes into a genotypes differing from ¼ are attributed to linkage.

As used herein, “linkage disequilibrium” is defined in the context of the relative frequency of gamete types in a population of many individuals in a single generation. If the frequency of allele A is p, a is p′, B is q and b is q′, then the expected frequency (with no linkage disequilibrium) of genotype AB is pq, Ab is pq′, aB is p′q and ab is p′q′. Any deviation from the expected frequency is called linkage disequilibrium. Two loci are said to be “genetically linked” when they are in linkage disequilibrium.

As used herein, “quantitative trait locus (QTL)” means a locus that controls to some degree numerically representable traits that are usually continuously distributed.

As used herein, the term “transgene” means nucleic acid molecules in the form of DNA, such as cDNA or genomic DNA, and RNA, such as mRNA or microRNA, which may be single or double stranded.

As used herein, the term “event” refers to a particular transformant. In a typical transgenic breeding program, a transformation construct responsible for a trait is introduced into the genome via a transformation method. Numerous independent transformants (events) are usually generated for each construct. These events are evaluated to select those with superior performance.

As used herein, the term “inbred” means a line that has been bred for genetic homogeneity. Without limitation, examples of breeding methods to derive inbreds include pedigree breeding, recurrent selection, single-seed descent, backcrossing, and doubled haploids.

As used herein, the term “hybrid” means a progeny of mating between at least two genetically dissimilar parents. Without limitation, examples of mating schemes include single crosses, modified single cross, double modified single cross, three-way cross, modified three-way cross, and double cross, wherein at least one parent in a modified cross is the progeny of a cross between sister lines.

As used herein, the term “tester” means a line used in a testcross with another line wherein the tester and the lines tested are from different germplasm pools. A tester may be isogenic or nonisogenic.

As used herein, the term “corn” means Zea mays or maize and includes all plant varieties that can be bred with corn, including wild maize species. More specifically, corn plants from the species Zea mays and the subspecies Zea mays L. ssp. Mays can be genotyped using the compositions and methods of the present invention. In an additional aspect, the corn plant is from the group Zea mays L. subsp. mays Indentata, otherwise known as dent corn. In another aspect, the corn plant is from the group Zea mays L. subsp. mays Indurata, otherwise known as flint corn. In another aspect, the corn plant is from the group Zea mays L. subsp. mays Saccharata, otherwise known as sweet corn. In another aspect, the corn plant is from the group Zea mays L. subsp. mays Amylacea, otherwise known as flour corn. In a further aspect, the corn plant is from the group Zea mays L. subsp. mays Everta, otherwise known as pop corn. Zea or corn plants that can be genotyped with the compositions and methods described herein include hybrids, inbreds, partial inbreds, or members of defined or undefined populations.

As used herein, the term “soybean” means Glycine max and includes all plant varieties that can be bred with soybean, including wild soybean species. More specifically, soybean plants from the species Glycine max and the subspecies Glycine max L. ssp. max or Glycine max ssp. formosana can be genotyped using the compositions and methods of the present invention. In an additional aspect, the soybean plant is from the species Glycine soja, otherwise known as wild soybean, can be genotyped using these compositions and methods. Alternatively, soybean germplasm derived from any of Glycine max, Glycine max L. ssp. max, Glycine max ssp. Formosana, and/or Glycine soja can be genotyped using compositions and methods provided herein.

As used herein, the term “canola” means Brassica napus and B. campestris and includes all plant varieties than can be bred with canola, including wild Brassica species and other agricultural Brassica species.

As used herein, the term “comprising” means “including but not limited to”.

As used herein, the term “elite line” means any line that has resulted from breeding and selection for superior agronomic performance. An elite plant is any plant from an elite line.

In accordance with the present invention, Applicants have discovered methods for identifying and associating genotypes having an effect on transgene performance. For example, in one embodiment, a method of the invention comprises screening a plurality of transgenic germplasm entries displaying a heritable variation for at least one transgenic trait wherein the heritable variation is linked to at least one genotype; and associating at least one genotype from the transgenic germplasm entries to at least one transgenic trait. In another embodiment, a method of the invention comprises crossing at least two germplasm entries with a test germplasm entry for the evaluation of performance of at least one transgene in order to determine preferred crossing schemes. The methods of the present invention can be used with traditional breeding techniques as described below to more efficiently screen and identify genotypes affecting transgene performance.

### A. Marker-Assisted Breeding

Breeding has advanced from selection for economically important traits in plants and animals based on phenotypic records of an individual and its relatives to the application of molecular genetics to identify genomic regions that contain valuable genetic traits. Inclusion of genetic markers in breeding programs has accelerated the genetic accumulation of valuable traits into a germplasm compared to that achieved based on phenotypic data only. Herein, “germplasm” includes breeding germplasm, breeding populations, collection of elite inbred lines, populations of random mating individuals, and biparental crosses. Genetic marker alleles (an “allele” is an alternative sequence at a locus) are used to identify plants that contain a desired genotype at multiple loci, and that are expected to transfer the desired genotype, along with a desired phenotype to their progeny. Genetic marker alleles can be used to identify plants that contain the desired genotype at one marker locus, several loci, or a haplotype, and that would be expected to transfer the desired genotype, along with a desired phenotype to their progeny. This process has been widely referenced and has served to greatly economize plant breeding by accelerating the fixation of advantageous alleles and also eliminating the need for phenotyping every generation.

### 1. Marker Technologies

The development of markers and the association of markers with phenotypes, or quantitative trait loci (QTL) mapping for marker-assisted breeding has advanced in recent years. Examples of genetic markers are Restriction Fragment Length Polymorphisms (RFLP), Amplified Fragment Length Polymorphisms (AFLP), Simple Sequence Repeats (SSR), Single Nucleotide Polymorphisms (SNP), Insertion/Deletion Polymorphisms (Indels), Variable Number Tandem Repeats (VNTR), and Random Amplified Polymorphic DNA (RAPD), and others known to those skilled in the art. Marker discovery and development in crops provides the initial framework for applications to marker-assisted breeding activities (US Patent Applications 2005/0204780, 2005/0216545, 2005/0218305, and 2006/00504538). The resulting “genetic map” is the representation of the relative position of characterized loci (DNA markers or any other locus for which alleles can be identified) along the chromosomes. The measure of distance on this map is relative to the frequency of crossover events between sister chromatids at meiosis.

As a set, polymorphic markers serve as a useful tool for fingerprinting plants to inform the degree of identity of lines or varieties (U.S. Pat. No. 6,207,367). These markers form the basis for determining associations with phenotype and can be used to drive genetic gain. The implementation of marker-assisted selection is dependent on the ability to detect underlying genetic differences between individuals.

Genetic markers for use in the present invention include “dominant” or “codominant” markers. “Codominant markers” reveal the presence of two or more alleles (two per diploid individual). “Dominant markers” reveal the presence of only a single allele. The presence of the dominant marker phenotype (e.g., a band of DNA) is an indication that one allele is present in either the homozygous or heterozygous condition. The absence of the dominant marker phenotype (e.g., absence of a DNA band) is merely evidence that “some other” undefined allele is present. In the case of populations where individuals are predominantly homozygous and loci are predominantly dimorphic, dominant and codominant markers can be equally valuable. As populations become more heterozygous and multiallelic, codominant markers often become more informative of the genotype than dominant markers.

Nucleic acid molecules or fragments thereof are capable of specifically hybridizing to other nucleic acid molecules under certain circumstances. As used herein, two nucleic acid molecules are capable of specifically hybridizing to one another if the two molecules are capable of forming an anti-parallel, double-stranded nucleic acid structure. A nucleic acid molecule is the “complement” of another nucleic acid molecule if they exhibit complete complementarity. As used herein, molecules exhibit “complete complementarity” when every nucleotide of one of the molecules is complementary to a nucleotide of the other. Two molecules are “minimally complementary” if they can hybridize to one another with sufficient stability to permit them to remain annealed to one another under at least conventional “low-stringency” conditions. Similarly, the molecules are “complementary” if they can hybridize to one another with sufficient stability to permit them to remain annealed to one another under conventional “high-stringency” conditions. Conventional stringency conditions are described by Sambrook et al., In: Molecular Cloning, A Laboratory Manual, 2nd Edition, Cold Spring Harbor Press, Cold Spring Harbor, N.Y. (1989), and by Haymes et al., In: Nucleic Acid Hybridization, A Practical Approach, IRL Press, Washington, D.C. (1985). Departures from complete complementarity are therefore permissible, as long as such departures do not completely preclude the capacity of the molecules to form a double-stranded structure. In order for a nucleic acid molecule to serve as a primer or probe it need only be sufficiently complementary in sequence to be able to form a stable double-stranded structure under the particular solvent and salt concentrations employed.

As used herein, a substantially homologous sequence is a nucleic acid sequence that will specifically hybridize to the complement of the nucleic acid sequence to which it is being compared under high stringency conditions. The nucleic-acid probes and primers of the present invention can hybridize under stringent conditions to a target DNA sequence. The term “stringent hybridization conditions” is defined as conditions under which a probe or primer hybridizes specifically with a target sequence(s) rather than with non-target sequences, as can be determined empirically. The term “stringent conditions” is functionally defined with regard to the hybridization of a nucleic-acid probe to a target nucleic acid (i.e., to a particular nucleic-acid sequence of interest) by the specific hybridization procedure discussed in Sambrook et al., 1989, at 9.52-9.55. See also, Sambrook et al., 1989 at 9.47-9.52, 9.56-9.58; Kanehisa 1984 Nucl. Acids Res. 12:203-213; and Wetmur et al. 1968 J. Mol. Biol. 31:349-370. Appropriate stringency conditions that promote DNA hybridization are known to those skilled in the art or can be found in Current Protocols in Molecular Biology, John Wiley & Sons, N.Y., 1989, 6.3.1-6.3.6.

A fragment of a nucleic acid molecule as used herein can be of any size. Illustrative fragments include, without limitation, fragments of nucleic acid sequences set forth in SEQ ID NO: 1-176 and complements thereof. In one aspect, a fragment can be between 15 and 25, 15 and 30, 15 and 40, 15 and 50, 15 and 100, 20 and 25, 20 and 30, and 40, 20 and 50, 20 and 100, 25 and 30, 25 and 40, 25 and 50, 25 and 100, 30 and 40, 30 and 50, and 30 and 100. In another aspect, the fragment can be greater than 10, 15, 20, 25, 30, 35, 40, 50, 100, or 250 nucleotides.

Additional genetic markers can be used in the methods of the present invention to select plants with an allele of a QTL associated with transgene modulating loci of the present invention. Examples of public marker databases include, for example: Maize Genome Database, Agricultural Research Service, United States Department of Agriculture or Soybase, an Agricultural Research Service, United States Department of Agriculture.

In another embodiment, markers, such as single sequence repeat markers (SSR), AFLP markers, RFLP markers, RAPD markers, phenotypic markers, isozyme markers, single nucleotide polymorphisms (SNPs), insertions or deletions (Indels), single feature polymorphisms (SFPs, for example, as described in Borevitz et al. 2003 Gen. Res. 13:513-523), microarray transcription profiles, DNA-derived sequences, and RNA-derived sequences that are genetically linked to or correlated with alleles of a QTL of the present invention can be utilized.

In one embodiment, nucleic acid-based analyses for the presence or absence of the genetic polymorphism can be used for the selection of seeds in a breeding population. A wide variety of genetic markers for the analysis of genetic polymorphisms are available and known to those of skill in the art. The analysis may be used to select for genes, portions of genes, QTL, alleles, or genomic regions (haplotypes) that comprise or are linked to a genetic marker.

Herein, nucleic acid analysis methods are known in the art and include, but are not limited to, PCR-based detection methods (for example, TaqMan assays), microarray methods, and nucleic acid sequencing methods. In one embodiment, the detection of polymorphic sites in a sample of DNA, RNA, or cDNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis, fluorescence detection methods, or other means.

A method of achieving such amplification employs the polymerase chain reaction (PCR) (Mullis et al. 1986 Cold Spring Harbor Symp. Quant. Biol. 51:263-273; European Patent 50,424; European Patent 84,796; European Patent 258,017; European Patent 237,362; European Patent 201,184; U.S. Pat. No. 4,683,202; U.S. Pat. No. 4,582,788; and U.S. Pat. No. 4,683,194), using primer pairs that are capable of hybridizing to the proximal sequences that define a polymorphism in its double-stranded form.

Polymorphisms in DNA sequences can be detected or typed by a variety of effective methods well known in the art including, but not limited to, those disclosed in U.S. Pat. Nos. 5,468,613, 5,217,863; 5,210,015; 5,876,930; 6,030,787; 6,004,744; 6,013,431; 5,595,890; 5,762,876; 5,945,283; 5,468,613; 6,090,558; 5,800,944; 5,616,464, 7,312,039, 7,238,476, 7,297,485, 7,282,355, 7,270,981, and 7,250,252 all of which are incorporated herein by reference in their entireties. However, the compositions and methods of the present invention can be used in conjunction with any polymorphism typing method to type polymorphisms in genomic DNA samples. These genomic DNA samples used include but are not limited to genomic DNA isolated directly from a plant, cloned genomic DNA, or amplified genomic DNA.

For instance, polymorphisms in DNA sequences can be detected by hybridization to allele-specific oligonucleotide (ASO) probes as disclosed in U.S. Pat. Nos. 5,468,613 and 5,217,863. U.S. Pat. No. 5,468,613 discloses allele specific oligonucleotide hybridizations where single or multiple nucleotide variations in nucleic acid sequence can be detected in nucleic acids by a process in which the sequence containing the nucleotide variation is amplified, spotted on a membrane and treated with a labeled sequence-specific oligonucleotide probe.

Target nucleic acid sequence can also be detected by probe ligation methods as disclosed in U.S. Pat. No. 5,800,944 where sequence of interest is amplified and hybridized to probes followed by ligation to detect a labeled part of the probe.

Microarrays can also be used for polymorphism detection, wherein oligonucleotide probe sets are assembled in an overlapping fashion to represent a single sequence such that a difference in the target sequence at one point would result in partial probe hybridization (Borevitz et al., Genome Res. 13:513-523 (2003); Cui et al., Bioinformatics 21:3852-3858 (2005). On any one microarray, it is expected there will be a plurality of target sequences, which may represent genes and/or noncoding regions wherein each target sequence is represented by a series of overlapping oligonucleotides, rather than by a single probe. This platform provides for high throughput screening a plurality of polymorphisms. A single-feature polymorphism (SFP) is a polymorphism detected by a single probe in an oligonucleotide array, wherein a feature is a probe in the array. Typing of target sequences by microarray-based methods is disclosed in U.S. Pat. No. 6,799,122; U.S. Pat. No. 6,913,879; and U.S. Pat. No. 6,996,476.

Target nucleic acid sequence can also be detected by probe linking methods as disclosed in U.S. Pat. No. 5,616,464, employing at least one pair of probes having sequences homologous to adjacent portions of the target nucleic acid sequence and having side chains which non-covalently bind to form a stem upon base pairing of the probes to the target nucleic acid sequence. At least one of the side chains has a photoactivatable group which can form a covalent cross-link with the other side chain member of the stem.

Other methods for detecting SNPs and Indels include single base extension (SBE) methods. Examples of SBE methods include, but are not limited, to those disclosed in U.S. Pat. No. 6,004,744; U.S. Pat. No. 6,013,431; U.S. Pat. No. 5,595,890; U.S. Pat. No. 5,762,876; and U.S. Pat. No. 5,945,283. SBE methods are based on extension of a nucleotide primer that is adjacent to a polymorphism to incorporate a detectable nucleotide residue upon extension of the primer. In certain embodiments, the SBE method uses three synthetic oligonucleotides. Two of the oligonucleotides serve as PCR primers and are complementary to sequence of the locus of genomic DNA which flanks a region containing the polymorphism to be assayed. Following amplification of the region of the enome containing the polymorphism, the PCR product is mixed with the third oligonucleotide (called an extension primer) which is designed to hybridize to the amplified DNA adjacent to the polymorphism in the presence of DNA polymerase and two differentially labeled dideoxynucleosidetriphosphates. If the polymorphism is present on the template, one of the labeled dideoxynucleosidetriphosphates can be added to the primer in a single base chain extension. The allele present is then inferred by determining which of the two differential labels was added to the extension primer. Homozygous samples will result in only one of the two labeled bases being incorporated and thus only one of the two labels will be detected. Heterozygous samples have both alleles present, and will thus direct incorporation of both labels (into different molecules of the extension primer) and thus both labels will be detected.

In another method for detecting polymorphisms, SNPs and Indels can be detected by methods disclosed in U.S. Pat. No. 5,210,015; U.S. Pat. No. 5,876,930; and U.S. Pat. No. 6,030,787 in which an oligonucleotide probe having a 5′ fluorescent reporter dye and a 3′ quencher dye covalently linked to the 5′ and 3′ ends of the probe. When the probe is intact, the proximity of the reporter dye to the quencher dye results in the suppression of the reporter dye fluorescence, e.g. by Forster-type energy transfer. During PCR forward and reverse primers hybridize to a specific sequence of the target DNA flanking a polymorphism while the hybridization probe hybridizes to polymorphism-containing sequence within the amplified PCR product. In the subsequent PCR cycle DNA polymerase with 5′→3′ exonuclease activity cleaves the probe and separates the reporter dye from the quencher dye resulting in increased fluorescence of the reporter.

In another embodiment, the locus or loci of interest can be directly sequenced using nucleic acid sequencing technologies. Methods for nucleic acid sequencing are known in the art and include technologies provided by 454 Life Sciences (Branford, Conn.), Agencourt Bioscience (Beverly, Mass.), Applied Biosystems (Foster City, Calif.), LI-COR Biosciences (Lincoln, Nebr.), NimbleGen Systems (Madison, Wis.), Illumina (San Diego, Calif.), and VisiGen Biotechnologies (Houston, Tex.). Such nucleic acid sequencing technologies comprise formats such as parallel bead arrays, sequencing by ligation, capillary electrophoresis, electronic microchips, “biochips,” microarrays, parallel microchips, and single-molecule arrays, as reviewed by R.F. Service Science 2006 311:1544-1546.

For the purpose of QTL mapping, the markers to be used in the methods of the present invention should preferably be diagnostic of origin in order for inferences to be made about subsequent populations. Experience to date suggests that SNP markers may be ideal for mapping because the likelihood that a particular SNP allele is derived from independent origins in the extant populations of a particular species is very low. As such, SNP markers appear to be useful for tracking and assisting introgression of QTLs, particularly in the case of haplotypes.

As used herein, a “nucleic acid molecule,” be it a naturally occurring molecule or otherwise may be “substantially purified”, if desired, referring to a molecule separated from substantially all other molecules normally associated with it in its native state. More preferably, a substantially purified molecule is the predominant species present in a preparation. A substantially purified molecule may be at least about 60% free, preferably at least about 75% free, more preferably at least about 90% free, and most preferably at least about 95% free from the other molecules (exclusive of solvent) present in the natural mixture. The term “substantially purified” is not intended to encompass molecules present in their native state.

The agents of the present invention will preferably be “biologically active” with respect to either a structural attribute, such as the capacity of a nucleic acid to hybridize to another nucleic acid molecule, or the ability of a protein to be bound by an antibody (or to compete with another molecule for such binding). Alternatively, such an attribute may be catalytic, and thus involve the capacity of the agent to mediate a chemical reaction or response.

The agents of the present invention may also be recombinant. As used herein, the term recombinant means any agent (e.g. DNA, peptide etc.), that is, or results, however indirect, from human manipulation of a nucleic acid molecule.

The agents of the present invention may be labeled with reagents that facilitate detection of the agent (e.g. fluorescent labels (Prober et al. 1987 Science 238:336-340; European Patent 144914), chemical labels (U.S. Pat. No. 4,582,789; U.S. Pat. No. 4,563,417), and modified bases (European Patent 119448).

### 2. Marker-Trait Associations

The present invention provides methods for identification of transgene modulating loci using mapping techniques. By establishing transgene performance as a phenotype, genotypes associated with preferred transgene performance are identified. The methods of the present invention are useful for comparing two or more transgenic events in one or more germplasm entries as well as comparing one or more transgenic events in two or more germplasm entries, depending on the phase of the transgene in the transgenic breeding pipeline. Exemplary methods for the detection of marker-trait associations are set forth below.

Because of allelic differences in genetic markers, QTL can be identified by statistical evaluation of the genotypes and phenotypes of segregating populations. Processes to map QTL are well-described (WO 90/04651; U.S. Pat. No. 5,492,547, U.S. Pat. No. 5,981,832, U.S. Pat. No. 6,455,758; reviewed in Flint-Garcia et al. 2003 Ann. Rev. Plant Biol. Ann. Rev. Plant Biol. 54:357-374). Methods for determining the statistical significance of a correlation between a phenotype and a genotype, whether a genetic marker or haplotype, may be determined by any statistical test known in the art and with any accepted threshold of statistical significance being required. The application of particular methods and thresholds of significance are well within the skill of the ordinary practitioner of the art. Notably, any type of marker can be correlated with the causative genotype and selection decisions can be made based on a genetic or phenotypic marker.

Using markers to infer a phenotype of interest results in the economization of a breeding program by substituting costly, time-intensive phenotyping with genotyping or a cheaper phenotyping platform, such as an early emerging phenotypic character. Further, breeding programs can be designed to explicitly drive the frequency of specific, favorable phenotypes by targeting particular genotypes (U.S. Pat. No. 6,399,855). Fidelity of these associations may be monitored continuously to ensure maintained predictive ability and, thus, informed breeding decisions (US Published Patent Application 2005/0015827).

An allele of a QTL can comprise multiple genes or other genetic factors even within a contiguous genomic region or linkage group, such as a haplotype. As used herein, an allele of a QTL or transgene modulating locus can therefore encompass more than one gene or other genetic factor where each individual gene or genetic component is also capable of exhibiting allelic variation and where each gene or genetic factor is also capable of eliciting a phenotypic effect on the quantitative trait in question. In an aspect of the present invention, the allele of a QTL comprises one or more genes or other genetic factors that are also capable of exhibiting allelic variation. The use of the term “an allele of a QTL” is thus not intended to exclude a QTL that comprises more than one gene or other genetic factor. Specifically, an “allele of a QTL” in the present invention can denote a haplotype within a haplotype window wherein a phenotype can be disease resistance. A haplotype window is a contiguous genomic region that can be defined, and tracked, with a set of one or more polymorphic markers wherein the polymorphisms indicate identity by descent. A haplotype within that window can be defined by the unique fingerprint of alleles at each marker. As used herein, an allele is one of several alternative forms of a gene occupying a given locus on a chromosome. When all the alleles present at a given locus on a chromosome are the same, that plant is homozygous at that locus. If the alleles present at a given locus on a chromosome differ, that plant is heterozygous at that locus. Plants of the present invention may be homozygous or heterozygous at any particular transgene modulating locus or for a particular polymorphic marker.

The identification of marker-trait associations has evolved to the application of genetic markers as a tool for the selection of “new and superior plants” via introgression of preferred genomic regions as determined by statistical analyses (U.S. Pat. No. 6,219,964). Marker-assisted introgression involves the transfer of a chromosomal region, defined by one or more markers, from one germplasm to a second germplasm. The initial step in that process is the localization of the genomic region or transgene by gene mapping, which is the process of determining the position of a gene or genomic region relative to other genes and genetic markers through linkage analysis. The basic principle for linkage mapping is that the closer together two genes are on a chromosome, the more likely they are to be inherited together. Briefly, a cross is generally made between two genetically compatible but divergent parents relative to the traits of interest. Genetic markers can then be used to follow the segregation of these traits in the progeny from the cross, often a backcross (BC1), F₂, or recombinant inbred population.

In plant breeding populations, linkage disequilibrium (LD) is the level of departure from random association between two or more loci in a population and LD often persists over large chromosomal segments. Although it is possible for one to be concerned with the individual effect of each gene in the segment, for a practical plant breeding purpose the emphasis is typically on the average impact the region has for the trait(s) of interest when present in a line, hybrid or variety. The amount of pair-wise LD is calculated (using the r² statistic) against the distance in centiMorgan (cM, one hundredth of a Morgan, on average one recombination per meiosis, recombination is the result of the reciprocal exchange of chromatid segments between homologous chromosomes paired at meiosis, and it is usually observed through the association of alleles at linked loci from different grandparents in the progeny) using a set of genetic markers and set of germplasm entries.

The genetic linkage of additional genetic marker molecules can be established by a gene mapping model such as, without limitation, the flanking marker model reported by Lander et al. (Lander et al. 1989 Genetics, 121:185-199), and the interval mapping, based on maximum likelihood methods described therein, and implemented in the software package MAPMAKER/QTL (Lincoln and Lander, Mapping Genes Controlling Quantitative Traits Using MAPMAKER/QTL, Whitehead Institute for Biomedical Research, Massachusetts, (1990). Additional software includes Qgene, Version 2.23 (1996), Department of Plant Breeding and Biometry, 266 Emerson Hall, Cornell University, Ithaca, N.Y.). Use of Qgene software is a particularly preferred approach.

A maximum likelihood estimate (MLE) for the presence of a genetic marker is calculated, together with an MLE assuming no QTL effect, to avoid false positives. A log₁₀ of an odds ratio (LOD) is then calculated as: LOD=log₁₀ (MLE for the presence of a QTL/MLE given no linked QTL). The LOD score essentially indicates how much more likely the data are to have arisen assuming the presence of a QTL versus in its absence. The LOD threshold value for avoiding a false positive with a given confidence, say 95%, depends on the number of genetic markers and the length of the genome. Graphs indicating LOD thresholds are set forth in Lander et al. (1989), and further described by Arús and Moreno-González, Plant Breeding, Hayward, Bosemark, Romagosa (eds.) Chapman & Hall, London, pp. 314-331 (1993).

Additional models can be used. Many modifications and alternative approaches to interval mapping have been reported, including the use of non-parametric methods (Kruglyak et al. 1995 Genetics, 139:1421-1428). Multiple regression methods or models can be also be used, in which the trait is regressed on a large number of genetic markers (Jansen, Biometrics in Plant Breed, van Oijen, Jansen (eds.) Proceedings of the Ninth Meeting of the Eucarpia Section Biometrics in Plant Breeding, The Netherlands, pp. 116-124 (1994); Weber and Wricke, Advances in Plant Breeding, Blackwell, Berlin, 16 (1994)). Procedures combining interval mapping with regression analysis, whereby the phenotype is regressed onto a single putative QTL at a given genetic marker interval, and at the same time onto a number of genetic markers that serve as ‘cofactors,’ have been reported by Jansen et al. (Jansen et al. 1994 Genetics, 136:1447-1455) and Zeng (Zeng 1994 Genetics 136:1457-1468). Generally, the use of cofactors reduces the bias and sampling error of the estimated QTL positions (Utz and Melchinger, Biometrics in Plant Breeding, van Oijen, Jansen (eds.) Proceedings of the Ninth Meeting of the Eucarpia Section Biometrics in Plant Breeding, The Netherlands, pp. 195-204 (1994), thereby improving the precision and efficiency of QTL mapping (Zeng 1994). These models can be extended to multi-environment experiments to analyze genotype-environment interactions (Jansen et al. 1995 Theor. Appl. Genet. 91:33-3). Association study approaches such as transmission disequilibrium tests may be useful for detecting marker-trait associations (Stich et al. 2006 Theor. Appl. Genet. 113:1121-1130).

An alternative to traditional QTL mapping involves achieving higher resolution by mapping haplotypes, versus individual genetic markers (Fan et al. 2006 Genetics 172:663-686). This approach tracks blocks of DNA known as haplotypes, as defined by polymorphic genetic markers, which are assumed to be identical by descent in the mapping population. This assumption results in a larger effective sample size, offering greater resolution of QTL. Methods for determining the statistical significance of a correlation between a phenotype and a genotype, in this case a haplotype, may be determined by any statistical test known in the art and with any accepted threshold of statistical significance being required. The application of particular methods and thresholds of significance are well with in the skill of the ordinary practitioner of the art.

Selection of appropriate mapping populations is important to map construction. The choice of an appropriate mapping population depends on the type of marker systems employed (Tanksley et al., Molecular mapping in plant chromosomes. chromosome structure and function: Impact of new concepts J. P. Gustafson and R. Appels (eds.). Plenum Press, New York, pp. 157-173 (1988)). Consideration must be given to the source of parents (adapted vs. exotic) used in the mapping population. Chromosome pairing and recombination rates can be severely disturbed (suppressed) in wide crosses (adapted×exotic) and generally yield greatly reduced linkage distances. Wide crosses will usually provide segregating populations with a relatively large array of polymorphisms when compared to progeny in a narrow cross (adapted×adapted).

An F₂ population is the first generation of selfing after the hybrid seed is produced. Usually a single F₁ plant is selfed to generate a population segregating for all the genes in Mendelian (1:2:1) fashion. Maximum genetic information is obtained from a completely classified F₂ population using a codominant genetic marker system (Mather, Measurement of Linkage in Heredity: Methuen and Co., (1938)). In the case of dominant markers, progeny tests (e.g. F₃, BCF₂) are required to identify the heterozygotes, thus making it equivalent to a completely classified F₂ population. However, this procedure is often prohibitive because of the cost and time involved in progeny testing. Progeny testing of F₂ individuals is often used in map construction where phenotypes do not consistently reflect genotype (e.g. disease resistance) or where trait expression is controlled by a QTL. Segregation data from progeny test populations (e.g. F₃ or BCF₂) can be used in map construction. Marker-assisted selection can then be applied to cross progeny based on marker-trait map associations (F₂, F₃), where linkage groups have not been completely disassociated by recombination events (i.e., maximum disequilibrium).

Recombinant inbred lines (RIL) (genetically related lines; usually >F₅, developed from continuously selfing F₂ lines towards homozygosity) can be used as a mapping population. Information obtained from dominant markers can be maximized by using RIL because all loci are homozygous or nearly so. Under conditions of tight linkage (i.e., about <10% recombination), dominant and co-dominant genetic markers evaluated in RIL populations provide more information per individual than either marker type in backcross populations (Reiter et al. 1992 Proc. Natl. Acad. Sci. (USA) 89:1477-1481). However, as the distance between markers becomes larger (i.e., loci become more independent), the information in RIL populations decreases dramatically.

Backcross populations (e.g., generated from a cross between a successful variety (recurrent parent) and another variety (donor parent) carrying a trait not present in the former) can be utilized as a mapping population. A series of backcrosses to the recurrent parent can be made to recover most of its desirable traits. Thus a population is created consisting of individuals nearly like the recurrent parent but each individual carries varying amounts of genomic regions from the donor parent. Backcross populations can be useful for mapping dominant genetic markers if all loci in the recurrent parent are homozygous and the donor and recurrent parent have contrasting polymorphic marker alleles (Reiter et al. 1992 Proc. Natl. Acad. Sci. (USA) 89:1477-1481). Information obtained from backcross populations using either codominant or dominant markers is less than that obtained from F₂ populations because one, rather than two, recombinant gametes are sampled per plant. Backcross populations, however, are more informative (at low marker saturation) when compared to RILs as the distance between linked loci increases in RIL populations (i.e. about 0.15% recombination). Increased recombination can be beneficial for resolution of tight linkages, but may be undesirable in the construction of maps with low marker saturation.

Near-isogenic lines (NIL) created by many backcrosses to produce an array of individuals that are nearly identical in genetic composition except for the trait or genomic region under interrogation can be used as a mapping population. In mapping with NILs, only a portion of the polymorphic loci are expected to map to a selected region.

Bulk segregant analysis (BSA) is a method developed for the rapid identification of linkage between genetic markers and traits of interest (Michelmore et al. 1991 Proc. Natl. Acad. Sci. (U.S.A.) 88:9828-9832). In BSA, two bulked DNA samples are drawn from a segregating population originating from a single cross. These bulks contain individuals that are identical for a particular trait (resistant or susceptible to particular disease) or genomic region but arbitrary at unlinked regions (i.e. heterozygous). Regions unlinked to the target region will not differ between the bulked samples of many individuals in BSA.

In another embodiment, plants can be screened for one or more markers associated with at least one transgene modulating locus using high throughput, non-destructive seed sampling. Apparatus and methods for the high-throughput, non-destructive sampling of seeds have been described which would overcome the obstacles of statistical samples by allowing for individual seed analysis. For example, published U.S. Patent Applications US 2006/0042527, US 2006/0046244, US 2006/0046264, US 2006/0048247, US 2006/0048248, US 2007/0204366, and US 2007/0207485, which are incorporated herein by reference in their entirety, disclose apparatus and systems for the automated sampling of seeds as well as methods of sampling, testing and bulking seeds. Thus, in a preferred embodiment, a method of the present invention comprises screening for markers in individual seeds of a population wherein only seed with at least one genotype of interest is advanced.

### 3. Plant Breeding

Plants of the present invention can be part of or generated from a breeding program. The choice of breeding method depends on the mode of plant reproduction, the heritability of the trait(s) being improved, and the type of cultivar used commercially (e.g., F₁ hybrid cultivar, pureline cultivar, etc). A cultivar is a race or variety of a plant species that has been created or selected intentionally and maintained through cultivation.

The present invention provides for parts of the plants of the present invention.

Selected, non-limiting approaches for breeding the plants of the present invention are set forth below. A breeding program can be enhanced using marker assisted selection (MAS) on the progeny of any cross. It is understood that nucleic acid markers of the present invention can be used in a MAS (breeding) program. It is further understood that any commercial and non-commercial cultivars can be utilized in a breeding program. Factors such as, for example, emergence vigor, vegetative vigor, stress tolerance, disease resistance, branching, flowering, seed set, seed size, seed density, standability, and threshability etc. will generally dictate the choice.

For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection. In a preferred aspect, a backcross or recurrent breeding program is undertaken.

The complexity of inheritance influences choice of the breeding method. Backcross breeding can be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes.

Breeding lines can be tested and compared to appropriate standards in environments representative of the commercial target area(s) for two or more generations. The best lines are candidates for new commercial cultivars; those still deficient in traits may be used as parents to produce new populations for further selection.

For hybrid crops, the development of new elite hybrids requires the development and selection of elite inbred lines, the crossing of these lines and selection of superior hybrid crosses. The hybrid seed can be produced by manual crosses between selected male-fertile parents or by using male sterility systems. Additional data on parental lines, as well as the phenotype of the hybrid, influence the breeder's decision whether to continue with the specific hybrid cross.

Pedigree breeding and recurrent selection breeding methods can be used to develop cultivars from breeding populations. Breeding programs combine desirable traits from two or more cultivars or various broad-based sources into breeding pools from which cultivars are developed by selfing and selection of desired phenotypes. New cultivars can be evaluated to determine which have commercial potential.

Backcross breeding has been used to transfer genes for a simply inherited, highly heritable trait into a desirable homozygous cultivar or inbred line, which is the recurrent parent. The source of the trait to be transferred is called the donor parent. After the initial cross, individuals possessing the phenotype of the donor parent are selected and repeatedly crossed (backcrossed) to the recurrent parent. The resulting plant is expected to have most attributes of the recurrent parent (e.g., cultivar) and, in addition, the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

The doubled haploid (DH) approach achieves isogenic plants in a shorter time frame. DH plants provide an invaluable tool to plant breeders, particularly for generating inbred lines and quantitative genetics studies. For breeders, DH populations have been particularly useful in QTL mapping, cytoplasmic conversions, and trait introgression. Moreover, there is value in testing and evaluating homozygous lines for plant breeding programs. All of the genetic variance is among progeny in a breeding cross, which improves selection gain.

Most research and breeding applications rely on artificial methods of DH production. The initial step involves the haploidization of the plant which results in the production of a population comprising haploid seed. Non-homozygous lines are crossed with an inducer parent, resulting in the production of haploid seed. Seed that has a haploid embryo, but normal triploid endosperm, advances to the second stage. That is, haploid seed and plants are any plant with a haploid embryo, independent of the ploidy level of the endosperm.

After selecting haploid seeds from the population, the selected seeds undergo chromosome doubling to produce doubled haploid seeds. A spontaneous chromosome doubling in a cell lineage will lead to normal gamete production or the production of unreduced gametes from haploid cell lineages. Application of a chemical compound, such as colchicine, can be used to increase the rate of diploidization. Colchicine binds to tubulin and prevents its polymerization into microtubules, thus arresting mitosis at metaphase, can be used to increase the rate of diploidization, i.e. doubling of the chromosome number These chimeric plants are self-pollinated to produce diploid (doubled haploid) seed. This DH seed is cultivated and subsequently evaluated and used in hybrid testcross production.

Descriptions of other breeding methods that are commonly used for different traits and crops can be found in one of several reference books (Allard, “Principles of Plant Breeding,” John Wiley & Sons, NY, U. of CA, Davis, Calif., 50-98, 1960; Simmonds, “Principles of crop improvement,” Longman, Inc., NY, 369-399, 1979; Sneep and Hendriksen, “Plant breeding perspectives,” Wageningen (ed), Center for Agricultural Publishing and Documentation, 1979; Fehr, In: Soybeans: Improvement, Production and Uses, 2nd Edition, Monograph., 16:249, 1987; Fehr, “Principles of variety development,” Theory and Technique, (Vol. 1) and Crop Species Soybean (Vol. 2), Iowa State Univ., Macmillan Pub. Co., NY, 360-376, 1987).

In one embodiment of the present invention, when conserved genetic segments, or haplotype windows, are coincident with segments in which transgene modulating QTL have been identified, the methods of the present invention allow for one skilled in the art to extrapolate, with high probability, QTL inferences to other germplasm having an identical haplotype or genetic marker allele in that haplotype window. This a priori information provides the basis to select for favorable QTLs prior to QTL mapping within a given population. In a preferred embodiment, the QTL are associated with transgene performance and expression.

For example, the methods of the present invention allow one skilled in the art to make plant breeding decisions regarding transgene modulating loci comprising:
a) Selection among new breeding populations to determine which populations have the highest frequency of favorable haplotypes or genetic marker alleles, wherein haplotypes and marker alleles are designated as favorable based on coincidence with previous QTL mapping; or
b) Selection of progeny containing the favorable haplotypes or genetic marker alleles in breeding populations prior to, or in substitution for, QTL mapping within that population, wherein selection could be done at any stage of breeding and could also be used to drive multiple generations of recurrent selection; or
c) Prediction of progeny performance for specific breeding crosses; or
d) S Selection of lines for germplasm improvement activities based on said favorable haplotypes or genetic marker alleles (as disclosed in PCT Patent Application Publication No. WO 2008/021413), including line development, hybrid development, selection among transgenic events based on the breeding value of the haplotype that the transgene is in linkage with (as disclosed in U.S. patent application Ser. No. 11/441,91), making breeding crosses, testing and advancing a plant through self fertilization, purification of lines or sublines, using plant or parts thereof for transformation, using plants or parts thereof for candidates for expression constructs, and using plant or parts thereof for mutagenesis.

In addition, when the methods of the present invention are used for gene identification along with the use of integrated physical and genetic maps and various nucleic acid sequencing approaches, one skilled in the art can practice the combined methods to select for specific genes or gene alleles. For example, when haplotype windows are coincident with segments in which genes have been identified, one skilled in the art can extrapolate gene inferences to other germplasm having an identical genetic marker allele or alleles, or haplotype, in that haplotype window. This a priori information provides the basis to select for favorable genes or gene alleles on the basis of haplotype(s) or marker allele(s) identification within a given population.

For example, the methods of the present invention allow one skilled in the art to make plant breeding decisions comprising:
a) Selection among new breeding populations to determine which populations have the highest frequency of favorable haplotypes or genetic marker alleles, wherein haplotypes or marker alleles are designated as favorable based on coincidence with previous gene mapping; or
b) Selection of progeny containing the favorable haplotypes or genetic marker alleles in breeding populations, wherein selection is effectively enabled at the gene level, wherein selection could be done at any stage of inbreeding and could also be used to drive multiple generations of recurrent selection; or
c) Prediction of progeny performance for specific breeding crosses; or
d) Selection of lines for germplasm improvement activities based on said favorable haplotypes or genetic marker alleles (as disclosed in PCT Patent Application Publication No. WO 2008/021413), including line development, hybrid development, selection among transgenic events based on the breeding value of the haplotype that the transgene is in linkage with (as disclosed in U.S. patent application Ser. No. 11/441,91), making breeding crosses, testing and advancing a plant through self fertilization, purification of lines or sublines, using plant or parts thereof for transformation, using plants or parts thereof for candidates for expression constructs, and using plant or parts thereof for mutagenesis.

Another preferred embodiment of the present invention provides for the selection of a composition of QTL wherein each QTL is associated with a phenotype for transgene performance or expression.

Another embodiment of this invention is a method for enhancing breeding populations by accumulation of one or more haplotypes in a germplasm. Genomic regions defined as haplotype windows include genetic information and provide phenotypic traits to the plant. Variations in the genetic information can result in variation of the phenotypic trait and the value of the phenotype can be measured. The genetic mapping of the haplotype windows allows for a determination of linkage across haplotypes. The haplotype of interest has a DNA sequence that is novel in the genome of the progeny plant and can in itself serve as a genetic marker of haplotype of interest. Notably, this marker can also be used as an identifier for a gene or QTL. For example, in the event of multiple traits or trait effects associated with the haplotype, only one genetic marker would be necessary for selection purposes. Additionally, the haplotype of interest may provide a means to select for plants that have the linked haplotype region. Selection may be due to tolerance to an applied phytotoxic chemical, such as an herbicide or antibiotic, or to pathogen resistance. Selection may be due to phenotypic selection means, such as, a morphological phenotype that is easy to observe such as seed color, seed germination characteristic, seedling growth characteristic, leaf appearance, plant architecture, plant height, and flower and fruit morphology.

Using this method, the present invention contemplates that haplotypes of interest are selected from a large population of plants, and these haplotypes can have a synergistic breeding value in the germplasm of a crop plant. Additionally, these haplotypes can be used in the described breeding methods to accumulate other beneficial and preferred haplotype regions and maintain these in a breeding population to enhance the overall germplasm of the crop plant. Crop plants considered for use in the method include but are not limited to maize (Zea mays), soybean (Glycine max), cotton (Gossypium hirsutum), peanut (Arachis hypogaea), barley (Hordeum vulgare); oats (Avena sativa); orchard grass (Dactylis glomerata); rice (Oryza sativa, including indica and japonica varieties); sorghum (Sorghum bicolor); sugar cane (Saccharum sp); tall fescue (Festuca arundinacea); turfgrass species (e.g. species: Agrostis stolonifera, Poa pratensis, Stenotaphrum secundatum); wheat (Triticum aestivum), and alfalfa (Medicago sativa), members of the genus Brassica, broccoli, cabbage, carrot, cauliflower, Chinese cabbage, cucumber, dry bean, eggplant, fennel, garden beans, gourd, leek, lettuce, melon, okra, onion, pea, pepper, pumpkin, radish, spinach, squash, sweet corn, tomato, watermelon, ornamental plants, and other fruit, vegetable, tuber, and root crops.

Non-limiting examples of elite corn inbreds that are commercially available to farmers include ZS4199, ZS02433, G3000, G1900, G0302, G1202, G2202, G4901, G3601, G1900 (Advanta Technology Ltd., Great Britain); 6TR512, 7RN401, 6RC172, 7SH382, MV7100, 3JP286, BE4207, 4VP500, 7SH385, 5XH755, 7SH383, 11084BM, 2JK221, 4XA321, 6RT321, BE8736, MV5125, MV8735, 3633BM (Dow, Michigan, USA); 8982-11-4-2, 8849, IT302, 9034, IT201, RR728-18, 5020, BT751-31 (FFR Cooperative, Indiana, USA); 1874WS, X532Y, 1784S, 1778S, 1880S (Harris Moran Seed Company, California, USA); FR3351, FR2108, FR3383, FR3303, FR3311, FR3361 (Illinois Foundation Seeds, Inc., Illinois, USA); NR109, JCRNR113, MR724, M42618, CI9805, JCR503, NR401, W60028, N16028, N10018, E24018, A60059, W69079, W23129 (J.C. Robinson Seed Company, Nebraska, USA); 7791, KW4773, KW7606, KW4636, KW7648, KW4U110, KWU7104, CB1, CC2 (KWS Kleinwanzlebener Saatzucgt A G, Germany); UBB3, TDC1, RAA1, VMM1, MNI1, RII1, RBO1 (Limagrain Genetics Grande Culture S. A., France); LH284, 7OLDL5, GM9215, 9OLDI1, 9OLDC2, 90QDD1, RDBQ2, 01HG12, 79314N1, 171N120, 17DHD7, 831N18, 83InI14, 01INL1, LH286, ASG29, ASG07, QH111, 09DSQ1, ASG09, 86AQV2, 86ISI5, ASG25, 01DHD16, ASG26, ASG28, 90LCL6, 22DHD11, ASG17, WDHQ2, ASG27, 90DJD28, WQCD10, 17DHD5, RQAA8, LH267, 29MIFI2, RQAB7, LH198Bt810, 3DHA9, LH200BT810, LH172Bt810, 01IZB2, ASG10, LH253, 861S127, 91ISI5, 22DHQ3, 91INI12, 861S126, 01IUL6, 89ADH11, 01HGI4, 161UL2, F307W, LH185Bt810, F351, LH293, LH245, 17DHD16, 90DHQ2, LH279, LH244, LH287, WDHQ11, 09DSS1, F6150, 17INI30, 4SCQ3, 01HF13, 87ATD2, 8M116, FBLL, 17QFB1, 83DNQ2, 94INK1A, NL054B, 6F545, F274, MBZA, 1389972, 94INK1B, 89AHD12, I889291, 3323, 16IUL6, 6077, I014738, 7180, GF6151, WQDS7, I465837, 3327, LH176Bt810, 181664, I362697, LH310, LH320, LH295, LH254, 5750, I390186, I501150, I363128, I244225, LH246, LH247, LH322, LH289, LH283BtMON810, 85DGD1, I390185, WDDQ1, LH331 (Monsanto Co., Missouri, USA); PH1B5, PH1CA, PHOWE, PH1GG, PH0CD, PH21T, PH224, PH0V0, PH3GR, PH1NF, PH0JG, PH189, PH12J, PH1EM, PH12C, PH55C, PH3EV, PH2V7, PH4TF, PH3 KP, PH2MW, PH2N0, PH1K2, PH226, PH2VJ, PH1M8, PH1B8, PH0WD, PH3GK, PH2VK, PH1MD, PH04G, PH2KN, PH2E4, PH0DH, PH1CP, PH3PO, PH1W0, PH45A, PH2VE, PH36E, PH50P, PH8V0, PH4TV, PH2JR, PH4PV, PH3DT, PH5D6, PH9KO, PH0B3, PH2EJ, PH4TW, PH77C, PH3HH, PH8W4, PH1GD, PH1BC, PH4V6, PH0R8, PH581, PH6WR, PH5HK, PH5W4, PH0KT, PH4GP, PHJ8R, PH7CP, PH6WG, PH54H, PH5DR, PH5WB, PH7CH, PH54M, PH726, PH48V, PH3PV, PH77V, PH7JB, PH70R, PH3RC, PH6 KW, PH951, PH6ME, PH87H, PH26N, PH9AH, PH51H, PH94T, PH7AB, PH5FW, PH75K, PH8CW, PH8PG, PH5TG, PH6JM, PH3AV, PH3PG, PH6WA, PH6CF, PH76T, PH6MN, PH7BW, PH890, PH876, PHAPV, PHB5R, PH8 DB, PH51K, PH87P, PH8KG, PH4CV, PH705, PH5DP, PH77N, PH86T, PHAVN, PHB6R, PH91C, PHCWK, PHC5H, PHACE, PHB6V, PH8JR, PH77P, PHBAB, PHB1V, PH3PR, PH8TN, PH5WA, PH58C, PH6HR, PH183, PH714, PHA9G, PH8BC, PHBBP, PHAKC, PHD90, PHACV, PHCEG, PHB18, PHB00, PNCND, PHCMV (Pioneer Hi-Bred International, Inc., Iowa, USA); GSC3, GSC1, GSC2, NP2138, 2227BT, ZS02234, NP2213, 2070BT, NP2010, NP2044BT, NP2073, NP2015, NP2276, NP2222, NP2052, NP2316, NP2171, WICY418C, NP2174, BX20010, BX20033, G6103, G1103, 291B, 413A, G1704 (Syngenta Participations AG, Switzerland). An elite plant is a representative plant from an elite line.

Examples of elite soybean varieties that are commercially available to farmers or soybean breeders such as HARTZ™ variety H4994, HARTZ™ variety H5218, HARTZ™ variety H5350, HARTZ™ variety H5545, HARTZ™ variety H5050, HARTZ™ variety H5454, HARTZ™ variety H5233, HARTZ™ variety H5488, HARTZ™ variety HLA572, HARTZ™ variety H6200, HARTZ™ variety H6104, HARTZ™ variety H6255, HARTZ™ variety H6586, HARTZ™ variety H6191, HARTZ™ variety H7440, HARTZ™ variety H4452 Roundup Ready™, HARTZ™ variety H4994 Roundup Ready™, HARTZ™ variety H4988 Roundup Ready™, HARTZ™ variety H5000 Roundup Ready™, HARTZ™ variety H5147 Roundup Ready™, HARTZ™ variety H5247 Roundup Ready™, HARTZ™ variety H5350 Roundup Ready™, HARTZ™ variety H5545 Roundup Ready™, HARTZ™ variety H5855 Roundup Ready™, HARTZ™ variety H5088 Roundup Ready™, HARTZ™ variety H5164 Roundup Ready™, HARTZ™ variety H5361 Roundup Ready™, HARTZ™ variety H5566 Roundup Ready™, HARTZ™ variety H5181 Roundup Ready™, HARTZ™ variety H5889 Roundup Ready™, HARTZ™ variety H5999 Roundup Ready™, HARTZ™ variety H6013 Roundup Ready™, HARTZ™ variety H6255 Roundup Ready™, HARTZ™ variety H6454 Roundup Ready™, HARTZ™ variety H6686 Roundup Ready™, HARTZ™ variety H7152 Roundup Ready™, HARTZ™ variety H7550 Roundup Ready™, HARTZ™ variety H8001 Roundup Ready™ (HARTZ SEED, Stuttgart, Ark., USA); A0868, AG0202, AG0401, AG0803, AG0901, A1553, A1900, AG1502, AG1702, AG1901, A1923, A2069, AG2101, AG2201, AG2205, A2247, AG2301, A2304, A2396, AG2401, AG2501, A2506, A2553, AG2701, AG2702, AG2703, A2704, A2833, A2869, AG2901, AG2902, AG2905, AG3001, AG3002, AG3101, A3204, A3237, A3244, AG3301, AG3302, AG3006, AG3203, A3404, A3469, AG3502, AG3503, AG3505, AG3305, AG3602, AG3802, AG3905, AG3906, AG4102, AG4201, AG4403, AG4502, AG4603, AG4801, AG4902, AG4903, AG5301, AG5501, AG5605, AG5903, AG5905, A3559, AG3601, AG3701, AG3704, AG3750, A3834, AG3901, A3904, A4045 AG4301, A4341, AG4401, AG4404, AG4501, AG4503, AG4601, AG4602, A4604, AG4702, AG4703, AG4901, A4922, AG5401, A5547, AG5602, AG5702, A5704, AG5801, AG5901, A5944, A5959, AG6101, AJW2600COR, FPG26932, QR4459 and QP4544 (Asgrow Seeds, Des Moines, Iowa, USA); DKB26-52, DKB28-51, DKB32-52, DKB08-51, DKB09-53, DKB10-52, DKB18-51, DKB26-53, DKB29-51, DKB42-51, DKB35-51 DKB34-51, DKB36-52, DKB37-51, DKB38-52, DKB46-51, DKB54-52 and DeKalb variety CX445 (DeKalb, Illinois, USA); 91B91, 92B24, 92B37, 92B63, 92B71, 92B74, 92B75, 92B91, 93B01, 93B1, 93B26, 93B34, 93B35, 93B41, 93B45, 93B51, 93B53, 93B66, 93B81, 93B82, 93B84, 94B01, 94B32, 94B53, 94M80 RR, 94M50 RR, 95B71, 95B95, 95M81 RR, 95M50 RR, 95M30 RR, 9306, 9294, 93M50, 93M93, 94B73, 94B74, 94M41, 94M70, 94M90, 95B32, 95B42, 95B43 and 9344 (Pioneer Hi-bred International, Johnston, Iowa, USA); SSC-251RR, SSC-273CNRR, AGRA 5429RR, SSC-314RR, SSC-315RR, SSC-311 STS, SSC-320RR, AGRA5432RR, SSC-345RR, SSC-356RR, SSC-366, SSC-373RR and AGRA5537CNRR (Schlessman Seed Company, Milan, Ohio, USA); 39-E9, 44-R4, 44-R5, 47-G7, 49-P9, 52-Q2, 53-K3, 56-J6, 58-V8, ARX A48104, ARX B48104, ARX B55104 and GP530 (Armor Beans, Fisher, Ark., USA); HT322STS, HT3596STS, L0332, L0717, L1309CN, L1817, L1913CN, L1984, L2303CN, L2495, L2509CN, L2719CN, L3997CN, L4317CN, RC1303, RC1620, RC1799, RC1802, RC1900, RC1919, RC2020, RC2300, RC2389, RC2424, RC2462, RC2500, RC2504, RC2525, RC2702, RC2964, RC3212, RC3335, RC3354, RC3422, RC3624, RC3636, RC3732, RC3838, RC3864, RC3939, RC3942, RC3964, RC4013, RC4104, RC4233, RC4432, RC4444, RC4464, RC4842, RC4848, RC4992, RC5003, RC5222, RC5332, RC5454, RC5555, RC5892, RC5972, RC6767, RC7402, RT0032, RT0041, RT0065, RT0073, RT0079, RT0255, RT0269, RT0273, RT0312, RT0374, RT0396, RT0476, RT0574, RT0583, RT0662, RT0669, RT0676, RT0684, RT0755, RT0874, RT0907, RT0929, RT0994, RT0995, RT1004, RT1183, RT1199, RT1234, RT1399, RT1413, RT1535, RT1606, RT1741, RT1789, RT1992, RT2000, RT2041, RT2089, RT2092, RT2112, RT2127, RT2200, RT2292, RT2341, RT2430, RT2440, RT2512, RT2544, RT2629, RT2678, RT2732, RT2800, RT2802, RT2822, RT2898, RT2963, RT3176, RT3200, RT3253, RT3432, RT3595, RT3836, RT4098, RX2540, RX2944, RX3444 and TS466RR (Croplan Genetics, Clinton, Ky., USA); 4340RR, 4630RR, 4840RR, 4860RR, 4960RR, 4970RR, 5260RR, 5460RR, 5555RR, 5630RR and 5702RR (Delta Grow, England, Ark., USA); DK3964RR, DK3968RR, DK4461RR, DK4763RR, DK4868RR, DK4967RR, DK5161RR, DK5366RR, DK5465RR, DK55T6, DK5668RR, DK5767RR, DK5967RR, DKXTJ446, DKXTJ448, DKXTJ541, DKXTJ542, DKXTJ543, DKXTJ546, DKXTJ548, DKXTJ549, DKXTJ54J9, DKXTJ54X9, DKXTJ554, DKXTJ555, DKXTJ55J5 and DKXTJ5K57 (Delta King Seed Company, McCrory, Ark., USA); DP 3861RR, DP 4331 RR, DP 4546RR, DP 4724 RR, DP 4933 RR, DP 5414RR, DP 5634 RR, DP 5915 RR, DPX 3950RR, DPX 4891RR, DPX 5808RR (Delta & Pine Land Company, Lubbock, Tex., USA); DG31T31, DG32C38, DG3362NRR, DG3390NRR, DG33A37, DG33B52, DG3443NRR, DG3463NRR, DG3481NRR, DG3484NRR, DG3535NRR, DG3562NRR, DG3583NRR, DG35B40, DG35D33, DG36M49, DG37N43, DG38K57, DG38T47, SX04334, SX04453 (Dyna-gro line, UAP-MidSouth, Cordova, Tenn., USA); 8374RR CYSTX, 8390 NNRR, 8416RR, 8492NRR and 8499NRR (Excel Brand, Camp Point, Illinois, USA); 4922RR, 5033RR, 5225RR and 5663RR (FFR Seed, Southhaven, Miss., USA); 3624RR/N, 3824RR/N, 4212RR/N, 4612RR/N, 5012RR/N, 5212RR/N and 5412RR/STS/N (Garst Seed Company, Slater, Iowa, USA); 471, 4R451, 4R485, 4R495, 4RS421 and 5R531 (Gateway Seed Company, Nashville, Ill., USA); H-3606RR, H-3945RR, H-4368RR, H-4749RR, H-5053RR and H-5492RR (Golden Harvest Seeds, Inc., Pekin, ill., USA); HBK 5324, HBK 5524, HBK R4023, HBK R4623, HBK R4724, HBK R4820, HBK R4924, HBK R4945CX, HBK R5620 and HBK R5624 (Hornbeck Seed Co. Inc., DeWitt, Ark., USA); 341 RR/SCN, 343 RR/SCN, 346 RR/SCN, 349 RR, 355 RR/SCN, 363 RR/SCN, 373 RR, 375 RR, 379 RR/SCN, 379+RR/SCN, 380 RR/SCN, 380+RR/SCN, 381 RR/SCN, 389 RR/SCN, 389+RR/SCN, 393 RR/SCN, 393+RR/SCN, 398 RR, 402 RR/SCN, 404 RR, 424 RR, 434 RR/SCN and 442 RR/SCN (Kruger Seed Company, Dike, Iowa, USA); 3566, 3715, 3875, 3944, 4010 and 4106 (Lewis Hybrids, Inc., Ursa, Ill., USA); C3999NRR (LG Seeds, Elmwood, Ill., USA); Atlanta 543, Austin RR, Cleveland VIIRR, Dallas RR, Denver RRSTS, Everest RR, Grant 3RR, Olympus RR, Phoenix IIIRR, Rocky RR, Rushmore 553RR and Washington IXRR (Merschman Seed Inc., West Point, Iowa, USA); RT 3304N, RT 3603N, RT 3644N, RT 3712N, RT 3804N, RT 3883N, RT 3991N, RT 4044N, RT 4114N, RT 4124N, RT 4201N, RT 4334N, RT 4402N, RT 4480N, RT 4503N, RT 4683N, RT 4993N, RT 5043N, RT 5204, RT 5553N, RT 5773, RT4731N and RTS 4824N (MFA Inc., Columbia, Mo., USA); 9A373NRR, 9A375XRR, 9A385NRS, 9A402NRR, 9A455NRR, 9A485XRR and 9B445NRS (Midland Genetics Group L.L.C., Ottawa, Kans., USA); 3605nRR, 3805nRR, 3903nRR, 3905nRR, 4305nRR, 4404nRR, 4705nRR, 4805nRR, 4904nRR, 4905nRR, 5504nRR and 5505nRR (Midwest Premium Genetics, Concordia, Mo., USA); S37-N4, S39-K6, S40-R9, S42-P7, S43-B1, S49-Q9, S50-N3, S52-U3 and S56-D7 (Syngenta Seeds, Henderson, Ky., USA); NT-3707 RR, NT-3737 RR/SCN, NT-3737+RR/SCN, NT-3737sc RR/SCN, NT-3777+RR, NT-3787 RR/SCN, NT-3828 RR, NT-3839 RR, NT-3909 RR/SCN/STS, NT-3909+RR/SCN/ST, NT-3909sc RR/SCN/S, NT-3919 RR, NT-3922 RR/SCN, NT-3929 RR/SCN, NT-3999 RR/SCN, NT-3999+RR/SCN, NT-3999sc RR/SCN, NT-4040 RR/SCN, NT-4040+RR/SCN, NT-4044 RR/SCN, NT-4122 RR/SCN, NT-4414 RR/SCN/STS, NT-4646 RR/SCN and NT-4747 RR/SCN (NuTech Seed Co., Ames, Iowa, USA); PB-3494NRR, PB-3732RR, PB-3894NRR, PB-3921NRR, PB-4023NRR, PB-4394NRR, PB-4483NRR and PB-5083NRR (Prairie Brand Seed Co., Story City, Iowa, USA); 3900RR, 4401RR, 4703RR, 4860RR, 4910, 4949RR, 5250RR, 5404RR, 5503RR, 5660RR, 5703RR, 5770, 5822RR, PGY 4304RR, PGY 4604RR, PGY 4804RR, PGY 5622RR and PGY 5714RR (Progeny Ag Products, Wynne, Ark., USA); R3595RCX, R3684Rcn, R3814RR, R4095Rcn, R4385Rcn and R4695Rcn (Renze Hybrids Inc., Carroll, Iowa, USA); S3532-4, S3600-4, S3832-4, S3932-4, S3942-4, S4102-4, S4542-4 and S4842-4 (Stine Seed Co., AdeI, Iowa, USA); 374RR, 398RRS (Taylor Seed Farms Inc., White Cloud, Kans., USA); USG 5002T, USG 510nRR, USG 5601T, USG 7440nRR, USG 7443nRR, USG 7473nRR, USG 7482nRR, USG 7484nRR, USG 7499nRR, USG 7504nRR, USG 7514nRR, USG 7523nRR, USG 7553nRS and USG 7563nRR (UniSouth Genetics Inc., Nashville, Tenn., USA); V38N5RS, V39N4RR, V42N3RR, V48N5RR, V284RR, V28N5RR, V315RR, V35N4RR, V36N5RR, V37N3RR, V40N3RR, V47N3RR, and V562NRR (Royster-Clark Inc., Washington C.H., Ohio, USA); RR2383N, 2525NA, RR2335N, RR2354N, RR2355N, RR2362, RR2385N, RR2392N, RR2392NA, RR2393N, RR2432N, RR2432NA, RR2445N, RR2474N, RR2484N, RR2495N and RR2525N (Willcross Seed, King City Seed, King City, Mo., USA); 1493RR, 1991NRR, 2217RR, 2301NRR, 2319RR, 2321NRR, 2341NRR, 2531NRR, 2541NRR, 2574RR, 2659RR, 2663RR, 2665NRR, 2671NRR, 2678RR, 2685RR, 2765NRR, 2782NRR, 2788NRR, 2791NRR, 3410RR, 3411NRR, 3419NRR, 3421NRR, 3425NRR, 3453NRR, 3461NRR, 3470CRR, 3471NRR, 3473NRR, 3475RR, 3479NRR, 3491NRR, 3499NRR, WX134, WX137, WX177 and WX300 (Wilken Seeds, Pontiac, Ill., USA). An elite plant is a representative plant from an elite variety.

TABLE 1
Examples of elite canola varieties that are commercially
available to farmers or breeders. An elite plant is a
representative plant from an elite variety.

| Canola variety | Supplier |
|---|---|
| 500 | Agriprogress Inc. |
| 601 | Agriprogress Inc. |
| 1492 | Agriprogress Inc. |
| 1604 | Agriprogress Inc. |
| 1841 | Agriprogress Inc. |
| 1768S | Agriprogress Inc. |
| 1878 V | Agriprogress Inc. |
| 99CH01 | Agriprogress Inc. |
| Baldur | Agriprogress Inc. |
| BIANCA | Agriprogress Inc. |
| BIANCA II | Agriprogress Inc. |
| DS-Roughrider | Agriprogress Inc. |
| Goliath | Agriprogress Inc. |
| Hudson | Agriprogress Inc. |
| HY-PER Star 100 | Agriprogress Inc. |
| Kronos | Agriprogress Inc. |
| LG3220 | Agriprogress Inc. |
| LG3222 | Agriprogress Inc. |
| Manor | Agriprogress Inc. |
| Reaper | Agriprogress Inc. |
| Rugby | Agriprogress Inc. |
| 2463 | Bayer CropScience Canada Co. |
| 2473 | Bayer CropScience Canada Co. |
| 2563 | Bayer CropScience Canada Co. |
| 2573 | Bayer CropScience Canada Co. |
| 2643 | Bayer CropScience Canada Co. |
| 2663 | Bayer CropScience Canada Co. |
| 2673 | Bayer CropScience Canada Co. |
| 2733 | Bayer CropScience Canada Co. |
| 2763 | Bayer CropScience Canada Co. |
| 5003 | Bayer CropScience Canada Co. |
| 5020 | Bayer CropScience Canada Co. |
| 5030 | Bayer CropScience Canada Co. |
| 5070 | Bayer CropScience Canada Co. |
| 5108 | Bayer CropScience Canada Co. |
| 5440 | Bayer CropScience Canada Co. |
| 8440 | Bayer CropScience Canada Co. |
| 9590 | Bayer CropScience Canada Co. |
| 1007 | Bonis and Co. Ltd. |
| 73P01 RR | Bonis and Co. Ltd. |
| 74P00 LL | Bonis and Co. Ltd. |
| 84S00 LL | Bonis and Co. Ltd. |
| CASH | Bonis and Co. Ltd. |
| Casino | Bonis and Co. Ltd. |
| DEFENDER | Bonis and Co. Ltd. |
| EAGLE | Bonis and Co. Ltd. |
| FAIRVIEW | Bonis and Co. Ltd. |
| FOOTHILLS | Bonis and Co. Ltd. |
| IMPULSE | Bonis and Co. Ltd. |
| Legacy | Bonis and Co. Ltd. |
| LoLinda | Bonis and Co. Ltd. |
| NORWESTER | Bonis and Co. Ltd. |
| OAC Hurricane | Bonis and Co. Ltd. |
| OAC Tornado | Bonis and Co. Ltd. |
| SENATOR | Bonis and Co. Ltd. |
| SPONSOR | Bonis and Co. Ltd. |
| SW 5001 | Bonis and Co. Ltd. |
| SW ARROW | Bonis and Co. Ltd. |
| SW BadgeRR | Bonis and Co. Ltd. |
| SW GladiatoRR | Bonis and Co. Ltd. |
| SW High Level | Bonis and Co. Ltd. |
| SW Peak RR | Bonis and Co. Ltd. |
| SW RazoR | Bonis and Co. Ltd. |
| SW RideR | Bonis and Co. Ltd. |
| SW Spirit River | Bonis and Co. Ltd. |
| SW WaRRior | Bonis and Co. Ltd. |
| Valleyview | Bonis and Co. Ltd. |
| WESTWIN | Bonis and Co. Ltd. |
| MillenniUM 03 | Bunge Canada |
| Red River 1826 | Bunge Canada |
| Red River 1852 | Bunge Canada |
| v1035 | Cargill Limited |
| v2010 | Cargill Limited |
| v2015 | Cargill Limited |
| Canterra 1867 | Cargill Specialty Canola Oils |
| Heritage | Cargill Specialty Canola Oils |
| IMC02 | Cargill Specialty Canola Oils |
| IMC03 | Cargill Specialty Canola Oils |
| IMC104 | Cargill Specialty Canola Oils |
| IMC105 | Cargill Specialty Canola Oils |
| IMC106RR | Cargill Specialty Canola Oils |
| IMC109RR | Cargill Specialty Canola Oils |
| IMC111RR | Cargill Specialty Canola Oils |
| IMC130 | Cargill Specialty Canola Oils |
| IMC140 | Cargill Specialty Canola Oils |
| IMC201 | Cargill Specialty Canola Oils |
| IMC203RR | Cargill Specialty Canola Oils |
| IMC204 | Cargill Specialty Canola Oils |
| IMC205 | Cargill Specialty Canola Oils |
| IMC206RR | Cargill Specialty Canola Oils |
| IMC207 | Cargill Specialty Canola Oils |
| IMC208RR | Cargill Specialty Canola Oils |
| IMC209RR | Cargill Specialty Canola Oils |
| IMC302 | Cargill Specialty Canola Oils |
| IMC303 | Cargill Specialty Canola Oils |
| IMC304RR | Cargill Specialty Canola Oils |
| Magellan | Cargill Specialty Canola Oils |
| v1010 | Cargill Specialty Canola Oils |
| v1030 | Cargill Specialty Canola Oils |
| v1031 | Cargill Specialty Canola Oils |
| v1032 | Cargill Specialty Canola Oils |
| CANTI CS | CAUSSADE SEMENCES S.A. |
| CHELSI | CAUSSADE SEMENCES S.A. |
| CINDI CS | CAUSSADE SEMENCES S.A. |
| JESPER | CEBECO SEMENCES S.A. |
| ARAWAK | CPB TWYFORD LTD |
| COMMANCHE | CPB TWYFORD LTD |
| HC 1217 | CPB TWYFORD LTD |
| HURON | CPB TWYFORD LTD |
| MOHICAN | CPB TWYFORD LTD |
| MS 692161 | CPB TWYFORD LTD |
| MS COMANCHE | CPB TWYFORD LTD |
| MS INCA | CPB TWYFORD LTD |
| NAVAJO | CPB TWYFORD LTD |
| NAVAJO MS | CPB TWYFORD LTD |
| RAPIER | CPB TWYFORD LTD |
| RPC 550 | CPB TWYFORD LTD |
| WH2112 | CPB TWYFORD LTD |
| CANNON | DANISCO SEED A/S |
| HERALD | DANISCO SEED A/S |
| INDUSTRY | DANISCO SEED A/S |
| MARINKA | DANISCO SEED A/S |
| SAHARA | DANISCO SEED A/S |
| ABILITY | DEUTSCHE SAATVEREDELUNG AG |
| BILLY | DEUTSCHE SAATVEREDELUNG AG |
| BRISE | DEUTSCHE SAATVEREDELUNG AG |
| CHARLY | DEUTSCHE SAATVEREDELUNG AG |
| DR 12 | DEUTSCHE SAATVEREDELUNG AG |
| EXOCET | DEUTSCHE SAATVEREDELUNG AG |
| FABIA | DEUTSCHE SAATVEREDELUNG AG |
| FUCHS | DEUTSCHE SAATVEREDELUNG AG |
| LIAISON | DEUTSCHE SAATVEREDELUNG AG |
| LIBOMIR | DEUTSCHE SAATVEREDELUNG AG |
| LICONGO | DEUTSCHE SAATVEREDELUNG AG |
| LICORNE | DEUTSCHE SAATVEREDELUNG AG |
| LICOSMOS | DEUTSCHE SAATVEREDELUNG AG |
| LICROWN | DEUTSCHE SAATVEREDELUNG AG |
| LIGHTNING | DEUTSCHE SAATVEREDELUNG AG |
| LIMBO | DEUTSCHE SAATVEREDELUNG AG |
| LIMPET | DEUTSCHE SAATVEREDELUNG AG |
| LION | DEUTSCHE SAATVEREDELUNG AG |
| LIONESS | DEUTSCHE SAATVEREDELUNG AG |
| LIPTON | DEUTSCHE SAATVEREDELUNG AG |
| LIZARD | DEUTSCHE SAATVEREDELUNG AG |
| OASE | DEUTSCHE SAATVEREDELUNG AG |
| QUEEN | DEUTSCHE SAATVEREDELUNG AG |
| V 140 OL | DEUTSCHE SAATVEREDELUNG AG |
| BRITTA | DLF-TRIFOLIUM A/S |
| CHANG | DLF-TRIFOLIUM A/S |
| CYCLONE | DLF-TRIFOLIUM A/S |
| HANSEN | DLF-TRIFOLIUM A/S |
| HELIOS | DLF-TRIFOLIUM A/S |
| JAZZ | DLF-TRIFOLIUM A/S |
| NIMBUS | DLF-TRIFOLIUM A/S |
| OLE | DLF-TRIFOLIUM A/S |
| OLSEN | DLF-TRIFOLIUM A/S |
| ORION | DLF-TRIFOLIUM A/S |
| PLUTO | DLF-TRIFOLIUM A/S |
| SI HANSEN | DLF-TRIFOLIUM A/S |
| SPOK | DLF-TRIFOLIUM A/S |
| STAR | DLF-TRIFOLIUM A/S |
| TAROK | DLF-TRIFOLIUM A/S |
| TRITOP | DLF-TRIFOLIUM A/S |
| UNICA | DLF-TRIFOLIUM A/S |
| Nex 500 | Dow AgroSciences |
| Nex 700 | Dow AgroSciences Canada Inc. |
| Nex 710 | Dow AgroSciences Canada Inc. |
| Nex 715 | Dow AgroSciences Canada Inc. |
| Nex 720 | Dow AgroSciences Canada Inc. |
| Nex 822 CL | Dow AgroSciences Canada Inc. |
| Nex 824 CL | Dow AgroSciences Canada Inc. |
| Nex 827 CL | Dow AgroSciences Canada Inc. |
| Nex 828 CL | Dow AgroSciences Canada Inc. |
| Nex 830 CL | Dow AgroSciences Canada Inc. |
| Nex 840 CL | Dow AgroSciences Canada Inc. |
| Nex 842 CL | Dow AgroSciences Canada Inc. |
| Nex 845 CL | Dow AgroSciences Canada Inc. |
| NEX170 | DOW AGROSCIENCES DENMARK A/S |
| NEX160 | DOW AGROSCIENCES LTD |
| 1812 | DSV Canada Inc. |
| 458RR | DSV Canada Inc. |
| 6045CL | DSV Canada Inc. |
| 624RR | DSV Canada Inc. |
| 811RR | DSV Canada Inc. |
| 829RR | DSV Canada Inc. |
| Agassiz | DSV Canada Inc. |
| Ascent | DSV Canada Inc. |
| LBD279 (USA 279) | DSV Canada Inc. |
| LBD449RR | DSV Canada Inc. |
| LBD561RR | DSV Canada Inc. |
| LBD588RR | DSV Canada Inc. |
| LBD612RR | DSV Canada Inc. |
| LBD644RR | DSV Canada Inc. |
| Prairie 715RR | DSV Canada Inc. |
| Prairie 717RR | DSV Canada Inc. |
| Prairie 719RR | DSV Canada Inc. |
| Thunder | DSV Canada Inc. |
| C2157 | EURALIS SEMENCES SAS |
| CALUMET | EURALIS SEMENCES SAS |
| ELBE | EURALIS SEMENCES SAS |
| ELEONORE | EURALIS SEMENCES SAS |
| ELLA | EURALIS SEMENCES SAS |
| ES ANTIGONE | EURALIS SEMENCES SAS |
| ES ASTRID | EURALIS SEMENCES SAS |
| ES BOURBON | EURALIS SEMENCES SAS |
| ES NECTAR | EURALIS SEMENCES SAS |
| H19381 | EURALIS SEMENCES SAS |
| OLIVINE | EURALIS SEMENCES SAS |
| OLPHI | EURALIS SEMENCES SAS |
| OLPOP | EURALIS SEMENCES SAS |
| R0029 | EURALIS SEMENCES SAS |
| R0435 | EURALIS SEMENCES SAS |
| R0437 | EURALIS SEMENCES SAS |
| R0438 | EURALIS SEMENCES SAS |
| R0440 | EURALIS SEMENCES SAS |
| R9609 | EURALIS SEMENCES SAS |
| R9925 | EURALIS SEMENCES SAS |
| MIKONOS | EURO GRASS B.V. |
| BIOS | HODOWLA ROSLIN STRZELCE SP. |
| | Z O.O. GRUPA IHAR |
| HUZAR | HODOWLA ROSLIN STRZELCE SP. |
| | Z O.O. GRUPA IHAR |
| MARKIZ | HODOWLA ROSLIN STRZELCE SP. |
| | Z O.O. GRUPA IHAR |
| LUTIN | INRA |
| CASTILLE | JEAN PIERRE DESPEGHEL |
| DOROTHY | JOHN A. TURNER |
| SUMMIT | JOHN A. TURNER |
| GRIFFIN | JOHN TURNER SEED DEVELOPMENTS |
| KABEL | KOIPESOL SEMILLAS S.A. |
| LUCIA | KOIPESOL SEMILLAS S.A. |
| TRACIA | KOIPESOL SEMILLAS S.A. |
| ADDER | KWS SAAT AG |
| ALASKA | KWS SAAT AG |
| ALIGATOR | KWS SAAT AG |
| FORMAT | KWS SAAT AG |
| KW1519 | KWS SAAT AG |
| PIROLA | KWS SAAT AG |
| RAMANO | KWS SAAT AG |
| REMY | KWS SAAT AG |
| ROBUST | KWS SAAT AG |
| RODEO | KWS SAAT AG |
| AC Sunbeam | Lacombe Research Centre |
| AC Sungold | Lacombe Research Centre |
| AKAMAR | LIMAGRAIN ADVANTA NEDERLAND B.V. |
| COURAGE | LIMAGRAIN ADVANTA NEDERLAND B.V. |
| DECATHLON | LIMAGRAIN ADVANTA NEDERLAND B.V. |
| PICASSO | LIMAGRAIN ADVANTA NEDERLAND B.V. |
| COLVERT | LIMAGRAIN VERNEUIL HOLDING S.A. |
| RAPID | LIMAGRAIN VERNEUIL HOLDING S.A. |
| 1818 | Monsanto Canada Inc. |
| 1849 | Monsanto Canada Inc. |
| 1862 | Monsanto Canada Inc. |
| 3235 | Monsanto Canada Inc. |
| 3311 | Monsanto Canada Inc. |
| 3345 | Monsanto Canada Inc. |
| 9550 | Monsanto Canada Inc. |
| 225RR | Monsanto Canada Inc. |
| 30-55 | Monsanto Canada Inc. |
| 32-75 | Monsanto Canada Inc. |
| 33-95 | Monsanto Canada Inc. |
| 34-55 | Monsanto Canada Inc. |
| 34-65 | Monsanto Canada Inc. |
| 35-85 | Monsanto Canada Inc. |
| Ebony | Monsanto Canada Inc. |
| RR Champion | Monsanto Canada Inc. |
| 110 | Monsanto Canada Inc. |
| 111 | Monsanto Canada Inc. |
| 330 | Monsanto Canada Inc. |
| 401 | Monsanto Canada Inc. |
| 420 | Monsanto Canada Inc. |
| 1000 | Monsanto Canada Inc. |
| 223RR | Monsanto Canada Inc. |
| 243CL | Monsanto Canada Inc. |
| 289CL | Monsanto Canada Inc. |
| 292CL | Monsanto Canada Inc. |
| 357RR | Monsanto Canada Inc. |
| 71-20 CL | Monsanto Canada Inc. |
| 71-25 RR | Monsanto Canada Inc. |
| 71-45 RR | Monsanto Canada Inc. |
| 71-85 RR | Monsanto Canada Inc. |
| AV 9440 | Monsanto Canada Inc. |
| AV 9505 | Monsanto Canada Inc. |
| AV 9512 | Monsanto Canada Inc. |
| D1035 | Monsanto Canada Inc. |
| G0118 | Monsanto Canada Inc. |
| MB41001 | Monsanto Canada Inc. |
| MB41007 | Monsanto Canada Inc. |
| S0097 | Monsanto Canada Inc. |
| SP 442 CL | Monsanto Canada Inc. |
| Y0276 | Monsanto Canada Inc. |
| Z0712 | Monsanto Canada Inc. |
| Z1845 | Monsanto Canada Inc. |
| ZSC 4042 | Monsanto Canada Inc. |
| CALIX | MONSANTO PLC |
| CABRIOLET | MONSANTO SAS |
| CAPVERT | MONSANTO SAS |
| CARACAS | MONSANTO SAS |
| CARTOON | MONSANTO SAS |
| CR 18 | MONSANTO SAS |
| CS12 | MONSANTO SAS |
| MLCH079 | MONSANTO SAS |
| | MONSANTO SAS |
| ARIAL | MONSANTO SAS |
| BRISTOL | MONSANTO SAS |
| BRISTOL MS | MONSANTO SAS |
| CABARET | MONSANTO SAS |
| CADDY | MONSANTO SAS |
| CADILLAC | MONSANTO SAS |
| CADOMA | MONSANTO SAS |
| CALIDA | MONSANTO SAS |
| CALIFORNIUM | MONSANTO SAS |
| CALISTO | MONSANTO SAS |
| CAMELIE | MONSANTO SAS |
| CANARY | MONSANTO SAS |
| CANASTA | MONSANTO SAS |
| CANBERRA | MONSANTO SAS |
| CANDO | MONSANTO SAS |
| CAPITOL | MONSANTO SAS |
| CAPTAIN | MONSANTO SAS |
| CARIBOU | MONSANTO SAS |
| CAROLUS | MONSANTO SAS |
| CAROUSEL | MONSANTO SAS |
| CARTEX | MONSANTO SAS |
| CARUSO | MONSANTO SAS |
| CASTILLE | MONSANTO SAS |
| CATALINA | MONSANTO SAS |
| CATONIC | MONSANTO SAS |
| CAVIAR | MONSANTO SAS |
| COHORT | MONSANTO SAS |
| COLUMBUS | MONSANTO SAS |
| COMODOR | MONSANTO SAS |
| CONTACT | MONSANTO SAS |
| CR02 | MONSANTO SAS |
| CR09 | MONSANTO SAS |
| CR10 | MONSANTO SAS |
| CR11 | MONSANTO SAS |
| CR12 | MONSANTO SAS |
| CR15 | MONSANTO SAS |
| CR16 | MONSANTO SAS |
| CRP 40-01 | MONSANTO SAS |
| CS 08 | MONSANTO SAS |
| CS 09 | MONSANTO SAS |
| CS 11 | MONSANTO SAS |
| CS 13 | MONSANTO SAS |
| CSH 01 | MONSANTO SAS |
| CSH23 | MONSANTO SAS |
| CSHP001 | MONSANTO SAS |
| CSHP008 | MONSANTO SAS |
| CSP 401 | MONSANTO SAS |
| DCH 23 | MONSANTO SAS |
| DCH33 | MONSANTO SAS |
| ENVOL | MONSANTO SAS |
| IDOL | MONSANTO SAS |
| M 133 | MONSANTO SAS |
| MLCH077 | MONSANTO SAS |
| MLCH089 | MONSANTO SAS |
| MLCH093 | MONSANTO SAS |
| MLCP30 | MONSANTO SAS |
| R 88421 | MONSANTO SAS |
| SPARK | MONSANTO SAS |
| SPIRAL | MONSANTO SAS |
| SPLIT | MONSANTO SAS |
| | MONSANTO SAS |
| CAMPO | MONSANTO TECHNOLOGY LLC |
| CARACO | MONSANTO TECHNOLOGY LLC |
| CARIOCA | MONSANTO TECHNOLOGY LLC |
| CATANA | MONSANTO TECHNOLOGY LLC |
| CR 25 | MONSANTO TECHNOLOGY LLC |
| CR 26 | MONSANTO TECHNOLOGY LLC |
| CR 27 | MONSANTO TECHNOLOGY LLC |
| CR 28 | MONSANTO TECHNOLOGY LLC |
| CS 28 | MONSANTO TECHNOLOGY LLC |
| EXAGONE | MONSANTO TECHNOLOGY LLC |
| MLCH 111 | MONSANTO TECHNOLOGY LLC |
| MLCH 126 | MONSANTO TECHNOLOGY LLC |
| MLCH 128 | MONSANTO TECHNOLOGY LLC |
| MLCH 129 | MONSANTO TECHNOLOGY LLC |
| MLCH 149 | MONSANTO TECHNOLOGY LLC |
| PBIF 03.1 | MONSANTO TECHNOLOGY LLC |
| SPECIAL | MONSANTO TECHNOLOGY LLC |
| SPLENDOR | MONSANTO TECHNOLOGY LLC |
| V 141 OL | MONSANTO TECHNOLOGY LLC |
| CACTUS | MONSANTO UK LTD |
| CAIMAN | MONSANTO UK LTD |
| CAMERA | MONSANTO UK LTD |
| CAMPALA | MONSANTO UK LTD |
| CANCAN | MONSANTO UK LTD |
| CAPRICORN MS | MONSANTO UK LTD |
| CAPTURE | MONSANTO UK LTD |
| CORNICHE | MONSANTO UK LTD |
| CORONET | MONSANTO UK LTD |
| ECUDOR | MONSANTO UK LTD |
| FRISBEE | MONSANTO UK LTD |
| HEARTY | MONSANTO UK LTD |
| MONARCH | MONSANTO UK LTD |
| | MONSANTO UK LTD |
| | MONSANTO UK LTD |
| SW Hymark 3944 | Newfield Seeds Co. Ltd. |
| ADRIANA | NICKERSON INTERNATIONAL |
| | RESEARCH GEIE |
| AGAPAN | NICKERSON INTERNATIONAL |
| | RESEARCH GEIE |
| AMPTON | NICKERSON INTERNATIONAL |
| | RESEARCH GEIE |
| ATLANTIC | NICKERSON INTERNATIONAL |
| | RESEARCH GEIE |
| BOSTON | NICKERSON INTERNATIONAL |
| | RESEARCH GEIE |
| CADWELL | NICKERSON INTERNATIONAL |
| | RESEARCH GEIE |
| COOPER | NICKERSON INTERNATIONAL |
| | RESEARCH GEIE |
| ESCORT | NICKERSON INTERNATIONAL |
| | RESEARCH GEIE |
| KARUN | NICKERSON INTERNATIONAL |
| | RESEARCH GEIE |
| LADOGA | NICKERSON INTERNATIONAL |
| | RESEARCH GEIE |
| LROC1132 | NICKERSON INTERNATIONAL |
| | RESEARCH GEIE |
| M94284B | NICKERSON INTERNATIONAL |
| | RESEARCH GEIE |
| M9442B | NICKERSON INTERNATIONAL |
| | RESEARCH GEIE |
| M981022 | NICKERSON INTERNATIONAL |
| | RESEARCH GEIE |
| MANITOBA | NICKERSON INTERNATIONAL |
| | RESEARCH GEIE |
| MONTEGO | NICKERSON INTERNATIONAL |
| | RESEARCH GEIE |
| OEJJ13982 | NICKERSON INTERNATIONAL |
| | RESEARCH GEIE |
| ONTARIO | NICKERSON INTERNATIONAL |
| | RESEARCH GEIE |
| PACIFIC | NICKERSON INTERNATIONAL |
| | RESEARCH GEIE |
| POTOMAC | NICKERSON INTERNATIONAL |
| | RESEARCH GEIE |
| SAVANNAH | NICKERSON INTERNATIONAL |
| | RESEARCH GEIE |
| SPR63 | NICKERSON INTERNATIONAL |
| | RESEARCH GEIE |
| SPR75 | NICKERSON INTERNATIONAL |
| | RESEARCH GEIE |
| TASMAN | NICKERSON INTERNATIONAL |
| | RESEARCH GEIE |
| TENNESSEE | NICKERSON INTERNATIONAL |
| | RESEARCH GEIE |
| NIC 1-95 MS | NICKERSON S.A. |
| ACCORD | NORDDEUTSCHE PFLANZENZUCHT |
| | HANS-GEORG |
| | LEMBKE KG |
| AHL810797 | NORDDEUTSCHE PFLANZENZUCHT |
| | HANS-GEORG |
| | LEMBKE KG |
| ARTUS | NORDDEUTSCHE PFLANZENZUCHT |
| | HANS-GEORG |
| | LEMBKE KG |
| BAROS | NORDDEUTSCHE PFLANZENZUCHT |
| | HANS-GEORG |
| | LEMBKE KG |
| BL643196 | NORDDEUTSCHE PFLANZENZUCHT |
| | HANS-GEORG |
| | LEMBKE KG |
| CAMPINO | NORDDEUTSCHE PFLANZENZUCHT |
| | HANS-GEORG |
| | LEMBKE KG |
| DAKINI | NORDDEUTSCHE PFLANZENZUCHT |
| | HANS-GEORG |
| | LEMBKE KG |
| EXPRESS | NORDDEUTSCHE PFLANZENZUCHT |
| | HANS-GEORG |
| | LEMBKE KG |
| HI 734802 | NORDDEUTSCHE PFLANZENZUCHT |
| | HANS-GEORG |
| | LEMBKE KG |
| JAGUAR | NORDDEUTSCHE PFLANZENZUCHT |
| | HANS-GEORG |
| | LEMBKE KG |
| JETTON | NORDDEUTSCHE PFLANZENZUCHT |
| | HANS-GEORG |
| | LEMBKE KG |
| JOCKEY | NORDDEUTSCHE PFLANZENZUCHT |
| | HANS-GEORG |
| | LEMBKE KG |
| LORENZ | NORDDEUTSCHE PFLANZENZUCHT |
| | HANS-GEORG |
| | LEMBKE KG |
| MENDEL | NORDDEUTSCHE PFLANZENZUCHT |
| | HANS-GEORG |
| | LEMBKE KG |
| MSL 004 C | NORDDEUTSCHE PFLANZENZUCHT |
| | HANS-GEORG |
| | LEMBKE KG |
| MSL 011C | NORDDEUTSCHE PFLANZENZUCHT |
| | HANS-GEORG |
| | LEMBKE KG |
| MSL 501C | NORDDEUTSCHE PFLANZENZUCHT |
| | HANS-GEORG |
| | LEMBKE KG |
| MSL 506 C | NORDDEUTSCHE PFLANZENZUCHT |
| | HANS-GEORG |
| | LEMBKE KG |
| MSL007C | NORDDEUTSCHE PFLANZENZUCHT |
| | HANS-GEORG |
| | LEMBKE KG |
| OLYMP | NORDDEUTSCHE PFLANZENZUCHT |
| | HANS-GEORG |
| | LEMBKE KG |
| PRONTO | NORDDEUTSCHE PFLANZENZUCHT |
| | HANS-GEORG |
| | LEMBKE KG |
| VIKING | NORDDEUTSCHE PFLANZENZUCHT |
| | HANS-GEORG |
| | LEMBKE KG |
| WOTAN | NORDDEUTSCHE PFLANZENZUCHT |
| | HANS-GEORG |
| | LEMBKE KG |
| ZEUS | NORDDEUTSCHE PFLANZENZUCHT |
| | HANS-GEORG |
| | LEMBKE KG |
| ACROPOLIS | PIONEER HI-BRED INTERNATIONAL INC. |
| EXPLORER | PIONEER HI-BRED INTERNATIONAL INC. |
| HOMER | PIONEER HI-BRED INTERNATIONAL INC. |
| NW1582 | PIONEER HI-BRED INTERNATIONAL INC. |
| NW1712M | PIONEER HI-BRED INTERNATIONAL INC. |
| PHOENIX | PIONEER HI-BRED INTERNATIONAL INC. |
| PR45D01 | PIONEER HI-BRED INTERNATIONAL INC. |
| PR45W04 | PIONEER HI-BRED INTERNATIONAL INC. |
| PR46W07 | PIONEER HI-BRED INTERNATIONAL INC. |
| PR46W09 | PIONEER HI-BRED INTERNATIONAL INC. |
| PR46W10 | PIONEER HI-BRED INTERNATIONAL INC. |
| PR46W31 | PIONEER HI-BRED INTERNATIONAL INC. |
| ROLLER | PIONEER HI-BRED INTERNATIONAL INC. |
| SUPERIOR | PIONEER HI-BRED INTERNATIONAL INC. |
| 41P55 | Pioneer Hi-Bred Ltd |
| 44A04 | Pioneer Hi-Bred Ltd |
| 44A53 | Pioneer Hi-Bred Ltd |
| 45A51 | Pioneer Hi-Bred Ltd |
| 45A54 | Pioneer Hi-Bred Ltd |
| 45A71 | Pioneer Hi-Bred Ltd |
| 45H20 | Pioneer Hi-Bred Ltd |
| 45H21 | Pioneer Hi-Bred Ltd |
| 45H22 | Pioneer Hi-Bred Ltd |
| 46A65 | Pioneer Hi-Bred Ltd |
| 46A76 | Pioneer Hi-Bred Ltd |
| 46H02 | Pioneer Hi-Bred Ltd |
| 43A56 | Pioneer Hi-Bred Production Ltd. |
| 43H57 | Pioneer Hi-Bred Production Ltd. |
| 45H24 | Pioneer Hi-Bred Production Ltd. |
| 45H25 | Pioneer Hi-Bred Production Ltd. |
| 45H26 | Pioneer Hi-Bred Production Ltd. |
| 45H72 | Pioneer Hi-Bred Production Ltd. |
| 45H73 | Pioneer Hi-Bred Production Ltd. |
| 45P70 | Pioneer Hi-Bred Production Ltd. |
| 46H23 | Pioneer Hi-Bred Production Ltd. |
| 46H70 | Pioneer Hi-Bred Production Ltd. |
| 46P50 | Pioneer Hi-Bred Production Ltd. |
| 46W09 | Pioneer Hi-Bred Production Ltd. |
| NW 4020 | PIONEER OVERSEAS CORPORATION |
| NW1931M | PIONEER OVERSEAS CORPORATION |
| NW4193BC | PIONEER OVERSEAS CORPORATION |
| NW4201BC | PIONEER OVERSEAS CORPORATION |
| NW4202BC | PIONEER OVERSEAS CORPORATION |
| CORPORAL | PLANT BREEDING INTERNATIONAL |
| | CAMBRIDGE LTD |
| PISCES | PLANT BREEDING INTERNATIONAL |
| | CAMBRIDGE LTD |
| SCORPIO | PLANT BREEDING INTERNATIONAL |
| | CAMBRIDGE LTD |
| GRIZZLY | RAGT 2N S.A.S. |
| DANTE | RAPS GBR SAATZUCHT LUNDSGAARD |
| FREDERIC | RAPS GBR SAATZUCHT LUNDSGAARD |
| HEROS | RAPS GBR SAATZUCHT LUNDSGAARD |
| HUNTER | RAPS GBR SAATZUCHT LUNDSGAARD |
| MO13392 | RAPS GBR SAATZUCHT LUNDSGAARD |
| PO1331 | RAPS GBR SAATZUCHT LUNDSGAARD |
| SISKA | RAPS GBR SAATZUCHT LUNDSGAARD |
| SLOGAN | RAPS GBR SAATZUCHT LUNDSGAARD |
| WINNER | RAPS GBR SAATZUCHT LUNDSGAARD |
| GERONIMO | RUSTICA PROGRAIN GENETIQUE SA. |
| NICKEL | RUSTICA PROGRAIN GENETIQUE SA. |
| RPG 314 | RUSTICA PROGRAIN GENETIQUE SA. |
| HENRY | SAATZUCHT DONAU GMBH & CO KG |
| EXPERT | SARL ADRIEN MOMONT ET FILS |
| FIDJI | SARL ADRIEN MOMONT ET FILS |
| FORZA | SARL ADRIEN MOMONT ET FILS |
| GELLO | SARL ADRIEN MOMONT ET FILS |
| HYBRIGOLD | SARL ADRIEN MOMONT ET FILS |
| HYBRISTAR | SARL ADRIEN MOMONT ET FILS |
| KADORE | SARL ADRIEN MOMONT ET FILS |
| KALIF | SARL ADRIEN MOMONT ET FILS |
| KOMANDO | SARL ADRIEN MOMONT ET FILS |
| KOSTO | SARL ADRIEN MOMONT ET FILS |
| LABRADOR | SARL ADRIEN MOMONT ET FILS |
| MAGISTER | SARL ADRIEN MOMONT ET FILS |
| MS ARAMIS | SARL ADRIEN MOMONT ET FILS |
| MS PORTHOS | SARL ADRIEN MOMONT ET FILS |
| OVATION | SARL ADRIEN MOMONT ET FILS |
| PIXEL | SARL ADRIEN MOMONT ET FILS |
| POLLEN | SARL ADRIEN MOMONT ET FILS |
| QUATTRO | SARL ADRIEN MOMONT ET FILS |
| SATORI | SARL ADRIEN MOMONT ET FILS |
| TENOR | SARL ADRIEN MOMONT ET FILS |
| TWINGO | SARL ADRIEN MOMONT ET FILS |
| Amulet | Saskatchewan Wheat Pool |
| Arid | Saskatchewan Wheat Pool |
| Dahinda | Saskatchewan Wheat Pool |
| Davin | Saskatchewan Wheat Pool |
| Estlin | Saskatchewan Wheat Pool |
| SP 451 RR | Saskatchewan Wheat Pool |
| SP Admirable RR | Saskatchewan Wheat Pool |
| SP Armada | Saskatchewan Wheat Pool |
| SP Banner | Saskatchewan Wheat Pool |
| SP Bucky | Saskatchewan Wheat Pool |
| SP Canwood | Saskatchewan Wheat Pool |
| SP Craven | Saskatchewan Wheat Pool |
| SP Deliver CL | Saskatchewan Wheat Pool |
| SP Desirable RR | Saskatchewan Wheat Pool |
| SP Dintinction CL | Saskatchewan Wheat Pool |
| AC Boreal | Saskatoon Research Centre |
| AC Elect | Saskatoon Research Centre |
| AC Parkland | Saskatoon Research Centre |
| AC Tristar | Saskatoon Research Centre |
| ACS-C7 | Saskatoon Research Centre |
| Profit | Saskatoon Research Centre |
| Westar | Saskatoon Research Centre |
| AC Excel | SeCan |
| OAC Dynamite | SeCan |
| OAC Summit | SeCan |
| Reward | SeCan |
| Foremost | Seed-Link Inc. |
| Fortune RR | Seed-Link Inc. |
| Skyhawk | Seed-Link Inc. |
| ASCONA | SEMUNDO SAATZUCHT GMBH |
| KAROLA | SEMUNDO SAATZUCHT GMBH |
| BAMBIN | SERASEM |
| BELCANTO | SERASEM |
| BRYAN | SERASEM |
| CROSSER | SERASEM |
| ECRIN | SERASEM |
| FANTASIO | SERASEM |
| GAMIN | SERASEM |
| IMOLA | SERASEM |
| ISH971P | SERASEM |
| ISLR3 | SERASEM |
| LEWIS | SERASEM |
| MENTION | SERASEM |
| MONZA | SERASEM |
| SALOMONT | SERASEM |
| SATURNIN | SERASEM |
| SUN | SERASEM |
| TRADITION | SERASEM |
| ZERUCA | SERASEM |
| ACROBAT | SVALÖF WEIBULL AB |
| ARIES | SVALÖF WEIBULL AB |
| AVISO | SVALÖF WEIBULL AB |
| CANYON | SVALÖF WEIBULL AB |
| CASINO | SVALÖF WEIBULL AB |
| CORONA | SVALÖF WEIBULL AB |
| CYMBAL | SVALÖF WEIBULL AB |
| ESTER | SVALÖF WEIBULL AB |
| ESTRADE | SVALÖF WEIBULL AB |
| MARS | SVALÖF WEIBULL AB |
| MASKOT | SVALÖF WEIBULL AB |
| MASTER | SVALÖF WEIBULL AB |
| MODENA | SVALÖF WEIBULL AB |
| MUSETTE | SVALÖF WEIBULL AB |
| ORINOCO | SVALÖF WEIBULL AB |
| REBEL | SVALÖF WEIBULL AB |
| SENATOR | SVALÖF WEIBULL AB |
| SPONSOR | SVALÖF WEIBULL AB |
| SPRINTER | SVALÖF WEIBULL AB |
| SW GOSPEL | SVALÖF WEIBULL AB |
| SW LANDMARK | SVALÖF WEIBULL AB |
| TEQUILA | SVALÖF WEIBULL AB |
| TOSCA | SVALÖF WEIBULL AB |
| VERONA | SVALÖF WEIBULL AB |
| SUNDAY | SW SEED HADMERSLEBEN GMBH |
| VISION | SW SEED HADMERSLEBEN GMBH |
| 1896 | SW Seed Ltd. |
| 9451 | SW Seed Ltd. |
| 9551 | SW Seed Ltd. |
| 1839 V | SW Seed Ltd. |
| 1851 H | SW Seed Ltd. |
| 1852H | SW Seed Ltd. |
| 1855H | SW Seed Ltd. |
| 821RR | SW Seed Ltd. |
| Café | SW Seed Ltd. |
| SW 3950 | SW Seed Ltd. |
| SW 6802 | SW Seed Ltd. |
| SW 9803 | SW Seed Ltd. |
| SW WIZZARD | SW Seed Ltd. |
| ALPINE | SYNGENTA CROP PROTECTION AG |
| AMBER | SYNGENTA CROP PROTECTION AG |
| APEX | SYNGENTA CROP PROTECTION AG |
| DJINN | SYNGENTA CROP PROTECTION AG |
| HEKTOR | SYNGENTA CROP PROTECTION AG |
| LASER | SYNGENTA CROP PROTECTION AG |
| MADRIGAL | SYNGENTA CROP PROTECTION AG |
| MAKILA | SYNGENTA CROP PROTECTION AG |
| METEOR | SYNGENTA CROP PROTECTION AG |
| NK BEAMER | SYNGENTA CROP PROTECTION AG |
| NK BOLD | SYNGENTA CROP PROTECTION AG |
| NK GRACE | SYNGENTA CROP PROTECTION AG |
| NK NEMAX | SYNGENTA CROP PROTECTION AG |
| NK OLEO | SYNGENTA CROP PROTECTION AG |
| NKBRAVOUR | SYNGENTA CROP PROTECTION AG |
| NKFAIR | SYNGENTA CROP PROTECTION AG |
| NKVICTORY | SYNGENTA CROP PROTECTION AG |
| RNX4002 | SYNGENTA CROP PROTECTION AG |
| RNX4201 | SYNGENTA CROP PROTECTION AG |
| RNX4401 | SYNGENTA CROP PROTECTION AG |
| RNX4801 | SYNGENTA CROP PROTECTION AG |
| RNX4901 | SYNGENTA CROP PROTECTION AG |
| RNX5002 | SYNGENTA CROP PROTECTION AG |
| RNX5902 | SYNGENTA CROP PROTECTION AG |
| RNX6001 | SYNGENTA CROP PROTECTION AG |
| RNX6101 | SYNGENTA CROP PROTECTION AG |
| ROXET | SYNGENTA CROP PROTECTION AG |
| SMART | SYNGENTA CROP PROTECTION AG |
| ZENITH | SYNGENTA CROP PROTECTION AG |
| ALAMO | SYNGENTA SEEDS GMBH |
| ARIETTA | SYNGENTA SEEDS GMBH |
| ETHNO | SYNGENTA SEEDS GMBH |
| GAMMA | SYNGENTA SEEDS GMBH |
| NEPAL | SYNGENTA SEEDS GMBH |
| RACER | SYNGENTA SEEDS GMBH |
| Roper | TEC Edmonton |
| Conquest | University of Alberta |
| Cougar CL | University of Alberta |
| Hi-Q | University of Alberta |
| Kelsey | University of Alberta |
| Peace | University of Alberta |
| Q2 | University of Alberta |
| Apollo | University of Manitoba |
| BE800397 | W. VON BORRIES-ECKENDORF |
| | GMBH & CO. KG |
| PLANET | W. VON BORRIES-ECKENDORF |
| | GMBH & CO. KG |

Non-limiting examples of elite cotton varieties that are commercially available to farmers include AFD Seed AFD 2485, AFD Seed AFD 3070 F, AFD Seed AFD 3074 F, AFD Seed AFD 351 I RR, AFD Seed AFD 3602 RR, AFD Seed AFD 5064 F, AFD Seed AFD 5065 B2F, AFD Seed AFD 5062 LL, AFD Seed EXPLORER. All-Tex Atlas, All-Tex Atlas RR, All-Tex Apex B2RF, All-Tex Excess RR, All-Tex Marathon B2RF, All-Tex Patriot, All-Tex Patriot RR, All-Tex Summit B2RF., All-Tex Titan B2RF, All-Tex Top-Pick, All-Tex Warrior, All-Tex Xpress, All-Tex Xpress RR, All-Tex 45039 BGRF, Americot AMX 262R, Americot AMX 427R, Americot AMX 821R, Americot AMX 1504 B2RF, Americot AMX 1532 B2RF, Americot AMX 1621, Americot AMX 8120, Bayer CropScience-Fibermax FM 800B2R, Bayer CropScience-Fibermax FM 800RR, Bayer CropScience-Fibermax FM 832, Bayer CropScience-Fibermax FM 832B, Bayer CropScience-Fibermax FM 832LL, Bayer CropScience-Fibermax FM 955LLB2, Bayer CropScience-Fibermax FM 958, Bayer CropScience-Fibermax FM 958B, Bayer CropScience-Fibermax FM 958LL, Bayer CropScience-Fibermax FM 960B2, Bayer CropScience-Fibermax FM 960B2R, Bayer CropScience-Fibermax FM 960BR, Bayer CropScience-Fibermax FM 960RR, Bayer CropScience-Fibermax FM 965LLB2, Bayer CropScience-Fibermax FM 966, Bayer CropScience-Fibermax FM 966LL, Bayer CropScience-Fibermax FM 981LL, Bayer CropScience-Fibermax FM. 988LLB2, Bayer CropScience-Fibermax FM 989, Bayer CropScience-Fibermax FM 989B2R, Bayer CropScience-Fibermax FM 989BR, Bayer CropScience-Fibermax FM 989RR. Bayer CropScience-Fibermax FM 991B2R, Bayer CropScience-Fibermax FM 991BR, Bayer CropScience-Fibermax FM 991RR, Bayer CropScience-Fibermax FM 5024BXN, Bayer CropScience-Fibermax FM 5035LL, Bayer CropScience-Fibermax FM. 9058F, Bayer CropScience-Fibermax FM 9060F, Bayer CropScience-Fibermax FM 9063B2F, Bayer CropScience-Fibermax FM 9068F, Beltwide Cotton Genetics BCG 24R, Beltwide Cotton Genetics BCG 28R, Beltwide Cotton Genetics BCG 30R, Beltwide Cotton Genetics BCG 50R, Beltwide Cotton Genetics BCG 245, Beltwide Cotton Genetics BCG 520R. Beltwide Cotton Genetics BW-1505RF, Beltwide Cotton Genetics BW-2038B2F, Beltwide Cotton Genetics BW-3255B2F., Beltwide Cotton Genetics BW-4021B2F, Beltwide Cotton Genetics BW-4630B2F, Beltwide Cotton Genetics BW-6896B2F, Beltwide Cotton Genetics BW-8391B2F, Beltwide Cotton Genetics BW-9775B2F, CPCSD Acala Daytona RF, CPCSD Acala Fiesta RR, CPCSD Acala NeinX, CPCSD Acala Riata RR, CPCSD Acala Sierra RR, CPCSD Acala Ultima, CPCSD Acala Ultima EF, CPCSD Acala Ultima RF, Croplan Genetics CG 3020 B2RF, Croplan Genetics CG 3520 B2RF, Croplan Genetics CG 4020 B2RF, Deltapine DeltaPEARL, Deltapine Deltapine Acala, Deltapine DP 20 B, Deltapine DP 108 RF. Deltapine DP 110 RF, Deltapine DP 113 B2RF, Deltapine DP 117 B2RF, Deltapine DP 143 B2RF, Deltapine DP 147 RF, Deltapine DP 156 B2RF., Deltapine DP 164 B2RF, Deltapine DP 167 RF, Deltapine DP 388, Deltapine DP 393, Deltapine DP 422 B/RR, Deltapine DP 424 BGII/RR, Deltapine DP 432 RR, Deltapine DP 434 RR, Deltapine DP 436 RR, Deltapine DP 444 BG/RR, Deltapine DP 445 BG/RR, Deltapine DP 448B. Deltapine DP 449 BG/RR, Deltapine DP 451 B/RR, Deltapine DP 454 BG/RR, Deltapine DP 455 BG/RR. Deltapine DP 458 B/RR, Deltapine DP 468 BGII/RR, Deltapine DP 488 BG/RR, Deltapine DP 491, Deltapine DP 493, Deltapine DP 494 RR, Deltapine DP 515 BG/RR, Deltapine DP 543 BG II/RR, Deltapine DP 555 BG/RR, Deltapine DP 565, Deltapine DP 655 B/RR, Deltapine DP 2379, Deltapine DP 5415, Deltapine DP 5415 RR, Deltapine DP 5690, Deltapine DP 5690 RR. Dyna-Gro DG OA265 BR, Dyna-Gro DG 21.00 B2RF, Dyna-Gro DG 2215 B2RF, Dyna-Gro DG 2242 B2RF, Dyna-Gro DG 2520 B2RF, Paymaster PM HS 26, Paymaster PM 280, Paymaster PM 1199 RR, Paymaster PM 1218 BG/RR, Paymaster PM 1560 BG/RR, Paymaster PM 2140 B2RF, Paymaster PM 2145 RR, Paymaster PM 2167 RR, Paymaster PM 2266 RR, Paymaster PM 2280 BG/RR, Paymaster PM 2326 BG/RR, Paymaster PM 2326 RR, Paymaster PM 2344 BG/RR, Paymaster PM 2379 RR, Phytogen NM 1517-99W Acala, Phytogen PHY 72 Acala, Phytogen PHY 78 Acala, Phytogen PHY 125 RF, Phytogen PHY 310 R, Phytogen PHY 370 WR, Phytogen PHY 410 R, Phytogen PHY 425 RF, Phytogen PHY 440 W, Phytogen PHY 470 WR, Phytogen PHY 480 WR, Phytogen PHY 485 WRF, Phytogen PHY 510 R, Phytogen PHY 710 R Acala, Phytogen PHY 715 RF, Phytogen PHY 725 RF, Phytogen PHY 745 WRF, Stoneville BXN 47, Stoneville MCS 0419 B2RF. Stoneville MCS 0420 B2RF, Stoneville MCS 0423 B2RF, Stoneville MCS 0426 B2RF, Stoneville NG 1553 R, Stoneville NG 2448 R, Stoneville NG 3273 B2RF, Stoneville NG 3550 RF, Stoneville NG 3969 R, Stoneville ST 457, Stoneville ST 474, Stoneville ST 1553 R, Stoneville ST 2448 R, Stoneville ST 2454 R, Stoneville ST 3539 BR, Stoneville ST 3636 B2R, Stoneville ST 4357 B2RF, Stoneville ST 4554 B2RF, Stoneville ST 4575 BR, Stoneville ST 4646 B2R, Stoneville ST 4664 RF, Stoneville ST 4700 B2RF, Stoneville ST 4793 R, Stoneville ST 4686 R, Stoneville ST 4892 BR, Stoneville ST 5007 B2RF, Stoneville ST 5454 B2R, Stoneville ST 5242 BR, Stoneville ST 5303 R, Stoneville ST 5599 BR, Stoneville ST 6611 B2RF, Stoneville ST 6622 RF, Stoneville ST 6848 R, Sure-Grow SG 96, Sure-Grow SG 105, Sure-Grow SG 215 BG/RR. Sure-Grow SG 501 BR, Sure-Grow SG 521 R, and Sure-Grow SG 821. An elite plant is a representative plant from an elite variety.

### B. Transgenic Breeding

### 1. Methods and Compositions for Recombinant Nucleic Acids

Nucleic acids for proteins disclosed as useful in the present invention can be expressed in plant cells by operably linking them to a promoter functional in plants Tissue specific and/or inducible promoters may be utilized for appropriate expression of a nucleic acid for a particular trait. The 3′ un-translated sequence, 3′ transcription termination region, or poly adenylation region means a DNA molecule linked to and located downstream of a structural polynucleotide molecule responsible for a trait and includes polynucleotides that provide polyadenylation signal and other regulatory signals capable of affecting transcription, mRNA processing or gene expression. The polyadenylation signal functions in plants to cause the addition of polyadenylate nucleotides to the 3′ end of the mRNA precursor. The polyadenylation sequence can be derived from the natural gene, from a variety of plant genes, or from T-DNA genes. A 5′ UTR that functions as a translation leader sequence is a DNA genetic element located between the promoter sequence and the coding sequence. The translation leader sequence is present in the fully processed mRNA upstream of the translation start sequence. The translation leader sequence may affect processing of the primary transcript to mRNA, mRNA stability or translation efficiency.

The nucleic acid of proteins encoding transgenic traits are operably linked to various expression elements to create an expression unit. Such expression units generally comprise (in 5′ to 3′ direction): a promoter, nucleic acid for a trait, a 3′ untranslated region (UTR). Several other expression elements such as 5′ UTRs, organellar transit peptide sequences, and introns may be added to facilitate expression of the trait.

In some embodiments, protein product of a nucleic acid responsible for a particular trait is targeted to an organelle for proper functioning. For example, targeting of a protein to chloroplast is achieved by using a chloroplast transit peptide sequences. These sequences can be isolated or synthesized from amino acid or nucleic acid sequences of nuclear encoded by chloroplast targeted genes such as small subunit (RbcS2) of ribulose-1,5,-bisphosphate carboxylase, ferredoxin, ferredoxin oxidoreductase, the light-harvesting complex protein I and protein II, and thioredoxin F proteins. Other examples of chloroplast targeting sequences include the maize cab-m7 signal sequence (Becker, et al., 1992; PCT WO 97/41228), the pea glutathione reductase signal sequence (Creissen, et al., 1995; PCT WO 97/41228), and the CTP of the Nicotiana tobaccum ribulose 1,5-bisphosphate carboxylase small subunit chloroplast transit peptide (NtSSU-CTP) (Mazur, et al., 1985).

The term “intron” refers to a polynucleotide molecule that may be isolated or identified from the intervening sequence of a genomic copy of a gene and may be defined generally as a region spliced out during mRNA processing prior to translation. Alternately, introns may be synthetically produced. Introns may themselves contain sub-elements such as cis-elements or enhancer domains that effect the transcription of operably linked genes. A “plant intron” is a native or non-native intron that is functional in plant cells. A plant intron may be used as a regulatory element for modulating expression of an operably linked gene or genes. A polynucleotide molecule sequence in a transformation construct may comprise introns. The introns may be heterologous with respect to the transcribable polynucleotide molecule sequence. Examples of introns include the corn actin intron and the corn HSP70 intron (U.S. Pat. No. 5,859,347, herein incorporated by reference).

Duplication of any expression element across various expression units is avoided due to trait silencing or related effects. Duplicated elements across various expression units are used only when they did not interfere with each other or did not result into silencing of a trait.

Methods are known in the art for assembling and introducing constructs into a cell in such a manner that the nucleic acid molecule for a trait is transcribed into a functional mRNA molecule that is translated and expressed as a protein product. For the practice of the present invention, conventional compositions and methods for preparing and using constructs and host cells are well known to one skilled in the art, see for example, Molecular Cloning: A Laboratory Manual, 3rd edition Volumes 1, 2, and 3 (2000) J. F. Sambrook, D. W. Russell, and N. Irwin, Cold Spring Harbor Laboratory Press. Methods for making transformation constructs particularly suited to plant transformation include, without limitation, those described in U.S. Pat. Nos. 4,971,908, 4,940,835, 4,769,061 and 4,757,011, all of which are herein incorporated by reference in their entirety. These types of vectors have also been reviewed (Rodriguez, et al., Vectors: A Survey of Molecular Cloning Vectors and Their Uses, Butterworths, Boston, 1988; Glick, et al., Methods in Plant Molecular Biology and Biotechnology, CRC Press, Boca Raton, Fla., 1993).

Normally, the expression units are provided between one or more T-DNA borders on a transformation construct. The transformation constructs permit the integration of the expression unit between the T-DNA borders into the genome of a plant cell. The constructs may also contain the plasmid backbone DNA segments that provide replication function and antibiotic selection in bacterial cells, for example, an Escherichia coli origin of replication such as ori322, a broad host range origin of replication such as oriV or oriRi, and a coding region for a selectable marker such as Spec/Strp that encodes for Tn7 aminoglycoside adenyltransferase (aada) conferring resistance to spectinomycin or streptomycin, or a gentamicin (Gm, Gent) selectable marker gene. For plant transformation, the host bacterial strain is often Agrobacterium tumefaciens AB1, C58, LBA4404, EHA101, and EHA105 carrying a plasmid having a transfer function for the expression unit. Other strains known to those skilled in the art of plant transformation can function in the present invention.

In another aspect, nucleic acids of interest may have their expression modified by double-stranded RNA-mediated gene suppression, also known as RNA interference s (“RNAi”), which includes suppression mediated by small interfering RNAs (“siRNA”), trans-acting small interfering RNAs (“ta-siRNA”), or microRNAs (“miRNA”). Examples of RNAi methodology suitable for use in plants are described in detail in U.S. patent application publications 2006/0200878 and 2007/0011775. Methods are known in the art for assembling and introducing constructs into a cell in such a manner that the nucleic acid molecule for a trait is transcribed into a functional mRNA molecule that is translated and expressed as a protein product.

The transgenes of the present invention are introduced into inbreds by transformation methods known to those skilled in the art of plant tissue culture and transformation. Any of the techniques known in the art for introducing expression units into plants may be used in accordance with the invention. Examples of such methods include electroporation as illustrated in U.S. Pat. No. 5,384,253; microprojectile bombardment as illustrated in U.S. Pat. No. 5,015,580; U.S. Pat. No. 5,550,318; U.S. Pat. No. 5,538,880; U.S. Pat. No. 6,160,208; U.S. Pat. No. 6,399,861; and U.S. Pat. No. 6,403,865; protoplast transformation as illustrated in U.S. Pat. No. 5,508,184; and Agrobacterium-mediated transformation as illustrated in U.S. Pat. No. 5,635,055; U.S. Pat. No. 5,824,877; U.S. Pat. No. 5,591,616; U.S. Pat. No. 5,981,840; and U.S. Pat. No. 6,384,301.

After effecting delivery of expression units to recipient cells, the next steps generally concern identifying the transformed cells for further culturing and plant regeneration. In order to improve the ability to identify transformants, one may desire to employ a selectable or screenable marker gene with a transformation construct prepared in accordance with the invention. In this case, one would then generally assay the potentially transformed cell population by exposing the cells to a selective agent or agents, or one would screen the cells for the desired marker gene trait. Examples of various selectable or screenable markers are disclosed in Miki and McHugh, 2004, Selectable marker genes in transgenic plants: applications, alternatives and biosafety, Journal of Biotechnology, 107, 193.

Cells that survive the exposure to the selective agent, or cells that have been scored positive in a screening assay, may be cultured in media that supports regeneration of plants. In an exemplary embodiment, any suitable plant tissue culture media, for example, MS and N6 media may be modified by including further substances such as growth regulators. Tissue may be maintained on a basic media with growth regulators until sufficient tissue is available to begin plant regeneration efforts, or following repeated rounds of manual selection, until the morphology of the tissue is suitable for regeneration, then transferred to media conducive to shoot formation. Cultures are transferred periodically until sufficient shoot formation had occurred. Once shoots are formed, they are transferred to media conducive to root formation. Once sufficient roots are formed, plants can be transferred to soil for further growth and maturity.

To confirm the presence of the DNA for a transgenic trait in the regenerating plants, a variety of assays may be performed. Such assays include, for example, “molecular biological” assays, such as Southern and Northern blotting and PCR™; “biochemical” assays, such as detecting the presence of a protein product, e.g., by immunological means (ELISAs and Western blots) or by enzymatic function; plant part assays, such as leaf or root assays; and also, by analyzing the phenotype of the whole regenerated plant.

Exemplary transgenes of the present invention are provided in Table 2.

TABLE 2
Non-limiting examples of transgenic traits that can be used in
accordance with the methods of the present invention to identify
preferred germplasm and transgene combinations.

| Trait | Gene/protein | Reference |
|---|---|---|
| Herbicide | 5-enolpyruvylshikimate-3- | U.S. Pat. Nos. 5,094,945, |
| tolerance | phosphate synthases | 5,554,798, 5,627,061, 5,633,435, |
| | | 6,040,497, 6,825,400; U.S. patent |
| | | application 20060143727; |
| | | WO04009761 |
| | glyphosate oxidoreductase (GOX) | U.S. Pat. No. 5,463,175 |
| | glyphosate decarboxylase | WO05003362; U.S. patent |
| | | application 20040177399 |
| | glyphosate-N-acetyl transferase | U.S. patent applications |
| | (GAT) | 20030083480, 20060200874 |
| | dicamba monooxygenase | U.S. patent applications |
| | | 20030115626, 20030135879 |
| | phosphinothricin acetyltransferase | U.S. Pat. Nos. 5,276,268, |
| | (bar) | 5,273,894, 5,561,236, 5,637,489, |
| | | 5,646,024; EP 275,957 |
| | 2,2-dichloropropionic acid | WO9927116 |
| | dehalogenase | |
| | acetohydroxyacid synthase or | U.S. Pat. Nos. 4,761,373, |
| | acetolactate synthase | 5,013,659, 5,141,870, 5,378,824, |
| | | 5,605,011, 5,633,437, 6,225,105, |
| | | 5,767,366, 6,613,963 |
| | haloarylnitrilase (Bxn) | U.S. Pat. No. 4,810,648 |
| | acetyl-coenzyme A carboxylase | U.S. Pat. No. 6,414,222 |
| | (seq IDs) | |
| | dihydropteroate synthase (sul I) | U.S. Patents 5,597,717, |
| | | 5,633,444, 5,719,046 |
| | 32 kD photosystem II polypeptide | Hirschberg et al., 1983, Science, |
| | (psbA) | 222: 1346-1349 |
| | anthranilate synthase | U.S. Pat. No. 4,581,847 |
| | phytoene desaturase (crtI) | JP06343473 |
| | hydroxy-phenyl pyruvate | U.S. Pat. No. 6,268,549 |
| | dioxygenase | |
| | protoporphyrinogen oxidase I | U.S. Pat. No. 5,939,602 |
| | (protox) | |
| | aryloxyalkanoate dioxygenase | WO05107437 |
| | (AAD-1)(Seq IDs) | |
| Male/female | Several | U.S. Patent Application |
| sterility system | | 20050150013 |
| | Glyphosate/EPSPS | U.S. Pat. No. 6,762,344 |
| | Male sterility gene linked to | U.S. Pat. No. 6,646,186 |
| | herbicide resistant gene | |
| | Acetylated toxins/deacetylase | U.S. Pat. No. 6,384,304 |
| | Antisense to an essential gene in | U.S. Pat. No. 6,255,564 |
| | pollen formation | |
| | DNAase or endonuclease/restorer | U.S. Pat. No. 6,046,382 |
| | protein | |
| | Ribonuclease/barnase | U.S. Pat. No. 5,633,441 |
| Intrinsic yield | glycolate oxidase or glycolate | U.S. Patent Application |
| | dehydrogenase, glyoxylate | 2006009598 |
| | carboligase, tartronic | |
| | semialdehyde reductase | |
| | eukaryotic initiation Factor 5A; | U.S. patent application |
| | deoxyhypusine synthase | 20050235378 |
| | zinc finger protein | U.S. patent application |
| | | 20060048239 |
| | methionine aminopeptidase | U.S. patent application |
| | | 20060037106 |
| | several | U.S. patent applications |
| | | 20060037106 |
| | 2,4-D dioxygenase | U.S. patent applications |
| | | 20060030488 |
| | serine carboxypeptidase | U.S. patent applications |
| | | 20060085872 |
| | several | USRE38,446; U.S. Pat. Nos. |
| | | 6,716,474, 6,663,906, 6,476,295, |
| | | 6,441,277, 6,423,828, 6,399,330, |
| | | 6,372,211, 6,235,971, 6,222,098, |
| | | 5,716,837, 6,723,897, 6,518,488 |
| Nitrogen use | fungal nitrate reductases, mutant | U.S. patent applications |
| efficiency | nitrate reductases lacking post- | 20050044585 |
| | translational regulation, glutamate | |
| | synthetase-1, | |
| | glutamate dehydrogenase, | |
| | aminotransferases, nitrate | |
| | transporters (high affinity and low | |
| | affinities), ammonia transporters | |
| | and amino acid transporters | |
| | glutamate dehydrogenase | U.S. patent applications |
| | | 20060090219 |
| | cytosolic glutamine synthetase; | EP0722494 |
| | root-specific glutamine | |
| | synthetase. | |
| | several | WO05103270; U.S. patent |
| | | applications 20070044172, |
| | | 20070107084 |
| | glutamate 2-oxoglutarate | U.S. Pat. No. 6,864,405 |
| | aminotransferase | |
| Abiotic Stress | succinate semialdehyde | U.S. patent applications |
| tolerance | dehydrogenase | 20060075522 |
| including cold, | several | U.S. Pat. Nos. 5,792,921, |
| heat, drought | | 6,051,755, 7,084,323, 6,229,069, |
| | | 6,534,446, 6,951,971, 6,376,747, |
| | | 6,624,139, 6,559,099, 6,455,468, |
| | | 6,635,803, 6,515,202, 6,960,709, |
| | | 6,706,866, 7,164,057, 7,141,720, |
| | | 6,756,526, 6,677,504, 6,689,939, |
| | | 6,710,229, 6,720,477, 6,818,805, |
| | | 6,867,351, 7,074,985, 7,091,402, |
| | | 7,101,828, 7,138,277, 7,154,025, |
| | | 7,161,063, 7,166,767, 7,176,027, |
| | | 7,179,962, 7,186,561, 7,186,563, |
| | | 7,186,887, 7,193,130; U.S. patent |
| | | applications 20030221224, |
| | | 20040128712, 20040187175, |
| | | 20050097640, 20050204431, |
| | | 20050235382, 20050246795, |
| | | 20050086718, 20060008874, |
| | | 20060015972, 20060021082, |
| | | 20060021091, 20060026716, |
| | | 20060064775, 20060064784, |
| | | 20060075523, 20060112454, |
| | | 20060123516, 20060137043, |
| | | 20060150285, 20060168692, |
| | | 20060162027, 20060183137, |
| | | 20060183137, 20060185038, |
| | | 20060253938, 20070006344, |
| | | 20070006348, 20070079400, |
| | | 20070028333, 20070107084; |
| | | WO06032708 |
| | transcription factor | U.S. patent applications |
| | | 20060162027 |
| Disease | CYP93C (cytochrome P450) | U.S. Pat. No. 7,038,113 |
| resistance | several | U.S. Pat. Nos. 5,304,730, |
| | | 5,516,671, 5,773,696, 5,850,023, |
| | | 6,013,864, 6,015,940, 6,121,436, |
| | | 6,215,048, 6,228,992, 6,316,407, |
| | | 6,506,962, 6,573,361, 6,608,241, |
| | | 6,617,496, 6,653,280, 7,038,113 |
| Insect resistance | several | U.S. Pat. Nos. 5,484,956, |
| | | 5,763,241, 5,763,245, 5,880,275, |
| | | 5,942,658, 5,942,664, 5,959,091, |
| | | 6,002,068, 6,023,013, 6,063,597, |
| | | 6,063,756, 6,093,695, 6,110,464, |
| | | 6,153,814, 6,156,573, 6,177,615, |
| | | 6,221,649, 6,242,241, 6,248,536, |
| | | 6,281,016, 6,284,949, 6,313,378, |
| | | 6,326,351, 6,468,523, 6,501,009, |
| | | 6,521,442, 6,537,756, 6,538,109, |
| | | 6,555,655, 6,593,293, 6,620,988, |
| | | 6,639,054, 6,642,030, 6,645,497, |
| | | 6,657,046, 6,686,452, 6,713,063, |
| | | 6,713,259, 6,809,078, 7,049,491; |
| | | U.S. patent applications |
| | | 20050039226, 20060021087, |
| | | 20060037095, 20060070139, |
| | | 20060095986; WO05059103 |
| Enhanced amino | glutamate dehydrogenase | U.S. Pat. No. 6,969,782 |
| acid content | threonine deaminase | U.S. patent applications |
| | | 20050289668 |
| | dihydrodipicolinic acid synthase | U.S. Pat. Nos. 5,258,300, |
| | (dap A) | 6,329,574, 7,157,281 |
| | chymotrypsin inhibitor | U.S. Pat. No. 6,800,726 |
| Enhanced | several | U.S. patent applications |
| protein | | 20050055746 |
| content | | |
| Modified fatty | several | U.S. Pat. Nos. 6,380,462, |
| acids | | 6,426,447, 6,444,876, 6,459,018, |
| | | 6,489,461, 6,537,750, 6,589,767, |
| | | 6,596,538, 6,660,849, 6,706,950, |
| | | 6,770,465, 6,822,141, 6,828,475, |
| | | 6,949,698 |
| Enhanced oil | several | U.S. Pat. Nos. 5,608,149, |
| content | | 6,483,008, 6,476,295, 6,822,141, |
| | | 6,495,739, 7,135,617 |
| Carbohydrate | raffinose saccharides | U.S. Pat. No. 6,967,262 |
| production | | |
| Starch | several | U.S. Pat. No. 5,750,876, 6,476,295, |
| production | | 6,538,178, 6,538,179, 6,538,181, |
| | | 6,951,969 |
| Phytic acid | inositol polyphosphate 2-kinase | WO06029296 |
| reduction | inositol 1,3,4-triphosphate 5/6- | U.S. patent applications |
| | kinases | 20050202486 |
| Processing | several | WO05096804; U.S. Pat. No. |
| enzymes | | 5,543,576 |
| production | | |
| Biopolymers | several | USRE37,543; U.S. Patents |
| | | 5,958,745, 6,228,623; U.S. patent |
| | | application 20030028917 |
| Enhanced | several | US Pat. Nos. 5,985,605, 6,171,640, |
| nutrition | | 6,541,259, 6,653,530, 6,723,837 |
| Pharmaceutical | several | U.S. Pat. Nos. 6,080,560, |
| peptides and | | 6,140,075, 6,774,283, 6,812,379 |
| secretable | | |
| peptides | | |
| Improved | sucrose phosphorylase | U.S. Pat. No. 6,476,295 |
| processing trait | | |
| Improved | thioredoxin and/or thioredoxin | U.S. Pat. No. 6,531,648 |
| digestibility | reductase | |

### 2. Trait Integration

The present invention anticipates that one skilled in the art can use the methods of the present invention to screen for transgene performance at any point after a transformant has been obtained. Germplasm that has been transformed with the at least one transgene or germplasm that has been converted, i.e., backcross conversion, can be evaluated. In another aspect, germplasm can be crossed with a transgenic tester and then evaluated. In certain aspects, two or more transgenic events are evaluated. In other aspects, two or more germplasm entries with one or more transgenic events are evaluated. In other aspects, two or more transgenes, i.e., stacks, are evaluated. Evaluation of transgene performance is accomplished by testing for the presence of one or more transgene modulating loci using marker-trait association techniques or by testing germplasm for transgene performance, i.e., using a two or more germplasm entries.

Once a transgene for a trait has been introduced into a plant, that gene can be introduced into any plant sexually compatible with the first plant by crossing, without the need for directly transforming the second plant. Therefore, as used herein the term “progeny” denotes the offspring of any generation of a parent plant prepared in accordance with the present invention. A “transgenic plant” may thus be of any generation.

Descriptions of breeding methods that are commonly used for different traits and crops can be found in one of several reference books (Allard, “Principles of Plant Breeding,” John Wiley & Sons, NY, U. of CA, Davis, Calif., 50-98, 1960; Simmonds, “Principles of crop improvement,” Longman, Inc., NY, 369-399, 1979; Sneep and Hendriksen, “Plant breeding perspectives,” Wageningen (ed), Center for Agricultural Publishing and Documentation, 1979; Fehr, In: Soybeans: Improvement, Production and Uses, 2nd Edition, Manograph., 16:249, 1987; Fehr, “Principles of variety development,” Theory and Technique, (Vol 1) and Crop Species Soybean (Vol 2), Iowa State Univ., Macmillian Pub. Co., NY, 360-376, 1987).

In general, two distinct breeding stages are used for commercial development of elite cultivars containing a transgenic trait. The first stage involves evaluating and selecting a superior transgenic event, while the second stage involves integrating the selected transgenic event in a commercial germplasm.

In a typical transgenic breeding program, a transformation construct responsible for a trait is introduced into the genome via a transformation method. Numerous independent transformants (events) are usually generated for each construct. These events are evaluated to select those with superior performance. The event evaluation process is based on several criteria including 1) transgene expression/efficacy of the trait, 2) molecular characterization of the trait, 3) segregation of the trait, 4) agronomics of the developed event, and 5) stability of the transgenic trait expression. Evaluation of large populations of independent events and more thorough evaluation result in the greater chance of success. The present invention anticipates the methods provided herein are especially useful for comparing performance of two or more events.

Events showing right level of protein expression that corresponds with right phenotype (efficacy) are selected for further use by evaluating the event for insertion site, transgene copy number, intactness of the transgene, zygosity of the transgene, level of inbreeding associated with a genotype, and environmental conditions. Events showing a clean single intact insert are found by conducting molecular assays for copy number, insert number, insert complexity, presence of the vector backbone, and development of event-specific assays and are used for further development. Segregation of the trait is tested to select transgenic events that follow a single-locus segregation pattern. A direct approach is to evaluate the segregation of the trait. An indirect approach is to assess the selectable marker segregation (associated with the transgenic trait).

Event instability over generations is often caused by transgene inactivation due to multiple transgene copies, zygosity level, highly methylated insertion sites, or level of stress. Thus, stability of transgenic trait expression is ascertained by testing in different generations, environments, and in different genetic backgrounds. Events that show transgenic trait silencing are discarded.

Generally, events with a single intact insert that inherited as a single dominant gene and follow Mendelian segregation ratios are used in commercial trait integration strategies such as backcrossing and forward breeding.

In a preferred embodiment, the methods of the present invention provide trait integration strategies comprising the evaluation of at least one event for at least one transgene in at least two different genetic backgrounds for the purpose of evaluating genotype interactions with the one or more transgenes. In other aspects, two or more events for a given transgene are evaluated in at least one germplasm entry. In still other aspects, two or more transgenes are evaluated. In one embodiment, the one or more transgenes are evaluated in mapping populations, that is, segregating progeny, and phenotyping of the transgene is accompanied by evaluation of agronomic traits and genome-wide fingerprinting involving a plurality of SNP markers. Subsequently, association studies are employed to determine the presence of one or more transgene modulating loci for the one or more transgenes for the germplasm entries. In another embodiment, additional markers may be used in selection decisions that are associated with the at least one transgene modulating loci and can be detected by means of visual assays, chemical or analytic assays, or some other type of phenotypic assay. The marker or markers directly or indirectly associated with the one or more transgene modulating loci can then be used to select lines with these loci or for introgressing transgene modulating loci into lines that do not have preferred alleles for transgene modulating loci.

In another aspect, testing may be expanded to assess at least one lead event in at least two different genetic backgrounds in at least two different locations for the purpose of evaluation of genotype interactions with the one or more transgenes in two or more locations.

In another aspect, testing may be expanded to assess at least one lead event in at least two different genetic backgrounds in at least two different conditions for at least one environmental factor for the purpose of evaluation of genotype interactions with the one or more transgenes in two or more environmental conditions.

In one embodiment, trait integration is accomplished using backcrossing to recover the genotype of an elite inbred with an additional transgenic trait. In each backcross generation, plants that contain the transgene are identified and crossed to the elite recurrent parent. Several backcross generations with selection for recurrent parent phenotype are generally used by commercial breeders to recover the genotype of the elite parent with the additional transgenic trait. During backcrossing the transgene is kept in a hemizygous state. Therefore, at the end of the backcrossing, the plants are self- or sib-pollinated to fix the transgene in a homozygous state. The number of backcross generations can be reduced by molecular assisted backcrossing (MABC). The MABC method uses genetic markers to identify plants that are most similar to the recurrent parent in each backcross generation. With the use of MABC and appropriate population size, it is possible to identify plants that have recovered over 98% of the recurrent parent genome after only two or three backcross generations. By eliminating several generations of backcrossing, it is often possible to bring a commercial transgenic product to market one year earlier than a product produced by conventional backcrossing.

In a preferred embodiment, MABC also targets markers corresponding at least one transgene modulating locus, previously identified from marker-trait mapping in a panel of germplasm entries segregating for transgene modulators. In another embodiment, MAS is used in activities related to line development in order to develop elite lines with preferred transgene modulating genotypes. In another aspect, additional markers may be used in selection decisions that are associated with the transgene modulating loci and can be detected by means of visual assays, chemical or analytic assays, or some other type of phenotypic assay.

Forward breeding is any breeding method that has the goal of developing a transgenic variety, inbred line, or hybrid that is genotypically different, and superior, to the parents used to develop the improved genotype. When forward breeding a transgenic crop, selection pressure for the efficacy of the transgene is usually applied during each generation of the breeding program. Additionally, it is usually advantageous to fix the transgene in a homozygous state during the breeding process as soon as possible to evaluate transgene×genotype interactions.

In a preferred embodiment, the present invention provides a method to evaluate transgene×genotype interactions in hybrid crops in one generation without directly forward breeding. Elite inbred lines are crossed with at least one tester with at least one transgene and the progeny are evaluated for genotype interactions, wherein preferred genotype-transgene combinations can be identified without the time and cost of MABC.

After integrating the transgenic traits into commercial germplasm, the final inbreds and hybrids are tested in multiple locations. Testing typically includes yield trials in trait neutral environments as well as typical environments of the target markets. If the new transgenic line has been derived from backcrossing, it is usually tested for equivalency by comparing it to the non-transgenic version in all environments.

In another aspect of the present invention, transgenic events are selected for further development in which the nucleic acids encoding for cost decreasing traits and/or end user traits are inserted and linked to genomic regions (defined as haplotypes) that are found to provide additional benefits to the crop plant. The transgene and the haplotype comprise a T-type genomic region. Methods for using haplotypes and T-type genomic regions for enhancing breeding are disclosed in U.S. patent application Ser. No. 11/441,915.

The present invention also provides for parts of the plants of the present invention. Plant parts, without limitation, include seed, endosperm, ovule and pollen. In a preferred embodiment of the present invention, the plant part is a seed. The invention also includes and provides transformed plant cells which comprise a nucleic acid molecule of the present invention.

### C. Commercial Applications

In other embodiments, the present invention provides methods for capturing commercial value from breeding activities. For example, the methods of the present invention allow for the licensing of combinations of transgenes and particular genotypes. Instead of licensing only transgenes, an entity can license packages of at least one transgene with at least one genotype, wherein the genotype may comprise a kit for detection of at least one transgene modulating locus, germplasm recommendations for deployment of at least one transgene, and/or germplasm sources for conversions to introgress at least one transgene modulating locus.

### EXAMPLES

The following examples are included to illustrate embodiments of the invention. It should be appreciated by those of skill in the art that the techniques disclosed in the examples that follow represent techniques discovered by the inventor to function well in the practice of the invention. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Mapping of Transgene Modulating Loci for Selection of Preferred Germplasm-Transgene Combinations in Corn

Monsanto developed a transgenic event known as LY038 providing elevated free lysine concentration in corn grain (U.S. Pat. No. 7,157,281). The event was accomplished through engineering a bacterial version of dihydrodipiccolinate synthase (DHDPS) that is insensitive to the feedback inhibition by lysine. Differences with respect to free lysine have been observed among different inbred conversions when crossed with the LY038 event. Interactions among inbred germplasm were small relative to the effect of the inbred background. The differences observed in the lysine levels were therefore presumably controlled by one or more modulating loci in the genome of the inbred germplasm, thereby comprising a genotype that can be measured and identified. In order to account for the observed lysine variation, a mapping (i.e., segregating) population was created for the purpose of measuring genotypic and phenotypic differences to identify putative associations between one or more genetic markers and lysine levels.

The initial stages of discovery of the lysine modulating genotypes was through linkage and trait mapping experiments from a controlled cross of an inbred with High lysine and an inbred with Low lysine for the identification of loci that modulate the lysine expression performance. Differences among lysine levels were measured as described in U.S. Pat. No. 7,157,281, which is incorporated herein by reference in its entirety, in ppm (parts per million) among the plurality of inbred conversions for the LY038 event that represent different genetic backgrounds of the inbred germplasm.

Following are examples of mapping approaches to detect transgene modulating loci, using inbred conversions demonstrating divergent lysine phenotypes. Each experiment used a marker density of approximately 100-200 SNP markers. QTL were designated based on approximately 5-20 cM windows. All markers reported herein are summarized and referenced to the sequence listing in Table 3.

TABLE 3

Summary of genetic markers associated with transgene
modulating QTL for LY038, affecting lysine
concentration and/or white seedling phenotype.

| | | | | | | |
|---|---|---|---|---|---|---|
| SEQ | | | | | | |
| ID | | | | | | SNP |
| NO | Marker | chr | position | allele 1 | allele 2 | position |

| | | | | | | |
|---|---|---|---|---|---|---|
| 1 | NC0000129 | 8 | 16.5 | A | G | 112 |
| 2 | NC0002635 | 1 | 254.8 | C | G | 199 |
| 3 | NC0002739 | 4 | 11.8 | * | C | 134 |
| 4 | NC0002905 | 3 | 123.9 | A | T | 106 |
| 5 | NC0003224 | 4 | 173.6 | C | G | 172 |
| 6 | NC0003226 | 4 | 173.6 | C | T | 402 |
| 7 | NC0004176 | 1 | 116.3 | A | C | 61 |
| 8 | NC0004371 | 3 | 164.2 | C | G | 325 |
| 9 | NC0004445 | 4 | 176.6 | C | T | 274 |
| 10 | NC0004504 | 8 | 95.6 | A | C | 469 |
| 11 | NC0004586 | 8 | 125.1 | A | G | 64 |
| 12 | NC0004605 | 5 | 78.5 | C | T | 74 |
| 13 | NC0004887 | 10 | 45.2 | A | G | 392 |
| 14 | NC0004953 | 7 | 131.2 | A | T | 154 |
| 15 | NC0005018 | 4 | 94.8 | C | T | 646 |
| 16 | NC0005088 | 2 | 147.6 | C | T | 112 |
| 17 | NC0005295 | 4 | 135.1 | C | T | 266 |
| 18 | NC0005467 | 2 | 94.3 | C | G | 76 |
| 19 | NC0008900 | 3 | 97.6 | A | G | 258 |
| 20 | NC0008911 | 3 | 19.9 | A | G | 165 |
| 21 | NC0009057 | 4 | 21.7 | G | T | 229 |
| 22 | NC0009102 | 2 | 130 | A | T | 366 |
| 23 | NC0009297 | 5 | 104.1 | A | G | 72 |
| 24 | NC0009364 | 2 | 71.6 | C | T | 185 |
| 25 | NC0009473 | 3 | 168.4 | C | T | 275 |
| 26 | NC0009620 | 4 | 109.2 | G | T | 175 |
| 27 | NC0009645 | 10 | 32.1 | A | G | 178 |
| 28 | NC0009701 | 1 | 207.9 | A | G | 352 |
| 29 | NC0009818 | 2 | 136.5 | A | T | 1 |
| 30 | NC0010232 | 3 | 198.7 | C | T | 353 |
| 31 | NC0012480 | 5 | 99.4 | A | C | 137 |
| 32 | NC0012830 | 9 | 33.1 | A | G | 361 |
| 33 | NC0012935 | 5 | 45.7 | A | G | 437 |
| 34 | NC0013086 | 9 | 87.3 | A | G | 365 |
| 35 | NC0013158 | 7 | 48.6 | G | T | 382 |
| 36 | NC0013833 | 4 | 175.6 | A | G | 441 |
| 37 | NC0014417 | 6 | 25 | A | G | 208 |
| 38 | NC0014479 | 9 | 0.8 | G | T | 309 |
| 39 | NC0015146 | 8 | 84 | A | G | 432 |
| 40 | NC0015344 | 1 | 221.1 | A | G | 420 |
| 41 | NC0015965 | 3 | 140.8 | A | T | 390 |
| 42 | NC0016868 | 5 | 122.6 | C | G | 338 |
| 43 | NC0017678 | 5 | 103.8 | A | C | 176 |
| 44 | NC0017828 | 4 | 144.7 | A | G | 341 |
| 45 | NC0017900 | 4 | 179.3 | A | G | 156 |
| 46 | NC0019003 | 4 | 45.3 | G | T | 405 |
| 47 | NC0019110 | 2 | 75.1 | A | C | 159 |
| 48 | NC0020502 | 10 | 30.3 | A | G | 172 |
| 49 | NC0020546 | 8 | 115.6 | A | G | 51 |
| 50 | NC0020971 | 3 | 13.9 | A | C | 57 |
| 51 | NC0021092 | 2 | 93.4 | A | G | 96 |
| 52 | NC0021585 | 5 | 175 | C | G | 234 |
| 53 | NC0021734 | 6 | 145.4 | G | T | 438 |
| 54 | NC0021772 | 3 | 154.1 | C | T | 259 |
| 55 | NC0022725 | 4 | 91.3 | C | T | 145 |
| 56 | NC0023779 | 9 | 56.1 | A | G | 343 |
| 57 | NC0024631 | 3 | 83.2 | G | T | 248 |
| 58 | NC0024647 | 4 | 52.5 | A | G | 191 |
| 59 | NC0025198 | 9 | 45.7 | C | T | 289 |
| 60 | NC0025863 | 1 | 96.7 | A | G | 129 |
| 61 | NC0027095 | 6 | 38.8 | A | G | 259 |
| 62 | NC0027262 | 2 | 57.3 | C | T | 363 |
| 63 | NC0027447 | 10 | 75.6 | C | G | 311 |
| 64 | NC0027567 | 1 | 179.4 | C | G | 79 |
| 65 | NC0027914 | 9 | 45 | A | G | 211 |
| 66 | NC0028110 | 5 | 90.2 | A | C | 488 |
| 67 | NC0028185 | 6 | 130.1 | C | G | 523 |
| 68 | NC0029487 | 4 | 171.1 | G | T | 159 |
| 69 | NC0030029 | 7 | 112.7 | C | T | 317 |
| 70 | NC0030576 | 4 | 153.8 | C | T | 880 |
| 71 | NC0030985 | 4 | 181.9 | *********** | ACTGTTCCAAG | 164 |
| 72 | NC0031025 | 8 | 108.5 | C | T | 393 |
| 73 | NC0031358 | 10 | 64.2 | ********* | CATTGTTGT | 507 |
| 74 | NC0031474 | 2 | 141.4 | A | T | 844 |
| 75 | NC0032034 | 6 | 57.6 | A | G | 498 |
| 76 | NC0032049 | 4 | 162.6 | C | T | 183 |
| 77 | NC0032200 | 2 | 71.6 | C | T | 318 |
| 78 | NC0033667 | 4 | 73.7 | C | G | 233 |
| 79 | NC0033977 | 5 | 29.3 | A | G | 476 |
| 80 | NC0034325 | 4 | 63.7 | C | G | 193 |
| 81 | NC0034552 | 8 | 51.8 | C | T | 260 |
| 82 | NC0035338 | 4 | 190.6 | C | G | 105 |
| 83 | NC0035408 | 7 | 89.5 | A | C | 221 |
| 84 | NC0035579 | 1 | 94.5 | A | G | 282 |
| 85 | NC0035961 | 1 | 206.7 | C | T | 264 |
| 86 | NC0036210 | 5 | 145.2 | G | T | 43 |
| 87 | NC0036239 | 4 | 112.1 | A | G | 341 |
| 88 | NC0036415 | 4 | 181 | C | T | 59 |
| 89 | NC0036534 | 4 | 147.9 | *** | TTA | 515 |
| 90 | NC0036637 | 5 | 100 | C | T | 699 |
| 91 | NC0036685 | 1 | 45.8 | A | G | 203 |
| 92 | NC0037062 | 4 | 59.7 | C | G | 66 |
| 93 | NC0037588 | 5 | 60.1 | ***** | CACAA | 188 |
| 94 | NC0037947 | 6 | 97.6 | A | G | 87 |
| 95 | NC0038475 | 1 | 168.3 | A | G | 58 |
| 96 | NC0039298 | 1 | 194.6 | C | T | 591 |
| 97 | NC0039511 | 4 | 121.5 | C | G | 560 |
| 98 | NC0039840 | 1 | 65.8 | C | G | 82 |
| 99 | NC0040371 | 4 | 67.8 | A | C | 202 |
| 100 | NC0040571 | 5 | 88.4 | C | G | 154 |
| 101 | NC0048567 | 4 | 146.9 | A | T | 117 |
| 102 | NC0049293 | 3 | 69.9 | A | C | 183 |
| 103 | NC0051919 | 8 | 71.1 | C | T | 347 |
| 104 | NC0053097 | 2 | 102.6 | A | T | 335 |
| 105 | NC0054460 | 4 | 131.7 | A | T | 411 |
| 106 | NC0054661 | 10 | 57.1 | A | G | 115 |
| 107 | NC0057210 | 2 | 104.1 | C | T | 191 |
| 108 | NC0059764 | 8 | 118.8 | C | T | 56 |
| 109 | NC0060681 | 4 | 164.4 | A | G | 107 |
| 110 | NC0060879 | 2 | 97.7 | C | G | 367 |
| 111 | NC0066143 | 7 | 57.1 | A | G | 171 |
| 112 | NC0066807 | 7 | 67.1 | A | G | 636 |
| 113 | NC0067075 | 6 | 98.9 | C | G | 457 |
| 114 | NC0067728 | 1 | 173.7 | C | T | 239 |
| 115 | NC0068434 | 7 | 76.5 | C | T | 573 |
| 116 | NC0069524 | 1 | 99.9 | A | C | 514 |
| 117 | NC0069570 | 4 | 92.4 | C | T | 640 |
| 118 | NC0070533 | 4 | 130.2 | C | T | 439 |
| 119 | NC0070996 | 6 | 81.9 | C | T | 769 |
| 120 | NC0077749 | 1 | 79.6 | C | T | 364 |
| 121 | NC0080031 | 2 | 33.1 | A | G | 164 |
| 122 | NC0080705 | 2 | 68.5 | C | G | 282 |
| 123 | NC0083876 | 5 | 124 | C | T | 513 |
| 124 | NC0104195 | 9 | 68.5 | A | G | 225 |
| 125 | NC0104512 | 10 | 57.3 | A | T | 79 |
| 126 | NC0104785 | 4 | 83.9 | A | G | 449 |
| 127 | NC0104858 | 8 | 96.2 | *** | GCT | 173 |
| 128 | NC0104963 | 5 | 159.8 | A | G | 269 |
| 129 | NC0105022 | 1 | 79.5 | A | G | 63 |
| 130 | NC0105497 | 6 | 67.6 | C | T | 465 |
| 131 | NC0105613 | 5 | 16.6 | C | G | 178 |
| 132 | NC0105696 | 2 | 94.3 | C | T | 149 |
| 133 | NC0105818 | 4 | 155.8 | A | T | 243 |
| 134 | NC0106263 | 4 | 133 | A | G | 204 |
| 135 | NC0106296 | 1 | 181 | A | G | 178 |
| 136 | NC0106341 | 6 | 29.5 | A | G | 234 |
| 137 | NC0107293 | 4 | 155.5 | A | C | 381 |
| 138 | NC0107905 | 9 | 63.4 | G | T | 376 |
| 139 | NC0108013 | 2 | 115.3 | C | T | 340 |
| 140 | NC0108089 | 3 | 106.3 | ** | AT | 274 |
| 141 | NC0108275 | 9 | 91.6 | A | T | 520 |
| 142 | NC0108727 | 3 | 77.4 | C | G | 241 |
| 143 | NC0108962 | 8 | 139.7 | C | G | 238 |
| 144 | NC0109283 | 4 | 175.5 | A | G | 82 |
| 145 | NC0109526 | 9 | 66.5 | C | G | 297 |
| 146 | NC0109795 | 10 | 53 | A | G | 362 |
| 147 | NC0110974 | 2 | 185.5 | C | T | 522 |
| 148 | NC0111388 | 5 | 66.6 | C | G | 64 |
| 149 | NC0111959 | 3 | 117.6 | ** | GT | 71 |
| 150 | NC0112604 | 6 | 38.4 | A | C | 156 |
| 151 | NC0112644 | 3 | 181.8 | C | T | 420 |
| 152 | NC0113172 | 5 | 43.8 | C | G | 327 |
| 153 | NC0143216 | 5 | 67.7 | A | C | 68 |
| 154 | NC0143251 | 5 | 11.6 | A | G | 222 |
| 155 | NC0143354 | 5 | 1.8 | C | G | 307 |
| 156 | NC0143380 | 5 | 148.1 | A | G | 330 |
| 157 | NC0143514 | 7 | 29 | A | G | 609 |
| 158 | NC0143819 | 7 | 7.1 | G | T | 184 |
| 159 | NC0143873 | 1 | 52.6 | A | G | 249 |
| 160 | NC0143969 | 3 | 187.5 | ** | TA | 100 |
| 161 | NC0144324 | 4 | 183 | C | G | 418 |
| 162 | NC0144363 | 8 | 91.1 | A | G | 454 |
| 163 | NC0146130 | 2 | 94.6 | A | G | 100 |
| 164 | NC0146215 | 6 | 106.6 | C | T | 224 |
| 165 | NC0146546 | 5 | 71.2 | C | T | 364 |
| 166 | NC0147302 | 1 | 27.6 | C | T | 152 |
| 167 | NC0147315 | 5 | 115 | C | T | 223 |
| 168 | NC0147417 | 9 | 153.2 | C | G | 83 |
| 169 | NC0147719 | 5 | 159.9 | G | T | 62 |
| 170 | NC0148181 | 4 | 183 | C | G | 1280 |
| 171 | NC0148208 | 7 | 126.9 | C | G | 232 |
| 172 | NC0151288 | 2 | 107.6 | A | G | 1420 |
| 173 | NC0153141 | 5 | 138.4 | A | G | 298 |
| 174 | NC0154151 | 3 | 109.3 | A | G | 189 |
| 175 | NC0155829 | 7 | 99 | A | G | 418 |
| 176 | NC0156284 | 5 | 74.1 | C | T | 388 |

A. Mapping LY038 Transgene Modulating Loci Associated with Lysine Concentration in Crosses of LY038 Inbreds with High or Low Lysine Phenotypes

The inbred conversion “High lysine,” herein referred to as “High 1,” exhibited a lysine level of 700.7 ppm (stdev.228.5) and the inbred conversion “Low lysine,” herein referred to as “Low 1,” exhibited a lysine level of 167.6 ppm (stdev. 87.9).

In one aspect, the High 1 and Low 1 inbred conversions were crossed and F1 hybrid seed was collected to test for the modulating loci. The F1 seed was planted, the F1 progeny plant was selfed, and the F2 progeny seed are generated and collected. Thus, this population was fixed for the LY038 transgene, but was segregating for loci modulating the levels of lysine, hence the performance of the transgenic trait.

Individual F2s are self-pollinated and test crossed to the hybrid. Lysine levels in ppm was measured on an F2 basis for the mapping population; on both the F3 seed (on ears of pollinated selfed F2 plant) and the test crossed seed pollinated by each F2 (on ears of hybrid). Each F2 in the segregating mapping population comprises 168 individuals that are analyzed with a set of 100 genetic markers. Proprietary markers are designed that can distinguish between High 1 and Low 1 inbreds. Markers are selected at 20 cM intervals across the genome and all individuals are genotyped. Progeny of the resultant F2 comprise a recombined population in which different genomic regions from either parent were reshuffled into unique combinations. The resultant set of recombined progeny allows for tests of correlations of lysine ppm to genotypic segregation of each marker locus. The data was analyzed via single factor analysis of variance (ANOVA) and via MAPMAKER/QTL; the latter performs similar tests of association with additional tests that are interpolated between markers. All tests are of the null hypothesis that the lysine level genotypic class means are equivalent.

For the test cross data, 7 of the 100 markers tested with ANOVA show a significant association at the P<0.05 level, with 2 of these markers showing a significant association at the P<0.01 level. These seven significant associations represent independent genomic regions. Significant LSDs (least significant difference) among the genotypic class means for the test cross data are 106-111 ppm. Significant R² values for the test-cross data range from 4.0 to 7.5%. MAPMAKER/QTL analysis essentially verified ANOVA results with significant LOD scores>2.0 (100:1 ratio) detecting the same regions of single factor ANOVA at P<0.01.

For the selfed data, 10 of the 100 markers tested with ANOVA showed a significant association at the P<0.05 level (Table 4). Significant LSDs among the genotypic class means for test cross data range from 138-158 ppm. Significant R² values for the selfed data, range form 3.4 to 7.2%. Of the 10 significantly associated regions among the selfed data, 4 are common with the testcross data. The MAPMAKER/QTL analysis essentially verifies the ANOVA results with LOD scores>2.0 (100:1 ratio) detecting the same regions of single factor ANOVA P<0.01.

TABLE 4
Single nucleotide polymorphism markers associated LY038
transgene modulator QTLs for lysine concentration and their
map positions in a F2 cross of High 1*Low 1 population.
Location, significance of the association, and allele associated
with the positive effect are indicated.

| QTL | Marker | chr | position | Sig | Fav parent | effect |
|---|---|---|---|---|---|---|
| 1 | NC0036685 | 1 | 45.8 | 0.0218 | High 1 | 177.23 |
| 3 | NC0039298 | 1 | 194.6 | 0.0165 | High 1 | 174.985 |
| 4 | NC0015344 | 1 | 221.1 | 0.0281 | High 1 | 167.19 |
| 6 | NC0060879 | 2 | 97.7 | 0.0312 | High 1 | 128.335 |
| 10 | NC0108727 | 3 | 77.4 | 0.0037 | High 1 | 126.35 |
| 10 | NC0024631 | 3 | 83.2 | 0.0002 | High 1 | 132.35 |
| 10 | NC0008900 | 3 | 97.6 | 0.0005 | High 1 | 121.895 |
| 11 | NC0002905 | 3 | 123.9 | 0.0147 | High 1 | 721.705 |
| 14 | NC0009057 | 4 | 21.7 | 0.0206 | High 1 | 167.19 |
| 15 | NC0019003 | 4 | 45.3 | 0.0244 | High 1 | 104.47 |

In the following examples, results are reported for additional populations that were evaluated on a single marker basis for LY038 transgene modulating loci. F2 mapping populations were evaluated that were homozygous for the LY038 transgene but segregating at all other genetic background regions. F2 mapping populations were generated from crosses of previously characterized as “High” genetic background or “Low” genetic background parents. Two newly evaluated F2 populations included the High 1*Low 2 population and High 1*High 2 population. These experiments describe the number, location, magnitude, and parental allele contribution of effects. Effects detected among the different populations are compared for commonality and exclusivity of map location. Additional mapping populations were evaluated that were derived from the crosses of non-transgenic lines, but were test-crossed to a homozygous LY038 conversion. This provided the evaluation of LY038 in the hemizygous state.

For the High 1*Low 2 population and High 1*High 2 population, individuals were sampled, genotyped with approximately 200 markers, and evaluated for lysine. Free lysine was evaluated on 50 kernels of the single selfed ear. Results are in Table 5 and 6 respectively. Summary results for significant markers for all three populations are reported in Table 7.

TABLE 5
Single nucleotide polymorphism markers associated LY038
transgene modulator QTLs for lysine concentration and their
map positions in a F2 cross of High 1 * High 2 population.
Significant (LOD > 2.4) effects ranged from 190.19 to
624.69 ppm and were detected on chromosomes
1, 2, 3, 4, 5, 6, 8, and 10. Location, significance of the association,
and allele associated with the positive effect are indicated.

| QTL | Marker | chr | position | sig | Fav parent | effect |
|---|---|---|---|---|---|---|
| 1 | NC0143873 | 1 | 52.6 | 0.0482 | High 2 | 303.3 |
| 2 | NC0035579 | 1 | 94.5 | 0.0173 | High 2 | 200.5 |
| 2 | NC0025863 | 1 | 96.7 | 0.0482 | High 2 | 202.8 |
| 2 | NC0069524 | 1 | 99.9 | 0.0066 | High 1 | 310.05 |
| 3 | NC0067728 | 1 | 173.7 | 0.0247 | High 2 | 260.1 |
| 3 | NC0027567 | 1 | 179.4 | 0.0141 | High 2 | 299.9 |
| 4 | NC0035961 | 1 | 206.7 | 0.0007 | High 1 | 219.5 |
| 6 | NC0060879 | 2 | 97.7 | 0.0003 | High 2 | 208.7 |
| 7 | NC0005088 | 2 | 147.6 | 0.0133 | 0 | 155.3 |
| 9 | NC0008911 | 3 | 19.9 | 0.0088 | High 1 | 214.5 |
| 10 | NC0049293 | 3 | 69.9 | 0.0258 | High 1 | 206.75 |
| 11 | NC0154151 | 3 | 109.3 | 0.0483 | High 1 | 163.3 |
| 12 | NC0015965 | 3 | 140.8 | 0.0039 | High 2 | 194.05 |
| 12 | NC0021772 | 3 | 154.1 | 0.0317 | High 1 | 195.75 |
| 12 | NC0004371 | 3 | 164.2 | 0.0037 | High 2 | 228.1 |
| 13 | NC0010232 | 3 | 198.7 | 0.0334 | High 2 | 569.7 |
| 15 | NC0024647 | 4 | 52.5 | 0.0086 | High 1 | 569.7 |
| 15 | NC0037062 | 4 | 59.7 | 0.0336 | High 1 | 254.5 |
| 15 | NC0037062 | 4 | 59.7 | 0.0359 | High 1 | 209.1 |
| 15 | NC0034325 | 4 | 63.7 | 0.0052 | High 2 | 205.25 |
| 16 | NC0033667 | 4 | 73.7 | 0.0165 | High 1 | 155.45 |
| 16 | NC0104785 | 4 | 83.9 | 0.0139 | High 1 | 178.95 |
| 17 | NC0009620 | 4 | 109.2 | 0.0281 | High 1 | 183.15 |
| 18 | NC0106263 | 4 | 133 | 0.0262 | High 2 | 230.85 |
| 19 | NC0003224 | 4 | 173.6 | 0.0113 | High 2 | 206.15 |
| 19 | NC0035338 | 4 | 190.6 | 0.0021 | High 1 | 1042.9 |
| 20 | NC0143354 | 5 | 1.8 | 0.008 | High 2 | 256.2 |
| 20 | NC0143251 | 5 | 11.6 | 0.0079 | High 2 | 1275.7 |
| 20 | NC0105613 | 5 | 16.6 | 0.0005 | High 1 | 195.65 |
| 21 | NC0113172 | 5 | 43.8 | 0.0388 | High 2 | 187.45 |
| 22 | NC0004605 | 5 | 78.5 | 0.0033 | High 2 | 320.35 |
| 23 | NC0009297 | 5 | 104.1 | 0.0237 | High 2 | 268.7 |
| 23 | NC0147315 | 5 | 115 | 0.0008 | High 1 | 259.3 |
| 24 | NC0143380 | 5 | 148.1 | 0.0006 | High 2 | 318.7 |
| 24 | NC0147719 | 5 | 159.9 | 0.0033 | High 1 | 186.05 |
| 27 | NC0105497 | 6 | 67.6 | 0.0004 | High 1 | 319.2 |
| 27 | NC0037947 | 6 | 97.6 | 0.0293 | High 1 | 291.5 |
| 28 | NC0146215 | 6 | 106.6 | 0.0001 | High 2 | 246.2 |
| 29 | NC0028185 | 6 | 130.1 | 0.0003 | High 2 | 212.65 |
| 30 | NC0066143 | 7 | 57.1 | 0.0018 | High 2 | 236.05 |
| 31 | NC0030029 | 7 | 112.7 | 0.0168 | High 2 | 296.65 |
| 32 | NC0051919 | 8 | 71.1 | 0.0002 | High 2 | 233.8 |
| 33 | NC0144363 | 8 | 91.1 | 0.0001 | High 2 | 152.15 |
| 34 | NC0004586 | 8 | 125.1 | 0.0036 | High 1 | 231 |
| 35 | NC0027914 | 9 | 45 | 0.0257 | High 2 | 399.75 |
| 37 | NC0147417 | 9 | 153.2 | 0.0487 | High 1 | 455.75 |
| 37 | NC0054661 | 10 | 57.1 | 0.0052 | High 2 | 293.55 |
| 37 | NC0104512 | 10 | 57.3 | 0.0088 | High 1 | 234 |
| 37 | NC0031358 | 10 | 64.2 | 0.0186 | High 2 | 241 |

TABLE 6
Single nucleotide polymorphism markers associated LY038
transgene modulator QTLs for lysine concentration and their
map positions in a F2 cross of High 1 * Low 2 population.
Location, significance of the association, and allele
associated with the positive effect are indicated.

| QTL | Marker | chr | position | sig | Fav parent | effect |
|---|---|---|---|---|---|---|
| 1 | NC0036685 | 1 | 45.8 | 0.0218 | High 1 | 177.23 |
| 3 | NC0039298 | 1 | 194.6 | 0.0165 | High 1 | 174.985 |
| 4 | NC0015344 | 1 | 221.1 | 0.0281 | High 1 | 167.19 |
| 6 | NC0060879 | 2 | 97.7 | 0.0312 | High 1 | 128.335 |
| 10 | NC0108727 | 3 | 77.4 | 0.0037 | High 1 | 126.35 |
| 10 | NC0024631 | 3 | 83.2 | 0.0002 | High 1 | 132.35 |
| 10 | NC0008900 | 3 | 97.6 | 0.0005 | High 1 | 121.895 |
| 11 | NC0002905 | 3 | 123.9 | 0.0147 | High 1 | 721.705 |
| 14 | NC0009057 | 4 | 21.7 | 0.0206 | High 1 | 167.19 |
| 15 | NC0019003 | 4 | 45.3 | 0.0244 | High 1 | 104.47 |
| 15 | NC0024647 | 4 | 52.5 | 0.0052 | High 1 | 112.29 |
| 15 | NC0034325 | 4 | 63.7 | 0.0016 | High 1 | 164.215 |
| 16 | NC0033667 | 4 | 73.7 | 0.017 | High 1 | 167.19 |
| 16 | NC0104785 | 4 | 83.9 | 0.0004 | High 1 | 207.19 |
| 16 | NC0005018 | 4 | 94.8 | <.0001 | High 1 | 202.215 |
| 17 | NC0009620 | 4 | 109.2 | <.0001 | High 1 | 308.225 |
| 17 | NC0039511 | 4 | 121.5 | <.0001 | High 1 | 255.195 |
| 18 | NC0005295 | 4 | 135.1 | 0.0002 | High 1 | 120.67 |
| 18 | NC0048567 | 4 | 146.9 | <.0001 | High 1 | 207.42 |
| 18 | NC0036534 | 4 | 147.9 | 0.0176 | High 1 | 166.215 |
| 19 | NC0032049 | 4 | 162.6 | 0.0393 | High 1 | 198.85 |
| 19 | NC0060681 | 4 | 164.4 | 0.002 | High 1 | 104.47 |
| 19 | NC0109283 | 4 | 175.5 | 0.0001 | High 1 | 138.8 |
| 26 | NC0112604 | 6 | 38.4 | 0.038 | Low 2 | 315.1 |
| 26 | NC0032034 | 6 | 57.6 | 0.0185 | High 1 | 151.625 |
| 33 | NC0015146 | 8 | 84 | <.0001 | High 1 | 350.11 |
| 33 | NC0104858 | 8 | 96.2 | <.0001 | High 1 | 310.515 |
| 33 | NC0031025 | 8 | 108.5 | 0.0033 | High 1 | 150.4 |
| 34 | NC0059764 | 8 | 118.8 | 0.0006 | High 1 | 108.9 |
| 35 | NC0025198 | 9 | 45.7 | 0.0525 | High 1 | 162.085 |
| 35 | NC0023779 | 9 | 56.1 | 0.0225 | Low 2 | 140.575 |
| 37 | NC0054661 | 10 | 57.1 | 0.0158 | High 1 | 150.23 |
| 37 | NC0027447 | 10 | 75.6 | 0.0495 | High 1 | 108.35 |

TABLE 7
Summary of genetic locations and significance (LOD > 2.4) for
interval mapping of LY038 transgene modulating loci associated
with lysine concentration for High 1*Low 1, High 1*Low 2, and High
1*High 2. Additive effect is reported with respect to the High 1 parent.

| | | | | Additive | |
|---|---|---|---|---|---|
| Population | Chrm | Pos. | LOD | effect* | R² |
| High 1*Low1 | 1 | 69.5 | 3.5 | 219.00 | 0.20 |
| High 1*Low1 | 4 | 35.5 | 2.9 | 166.49 | 0.14 |
| High 1*Low1 | 4 | 68 | 2.7 | 149.50 | 0.11 |
| High 1*Low1 | 4 | 88.6 | 2.4 | 148.22 | 0.11 |
| High 1*Low1 | 4 | 112.3 | 2.5 | 162.91 | 0.12 |
| High 1*Low1 | 4 | 127.9 | 2.4 | 151.08 | 0.10 |
| High 1*Low1 | 9 | 36.7 | 2.5 | 115.58 | 0.08 |
| High 1*Low 2 | 3 | 74.6 | 2.5 | 246.55 | 0.10 |
| High 1*Low 2 | 4 | 42.7 | 2.7 | 150.18 | 0.04 |
| High 1*Low 2 | 4 | 55.9 | 2.4 | 121.44 | 0.03 |
| High 1*Low 2 | 4 | 82.1 | 4.0 | 198.95 | 0.08 |
| High 1*Low 2 | 4 | 99.4 | 10.6 | 345.21 | 0.21 |
| High 1*Low 2 | 4 | 131.3 | 4.6 | 235.03 | 0.11 |
| High 1*Low 2 | 4 | 138.1 | 4.7 | 232.92 | 0.10 |
| High 1*Low 2 | 4 | 159.3 | 4.6 | 173.23 | 0.06 |
| High 1*Low 2 | 4 | 163.8 | 3.4 | 130.83 | 0.03 |
| High 1*Low 2 | 8 | 71.5 | 12.8 | 360.70 | 0.22 |
| High 1*Low 2 | 8 | 81.7 | 10.4 | 364.34 | 0.22 |
| High 1*High 2 | 1 | 63.9 | 3.5 | 409.97 | 0.09 |
| High 1*High 2 | 1 | 200.2 | 3.2 | −264.35 | 0.06 |
| High 1*High 2 | 2 | 16.8 | 2.6 | −320.08 | 0.07 |
| High 1*High 2 | 2 | 73.6 | 3.9 | 278.08 | 0.05 |
| High 1*High 2 | 2 | 96.2 | 3.0 | 262.39 | 0.05 |
| High 1*High 2 | 3 | 10.0 | 2.3 | 225.91 | 0.04 |
| High 1*High 2 | 4 | 19.9 | 2.7 | 624.69 | 0.11 |
| High 1*High 2 | 4 | 56.7 | 2.1 | 321.86 | 0.06 |
| High 1*High 2 | 4 | 65.9 | 2.3 | 276.58 | 0.05 |
| High 1*High 2 | 4 | 77.9 | 2.4 | 242.02 | 0.04 |
| High 1*High 2 | 5 | 12.51 | 2.7 | 472.97 | 0.14 |
| High 1*High 2 | 5 | 113.2 | 3.9 | −316.00 | 0.07 |
| High 1*High 2 | 5 | 150.3 | 2.6 | −190.19 | 0.03 |
| High 1*High 2 | 6 | 42.6 | 3.2 | −284.25 | 0.05 |
| High 1*High 2 | 6 | 87.6 | 5.0 | −366.04 | 0.10 |
| High 1*High 2 | 8 | 10.0 | 7.9 | 395.68 | 0.11 |
| High 1*High 2 | 8 | 45.3 | 4.7 | 403.42 | 0.13 |
| High 1*High 2 | 8 | 65.3 | 5.1 | 403.52 | 0.13 |
| High 1*High 2 | 10 | 48.5 | 5.3 | 305.64 | 0.07 |
| High 1*High 2 | 10 | 50.7 | 5.5 | 755.90 | 0.34 |
| High 1*High 2 | 10 | 52.9 | 6.9 | 883.48 | 0.17 |

B. Mapping LY038 Transgene Modulating Loci Associated with White Seedling Phenotype in Crosses of LY038 Inbreds with High or Low Lysine Phenotypes

An additional correlated trait of white seedling color was also scored on a subset of individuals in each of these F2 populations. In the High 1*Low 2 population, F₂, approximately half of the plants were green, half of the plants were green and white striped, but there was an occasional all white plant (at approximately 5% frequency). In the High 1*High 2 population, there was nearly equivalent distribution among different color classes: ⅓ all green, ⅓ green and white striped, ⅓ all white. Color phenotypes were assigned a categorical class number (1, 2, or 3) and analyzed with respect to marker data. Notably, this character represents a marker that is a phenotype that can be used as the basis for breeding decisions.

In addition, the populations were genotyped to also identify one or more genetic markers associated with a LY038 transgene modulating locus associated with white seedling phenotype. Data for the High 1*High 2 and High 1*Low 2 populations are reported in Tables 8 and 9. Summary results for significant markers for all three populations are reported in Table 10.

TABLE 8
Single nucleotide polymorphism markers associated LY038
transgene modulator QTLs for white seedling phenotype and
their map positions in a F2 cross of High 1*High 2 population.
Location, significance of the association, and allele
associated with the positive effect are indicated.

| QTL | Marker | chr | position | Signif | Fav parent | effect |
|---|---|---|---|---|---|---|
| 2 | NC0105022 | 1 | 79.5 | 0.0325 | High 1 | 0.213 |
| 2 | NC0035579 | 1 | 94.5 | 0.049 | High 2 | 0.192 |
| 3 | NC0067728 | 1 | 173.7 | 0.0103 | High 2 | 0.24735 |
| 3 | NC0027567 | 1 | 179.4 | 0.0158 | High 2 | 0.27645 |
| 3 | NC0039298 | 1 | 194.6 | 0.0118 | High 2 | 0.2424 |
| 4 | NC0080031 | 2 | 33.1 | 0.028 | High 2 | 0.2152 |
| 4 | NC0027262 | 2 | 57.3 | 0.0253 | High 2 | 0.20375 |
| 4 | NC0019110 | 2 | 75.1 | 0.0115 | High 2 | 0.2096 |
| 5 | NC0060879 | 2 | 97.7 | 0.0366 | High 2 | 0.25665 |
| 5 | NC0009818 | 2 | 136.5 | 0.0006 | High 2 | 0.2704 |
| 5 | NC0005088 | 2 | 147.6 | 0.0005 | High 2 | 0.25885 |
| 6 | NC0020971 | 3 | 13.9 | 0.0013 | High 2 | 0.32145 |
| 6 | NC0008911 | 3 | 19.9 | 0.0001 | High 2 | 0.33815 |
| 7 | NC0049293 | 3 | 69.9 | 0.0287 | High 2 | 0.2122 |
| 7 | NC0004371 | 3 | 164.2 | 0.0389 | High 2 | −0.2031 |
| 8 | NC0037062 | 4 | 59.7 | 0.0254 | High 2 | 0.26085 |
| 8 | NC0034325 | 4 | 63.7 | 0.0134 | High 2 | 0.25895 |
| 8 | NC0033667 | 4 | 73.7 | 0.0033 | High 2 | 0.2859 |
| 8 | NC0104785 | 4 | 83.9 | 0.0004 | High 2 | 0.35715 |
| 9 | NC0009620 | 4 | 109.2 | 0.0079 | High 2 | 0.2661 |
| 10 | NC0017828 | 4 | 144.7 | 0.0069 | High 1 | 0.27855 |
| 11 | NC0060681 | 4 | 164.4 | 0.019 | High 2 | 0.2614 |
| 11 | NC0029487 | 4 | 171.1 | 0.0037 | High 2 | 0.2875 |
| 11 | NC0003224 | 4 | 173.6 | 0.0026 | High 2 | 0.28015 |
| 11 | NC0003226 | 4 | 173.6 | 0.0105 | High 1 | 0.24355 |
| 11 | NC0013833 | 4 | 175.6 | 0.0037 | High 2 | 0.35295 |
| 11 | NC0004445 | 4 | 176.6 | 0.0022 | High 2 | 0.2594 |
| 11 | NC0017900 | 4 | 179.3 | 0.0058 | High 2 | −0.48865 |
| 11 | NC0036415 | 4 | 181 | 0.004 | High 2 | 0.25415 |
| 11 | NC0144324 | 4 | 183 | 0.03 | High 1 | 0.17815 |
| 11 | NC0148181 | 4 | 183 | 0.0396 | High 2 | 0.19335 |
| 11 | NC0035338 | 4 | 190.6 | 0.002 | High 1 | 0.29335 |
| 12 | NC0143354 | 5 | 1.8 | 0.044 | High 2 | 0.1908 |
| 12 | NC0105613 | 5 | 16.6 | 0.0015 | High 2 | 0.323 |
| 12 | NC0033977 | 5 | 29.3 | 0.0014 | High 2 | 0.233 |
| 12 | NC0113172 | 5 | 43.8 | 0.0214 | High 1 | 0.222 |
| 13 | NC0004605 | 5 | 78.5 | 0.0005 | High 2 | 0.27535 |
| 16 | NC0014417 | 6 | 25 | 0.0058 | High 2 | 0.21645 |
| 16 | NC0105497 | 6 | 67.6 | 0.0117 | High 1 | 0.24715 |
| 17 | NC0143819 | 7 | 7.1 | 0.0227 | High 1 | 0.23515 |
| 18 | NC0068434 | 7 | 76.5 | 0.015 | High 2 | 0.2289 |
| 19 | NC0004953 | 7 | 131.2 | 0.03 | High 2 | 0.2184 |
| 21 | NC0051919 | 8 | 71.1 | 0.0002 | High 2 | 0.48 |
| 21 | NC0020546 | 8 | 115.6 | 0.0173 | High 2 | 0.2202 |
| 22 | NC0014479 | 9 | 0.8 | 0.0179 | High 1 | 0.312 |
| 23 | NC0147417 | 9 | 153.2 | 0.0005 | High 1 | 0.321 |
| 24 | NC0009645 | 10 | 32.1 | 0.0244 | High 1 | 0.214 |
| 24 | NC0004887 | 10 | 45.2 | 0.0067 | High 1 | 0.31 |
| 24 | NC0109795 | 10 | 53 | 0.0109 | High 1 | 0.251 |

TABLE 9
Single nucleotide polymorphism markers associated LY038
transgene modulator QTLs for white seedling phenotype and
their map positions in a F2 cross of High 1*Low 2 population.
Location, significance of the association, and allele associated
with the positive effect are indicated.

| QTL | Marker | chr | position | Signif | Fav parent | effect |
|---|---|---|---|---|---|---|
| 1 | NC0147302 | 1 | 27.6 | <.0001 | High 1 | 0.365 |
| 1 | NC0036685 | 1 | 45.8 | 0.0402 | Low 2 | 0.179 |
| 2 | NC0039840 | 1 | 65.8 | 0.021 | Low 2 | 0.231 |
| 7 | NC0024631 | 3 | 83.2 | 0.0027 | Low 2 | 0.237 |
| 7 | NC0008900 | 3 | 97.6 | 0.0189 | High 1 | 0.187 |
| 8 | NC0019003 | 4 | 45.3 | 0.0464 | High 1 | 0.211 |
| 8 | NC0024647 | 4 | 52.5 | 0.0338 | Low 2 | 0.255 |
| 8 | NC0033667 | 4 | 73.7 | 0.0061 | Low 2 | 0.258 |
| 8 | NC0104785 | 4 | 83.9 | <.0001 | Low 2 | 0.366 |
| 9 | NC0005018 | 4 | 94.8 | 0.0002 | Low 2 | 0.329 |
| 9 | NC0009620 | 4 | 109.2 | <.0001 | Low 2 | 0.308 |
| 9 | NC0039511 | 4 | 121.5 | 0.0004 | Low 2 | 0.3 |
| 10 | NC0005295 | 4 | 135.1 | 0.0065 | Low 2 | 0.248 |
| 10 | NC0048567 | 4 | 146.9 | 0.0477 | Low 2 | 0.196 |
| 10 | NC0036534 | 4 | 147.9 | 0.0261 | Low 2 | 0.211 |
| 10 | NC0030576 | 4 | 153.8 | 0.0038 | Low 2 | 0.271 |
| 10 | NC0107293 | 4 | 155.5 | 0.0055 | Low 2 | 0.259 |
| 10 | NC0105818 | 4 | 155.8 | 0.0191 | Low 2 | 0.232 |
| 11 | NC0032049 | 4 | 162.6 | 0.028 | Low 2 | 0.223 |
| 11 | NC0036415 | 4 | 181 | 0.0204 | Low 2 | −0.389 |
| 11 | NC0030985 | 4 | 181.9 | 0.0098 | Low 2 | 0.244 |
| 11 | NC0144324 | 4 | 183 | 0.0222 | Low 2 | 0.231 |
| 12 | NC0012935 | 5 | 45.7 | 0.0041 | High 1 | 0.283 |
| 13 | NC0037588 | 5 | 60.1 | 0.0007 | High 1 | 0.296 |
| 13 | NC0111388 | 5 | 66.6 | <.0001 | Low 2 | 0.372 |
| 14 | NC0040571 | 5 | 88.4 | <.0001 | Low 2 | 0.36 |
| 14 | NC0036637 | 5 | 100 | 0.0001 | Low 2 | 0.332 |
| 15 | NC0083876 | 5 | 124 | 0.0001 | Low 2 | 0.373 |
| 15 | NC0153141 | 5 | 138.4 | 0.0539 | Low 2 | 0.281 |
| 15 | NC0143380 | 5 | 148.1 | 0.0014 | Low 2 | 0.279 |
| 16 | NC0014417 | 6 | 25 | 0.044 | Low 2 | 0.188 |
| 16 | NC0032034 | 6 | 57.6 | | Low 2 | 0.029 |
| 17 | NC0143514 | 7 | 29 | 0.0001 | Low 2 | 0.313 |
| 17 | NC0013158 | 7 | 48.6 | <.0001 | Low 2 | 0.314 |
| 18 | NC0066807 | 7 | 67.1 | <.0001 | Low 2 | 0.378 |
| 18 | NC0068434 | 7 | 76.5 | <.0001 | Low 2 | 0.357 |
| 19 | NC0035408 | 7 | 89.5 | 0.0108 | Low 2 | 0.287 |
| 19 | NC0004953 | 7 | 131.2 | 0.0083 | Low 2 | 0.248 |
| 20 | NC0000129 | 8 | 16.5 | 0.0416 | Low 2 | 0.187 |
| 22 | NC0025198 | 9 | 45.7 | 0.0114 | High 1 | 0.211 |
| 24 | NC0054661 | 10 | 57.1 | 0.0146 | High 1 | 0.318 |

TABLE 10
Summary of genetic locations and significance (LOD > 2.4) for
interval mapping of LY038 transgene modulating loci associated
with white seedling phenotype for High 1*Low 1, High 1*Low2,
and High 1*High 2. Additive effect is reported with respect to the
High 1 parent.

| Population | Chrm | Pos. | LOD | Additive effect | R² |
|---|---|---|---|---|---|
| High 1*Low 2 | 4 | 78.1 | 4.49 | −3.18 | 0.113 |
| High 1*Low 2 | 4 | 87 | 4.29 | −3.12 | 0.105 |
| High 1*Low 2 | 4 | 107.4 | 3.1 | −2.36 | 0.06 |
| High 1*Low 2 | 4 | 111.7 | 3.07 | −2.47 | 0.066 |
| High 1*Low 2 | 4 | 142 | 2.52 | −2.86 | 0.083 |
| High 1*Low 2 | 4 | 144 | 2.27 | −2.68 | 0.072 |
| High 1*Low 2 | 4 | 170.1 | 2.01 | −2.33 | 0.058 |
| High 1*Low 2 | 5 | 51.9 | 3.04 | −3.54 | 0.126 |
| High 1*Low 2 | 5 | 60.3 | 2.98 | −4.38 | 0.203 |
| High 1*Low 2 | 5 | 88.6 | 4.74 | −4.45 | 0.203 |
| High 1*Low 2 | 5 | 106.2 | 4.33 | −4.55 | 0.21 |
| High 1*Low 2 | 5 | 126.2 | 3.68 | −3.69 | 0.126 |
| High 1*Low 2 | 7 | 29.61 | 5.92 | −3.62 | 0.145 |
| High 1*Low 2 | 7 | 49.51 | 3.82 | −2.54 | 0.066 |
| High 1*Low 2 | 7 | 66.51 | 2.3 | −2.22 | 0.051 |
| High 1*Low 2 | 7 | 108.21 | 2.06 | −2.67 | 0.078 |
| High 1*High 2 | 1 | 122.4 | 2.13 | −3.17 | 0.12 |
| High 1*High 2 | 3 | 4.0 | 4.17 | −3.55 | 0.11 |
| High 1*High 2 | 4 | 19.9 | 4.64 | −8.05 | 0.16 |
| High 1*High 2 | 4 | 67.9 | 2.43 | −2.82 | 0.06 |
| High 1*High 2 | 4 | 77.9 | 2.88 | −3.09 | 0.08 |
| High 1*High 2 | 4 | 84.1 | 2.65 | −3.02 | 0.08 |
| High 1*High 2 | 4 | 142.9 | 2.29 | 2.18 | 0.03 |
| High 1*High 2 | 4 | 169.3 | 2.46 | −2.74 | 0.05 |
| High 1*High 2 | 4 | 181.2 | 2.15 | −2.40 | 0.04 |
| High 1*High 2 | 4 | 188.3 | 2.99 | 3.55 | 0.11 |
| High 1*High 2 | 5 | 8.0 | 6.84 | −12.28 | 0.48 |
| High 1*High 2 | 5 | 14.8 | 2.18 | −3.24 | 0.08 |
| High 1*High 2 | 5 | 29.5 | 2.21 | −3.14 | 0.05 |
| High 1*High 2 | 5 | 33.8 | 11.88 | −11.12 | 0.30 |
| High 1*High 2 | 5 | 113.2 | 4.24 | −3.298 | 0.09 |
| High 1*High 2 | 10 | 52.9 | 8.42 | −10.9 | 0.22 |

To assist in the understanding of the genetic correlation of the control of lysine across populations, the correlation of the additive effect values were run among the three F2 mapping populations (High 1*High 2, High 1*Low 1, High 1*Low 2). To do this, effects were assigned on the basis of map position in 10cM windows and the correlation was run on effect estimates in common windows. Correlations were evaluated for the effects of the white seedling color trait as well as lysine (Table 11).

TABLE 11
Correlations among F2 population additive effects for lysine and
seedling color phenotypic traits.

| | Color | | Lysine | Lysine | Lysine |
|---|---|---|---|---|---|
| | High | Color | ppm | ppm | ppm |
| | 1*High | High | High | High | High |
| | 2 | 1*High 2 | 1*High 2 | 1*Low 2 | 1*Low 2 |
| Color | 1 | 0.0693 | −0.135 | −0.189 | −0.086 |
| High 1*High 2 | | 0.444 | 0.128 | 0.0347 | 0.4545 |
| | | 124 | 129 | 124 | 78 |
| Color | | 1 | 0.166 | −0.068 | 0.0754 |
| High 1*High 2 | | | 0.0648 | 0.4475 | 0.5261 |
| | | | 124 | 124 | 73 |
| Lysine ppm | | | 1 | 0.3597 | 0.31296 |
| High 1*High 2 | | | | <0.0001 | 0.0053 |
| | | | | 124 | 78 |
| Lysine ppm | | | | 1 | 0.5293 |
| High 1*Low 2 | | | | | <0.0001 |
| | | | | | 73 |
| Lysine ppm | | | | | 1 |
| High 1*Low 2 | | | | | |

Significant modifier effects across populations were found in the same chromosomal regions, indicating common genetic control (chromosome #4 across all three populations, chromosome 1 and 8 across two of the three populations). Commonality of genetic control is further indicated by significant correlations among additive effects across populations. However, data also suggest there are population specific modifiers. While optimizing a specific genetic background for the lysine trait may require breeding with more than one modifying locus, experience has shown that some effects have greater magnitudes of effect than others.

C. Mapping LY038 Transgene Modulating Loci Associated with Lysine Concentration in Crosses of LY038 Inbreds with LY038 Null Inbreds to Evaluate Effect of LY038 Hemizygosity

In another aspect, copy number may impact transgene modulating loci. Additional populations (Low 1 conversion without LY038 or F2:F3s without LY038 were testcrossed to LY038 tester, either High 1 or Low 2) were evaluated for lysine concentration and presence of LY038 transgene modulating QTL when the transgene was in the hemizygous state.

Data analysis of association of marker genotypes on free lysine included single factor ANOVA and multiple regression analyses in SAS, and interval mapping with QTL CARTOGRAPHER. In the previously characterized High 1*Low 1 cross, significant (LOD>2.4) effects ranged from 115.58 and 219.00 ppm and were found on chromosomes 1, 4, and 9 (Table 4). In the newly evaluated High 1*Low 1 cross, with a non-LY038 version of Low 1, significant (LOD>2.4) effects ranged from 121.44 to 364.34 ppm and were detected on chromosomes 3, 4, and 8 (Table 12).

TABLE 12
Single nucleotide polymorphism markers associated LY038
transgene modulator QTLs for lysine concentration and their
map positions in a cross of High 1 by Low 1 conversion
without LY038. Location, significance of the association, and
allele associated with the positive effect are indicated.

| | | | | | Fav | |
|---|---|---|---|---|---|---|
| QTL | Marker | chr | position | sig | parent | effect |
| 1 | NC0036685 | 1 | 45.8 | 0.0049 | Low 1 | −102 |
| 2 | NC0077749 | 1 | 79.6 | 6E−05 | High 1 | 133.2 |
| 3 | NC0106296 | 1 | 181 | 0.0403 | High 1 | 67.99 |
| 5 | NC0080705 | 2 | 68.5 | 0.0222 | High 1 | 75.98 |
| 5 | NC0009364 | 2 | 71.6 | 0.012 | High 1 | 83.34 |
| 6 | NC0005467 | 2 | 94.3 | 0.0114 | High 1 | 82.95 |
| 6 | NC0108013 | 2 | 115.3 | 0.0052 | High 1 | 96.37 |
| 7 | NC0009102 | 2 | 130 | 0.0098 | High 1 | 90.08 |
| 10 | NC0108727 | 3 | 77.4 | 0.0088 | High 1 | 90.74 |
| 12 | NC0009473 | 3 | 168.4 | 0.0272 | High 1 | 75.65 |
| 14 | NC0002739 | 4 | 11.8 | 0.0399 | High 1 | 66.97 |
| 15 | NC0019003 | 4 | 45.3 | 0.0002 | High 1 | 127.4 |
| 15 | NC0040371 | 4 | 67.8 | 0.0008 | High 1 | 112.7 |
| 16 | NC0069570 | 4 | 92.4 | 0.0015 | High 1 | 108.1 |
| 17 | NC0036239 | 4 | 112.1 | 0.002 | High 1 | 107.3 |
| 18 | NC0054460 | 4 | 131.7 | 0.0015 | High 1 | 116.1 |
| 18 | NC0030576 | 4 | 153.8 | 0.0032 | High 1 | 105 |
| 26 | NC0027095 | 6 | 38.8 | 0.0348 | Low 1 | −66.9 |
| 29 | NC0021734 | 6 | 145.4 | 0.0406 | High 1 | 68.42 |
| 31 | NC0155829 | 7 | 99 | 0.0588 | High 1 | −66.6 |
| 32 | NC0034552 | 8 | 51.8 | 0.0522 | High 1 | −54.1 |
| 35 | NC0012830 | 9 | 33.1 | 0.0068 | High 1 | 84.19 |
| 35 | NC0027914 | 9 | 45 | 0.0007 | High 1 | 104.1 |
| 35 | NC0104195 | 9 | 68.5 | 0.0327 | High 1 | 72.72 |
| 36 | NC0108275 | 9 | 91.6 | 0.0553 | High 1 | 67.51 |
| 37 | NC0020502 | 10 | 30.3 | 0.0203 | High 1 | −74.8 |

Additional mapping experiments were performed where the lysine transgene was in the hemizygous condition. Four F2:F3 populations, designated herein populations 1-4, were evaluated and crossed to either Low 2 or High 1. Testcross progenies derived from segregating lines and a homozygous transgenic lysine tester were evaluated in two locations. Data analysis of association of marker genotypes on free lysine included single factor ANOVA and multiple regression in SAS. Because some populations had a single marker residing on one or more chromosomes, interval mapping was not completed on all populations. Results are reported in Tables 13, 14, 15, and 16.

TABLE 13
Single nucleotide polymorphism markers associated LY038
transgene modulator QTLs for lysine concentration and their
map positions in a cross of population 1 testcrossed with High 1.
Location, significance of the association, and allele associated
with the positive effect are indicated.

| QTL | Marker | chr | position | sig | effect |
|---|---|---|---|---|---|
| 23 | NC0017678 | 5 | 103.8 | 0.0236 | 74.65 |
| 23 | NC0016868 | 5 | 122.6 | 0.0248 | 74.2 |
| 24 | NC0143380 | 5 | 148.1 | 0.0416 | −67.4 |
| 26 | NC0106341 | 6 | 29.5 | 0.0482 | −66.2 |
| 34 | NC0108962 | 8 | 139.7 | 0.0435 | −67.6 |

TABLE 14
Single nucleotide polymorphism markers associated LY038
transgene modulator QTLs for lysine concentration and their
map positions in a cross of population 2 testcrossed with High 1.
Location, significance of the association, and allele associated
with the positive effect are indicated.

| QTL | Marker | chr | position | sig | effect |
|---|---|---|---|---|---|
| 3 | NC0038475 | 1 | 168.3 | 0.0194 | 65.309 |
| 5 | NC0080705 | 2 | 68.5 | 0.0045 | −86.78 |
| 6 | NC0021092 | 2 | 93.4 | 0.0031 | −86.05 |
| 6 | NC0105696 | 2 | 94.3 | 0.0122 | −74.58 |
| 6 | NC0146130 | 2 | 94.6 | 0.0083 | −78.99 |
| 12 | NC0004371 | 3 | 164.2 | 0.0174 | −72.87 |
| 13 | NC0112644 | 3 | 181.8 | 0.0477 | −58.27 |
| 13 | NC0143969 | 3 | 187.5 | 0.0097 | −75.35 |
| 21 | NC0111388 | 5 | 66.6 | 0.0019 | 86.473 |
| 29 | NC0021734 | 6 | 145.4 | 0.0409 | 60.545 |

TABLE 15
Single nucleotide polymorphism markers associated LY038
transgene modulator QTLs for lysine concentration and their
map positions in a cross of population 3 testcrossed with High 1.
Location, significance of the association, and allele associated
with the positive effect are indicated.

| QTL | Marker | chr | position | sig | Effect |
|---|---|---|---|---|---|
| 2 | NC0004176 | 1 | 116.3 | 0.0408 | 42.639 |
| 6 | NC0053097 | 2 | 102.6 | 0.0167 | −48.61 |
| 6 | NC0057210 | 2 | 104.1 | 0.0305 | −44.49 |
| 11 | NC0008900 | 3 | 97.6 | 0.0287 | −43.2 |
| 11 | NC0108089 | 3 | 106.3 | 0.0038 | 55.301 |
| 11 | NC0111959 | 3 | 117.6 | 0.0032 | 58.613 |
| 14 | NC0002739 | 4 | 11.8 | 0.0209 | 45.248 |
| 18 | NC0070533 | 4 | 130.2 | 0.0218 | −46.01 |
| 21 | NC0111388 | 5 | 66.6 | 0.0229 | 48.783 |
| 21 | NC0143216 | 5 | 67.7 | 0.0082 | −55.32 |
| 22 | NC0146546 | 5 | 71.2 | 0.0198 | 51.257 |
| 22 | NC0040571 | 5 | 88.4 | 0.0006 | −68.96 |
| 23 | NC0012480 | 5 | 99.4 | 8E−05 | 77.314 |
| 24 | NC0036210 | 5 | 145.2 | 0.0005 | −70.21 |
| 24 | NC0104963 | 5 | 159.8 | 0.0009 | −65 |
| 25 | NC0021585 | 5 | 175 | 0.0169 | 48.054 |
| 27 | NC0067075 | 6 | 98.9 | 0.0015 | 68.252 |
| 31 | NC0148208 | 7 | 126.9 | 0.0339 | 45.265 |
| 35 | NC0012830 | 9 | 33.1 | 0.0276 | 48.615 |
| 35 | NC0107905 | 9 | 63.4 | 0.0016 | −67.34 |
| 35 | NC0109526 | 9 | 66.5 | 0.0002 | −80.14 |
| 36 | NC0013086 | 9 | 87.3 | 0.0081 | 54.565 |
| 36 | NC0108275 | 9 | 91.6 | 0.0152 | −49.3 |

TABLE 16
Single nucleotide polymorphism markers associated LY038
transgene modulator QTLs for lysine concentration and their
map positions in a cross of population 4 testcrossed with Low 2.
Location, significance of the association, and allele associated
with the positive effect are indicated.

| QTL | Marker | chr | position | sig | effect |
|---|---|---|---|---|---|
| 4 | NC0009701 | 1 | 207.9 | 0.0447 | 24.109 |
| 4 | NC0015344 | 1 | 221.1 | 2E−05 | 53.089 |
| 4 | NC0002635 | 1 | 254.8 | 7E−08 | 62.67 |
| 5 | NC0032200 | 2 | 71.6 | 2E−16 | 100.14 |
| 6 | NC0005467 | 2 | 94.3 | 1E−08 | 62.6 |
| 6 | NC0151288 | 2 | 107.6 | 7E−10 | 70.406 |
| 7 | NC0031474 | 2 | 141.4 | 7E−06 | 51.529 |
| 8 | NC0110974 | 2 | 185.5 | 0.0477 | 21.952 |
| 16 | NC0022725 | 4 | 91.3 | 0.0164 | 29.784 |
| 22 | NC0156284 | 5 | 74.1 | 0.0151 | −29.75 |
| 23 | NC0028110 | 5 | 90.2 | 0.0167 | −28.68 |
| 27 | NC0070996 | 6 | 81.9 | 0.0151 | −27.27 |
| 33 | NC0015146 | 8 | 84 | 0.0409 | 24.173 |
| 33 | NC0004504 | 8 | 95.6 | 0.0037 | 35.068 |

Significant effects were detected in the hemizygous test-crossed populations. Additive effects were found in both common and exclusive regions across the four populations. Significant single factor ANOVA effects ranged from 35.06-100.14. Common regions among two of the hemizygous populations were found for effects on chromosome 2 and chromosome 5.

D. Exemplary Methods for Detection of Genetic Markers Associated with Transgene Modulating Loci

Oligonucleotides can also be used to detect or type the polymorphisms associated with transgene modulating loci disclosed herein by hybridization-based SNP detection methods. Oligonucleotides capable of hybridizing to isolated nucleic acid sequences which include the polymorphism are provided. It is within the skill of the art to design assays with experimentally determined stringency to discriminate between the allelic states of the polymorphisms presented herein. Exemplary assays include Southern blots, Northern blots, microarrays, in situ hybridization, and other methods of polymorphism detection based on hybridization Exemplary oligonucleotides for use in hybridization-based SNP detection are provided in Table 17. These oligonucleotides can be detectably labeled with radioactive labels, fluorophores, or other chemiluminescent means to facilitate detection of hybridization to samples of genomic or amplified nucleic acids derived from one or more plants using methods known in the art.

TABLE 17

Exemplary oligonucleotides for the amplification and detection of
SNPs of the present invention. For Type, F = forward primer, P =
probe, and R = reverse primer. It is within the skill in the art
to design similar oligonucleotides for the other polymorphisms
described herein, as well as design alternative assays for the
detection of SNPs using the references described herein.

| Marker | | | | | |
|---|---|---|---|---|---|
| SEQ | | SEQ | | | |
| ID | marker | ID | type | sequence | allele |
| 10 | NC0004504 | 177 | F | TCCTACCAAAACGATCATAGATCAAG | |
| 10 | NC0004504 | 178 | P | CTACCAACGCAATCA | C |
| 10 | NC0004504 | 179 | P | ACCAAAGCAATCAT | A |
| 10 | NC0004504 | 180 | R | GACTGTTTTGGCAGGAACCATAC | |
| 29 | NC0009818 | 181 | F | CGGAGCTCTGTTTGTTGCG | |
| 29 | NC0009818 | 182 | P | TTTGCTCGGCATGC | T |
| 29 | NC0009818 | 183 | P | TTTGCACGGCATGC | A |
| 29 | NC0009818 | 184 | R | GCGCTATGTGGCGTCAGAA | |
| 37 | NC0014417 | 185 | F | AGGAGCTATAGCAGCAGCACACT | |
| 37 | NC0014417 | 186 | P | ACTCATCCCTTACTGCT | G |
| 37 | NC0014417 | 187 | P | CTCATCCCTTATTGCT | A |
| 37 | NC0014417 | 188 | R | TTCCACCTCCTCCTCATCCA | |
| 65 | NC0027914 | 189 | F | AAAGCAAAGCAAAAACACAACTGA | |
| 65 | NC0027914 | 190 | P | AGGAACCACATATGC | A |
| 65 | NC0027914 | 191 | P | AGGAACCACATGTGC | G |
| 65 | NC0027914 | 192 | R | GTCCTGACTATCCCTTTGTTTCTTG | |
| 71 | NC0030985 | 193 | F | TTGCCTTTTATTTCTCCCTTGATTT | |
| 71 | NC0030985 | 194 | P | ACGCCTTGTAGCTTA | *********** |
| 71 | NC0030985 | 195 | P | CCTTGTAGACTGTTCC | ACTGTTCCAAG |
| 71 | NC0030985 | 196 | R | ACGCATTGTTTATCTTCATAATACTACCA | |
| 73 | NC0031358 | 197 | F | CAGGGTTTAGTCTGCAATCAGGTT | |
| 73 | NC0031358 | 198 | P | TGTTGTGTCAAAGGA | ********* |
| 73 | NC0031358 | 199 | P | CATGTTGTCATTGTTG | CATTGTTGT |
| 73 | NC0031358 | 200 | R | CTATGGTAGTAGTATTTTTTCTTGTTATTTTGTG | |

This was the first attempt to map the inheritance of regions that modulate the expression or phenotypic performance of a transgenic trait. Highly significant regions were identified and characterized that modulate the transgenic trait. Common regions were found to be significantly associated among the self and testcross populations evaluated. This method provides the identification and utilization of modulating regions for the enhancement of any transgenic trait and more specifically that of the lysine transgenic trait of this example. Relevant methods for the identification of transgene modulating genetic elements include genetic mapping, linkage disequilibrium analysis, transmission disequilibrium tests, targeted modification of key regulatory enzymes in the same or related biosynthetic pathways, and transcript profiling in combination with one or more mapping methods. Methodologies herein and in the future may be applicable to any transgene that encodes a product in an endogenously encoded biosynthetic pathway and/or that interacts with the host plant physiology.

### E. Alternative Markers for Making Breeding Decisions Related to Transgene Modulating Loci.

As already provided, phenotypic and genetic markers are useful for identification of, and making breeding decisions regarding, transgene modulating loci. In another embodiment, metabolites are useful as markers. In one aspect, different tissues are assessed for the profile of at least one metabolite. In a preferred aspect, the tissue expressing the at least one transgenic event is sampled. For example, a corn root worm transgene is evaluated for associated metabolic markers by sampling root tissue and a grain quality trait is evaluated in seed tissue. In another aspect, different developmental stages are assessed. Tissue is prepared for analysis using methods known in the art and analyzed using techniques known in the art, i.e., GC-MS or HPLC. Metabolite profiles are scored and analyzed as a “marker” and analyzed against population structure and corresponding phenotypic data to identify heritable metabolic markers associated with the phenotype of interest, i.e., transgene performance using the methods disclosed herein. This invention anticipates this approach can be used to evaluate 2 or more events, and/or 2 or more germplasm entries, and/or 2 or more transgenes (i.e., stacks).

### Example 2

### Evaluation of Genetic Background Effect on Trait Performance

A key goal of hybrid breeding programs is to maximize yield via complementary crosses. Crosses from distinct germplasm pools that result in a yield advantage constitute heterotic groups. The identification of heterotic groups facilitates informed crosses for a yield advantage. During inbred line development, advanced inbred lines are crossed with different tester lines in order to determine how the inbred line performs in hybrid combinations. The effect of a single cross reflects the specific combining ability (SCA) and the effect of the inbred in multiple crosses with different testers (typically in multiple locations) reflects the general combining ability (GCA).

In the context of a hybrid breeding program that includes one or more transgenic traits, it may be useful to evaluate the combining ability of the trait in different hybrid backgrounds. The present invention provides methods for evaluation of “transgene combining ability” and its application to making breeding decisions in cases where differences in trait performance are observed, which may be related to the direction of the cross, the parent(s), which parent is traited, and/or copy number of the transgene.

In the present example, a transgene with known variation was evaluated to determine the effect of genetic background on transgene performance. Transgenic trait performance was evaluated in different genetic backgrounds of lysine conversions (‘Trait Parents’) crossed to 40 different ‘Test Inbreds’ to evaluate LY038 efficacy in F1 grain. In the analysis there were three ‘Trait Parents’ analyzed; two ‘Trait Parents’ are the inbred conversions (High 1 and Low 2) and one is the hybrid of the two inbred conversions (Table 18). Lysine ‘Trait Parents’ were crossed to non-transgenic ‘Test Inbreds’ for LY038 efficacy in F1 grain. Two inbred conversions were evaluated as part of the efficacy test (High 1 and Low 2) as well as the hybrid of the two inbred conversions. The conversions and the hybrid were reciprocally crossed to 40 non-transgenic Test Inbreds which represent 23 male and 17 female lines. Thus, 240 crosses, including reciprocals, were evaluated. Approximately one-quarter of the crosses were replicated. Lysine was evaluated on 50 kernels of F1 grain.

TABLE 18
Experimental design for evaluation of LY038 performance across
genetic backgrounds using three transgenic testers.

| LY038 Trait Parent | | |
|---|---|---|
| Low 2 | High 1*Low 2 | High 1 |

Test Inbred Used as

| | F | M | F | M | F | M |
|---|---|---|---|---|---|---|
| 1 | 235 | 362 | 733 | 1030 | 806 | 559 |
| 2 | 641 | 306 | 1010 | 1224 | 1357 | 1429 |
| 3 | 422 | 231 | 656 | 1225 | 932 | 1607 |
| 4 | 632 | 242 | 363 | 675 | 483 | 850 |
| 5 | 373 | 297 | 1000 | 1325 | 940 | 1157 |
| 6 | 330 | 295 | 693 | 1289 | 751 | 995 |
| 7 | 574 | 114 | 1095 | 848 | 1171 | 593 |
| 8 | 209 | 131 | 631 | 617 | 498 | 455 |
| 9 | 131 | 179 | 286 | 861 | 639 | 1086 |
| 10 | 156 | 133 | 365 | 388 | 359 | 244 |
| 11 | 84 | 167 | 572 | 796 | 794 | 759 |
| 12 | 397 | 328 | 588 | 1366 | 839 | 714 |
| 13 | 70 | 97 | 252 | 594 | 599 | 809 |
| 14 | 488 | 287 | 779 | 738 | 1268 | 397 |
| 15 | 562 | 465 | 1216 | 1619 | 1021 | 1134 |
| 16 | 517 | 343 | 1718 | 1857 | 1383 | 597 |
| 17 | 291 | 243 | 870 | 1138 | 790 | 997 |
| 18 | 250 | 436 | 502 | 1056 | 865 | 1039 |
| 19 | 455 | 132 | 1192 | 628 | 860 | 662 |
| 20 | 357 | 278 | 814 | 1197 | 937 | 587 |
| 21 | 384 | 337 | 675 | 952 | 811 | 1394 |
| 22 | 795 | 337 | 1406 | 842 | 1277 | 328 |
| 23 | 640 | 333 | 1191 | 1260 | 1863 | 566 |
| 24 | 729 | 289 | 1069 | 1066 | 930 | 1013 |
| 25 | 220 | 366 | 525 | 1264 | 1057 | 1155 |
| 26 | 521 | 397 | 308 | 1295 | 458 | 1141 |
| 27 | 532 | 185 | 658 | 1118 | 643 | 747 |
| 28 | 415 | 309 | 689 | 1140 | 841 | 591 |
| 29 | 193 | 307 | 598 | 518 | 684 | 659 |
| 30 | 238 | 382 | 385 | 1247 | 680 | 765 |
| 31 | 111 | 174 | 223 | 734 | 512 | 320 |
| 32 | 573 | 232 | 746 | 1131 | 550 | 607 |
| 33 | 297 | 354 | 695 | 1337 | 1084 | 1543 |
| 34 | 572 | 428 | 1163 | 1556 | 1489 | 1265 |
| 35 | 456 | 302 | 776 | 1040 | 848 | 1172 |
| 36 | 454 | 144 | 683 | 521 | 997 | 721 |
| 37 | 381 | 182 | 450 | 661 | 596 | 485 |
| 38 | 678 | 271 | 1097 | 1472 | 970 | 1482 |
| 39 | 204 | 240 | 416 | 705 | 487 | 728 |
| 40 | 668 | 306 | 1347 | 1037 | 1540 | 877 |
| Average | 406 | 273 | 761 | 1034 | 890 | 856 |
| High | 795 | 465 | 1718 | 1857 | 1863 | 1607 |
| Low | 70 | 97 | 223 | 388 | 359 | 244 |

ANOVA was performed on the data to evaluate mixed models for the role of the parent, the cross, the tester, and heterotic group on lysine levels (design shown in Table 19).

TABLE 19
ANOVA design, degrees of freedome (DF), and F tests.

| Source | DF | DF | F Test |
|---|---|---|---|
| TraitParent | 2 | TP-1 | TP_MS/ |
| | | | TP*HG_MS |
| HetGroup | 1 | HG-1 | HG_MS/ |
| | | | TP*HG_MS |
| TraitParent*HetGroup | 2 | TP-1*HG-1 | TP*HG_MS/CD- |
| | | | 1*TP-1*HG-1 |
| CrossDir | 1 | CD-1 | CD_MS/ |
| | | | CD*TP_MS |
| CrossDir* TraitIParent | 2 | CD-1*TP-1 | CD*TPI_MS/ |
| | | | CD*TPI*HG_MS |
| CrossDir*HetGroup | 1 | CD- | CD* HG_MS/ |
| | | 1*HG-1 | CD*TP*HG_MS |
| CrossDir* TraitParent | 2 | CD-1*TP- | CD*TE*HG_MS/ |
| *HetGroup | | 1*HG-1 | MSE |
| TestInbred(HetGroup) | 38 | HG (TI-1) | TI (HG)_MS/ |
| | | | CT*TI(HG_MS) |
| TraitParent*- | 76 | TP-1* HG | TI (HG)_MS/CD-1* |
| TestInbred(HetGroup) | | (TI-1) | TP-1*HG (TI-1) |
| CrossDir*- | 38 | CD-1* | TI (HG)_MS/CD-1* |
| TestInbred(HetGroup) | | HG (TI-1) | TP-1*HG (TI-1) |
| CrossDir*TraitParent*- | 76 | CD-1* | CD-1* TP-1*HG (TI- |
| Test(InbredHetGroup) | | TP-1*HG | 1)/MSE |
| | | (TI-1) | |

The results show the ‘Trait Parent’ used is the most significant factor controlling lysine efficacy (Table 20). Means range from: Low 2 inbred=339.6; High 1 inbred=872.9; and the High 1*Low 2 hybrid=897.4.

TABLE 20
Proc GLM Analysis of Variance of Reciprocal Crosses of Trait
Parents by Test Inbreds.

| | | | Mean | | P- |
|---|---|---|---|---|---|
| Source | DF | Squares | Square | F | value |
| TraitParent | 2 | 18680835.15 | 9340417.57 | 27.70 | <.0001 |
| HetGroup | 1 | 1840070.85 | 1840070.85 | 5.45 | <.010 |
| TraitParent*HetGroup | 2 | 674250.14 | 337125.07 | 0.25 | ns |
| CrossDir | 1 | 60319.29 | 60319.29 | 0.045 | ns |
| CrossDir*TraitIParent | 2 | 2673787.37 | 1336893.68 | 23.74 | <.0001 |
| CrossDir*HetGroup | 1 | 1321063.31 | 1321063.31 | 23.46 | <.0001 |
| CrossDir*TraitParent*HetGroup | 2 | 112611.01 | 56305.51 | 0.887 | ns |
| TestInbred (HetGroup) | 38 | 8749843.13 | 230259.03 | 4.535 | <.0001 |
| TraitParent*TestInbred (HetGroup) | 76 | 3858451.52 | 50769.1 | 1.344 | ns |
| CrossDir*TestInbred (HetGroup) | 38 | 4172015.08 | 109789.87 | 2.90 | <.0001 |
| CrossDir*TraitParent*Test (InbredHetGroup) | 76 | 2870094.09 | 37764.4 | 0.595 | ns |
| Total/MSE | | 48756263.43 | 63439.36 | | |

In general, the High 1 inbred and most of the female heterotic lines have more efficacious germplasm, and the Low 2 inbred has lower efficacy. (Table 21) The decreased efficacy of Low 2 appears to be associated to the base germplasm (as evident form effects of ‘Trait Parent’ and ‘Test Inbred’) as well as a compromised maternally-associated factor that is particularly suboptimal when the line is used as a female. Possible explanations for this maternally-associated factor could include embryo physiology, cytoplasm, or imprinting.

TABLE 21
Class means of Trait Parents and heterotic group of Test Inbreds by Cross Direction.

| Trait Parent, Heterotic | | | | |
|---|---|---|---|---|
| Group of Test Inbred, | | Proc GLM | Proc Mixed | Proc Mixed |
| and Cross Direction | | Lysine ppm | Lysine ppm | Group |
| Female × Male | Number | (Std Dev) | (Std Err) | P < (0.05) |
| Male Heterotic Group + Low 2 | 22 | 438 (264) | 436.45 (61) | F |
| Female Heterotic Group + | 24 | 371 (178) | 390.52 (64) | FG |
| Low 2 | | | | |
| Low 2 + | 27 | 224 (95) | 237.53 (59) | G |
| Male Heterotic Group | | | | |
| Low 2 + | 23 | 319 (71) | 324.72 (63) | FG |
| Female Heterotic Group | | | | |
| Male Heterotic Group + | 31 | 870 (429) | 839.71 (58) | CD |
| High 1 | | | | |
| Female Heterotic Group + | 23 | 924 (251) | 923.79 (63) | BC |
| High 1 | | | | |
| High 1 + | 27 | 642 (250) | 656.19 (59) | E |
| Male Heterotic | | | | |
| Group | | | | |
| High 1 + | 23 | 1050 (380) | 1055.41 (63) | B |
| Female | | | | |
| Heterotic Group | | | | |
| Male Heterotic Group + | 24 | 683 (370) | 703.11 (58) | E |
| (High 1 + Low 2) | | | | |
| Female Heterotic Group + | 25 | 781 (312) | 787.09 (62) | DE |
| (High 1 + Low 2) | | | | |
| (High 1 + Low 2) + | 27 | 843 (268) | 856.97 (59) | CD |
| Male Heterotic Group | | | | |
| (High 1 + Low 2) + | 23 | 1219 (275) | 1224.96 (63) | A |
| Female Heterotic Group | | | | |
| Average | | 697.5 (261.5) | | |

It is further contemplated by this invention that the crossing scheme can be run across locations and environmental conditions in order to evaluate location effects and environment effects as needed for a product concept.

### Example 3

### Breeding for Transgene Modulating Loci

In the present example, breeding activities are provided to evaluate whether variation in transgene performance was due to genetic background. In one aspect, an experimental study was conducted wherein significant associations for transgene modulating loci were identified via QTL mapping and/or association study methods using segregating populations. Other methods for association studies are known in the art.

In another aspect, historical marker genotype data and trait phenotype data were used to identify transgene modulating loci. In yet another aspect, both historical data and experimental data from mapping populations were used to identify transgene modulating loci.

Markers associated with these loci can be employed in a marker-assisted selection program in order to accumulate at least one transgene modulating locus into at least one corn inbred of interest for the development of elite corn hybrids with the LY038 transgene. At least one marker allele associated with a LY038 modulating locus was used as the basis for selection decisions at each generation during the inbred and/or hybrid development process.

The selection decision may be based on selecting for or against a specific transgene modulating locus. The marker genotype information for the transgene modulating locus may be used as the basis to determine soybean varieties to be used in breeding crosses. Further, the markers associated with one or more transgene modulating loci will facilitate the introgression of one or more such genomic regions into varieties lacking the transgene modulating loci, i.e., elite varieties with High agronomic performance.

The marker allele may comprise a SNP allele, a haplotype, a specific transcriptional profile, and a specific nucleic acid sequence. Further, an association with the marker allele and a secondary trait may be identified and the secondary trait may provide the basis for selection decisions. Secondary traits include metabolic profiles, nutrient composition profiles, protein expression profiles, and phenotypic characters such as ear height or plant height.

Further, crossing schemes for preferred transgene combining ability are identified by the evaluation of reciprocal crosses and LY038 copy number on trait performance. Subsequent crosses from the germplasm pool are informed by these initial studies and breeding decisions for a preferred LY038 product concept are enabled with this information. For example, this information will inform which parent in the cross will perform at the product concept when traited and what copy number to use to achieve the product concept. It is further contemplated by this invention that the crossing scheme can be run across locations and environmental conditions in order to evaluate location effects and environment effects as needed for the product concept.

As additional transgenic traits are included in a product concept, association studies can be conducted to determine whether additional loci in the genetic background of one or more germplasm entries are modulating the performance of one or more of the transgenes. Significant interactions are identified as described above and markers, such as genetic markers or secondary traits, are used as the basis for selection as described above in order to develop germplasm entries consistent with the product concept.

### Example 4

### Use of Transgenic Testers for Evaluation of Preferred Genetic Backgrounds for at Least One Transgenic Event

The present example provides alternative methods for evaluation of the performance of at least one transgenic event in multiple germplasm backgrounds, including evaluation of copy number effects and performance in male vs. female germplasm in hybrid crops. Further, the present example provides the use of transgenic testers to facilitate this testing without necessarily requiring transgenic conversions of germplasm lacking the at least one transgenic event.

In the case of transgenes with “quantitative” phenotypes, such as yield or stress tolerance, it is useful to determine whether specific transgenic events perform better in specific genetic backgrounds. Unfortunately, traditional trait integration relies on backcrossing followed by selection across multiple generations to recover the recurrent parent. In order to quickly evaluate whether specific genetic backgrounds show improved or preferred transgene performance in hybrid crops, a novel approach is to cross inbred lines with a transgenic tester followed by performance evaluation of the hybrid plant. This method can also be used to evaluate the effect of transgene copy number on transgene performance. This method can be employed in conjunction with selection and introgression of transgene modulating loci. This method will reduce the number of converted inbreds and thus reduce the number of regulated plots, resulting in a reduction of resource allocation to this aspect of transgenic breeding.

Germplasm base and environmental conditions may modulate transgene expression, such as the case of the association of stress tolerance and grain yield. For example, secondary traits in base germplasm have the potential to expand opportunities for specific germplasm to perform better with a drought tolerance transgene. Specifically, heat stress tolerance and a reduction in ASI (anthesis silking interval) under stress need to go hand in hand with a drought tolerance trait. Thus, it is useful to determine whether the one or more transgenic events interact with specific backgrounds and, if so, to identify backgrounds, and events, with optimal performance. As such, it is further contemplated by this invention that the crossing scheme can be run across locations and environmental conditions in order to evaluate location effects and environment effects as needed for the product concept.

For example, in order to determine preferred genetic backgrounds for a transgenic event, 11 inbreds are available as BC2F3s for evaluation of transgene performance. In addition, conventional lines are selected to expand the heterotic groups assessed. The present invention anticipates fewer or more germplasm entries can be evaluated with these methods and the number of entries chosen herein are for the purpose of illustration.

This approach examines hybrids that are homozygous, hemizygous (in combinations on both sides of the cross) and null. This approach can be used to evaluate transgene performance across heterotic groups and in reciprocal crosses. Crosses are generated using bulks across BC2F2s and genotype data for percent recurrent parent is generated for bulked ears. Further, the allele frequency of the transgene can be measured using an assay that detects the presence of the promoter. Given that BC2F3s are used, negative isolines from trait conversion can be included as check comparisons. Relevant analyses include: 1) Quantify and compare interactions of specific germplasm backgrounds with at least one transgene; 2) Obtain balanced transgene combining ability estimates for all male and female inbreds; 3) Compare transgene performance of homozygous, hemizygous (in combinations on both sides of the cross) and null versions of hybrids; 4) Estimate relationship between transgene performance and associated agronomic traits.

The approach described herein uses a balanced mating design though other approaches are possible. Tables 22 illustrates a diallel crossing scheme. Alternative crossing designs are shown in Table 23 and Table 24. In any of these crossing schemes, it is possible to evaluate crosses where one, both, or none of the parents has one or more transgenes. Notably, Table 24 incorporates two entries for a single background wherein one version is transgenic and the other is conventional or transgenic but lacking the at least one transgene that is being evaluated.

TABLE 22
Diallel experiment. Crossing scheme for diallel experiment where
number of crosses = p(p − 1)/2, with reciprocals = p(p − 1).
Self of each genotype is maintained for evaluation and estimates of
additive variance from GCA and SCA are obtained. X = cross, S = self
pollinate, and R = reciprocal cross. The reciprocal cross allows for the
evaluation of maternal effects. Thus, the diallel design allows for
within heterotic group crosses and the evaluations of selfs.
Diallel

| | |
|---|---|
| Parents | Parents (males) |

| (females) | P1 | P2 | P3 | P4 | P5 | P6 | P7 | P8 |
|---|---|---|---|---|---|---|---|---|
| P1 | S | R | R | R | R | R | R | R |
| P2 | X | S | R | R | R | R | R | R |
| P3 | X | X | S | R | R | R | R | R |
| P4 | X | X | X | S | R | R | R | R |
| P5 | X | X | X | X | S | R | R | R |
| P6 | X | X | X | X | X | S | R | R |
| P7 | X | X | X | X | X | X | S | R |
| P8 | X | X | X | X | X | X | X | S |

TABLE 23
Design II experiment. Genetic information obtained is similar to
diallel. Different sets of parents used as males and females;
notably, twice as many parents can be included with same
number of crosses as diallel. Similar to the diallel, two estimates
of additive variance (male and female) are obtained.
Design II

| Parents | Parents (males) | | | | | | | |
|---|---|---|---|---|---|---|---|---|
| (females) | P1 | P2 | P3 | P4 | P5 | P6 | P7 | P8 |
| P9 | X91 | x92 | X93 | x94 | x95 | x96 | x97 | x98 |
| P10 | x101 | x102 | x103 | x104 | x105 | x106 | x107 | x108 |
| P11 | x111 | x112 | x113 | x114 | x115 | x116 | x117 | x118 |
| P12 | x121 | x122 | x123 | x124 | x125 | x126 | x127 | x128 |
| P13 | x131 | x132 | x133 | x134 | x135 | x136 | x137 | x138 |
| P14 | x141 | x142 | x143 | x144 | x145 | x146 | x147 | x148 |
| P15 | x151 | x152 | x153 | x154 | x155 | x156 | x157 | x158 |
| P16 | x161 | x162 | x163 | x164 | x165 | x166 | x167 | x168 |

TABLE 24
14 × 14 Design II experiment. In this 14 × 14 mating design, 28 parents are
included, but representing 18 inbred backgrounds (9 male and 9 female) wherein “+”
indicates a transgenic version and “−” indicates a nontransgenic version of the same
inbred. This approach includes 196 crosses, with no bias from selfs or within-heterotic
group crosses, with 288 entries in a 24 column × 12 range test.
14 × 14 Design II

| Parents | Parents (males) | | | | | | | | | | | | | |
|---|---|---|---|---|---|---|---|---|---|---|---|---|---|---|
| (females) | P1− | P1+ | P2− | P2+ | P3− | P3+ | P4− | P4+ | P5− | P5+ | P6 | P7 | P8 | P9 |
| P10− | X | X | X | X | X | X | X | X | X | X | X | X | X | X |
| P10+ | X | X | X | X | X | X | X | X | X | X | X | X | X | X |
| P2− | X | X | X | X | X | X | X | X | X | X | X | X | X | X |
| P2+ | X | X | X | X | X | X | X | X | X | X | X | X | X | X |
| P3− | X | X | X | X | X | X | X | X | X | X | X | X | X | X |
| P3+ | X | X | X | X | X | X | X | X | X | X | X | X | X | X |
| p4− | X | X | X | X | X | X | X | X | X | X | X | X | X | X |
| P4+ | X | X | X | X | X | X | X | X | X | X | X | X | X | X |
| P5− | X | X | X | X | X | X | X | X | X | X | X | X | X | X |
| P5+ | X | X | X | X | X | X | X | X | X | X | X | X | X | X |
| P16 | X | X | X | X | X | X | X | X | X | X | X | X | X | X |
| P17 | X | X | X | X | X | X | X | X | X | X | X | X | X | X |
| P18 | X | X | X | X | X | X | X | X | X | X | X | X | X | X |
| P19 | X | X | X | X | X | X | X | X | X | X | X | X | X | X |

Analyses include determining the combining ability effects of traited versus conventional versions of inbreds as well as balanced comparisons across different heterotic groups. By identifying key genetic backgrounds for the at least one transgene of interest, the transgenic breeding activities can be directed to optimal genetic backgrounds in the case of traits with performance variation. Further, in the case of a transgene with performance variation, evaluation of genetic background effects at the front end of a breeding program permits a breeding program to be economized by reducing the number of lines to be converted, the number of regulated plots, and, ultimately, the production of a superior transgenic product.

### Example 5

### Mapping of Transgene Modulating Loci for Selection of Preferred Germplasm-Transgene Combinations in Soybean

When breeding with a transgene that has a quantitative phenotype, it is useful to determine whether certain genetic backgrounds will show preferred expression for the transgene. Herein, such an approach is outlined for a yield transgene in soybean.

The transgene is bred into genetically distinct, i.e., segregating, populations of soybean using traditional backcross methods or forward breeding. Transgenic populations are made that are null for the transgene (as a control), hemizygous, and homozygous. Populations are grown out and phenotype for transgene performance as well as additional agronomic traits. In addition, lines are genotyped with a plurality of markers distributed throughout the genome in intervals of 20 cM. In a preferred aspect, markers are distributed at intervals of 5 to 12 cM. In a more preferred aspect, markers are distributed at intervals of 0-8 cM

In another aspect, historical marker genotype data and trait phenotype data are used to identify transgene modulating loci. In yet another aspect, both historical data and experimental data from mapping populations are used to identify transgene modulating loci.

Subsequently, genotype and phenotype data are analyzed for association of specific loci with, at least, transgene performance using methods such as ANOVA, MAPMAKER/QTL, gene, and other methods for association study known in the art.

Significant associations for transgene modulating loci (i.e., LOD greater than 2, p value less than 0.05) can be subsequently validated in soybean populations segregating for such loci. Markers associated with these loci can be employed in a marker-assisted selection program in order to accumulate at least one transgene modulating locus into at least one soybean variety of interest for the development of elite transgenic soybean varieties.

At least one marker allele associated with a transgene modulating locus will be used as the basis for selection decisions at each generation during the variety development process. The selection decision may be based on selecting for or against a specific transgene modulating locus. The marker genotype information for the transgene modulating locus may be used as the basis to determine soybean varieties to be used in breeding crosses. Further, the markers associated with one or more transgene modulating loci will facilitate the introgression of one or more such genomic regions into varieties lacking the transgene modulating loci, i.e., elite varieties with High agronomic performance.

The marker allele may comprise a SNP allele, a haplotype, a specific transcriptional profile, and a specific nucleic acid sequence. Further, an association with the marker allele and a secondary trait may be identified and the secondary trait may provide the basis for selection decisions. Secondary traits include metabolic profiles, nutrient composition profiles, protein expression profiles, and phenotypic characters such as pod color or plant height.

As additional transgenic traits are included in the product concept, marker-trait association studies are conducted to determine whether additional loci in the genetic background of one or more germplasm entries are modulating the performance of one or more of the transgenes. In another aspect, testing can be conducted across locations and environmental conditions in order to evaluate location effects and environment effects as needed for the product concept. Significant interactions are identified as described above and markers, such as genetic markers or secondary traits, are used as the basis for selection as described above in order to develop germplasm entries consistent with the product concept.

### Example 6

### Methods of Mapping Transgene Modulating Loci Associated with a Gene Suppression Construct

This invention further anticipates that gene suppression constructs may be affected by transgene modulating loci. The following example provides methods and compositions for the selection of transgene modulating loci for a DNA construct capable of suppression of alpha zein genes, as provided in U.S. Patent Application Ser. Nos. 61/041,035 and 61/072,633, filed Mar. 31, 2008 and Apr. 1, 2008 respectively.

In one aspect, certain genotypes of corn seed display an opaque kernel phenotype when they comprise transgenes or other genetic loci that provide for reduced alpha-zein storage protein content. A variety of transgenes can provide for reduced alpha-zein storage protein content can be used to reduce expression of one or more endogenous alpha-zein genes. DNA constructs that are particularly suitable for suppression of both the 19-kD and 22 kD alpha-zein genes are disclosed in U.S. Patent Application Publication Number 2006/0075515. DNA constructs that provide for suppression of only the 19-kD alpha-zein are described in U.S. Patent Application Publication Number 2006/0075515.

Transgene modulating loci, in the present example termed “opaque modifier loci,” that can restore a vitreous phenotype to opaque corn seed, including genetic markers and germplasm sources, are provided in U.S. Patent Application Ser. Nos. 61/041,035 and 61/072,633. An opaque modifier locus or opaque modifier loci can be obtained from a variety of corn germplasm sources including, but not limited to, hybrids, inbreds, partial inbreds, or members of defined or undefined populations. Germplasm characterized by a high kernel density is one source of the opaque modifier loci. Germplasm characterized by a seed density of at least about 1.24 grams/milliliter is considered to have a high kernel density. Certain inbred lines have also been shown to contain one or more opaque modifier loci that act either alone or in combination to restore a vitreous phenotype on opaque seed reduced alpha-zein storage protein content. In practicing the methods of the invention, the corn line comprising the transgene that reduces the alpha-zein storage content is typically crossed to a genetically distinct corn line. It is understood that the corn line comprising the transgene and the genetically distinct corn line can each be used as either pollen donors or pollen recipients in the methods of the invention.

Corn germplasm that can be used as a source of the opaque modifier locus or opaque modifier loci of the invention can also be identified by use of molecular markers. More specifically, opaque modifier loci that are linked to molecular markers identified in U.S. Patent Application Ser. Nos. 61/041,035 and 61/072,633 can be identified by determining if a given germplasm comprises an allele of the marker that is associated with the linked opaque modifier locus.

It is further contemplated that the opaque modifier loci that restore the vitreous phenotype to opaque seeds and that are linked to molecular markers can be separated from other loci present in the source germplasm that do not contribute to restoration of the vitreous phenotype. Separation of the opaque modifier loci from other undesired loci can be accomplished by molecular breeding techniques whereby additional markers to the undesired genetic regions derived from the source germplasm are used. It is thus contemplated that seed comprising one or more opaque modifier loci can comprise just the locus or loci, or can comprise the locus or loci and an associated molecular marker

Once progeny of the cross between a corn line comprising an opaque kernel phenotype and a transgene that reduces expression of an alpha-zein storage protein with a genetically distinct corn line are obtained, a seed comprising a vitreous kernel phenotype and the transgene that confers reduced alpha-zein storage protein content is selected. Selection of such seed can be accomplished in a variety of ways. The vitreous phenotype can usually be selected by visual screening. Such visual screening can be facilitated by placing the seed of the cross on a light source. Selection for the vitreous phenotype could also be accomplished by other methods that include, but are not limited to, selection of seed for increased density. Density can at be determined by a variety of methods that include but are not limited to Near Infared Transmittance (NIT). It is further contemplated that either manual, semi-automated, or fully automated methods where vitreous seed are screened and selected on the basis of density, light transmittance, or other physical characteristics are also contemplated herein.

In another aspect, genetic markers and methods for the introduction of one or more opaque modifier loci conferring a vitreous phenotype on corn seed kernels that display an opaque phenotype in the absence of the modifier loci are provided in U.S. Patent Application Ser. Nos. 61/041,035 and 61/072,633.

Marker assisted introgression involves the transfer of a chromosome region defined by one or more markers from one germplasm to a second germplasm. The initial step in that process is the genetic localization of the opaque modifier loci as previously described. When an opaque modifier locus that is a QTL (quantitative trait locus) has been localized in the vicinity of molecular markers, those markers can be used to select for improved values of the trait without the need for phenotypic analysis at each cycle of selection. Values that can be associated with the vitreous phenotype conferred by the opaque modifier include but are not limited to light transmittance measurements or density determinations. In marker-assisted breeding and marker-assisted selection, associations between the QTL and markers are established initially through genetic mapping analyses, using either historical or de novo genotypic and phenotypic data.

Molecular markers can also be used to accelerate introgression of the opaque modifier loci into new genetic backgrounds (i.e. into a diverse range of germplasm). Simple introgression involves crossing an opaque modifier line to an opaque line with reduced alpha-zein content and then backcrossing the hybrid repeatedly to the opaque line (recurrent) parent, while selecting for maintenance of the opaque modifier locus. Over multiple backcross generations, the genetic background of the original opaque modifier line is replaced gradually by the genetic background of the opaque line through recombination and segregation. This process can be accelerated by selection on molecular marker alleles that derive from the recurrent parent.

Alternatively, a transgene that confers an opaque phenotype (and reduced alpha zein content) can be introgressed into an elite inbred genetic background that comprises one or more opaque modifiers. Simple introgression involves crossing a transgenic line to an elite inbred line with an opaque modifier and then backcrossing the hybrid repeatedly to the elite inbred line (recurrent) parent, while selecting for maintenance of the transgene and the opaque modifier locus (i.e. a vitreous phenotype in the presence of reduced alpha zein content and/or a linked transgenic trait). Linkage of the transgene to a selectable or scoreable marker gene could, in certain embodiments, further facilitate introgression of the transgene into the elite inbred genetic background. Over multiple backcross generations, the genetic background of the original transgenic line is replaced gradually by the genetic background of the elite opaque line modifier line through recombination and segregation. This process can be accelerated by selection on molecular marker alleles that derive from the recurrent parent.

We claim:

1. A method for identifying an association of a plant genotype with a performance of one or more transgenic traits comprising
screening a plurality of transgenic germplasm entries displaying a heritable variation for at least one transgenic event wherein the heritable variation is linked to at least one genotype; and
associating at least one genotype from the transgenic germplasm entries to at least one transgenic event.

2. The method of claim 1, wherein the at least one genotype comprises at least one marker selected from the group consisting of a genetic marker, a haplotype, a nucleic acid sequence, a transcriptional profile, a metabolic profile, a nutrient composition profile, a protein expression profile, and a phenotypic character.

3. The method of claim 1, wherein the at least one genotype comprises at least one genetic marker having a physical and/or genetic map position, wherein at least one chromosomal region that modulates the performance of at least one transgenic event is identified.

4. The method of claim 1, wherein the method further comprises making a plant breeding decision based on the association of the at least one genotype with the performance of the at least one transgenic event.

5. The method of claim 4, wherein the plant breeding decision comprises:
selecting at least one germplasm entry based on the presence of at least one genotype that modulates the performance of a transgenic event;
using the selected germplasm entry for a breeding activity; and
producing a progeny seed and plants thereof from the breeding activity.

6. The method of claim 5, wherein the germplasm entry is selected based on the presence of a genotype that enhances performance of the transgenic event relative to the performance of the event measured in a transgenic tester.

7. The method of claim 5, wherein the selected germplasm entry is used in making a breeding cross.

8. The method of claim 5, wherein the selected germplasm entry is used as a donor to introgress a genomic region into at least one recipient germplasm entry.

9. The method of claim 6, wherein the selected germplasm entry is crossed with a transgenic tester line expressing one or more transgenic events to evaluate a traited hybrid.

10. The method of claim 6, wherein a plurality of selected germplasm entries is crossed with one or more transgenic tester lines to accumulate a plurality of traited hybrids for evaluation of event performance.

11. The method of claim 6, wherein the method further comprises inducing haploidization in the selected germplasm entry; and subjecting the haploid germplasm to doubling to create an isogenic population fixed for at least one genotype associated with a transgenic event performance.

12. The method of claim 6, wherein the selected germplasm entry is used for a trait integration activity.

13. The method of claim 12, wherein the method further comprises
backcrossing the selected germplasm entry with a transgene donor; and
performing a recurrent selection step

14. The method of claim 12, wherein the method further comprises forward breeding the selected germplasm entry.

15. The method of claim 13 wherein the method further comprises selecting the progeny seed or plant of the breeding activities to identify one or more of the genotypes and one or more transgenic event combinations, wherein the combination modulates the performance of the transgenic event in the seed or plant.

16. The method of claim 1, wherein the plant is a crop plant selected from the group consisting of maize (Zea mays), soybean (Glycine max), cotton (Gossypium hirsutum), peanut (Arachis hypogaea), barley (Hordeum vulgare); oats (Avena sativa); orchard grass (Dactylis glomerata); rice (Oryza sativa, including indica and japonica varieties); sorghum (Sorghum bicolor); sugar cane (Saccharum sp); tall fescue (Festuca arundinacea); turfgrass species (e.g. species: Agrostis stolonifera, Poa pratensis, Stenotaphrum secundatum); wheat (Triticum aestivum), and alfalfa (Medicago sativa), members of the genus Brassica, broccoli, cabbage, carrot, cauliflower, Chinese cabbage, cucumber, dry bean, eggplant, fennel, garden beans, gourd, leek, lettuce, melon, okra, onion, pea, pepper, pumpkin, radish, spinach, squash, sweet corn, tomato, watermelon, ornamental plants, and other fruit, vegetable, tuber, and root crops.

17. The method of claim 1, wherein the transgenic event is selected from the group consisting of herbicide tolerance, disease resistance, insect or pest resistance, altered fatty acid, protein or carbohydrate metabolism, increased grain yield, increased oil, enhanced nutritional content, increased growth rates, enhanced stress tolerance, preferred maturity, enhanced organoleptic properties, altered morphological characteristics, sterility, other agronomic traits, traits for industrial uses, or traits for improved consumer appeal.

18. A method of identifying a plant germplasm entry with a genotype that modulates a performance of a transgenic trait, the method comprising:
crossing at least two germplasm entries with a test germplasm entry comprising at least one transgenic trait;
evaluating the performance of at least one transgenic trait in a progeny of each cross.

19. The method of claim 18, wherein the performance of at least one transgenic trait in the progeny of the cross is evaluated in at least two testing environments differing in at least one condition.

20. The method of claim 18, wherein the method comprises crossing at least two germplasm entries with a plurality of test transgenic germplasm entries comprising at least one transgenic trait.

21. The method of claim 20, wherein the test transgenic germplasm entries comprise a stack of at least two transgenic traits.

22. The method of claim 20, wherein the test transgenic germplasm entries are selected from the group consisting of a tester, an inbred, and a hybrid.

23. The method of claim 20, wherein the germplasm entries are evaluated in reciprocal crosses.

24. The method of claim 18, wherein the method comprises evaluating an effect of different copy number for at least one transgenic trait.

25. The method of claim 18, wherein the modulated performance of the transgenic trait is enhanced relative to the performance of the trait measured in a test transgenic germplasm entry.

26. The method of claim 18, wherein method further comprises using the plant germplasm entries associated with modulated performance of a transgenic trait in plant breeding activities.

27. The method of claim 26, wherein the plant breeding activities comprise crossing at least one preferred germplasm entry with a germplasm entry with at least one transgenic trait.

28. The method of claim 27, wherein the cross produces a hybrid seed or plant comprising one or more transgenic traits, wherein the performance of the trait is modulated in the seed or plant.

29. The method of claim 26, wherein the plant breeding activities comprise crossing at least two germplasm entries to accumulate at least two preferred genotypes for performance of at least one transgene followed by crossing with a germplasm entry with the at least one transgene.

30. The method of claim 26, wherein the plant breeding activities comprise crossing at least one preferred transgenic germplasm entry, containing at least one transgene, with a germplasm entry containing at least one transgene.

31. The method of claim 26, wherein the plant breeding activities comprise crossing at least two preferred transgenic germplasm entries to accumulate at least two preferred genotypes for performance of at least one transgene followed by crossing with a transgenic germplasm entry with at least one transgene.

32. The method of claim 18, wherein the plant germplasm entry is a crop plant selected from the group consisting of maize (Zea mays), soybean (Glycine max), cotton (Gossypium hirsutum), peanut (Arachis hypogaea), barley (Hordeum vulgare); oats (Avena sativa); orchard grass (Dactylis glomerata); rice (Oryza sativa, including indica and japonica varieties); sorghum (Sorghum bicolor); sugar cane (Saccharum sp); tall fescue (Festuca arundinacea); turfgrass species (e.g. species: Agrostis stolonifera, Poa pratensis, Stenotaphrum secundatum); wheat (Triticum aestivum), and alfalfa (Medicago sativa), members of the genus Brassica, broccoli, cabbage, carrot, cauliflower, Chinese cabbage, cucumber, dry bean, eggplant, fennel, garden beans, gourd, leek, lettuce, melon, okra, onion, pea, pepper, pumpkin, radish, spinach, squash, sweet corn, tomato, watermelon, ornamental plants, and other fruit, vegetable, oilseed, beverage, forest, tuber, and root crops.

33. The method of claim 18, wherein the transgenic trait is selected from the group consisting of herbicide tolerance, disease resistance, insect or pest resistance, altered fatty acid, protein or carbohydrate metabolism, increased grain yield, increased oil, enhanced nutritional content, increased growth rates, enhanced stress tolerance, preferred maturity, enhanced organoleptic properties, altered morphological characteristics, sterility, other agronomic traits, traits for industrial uses, or traits for improved consumer appeal.

34. A plant, seed or part thereof containing at least one genomic region identified to modulate the performance of a transgenic event.

35. A plant, seed, or part thereof containing at least one genomic region for an enhanced performance of two or more transgenes.

36. A method of introgressing at least one transgene modulating locus into a plant comprising
crossing at least one first plant with at least one second plant in order to form a population,
genotyping at least one plant in the population with respect to a genomic nucleic acid marker selected from the group SEQ ID NOs:1-176, and selecting from the population at least one plant comprising at least one nucleic acid molecule selected from the group consisting of SEQ ID NOs:1-176.

37. The method of claim 36, wherein the genotype is determined by an assay which is selected from the group consisting of single base extension (SBE), allele-specific primer extension sequencing (ASPE), DNA sequencing, RNA sequencing, microarray-based analyses, universal PCR, allele specific extension, hybridization, mass spectrometry, ligation, extension-ligation, and Flap Endonuclease-mediated assays.

38. The method of claim 36, further comprising the step of crossing the plant selected in step (c) to another plant.

39. The method of claim 36, further comprising the step of obtaining seed from the plant selected in step (c).

40. An elite plant produced by:
a) crossing at least one first plant comprising a nucleic acid molecule selected from the group consisting of SEQ ID NOs:1-176 with at least one second plant in order to form a population,
b) genotyping at least one plant in the population with respect to a genomic nucleic acid marker selected from the group SEQ ID NOs:1-176, and
c) selecting from the population at least one plant comprising at least one nucleic acid molecule selected from the group consisting of SEQ ID NOs:1-176.

41. A substantially purified nucleic acid molecule for the detection of transgene modulating loci comprising a nucleic acid molecule selected from the group consisting of SEQ ID NOs:1-176 and complements thereof.
